(19) United States
(12) Patent Application Publication
(10) Pub. No.: US 2025/0027085 A1
(43) Pub. Date: Jan. 23, 2025

(54) COMPOSITIONS AND METHODS FOR TREATING ALZHEIMER'S DISEASE UTILIZING A TRANSCRIPTIONAL MASTER REGULATOR OF THE RETROMER COMPLEX

(71) Applicant: THE TRUSTEES OF COLUMBIA UNIVERSITY IN THE CITY OF NEW YORK, New York, NY (US)

(72) Inventors: Ulrich Hengst, New York, NY (US); Claudio Andre Gouveia Martins Roque, New York, NY (US); Jimena Baleriola, New York, NY (US)

(21) Appl. No.: 18/906,851

(22) Filed: Oct. 4, 2024

Related U.S. Application Data
(63) Continuation of application No. PCT/US2023/065614, filed on Apr. 11, 2023.
(60) Provisional application No. 63/329,999, filed on Apr. 12, 2022.
(60) Provisional application No. 63/386,098, filed on Dec. 5, 2022.

(51) Int. Cl. C12N 15/113; C12Q 1/6874; C12Q 1/6883; G01N 33/68

(52) U.S. Cl. CPC C12N 15/113; C12Q 1/6874; C12Q 1/6883; G01N 33/6896; C12N 2310/14; C12Q 2600/158

(57) ABSTRACT

The present disclosure provides, inter alia, methods for treating neurodegenerative disease including Alzheimer's disease. Methods for restoring retromer complex function in a subject, methods for determining the progression of a neurodegenerative disease in a subject, and in vivo methods for identifying a DNA-binding profile of a dimeric transcription factor complex such as the retromer complex are also provided. Further provided are methods for restoring amyloid precursor protein (APP) homeostasis in a subject in need thereof, methods for restoring tau metabolism in a subject in need thereof, compositions and methods for preventing CREB3L2-ATF4 heterodimerization in a subject using such compositions, and methods for rescuing AP42-induced neuronal cell death in a subject using such compositions.

### CROSS REFERENCE TO RELATED APPLICATIONS

The present application is a continuation of PCT international application no. PCT/US2023/065614, filed on Apr. 11, 2023, which claims benefit of U.S. Provisional Patent Application Ser. No. 63/329,999, filed on Apr. 12, 2022, and U.S. Provisional Patent Application Ser. No. 63/386,098, filed on Dec. 5, 2022. The contents of above applications are incorporated by reference herein in their entireties.

### GOVERNMENT FUNDING

This disclosure was made with government support under grant nos. AG008702 and NS109607, awarded by National Institutes of Health. The government has certain rights in the disclosure.

### FIELD OF DISCLOSURE

The present disclosure provides, inter alia, methods for treating neurodegenerative diseases including Alzheimer's disease. Also provided are in vivo methods for identifying DNA-binding profiles of dimeric transcription factor complexes such as, e.g., CREB3L2-ATF4.

### INCORPORATION BY REFERENCE OF SEQUENCE LISTING

This application contains references to amino acids and/or nucleic acid sequences that have been filed concurrently herewith as sequence listing XML file “CU22191-seq.xml”, file size of 87,248 bytes, created on Dec. 5, 2022. The aforementioned sequence listing is hereby incorporated by reference in its entirety pursuant to 37 C.F.R. § 1.52(e)(5).

### BACKGROUND OF THE DISCLOSURE

Alzheimer's disease (AD) is a prevalent and irreversible neurodegenerative disorder characterized by a gradual decline in cognition that most commonly affects the elderly (Long and Holtzman, 2019). Its complex biological presentation has long curbed treatment strategies, but recent genome-wide genetic association studies have provided a more uniform framework for understanding its etiology, with cholesterol metabolism, immune responses, and endosomal trafficking emerging as new focal pathways that interact with the primary pathogenic hallmarks of AD, extracellular β-amyloid deposition and intraneuronal neurofibrillary tangles of hyperphosphorylated tau protein (Scheltens et al., 2016; Small et al., 2017; Verheijen and Sleegers, 2018). It is also increasingly evident that AD pathophysiology reconfigures the expression of specific groups of genes, indicating a role for network regulators such as transcription factors (TFs), cofactors, and chromatin remodelers in the progression of the condition (Mathys et al., 2019; Mostafavi et al., 2018; Zhang et al., 2013).

TFs typically work together to achieve specific transcriptional responses. This combinatorial regulation can occur, for example, through the mutual stabilization of DNA associations, the promotion of more accessible chromatin states, or direct protein-protein interactions between TFs (Amoutzias et al., 2008; Reiter et al., 2017). The latter mechanism is seen within the basic-region leucine zipper (bZIP) family through dimerization (Newman and Keating, 2003). Because multiple binding possibilities commonly exist, TF heterodimerization can generate significant variability in DNA-binding profiles, which cells exploit to expand their regulatory repertoire and orchestrate differential gene expression programs in a context-dependent fashion. On a global scale, however, these synergistic relationships remain largely unmapped (Lambert et al., 2018b), not least due to the lack of methodologies to do so comprehensively across the genome, limiting our understanding of how TFs regulate gene expression and hence also their contribution to disease states.

Emerging evidence pinpoints malfunction at the level of the retromer, a protein complex that coordinates cargo-sorting events within the endosomal network, as a driver of endosomal dysfunction and AD pathogenesis (Cullen and Steinberg, 2018; Nixon, 2017; Small and Petsko, 2015). A role for retromer in AD was first suggested by the finding that two of its core subunits, VPS26 and VPS35, involved in endosomal cargo selection, are deficiently expressed in the brain of affected individuals in an age-independent fashion (Small et al., 2005). Since then, genetic and functional studies have confirmed that retromer-mediated processes are disrupted in AD and other neurodegenerative disorders, such as Parkinson's disease (Small and Petsko, 2015; Vilarino-Guell et al., 2011; Wang et al., 2013; Zimprich et al., 2011). Most significantly, the trafficking and processing of amyloid precursor protein (APP) is altered by retromer-related defects, leading to the accumulation of neurotoxic β-amyloid peptides (Small and Petsko, 2015). However, despite much progress in understanding the consequences of retromer malfunction, it remains unknown why retromer deregulation first develops in late-onset AD (LOAD), the most prevalent form of AD.

Accordingly, there is a need for identifying and characterizing pathological TF complexes associated with neurodegenerative diseases as this can lead for example to a mechanistic understanding of the role of retromer transcriptional misregulation in LOAD. This disclosure is directed to meeting these and other needs.

### SUMMARY OF THE DISCLOSURE

Transcription factors (TFs) regulate gene expression and define cellular homeostasis via cooperative interactions. Transcriptional deregulation can drive pathologies, including Alzheimer's disease (AD). The present disclosure revealed that the bZIP TF heterodimer CREB3L2-ATF4 formed in neurons in response to β-amyloid exposure and accumulates in AD brain. Using ChIP-seq on human prefrontal cortex, several transcriptional networks deregulated by CREB3L2 in AD were identified. A method was developed to resolve the transcriptional program of defined dimeric TFs and uncovered the cause of this deregulation in the combination of the transcriptional properties of CREB3L2 and ATF4, i.e., the former's boarder scope of targets and the latter's stronger activity. Among other AD susceptibility genes, the retromer was identified as a target of CREB3L2-ATF4 transcriptional regulation and connects heterodimer formation to retromer dysfunction in AD and β-amyloid metabolism. The data established CREB3L2-ATF4 as a transcriptional hub of AD pathology and highlighted TF combinatorics as a relevant disease mechanism.

Accordingly, one embodiment of the present disclosure is a method for treating or ameliorating the effects of a neurodegenerative disease in a subject, comprising: (a) determining the level of CREB3L2-ATF4 transcription factor (TF) complex in a sample obtained from the subject; and (b) administering to the subject an effective amount of an agent that modulates the association between CREB3L2 and ATF4, if the level of CREB3L2-ATF4 complex determined in step (a) is significantly different from that of a control subject.

Another embodiment of the present disclosure is a method for restoring retromer complex function in a subject, comprising administering to the subject an effective amount of an agent that modulates CREB3L2 expression.

Another embodiment of the present disclosure is a method for determining the progression of a neurodegenerative disease in a subject, comprising: (a) determining the level of CREB3L2-ATF4 transcription factor (TF) complex in a sample obtained from the subject; and (b) concluding that the neurodegenerative disease in the subject is progressing, if the level of CREB3L2-ATF4 complex determined in step (a) is significantly increased from that of a control subject.

Another embodiment of the present disclosure is method for identifying the DNA-binding profile of a dimeric transcription factor complex in vivo, comprising: (a) generating a DNA construct of a first transcription factor comprising: (i) fusing a specific first dimerization domain to the C-terminal of the first transcription factor; and (ii) adding a first N-terminal epitope tag to the first transcription factor; (b) generating a DNA construct of a second transcription factor comprising: (i) fusing a specific second dimerization domain to the C-terminal of the second transcription factor, wherein the second dimerization domain is different from the first dimerization domain; and (ii) adding a second N-terminal epitope tag to the second transcription factor, wherein the second N-terminal epitope tag is different from the first N-terminal epitope tag; (c) identifying a bivalent ligand that recognizes both dimerization domains from steps (a) and (b); (d) co-transfecting a host cell with the DNA constructs generated in steps (a)-(b) and co-expressing polypeptides encoded by the DNA constructs in the presence of the bivalent ligand identified in step (c) to form the dimeric transcription factor complex; and (e) identifying the DNA-binding profile of the dimeric transcription factor complex by determining the complex's binding sites to the genome using ChIP-sequencing (ChIP-seq).

A further embodiment of the present disclosure is a method for restoring amyloid precursor protein (APP) homeostasis in a subject in need thereof, comprising: (a) determining the Aβ₁₋₄₂/Aβ₁₋₄₀ ratio in a sample obtained from the subject; and (b) administering to the subject an effective amount of an agent that increases the expression level of CREB3L2 or prevents the dimerization of CREB3L2 with ATF4, if the Aβ₁₋₄₂/Aβ₁₋₄₀ ratio determined in step (a) is significantly higher that a predetermined reference.

An additional embodiment of the present disclosure is a method for restoring tau metabolism in a subject in need thereof, comprising administering to the subject an effective amount of an agent that modulates the association between CREB3L2 and ATF4.

Another embodiment of the present disclosure is a composition comprising a nucleotide of SEQ ID NO: 68 or SEQ ID NO: 69.

Another embodiment of the present disclosure is a method for preventing CREB3L2-ATF4 heterodimerization in a subject, comprising administering to the subject an effective amount of the composition comprising a nucleotide of SEQ ID NO: 68 or SEQ ID NO: 69.

Another embodiment of the present disclosure is a method for rescuing Aβ₄₂-induced neuronal cell death in a subject, comprising administering to the subject an effective amount of the composition comprising a nucleotide of SEQ ID NO: 68 or SEQ ID NO: 69.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G show the identification of CREB3L2-ATF4 transcription factor complex.

FIG. 1A shows the assessment of somatic degeneration by TUNEL staining of rat hippocampal neurons after Aβ₁₋₄₂ stimulation. Cells were grown until DIV 10-12 in tripartite microfluidic chambers, which allow for the fluidic isolation of axons from cell bodies and dendrites. Axons or cell bodies were transfected with control or Creb3/2-targeting siRNAs before axonal or somatic treatment with AP1-42 for 48 hours, respectively. Data are presented as mean±SEM of n=10-12 biological replicates. Axonal dataset: *P-value=0.0357; somatic dataset: ***P-value=0.0003; statistical significance was determined by one-way ANOVA test with post hoc Bonferroni multiple comparison correction. Scale bar, 15 μm. As used herein, “DIV” refers to cell division, i.e., the division of a mother cell into two daughter cells (1->2->4->8, etc.).

FIG. 1B shows the experimental outline that is the same as in FIG. 1A, except that nuclear CHOP protein expression levels were instead measured by quantitative immunofluorescence after axonal siRNA delivery and Aβ₁₋₄₂ treatment. Data are presented as mean±SEM of 150-184 optical fields per condition, obtained over n=8 independent replicates; ***P-value=0.0008, one-way ANOVA test with post hoc Bonferroni multiple comparison correction. Scale bar, 15 μm. CHOP: C/EBP homologous protein.

FIG. 1C shows the coimmunoprecipitation of CREB3L2 with ATF4. Both proteins were produced by in vitro translation using rabbit reticulocyte lysates.

FIG. 1D shows the in situ visualization of endogenous CREB3L2-ATF4 heterodimers by PLA in vehicle or Aβ₁₋₄₂-treated hippocampal neurons. Oligomeric Aβ₁₋₄₂ was bath-applied for 12 hours. βIII-tubulin and DAPI, a nuclear marker, were used as counterstains. Box-plots display a summary of relative interaction events obtained over n=3 independent experiments (nuclear: ***P-value=0.0007; somatic: **P-value=0.0016, unpaired t-tests); box-plot whiskers are drawn to the 10^{th} and 90^{th} percentiles. Scale bar, 10 μm.

FIG. 1E shows the axonal CREB3L2-ATF4 heterodimers detected by PLA in hippocampal neurons cultured in microfluidic chambers. Axons were treated with vehicle or Aβ₁₋₄₂ oligomers for 12 hours. Box-plots display a summary of relative interaction events obtained over n=3 independent experiments (***P-value <0.0001, unpaired t-test); box-plot whiskers are drawn to the 10^{th} and 90^{th} percentiles. PLA signals were normalized to 100-μm axon segments. Scale bar, 10 μm.

FIG. 1F shows the coimmunoprecipitation analysis of CREB3L2 and ATF4 association in neuritic extracts after Aβ₁₋₄₂ challenge. Cortico-hippocampal neurons were grown on transwell inserts and allowed to mature for 10-12 DIV before stimulation protocol. ATF4 levels in CREB3L2 immunoprecipitates are normalized against input βIII-tubulin. Data are presented as mean±SEM of n=3 independent experiments (*P-value=0.0418, unpaired t-test).

FIG. 1G shows the axonal CREB3L2-ATF4 complexes detected by a proximity ligation assay. Emetine was specifically delivered to hippocampal axons using a microfluidic chamber system. Approximately 60 axonal fields were analyzed per condition, over n=3 independent replicates; ***P-value <0.0001, one-way ANOVA test with post hoc Bonferroni multiple comparison correction. Scale bar, 10 μm.

FIGS. 2A-2C show that local translation and S2P mediate CREB3L2-ATF4 complex activation in axons

FIG. 2A shows the TUNEL staining of rat hippocampal neurons. Hippocampal neurons were cultured in microfluidic chambers for 10-12 DIV and Aβ₁₋₄₂ oligomers were added for 36 hours; nelfinavir, a S2P inhibitor, was applied in the last 24 hours of the Aβ₁₋₄₂ stimulus. Both treatments were specifically given to axons. Mean±SEM of n=3 independent experiments; per replicate, each condition was sampled from 10 different optical fields; ***P-value <0.0001, one-way ANOVA test with post hoc Bonferroni multiple comparison correction. Scale bar, 15 μm.

FIG. 2B shows the quantitative immunofluorescence analysis of somatic CHOP protein expression levels. Experimental outline same as described in FIG. 2A. Mean±SEM of n=3 independent experiments; per replicate, each condition was sampled from 10 different optical fields; ***P-value <0.0001, one-way ANOVA test with post hoc Bonferroni multiple comparison correction. Scale bar, 15 μm.

FIG. 2C shows the detection of axonal CREB3L2-ATF4 heterodimers by PLA after local inhibition of retrograde transport. Ciliobrevin A was specifically delivered to axons in the last 6 hours of an 18-hour Aβ₁₋₄₂ treatment period using a microfluidic chamber system. Box-plots display a summary of a summary of relative interaction events obtained over n=3 independent replicates (***P-value <0.0001, one-way ANOVA test with post hoc Bonferroni multiple comparison correction); box-plot whiskers are drawn to the 10^{th} and 90^{th} percentiles. PLA signals were normalized to 100-μm axon segments. Scale bar, 10 μm.

FIGS. 3A-3F show that the CREB3L2-ATF4 complex accumulates in LOAD.

FIG. 3A shows a Western blot analysis of CREB3L2 and ATF4 protein levels in the prefrontal cortex (PFC) of control and LOAD cases. Each lane represents an individual case. Controls, n=5; LOAD n=8; CREB3L2, *P-value=0.0195; ATF4, **P-value=0.0015, unpaired t-tests.

FIG. 3B shows a heatplot of CREB3L2 and ATF4 mRNA expression in control and LOAD PFC. Columns differentiate between CREB3L2 and ATF4 expression profiles, while rows denote individual cases. Controls, n=101; LOAD n=129; CREB3L2, P-value=1.21×10⁻²⁰; ATF4, P-value=1.46×10⁻⁷.

FIG. 3C shows a scatter plot of CREB3L2 (X axis) and ATF4 (Y axis) mRNA expression in control and LOAD PFC. Their expression correlation was found to significantly increase in LOAD cases. Controls, Pearson r=0.1325, P-value=0.19 (not significant), linear regression slope=0.07; LOAD, Pearson r=0.3972, ***P-value <0.0001, linear regression slope=0.47.

FIG. 3D shows representative CREB3L2 and ATF4 mRNA expression in control and LOAD PFC stratified by sex and condition.

FIG. 3E shows the immunohistochemical labeling of CREB3L2 protein in LOAD PFC. See also FIGS. 9F-9H. Scale bar, 50 μm.

FIG. 3F shows a coimmunoprecipitation analysis of CREB3L2 and ATF4 association in control and LOAD PFC. Stratification reveals significant CREB3L2-ATF4 complex upregulation in moderate pathology cases. Controls, n=5; LOAD n=8 (mild LOAD n=2, moderate LOAD n=6). Note that these are the same autopsy subjects examined in FIG. 3A; sample loading order was kept constant.

FIGS. 4A-4G show the CREB3L2-ATF4 transcriptional program according to one embodiment of the present disclosure.

FIG. 4A shows an experimental outline according to one embodiment of the present disclosure. ChIPmera exploits chemically induced proximity to induce the interaction of specific TF pairs. Each monomer is engineered with two unique features: a specific dimerization domain (FRB or FKBP, C-terminally fused), and an N-terminal epitope tag (HA or V5). The monomers can then be brought together in cells using a bivalent ligand that recognizes both the FRB and the FKBP domains with high affinity. Dimer-specific DNA-binding programs are elucidated from parallel HA and V5 ChIP-seq analyses. DNA constructs were transiently transfected into HEK293 cells with simultaneous dimerizer delivery, and ChIPmera system incubation for 24 hours. ChIPmera backgrounds signals were established using a chemically induced Renilla luciferase homodimer.

FIG. 4B shows a representative motif analysis of CREB3L2-CREB3L2, ATF4-ATF4, and CREB3L2-ATF4 binding sites. Heterodimer-bound sequences were either enriched in canonical CREB3L2 or ATF4 recognition sites, and did not include new binding motifs.

FIG. 4C shows representative binding behaviors displayed by CREB3L2-CREB3L2, ATF4-ATF4 and CREB3L2-ATF4 dimers. Also shown are ENCODE-generated histone H3 lysine 27 acetylation (H3K27Ac) and DNaseI hypersensitivity profiles, which mark important regulatory regions, such as gene promoters; in both cases, signal strength is indicated by increasingly darker shades.

FIG. 4D shows the differential enrichment analysis of genomic regions bound by CREB3L2-ATF4 and ATF4-ATF4 dimers. Enrichment fold-change and statistical significance are plotted along the x- and y-axes, respectively, from n=2 independent replicates. Magenta data points: false discovery rate (FDR) s 0.01; blue data points: FDR >0.01.

FIG. 4E shows the differential enrichment analysis of genomic regions bound by CREB3L2-ATF4 and CREB3L2-CREB3L2 dimers. Axes layout and data point codification as in (d).

FIG. 4F shows the gene ontology functional analysis of promoter-associated CREB3L2-ATF4 targets (biological process).

FIG. 4G shows the genome browser tracks in AD susceptibility loci juxtaposed with ENCODE-produced H3K27Ac and DNaseI hypersensitivity profiles.

FIGS. 5A-5H show that retromers may be transcriptionally targeted by CREB3L2-ATF4.

FIG. 5A shows the STRING protein interaction network of CREB3L2-bound genes identified by ChIP-seq on PFC LOAD chromatin. Nodes represent proteins and edges denote predicted functional associations. STRING analysis identified 107 interconnected CREB3L2-regulated proteins (n=1).

FIG. 5B shows the distribution of CREB3L2 ChIP-seq signals across the LOAD genome. Cutoff for promoter regions was defined as ±3 kb from a TSS.

FIG. 5C shows the gene ontology term enrichment analysis of promoter-associated CREB3L2 targets (cellular component).

FIG. 5D shows the LOAD CREB3L2 ChIP-seq genome browser tracks in SEC31A and SNX3 loci juxtaposed with ENCODE-produced H3K27Ac and DNaseI hypersensitivity profiles. SEC31A encodes a component of the COPII protein complex and participates in vesicle budding from the ER; SNX3 participates in retromer targeting to the early endosome. For H3K27Ac and DNaseI hypersensitivity profiles, signal strength is indicated by increasingly darker shades.

FIG. 5E shows the endosome-specific gene ontology term breakdown of ATF4-bound protein-coding genes. This ATF4 ChIP-seq dataset was produced by the ENCODE Consortium using human K562 cells.

FIG. 5F shows the LOAD CREB3L2 and ENCODE ATF4 ChIP-seq signals for RAB7A, a mediator of retromer-late endosome interactions.

FIG. 5G shows the unresolved CREB3L2 ChIP-seq peaks from LOAD PFC chromatin juxtaposed with ChIPmera signals. For clarity, LOAD ChIP-seq and ChIPmera tracks are displayed using different viewing ranges.

FIG. 5H is a diagram showing overlapping and unique sets of homodimer- and/or heterodimer-occupied retromer genes. RAB7A is the only retromer-associated gene that is commonly targeted by CREB3L2-CREB3L2, ATF4-ATF4 and CREB3L2-ATF4 dimers.

FIGS. 6A-6L show a transcriptional mechanism of retromer deregulation.

FIG. 6A shows a heatplot of retromer mRNA expression in dorsolateral PFC areas of non-demented control and LOAD human cohorts contrasted against CREB3L2. Data are presented as log fold change and originate from the same population as in FIG. 3b-d. Rows denote individual cases (Controls, n=101; LOAD n=129).

FIG. 6B shows a list of the ten most significantly altered retromer subunits in LOAD dorsolateral PFC.

FIG. 6C shows the expression correlation analysis between CREB3L2 and retromer.

FIGS. 6D-6H show the relative firefly luciferase reporter activities in homodimer- or heterodimer-expressing HEK293 cells. For each reporter, a Renilla luciferase homodimer, also induced using chemically induced proximity, was used to establish control background signals and replicate normalization (dashed lines). Individual data points (and mean±SEM summaries) of independently replicated experiments are shown: n=4 (VPS29, SNX1, RAB7A), n=6 (EHD1) and n=3 (PTBP1). Statistical significance determined by one-way ANOVA test with post hoc Bonferroni multiple comparison correction.

FIG. 61 shows that amyloid precursor protein (APP) membrane and cytosolic levels were analyzed by western blot in Creb3/2-suppressed rat cortical neurons. Surface proteomes were marked using a cell-impermeable biotin label. Efficient surface proteome capture is shown by glutamate receptor subunit staining (GluN2 A/B). βIII-tubulin levels were used for normalization between conditions (Wolff, 2009). Box-plots display a summary of six independent experiments (***P-value=0.0086, unpaired t-test); box-plot whiskers locate minimum and maximum values.

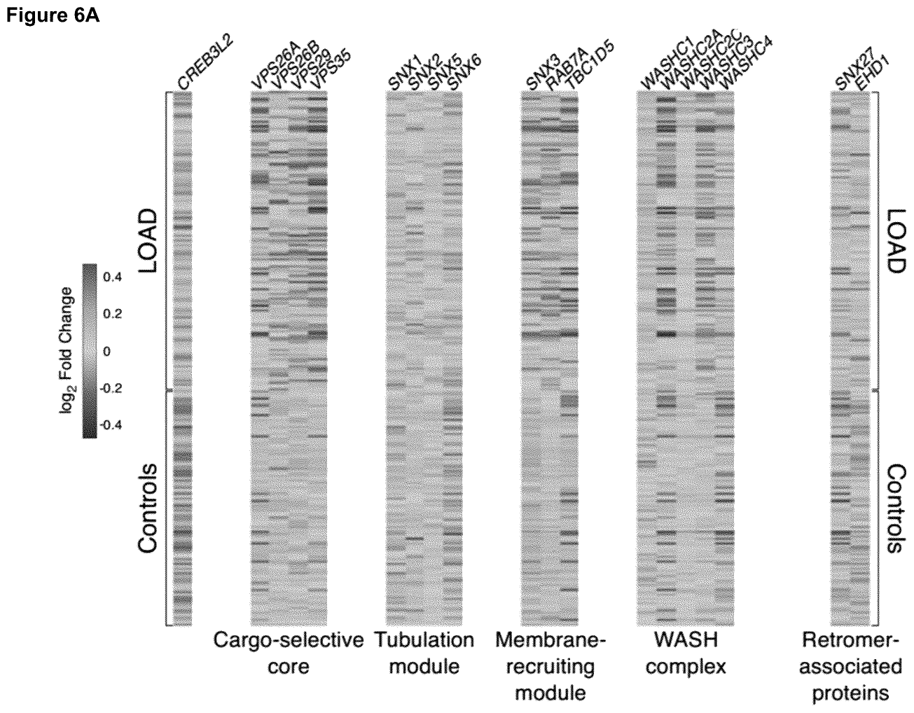

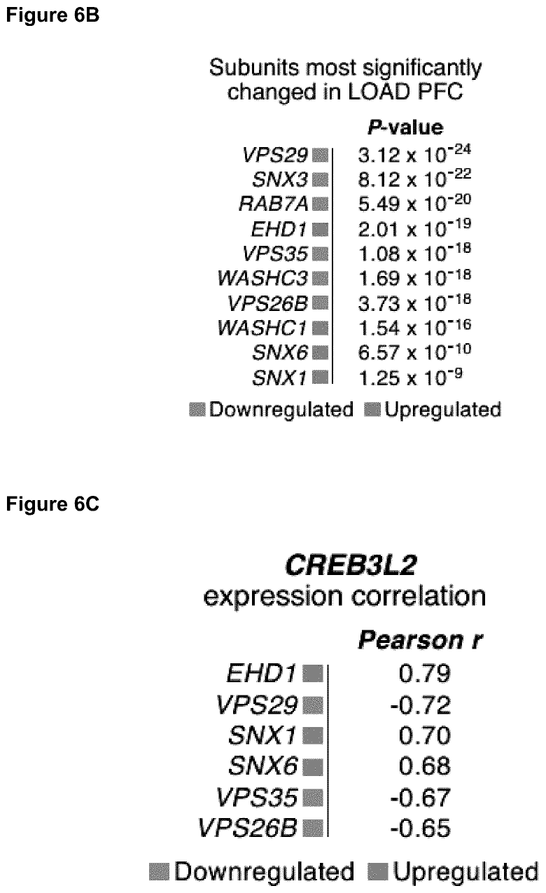

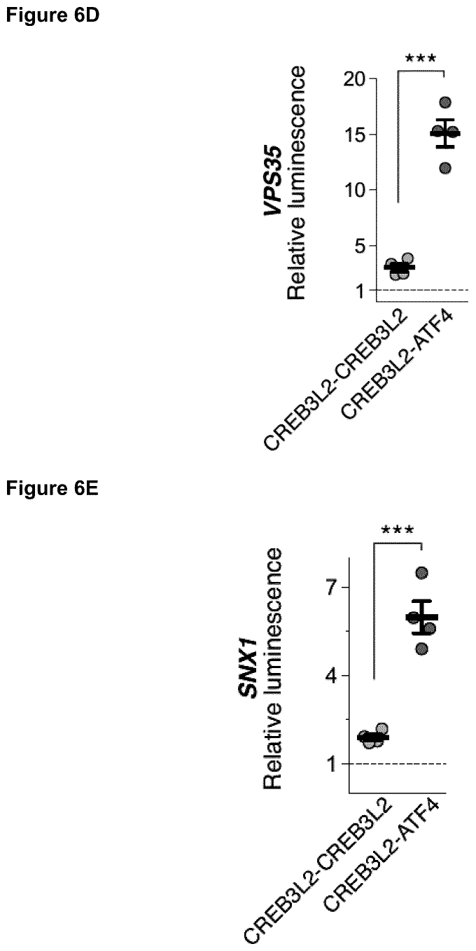

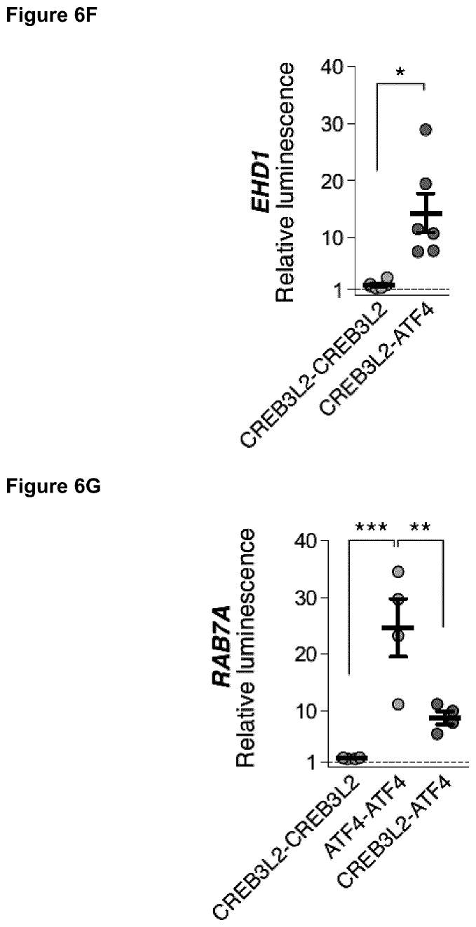

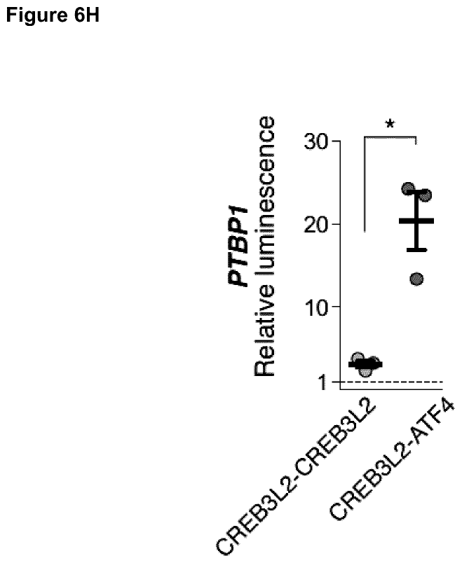

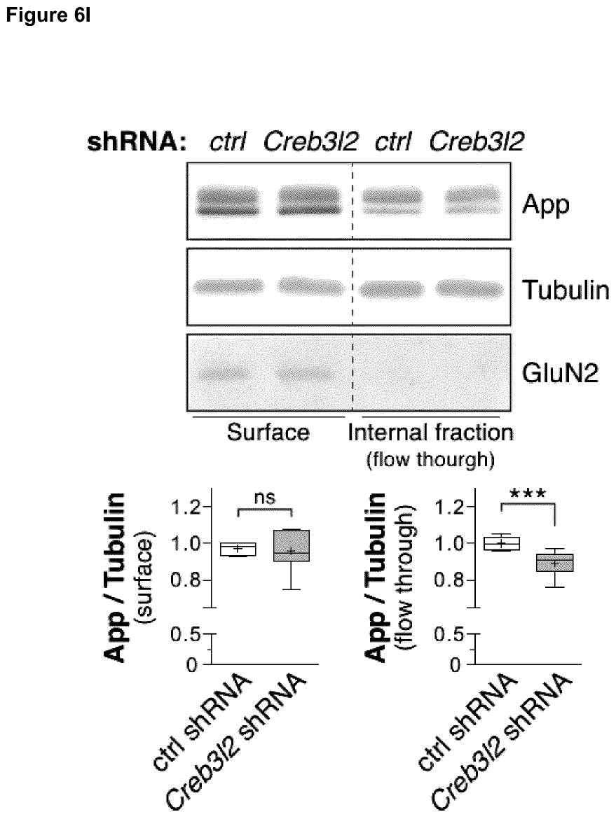

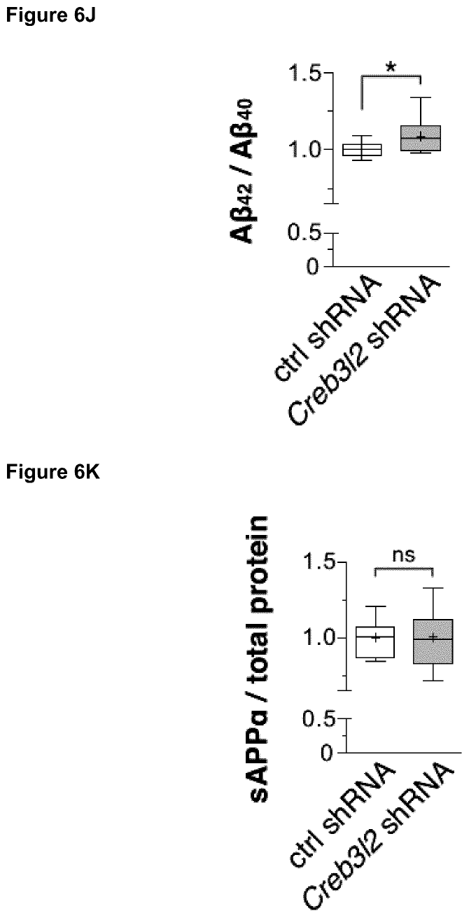

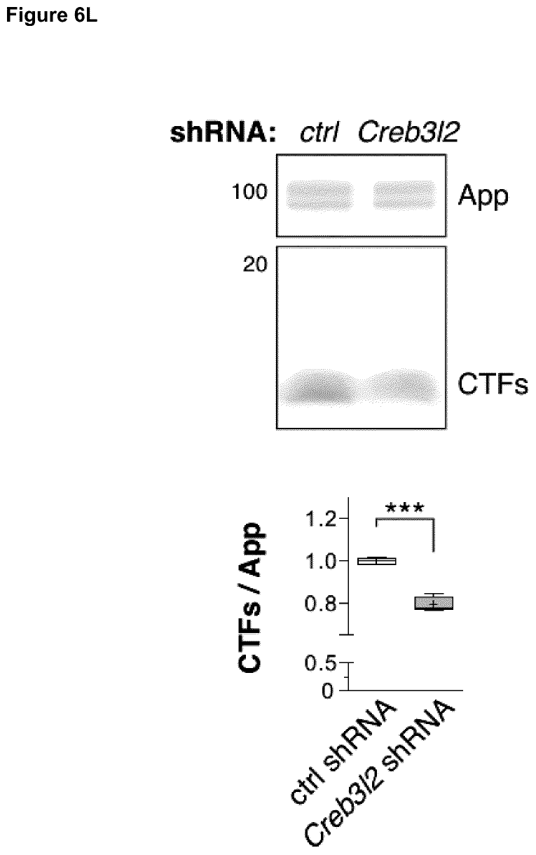

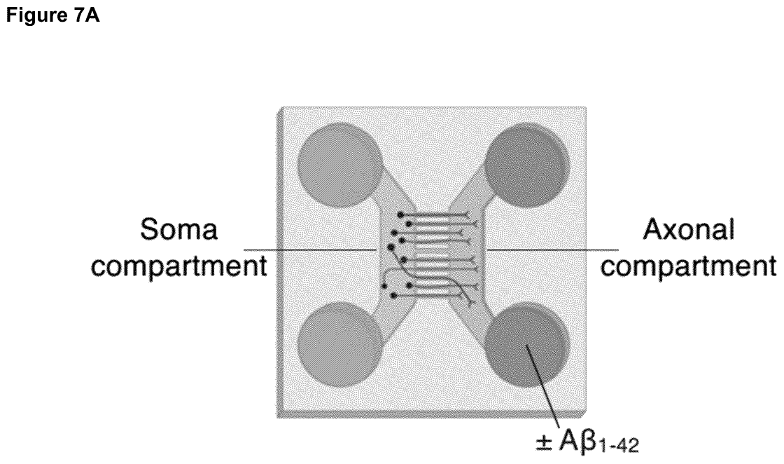

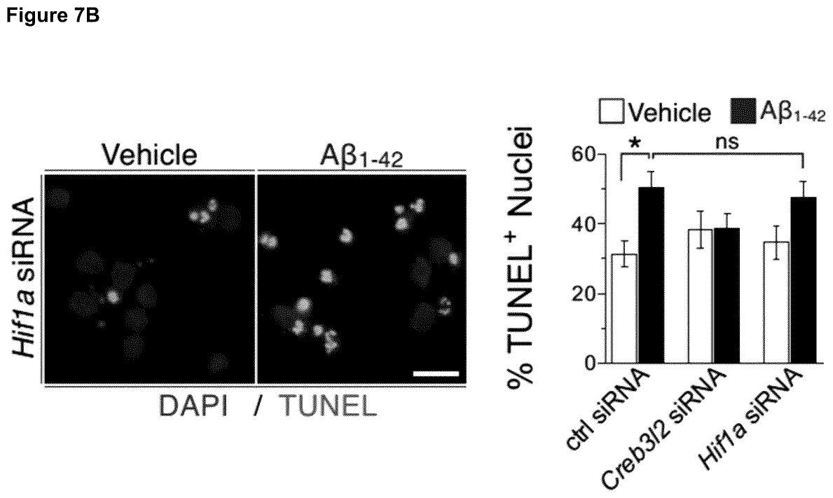

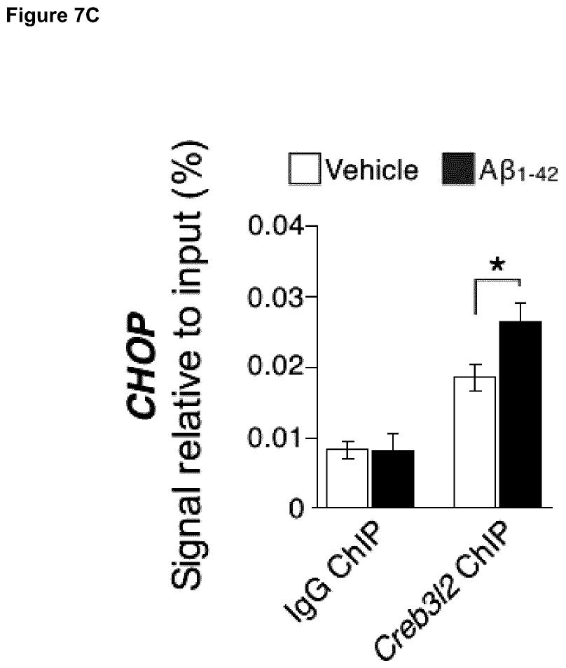

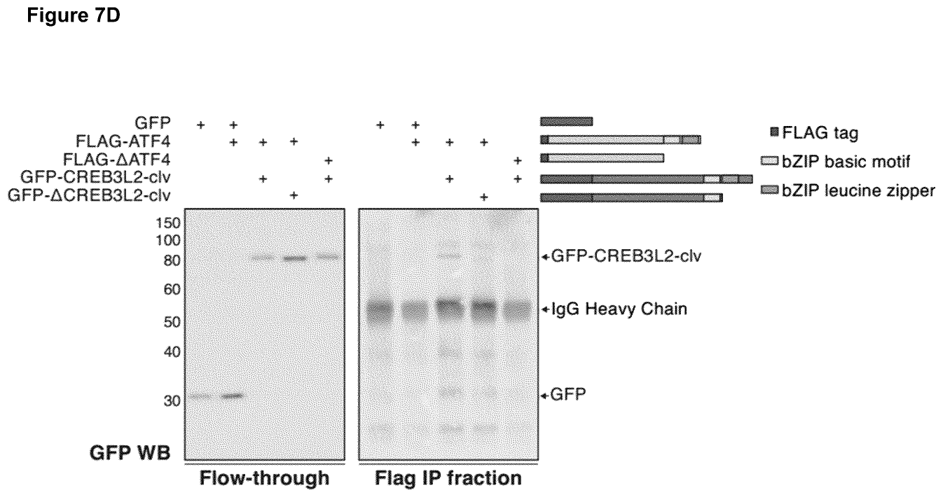

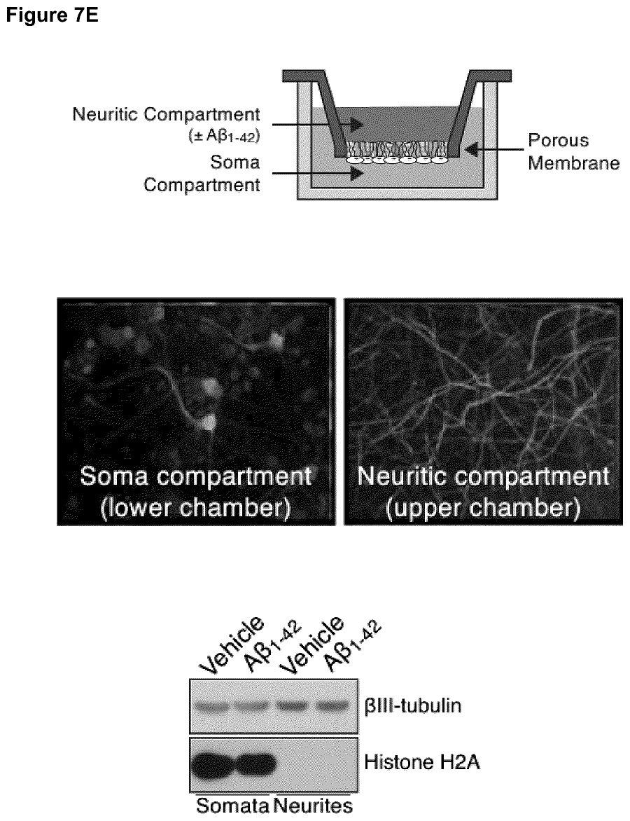

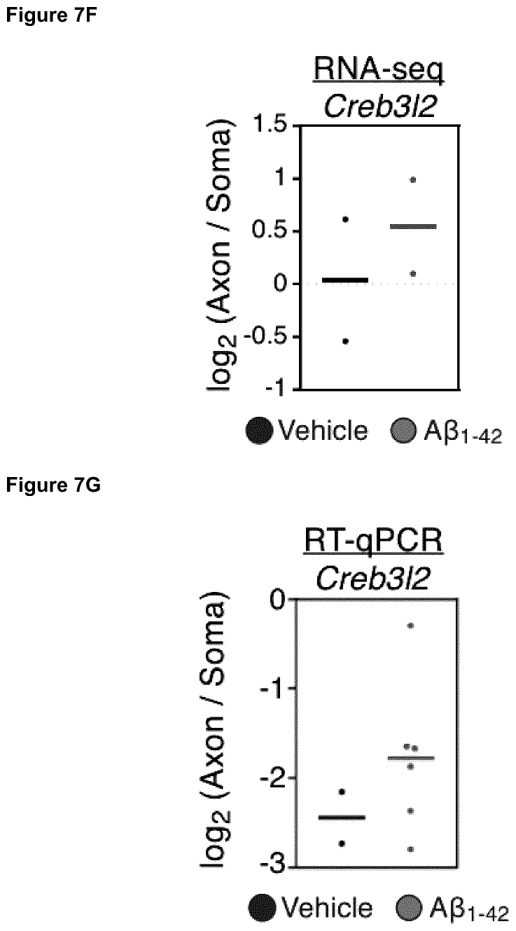

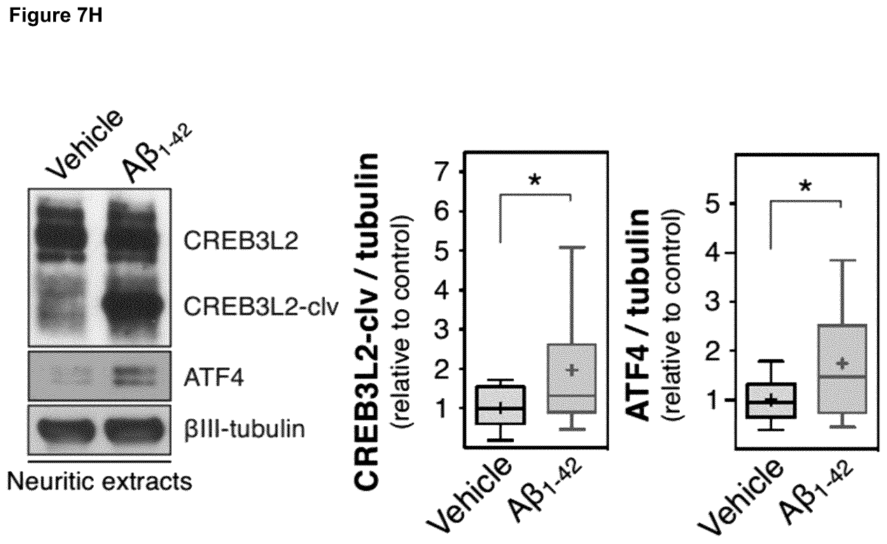

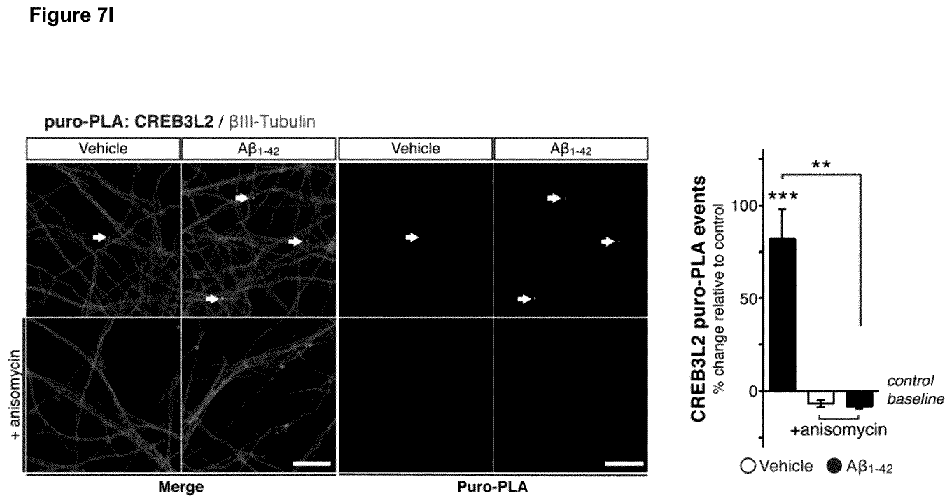

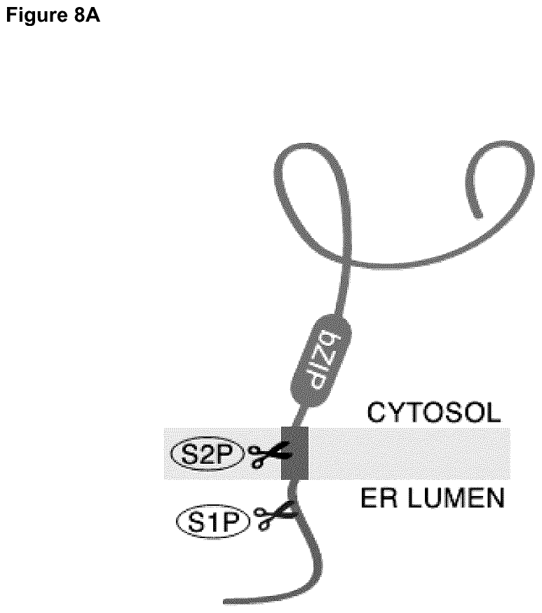

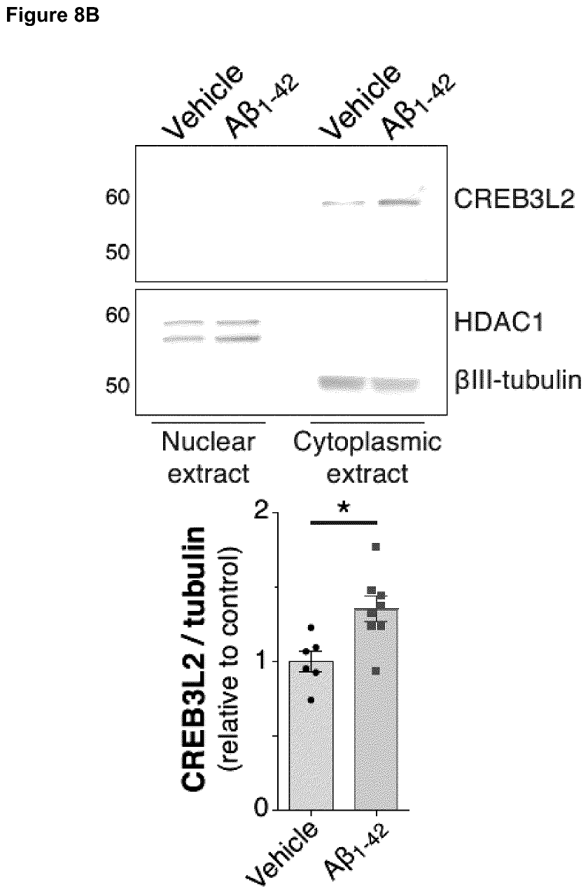

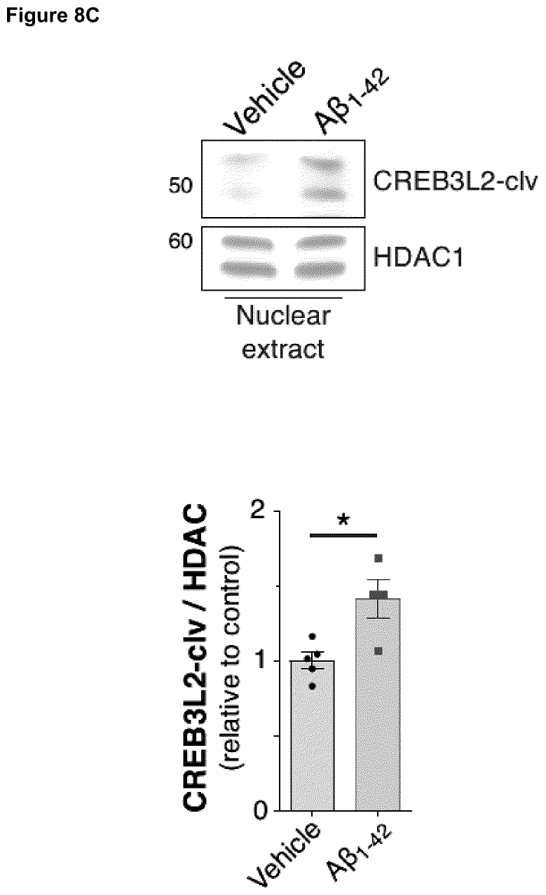

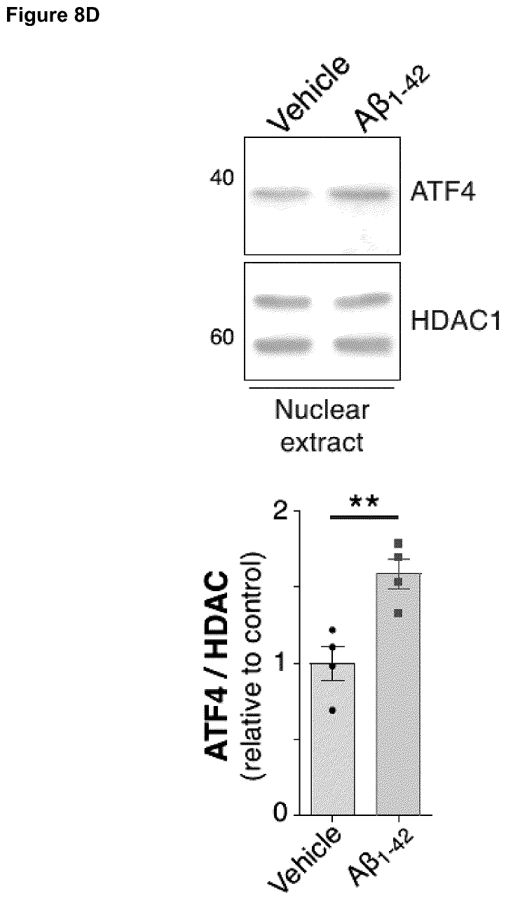

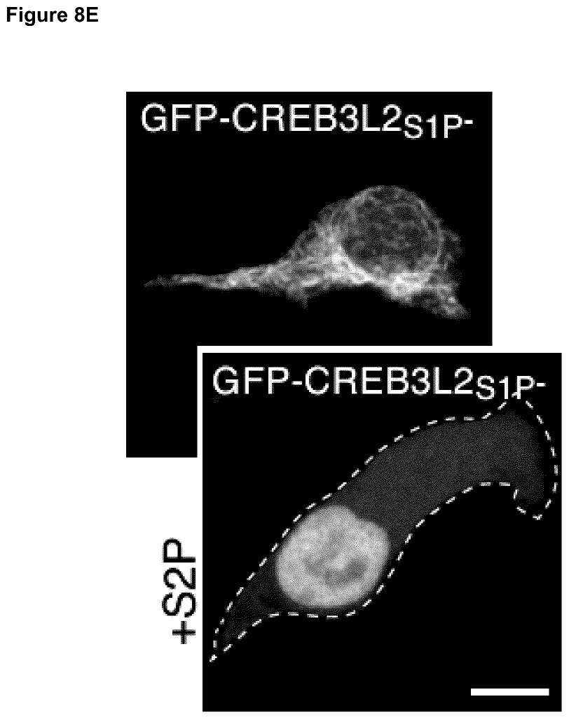

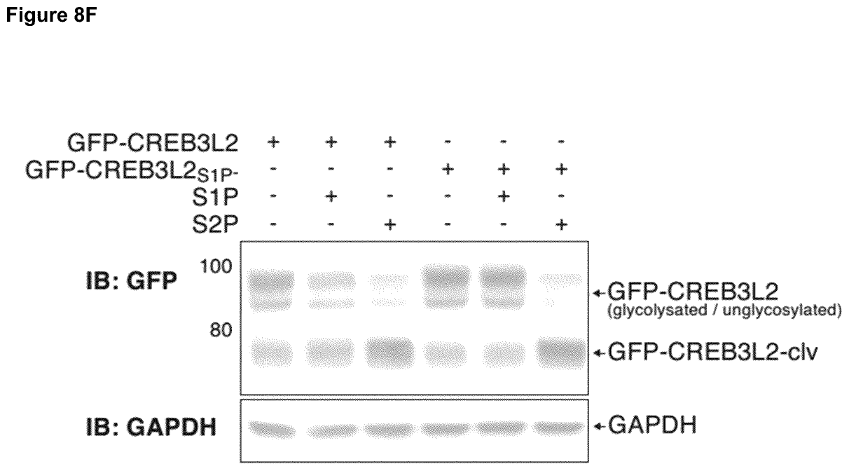

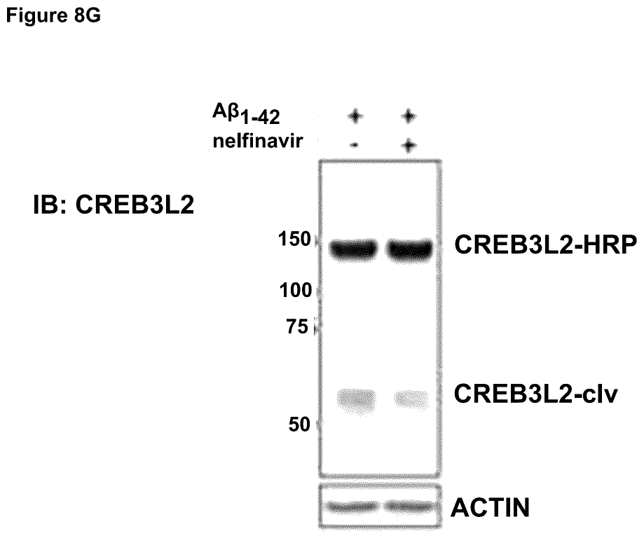

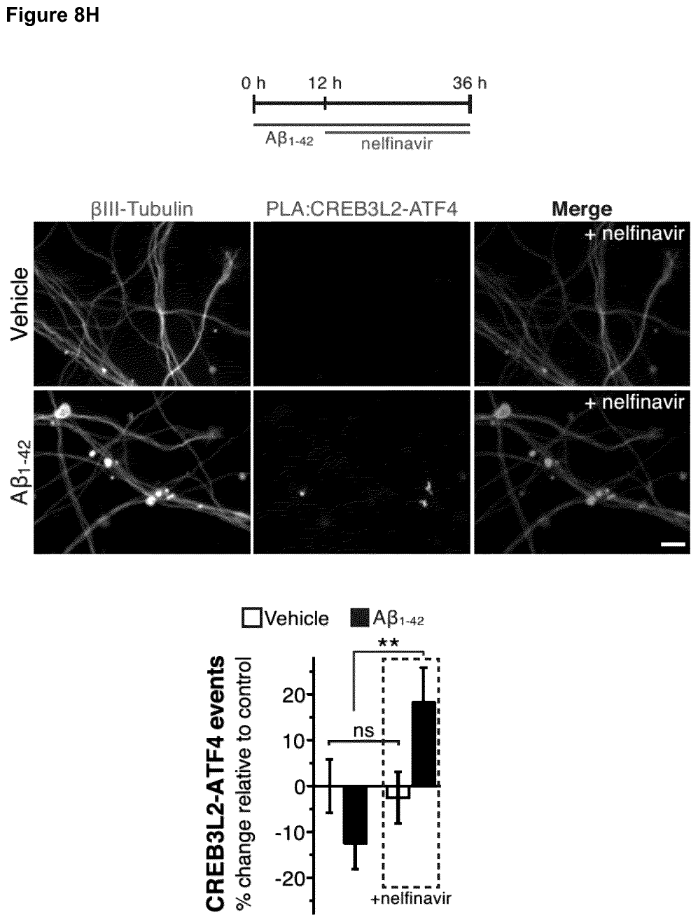

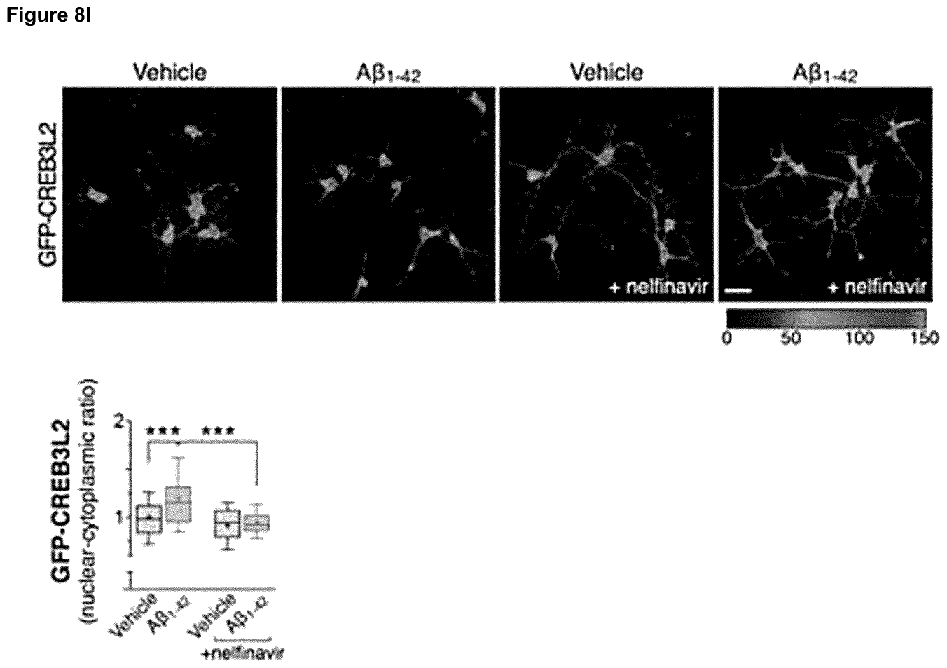

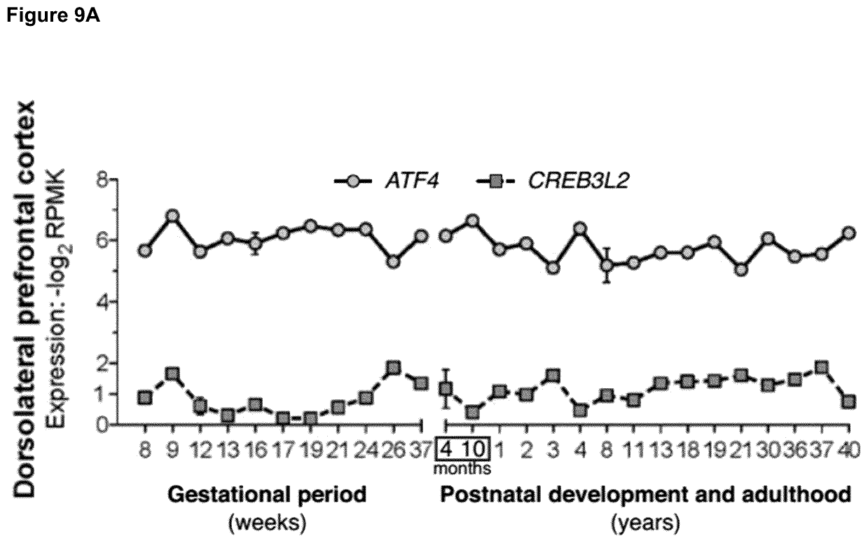

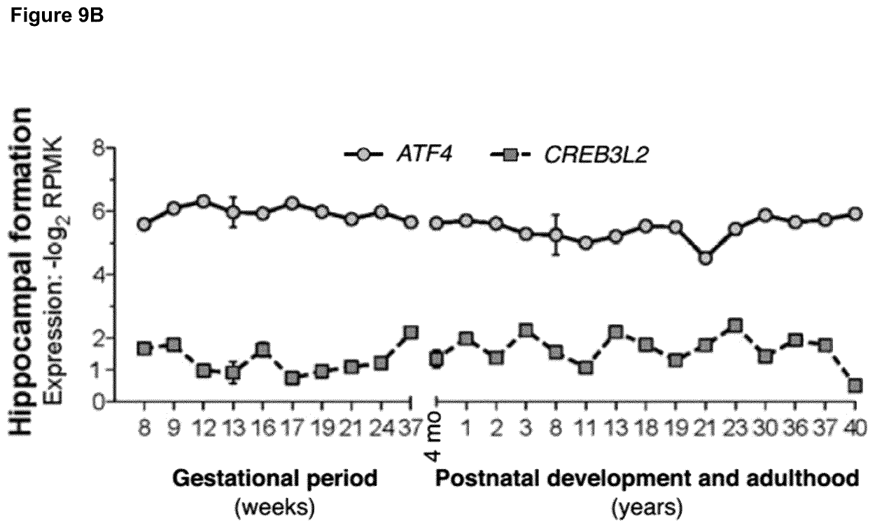

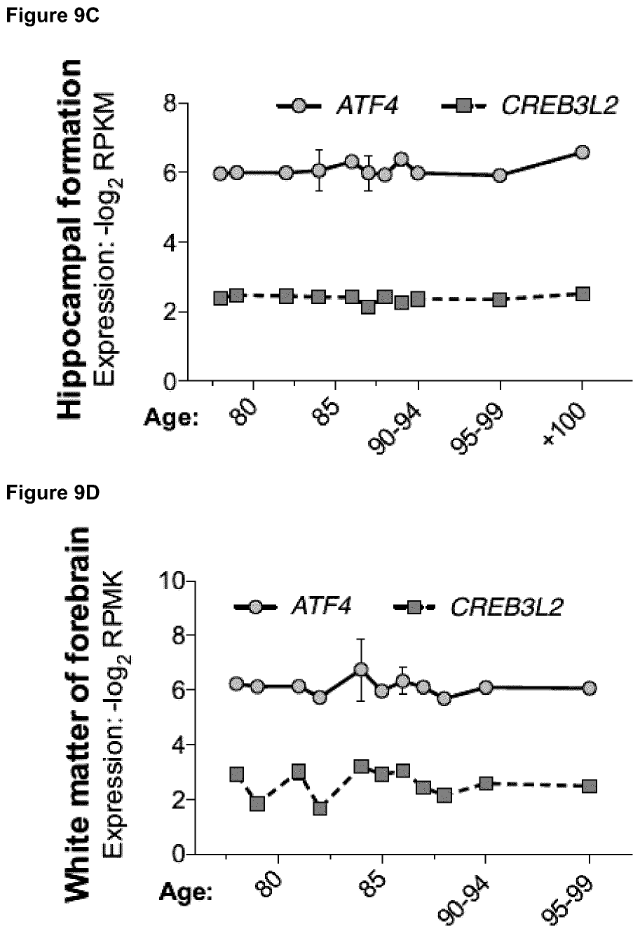

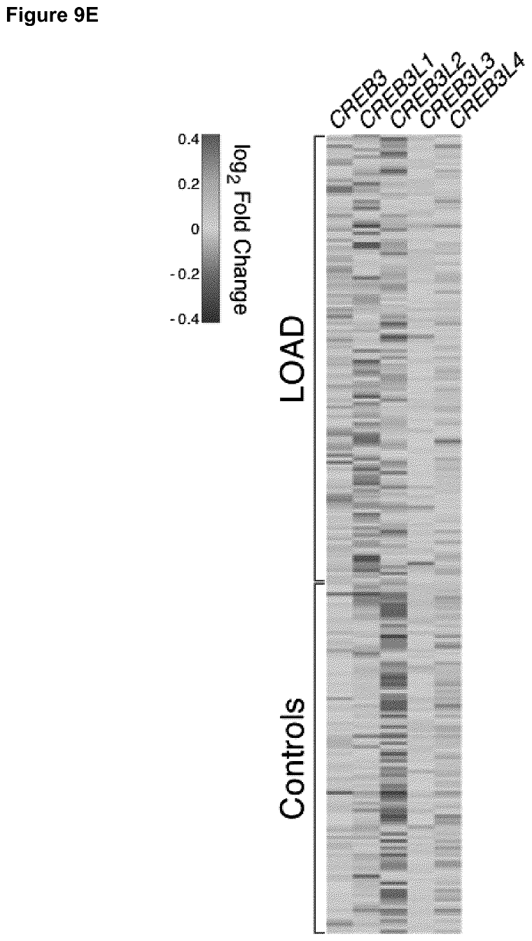

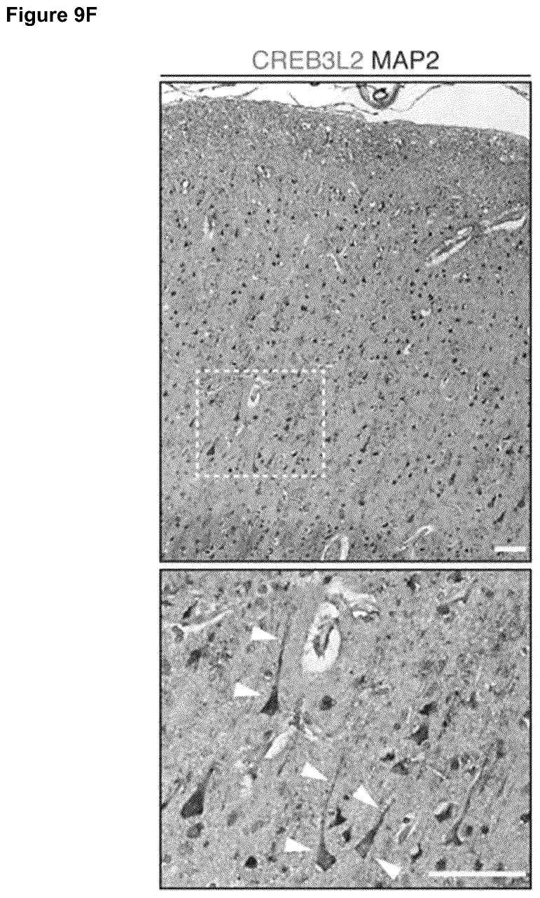

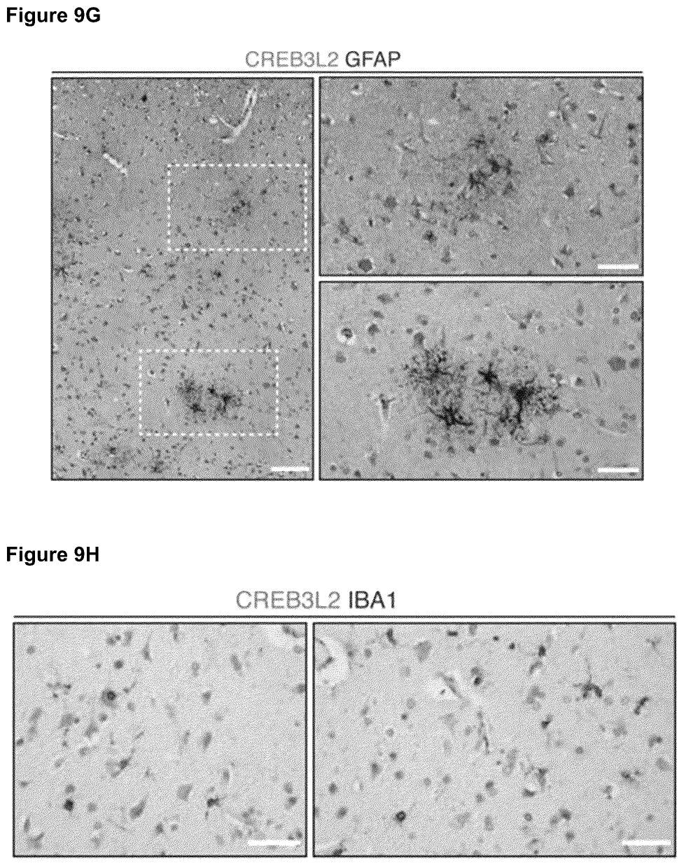

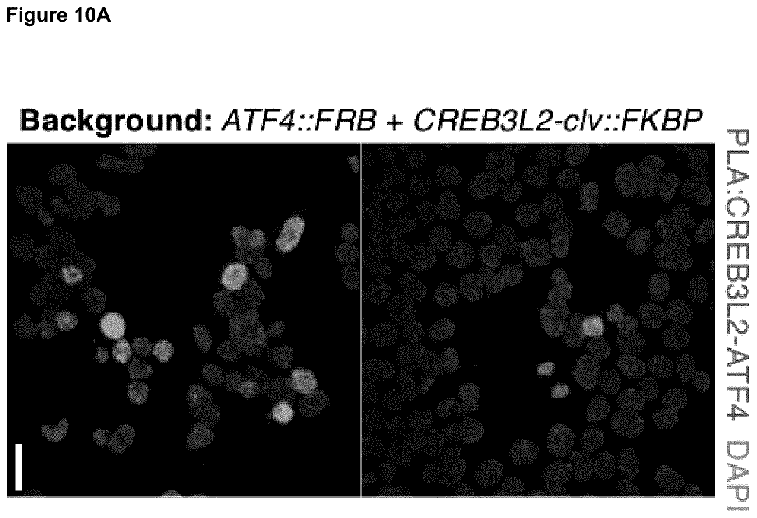

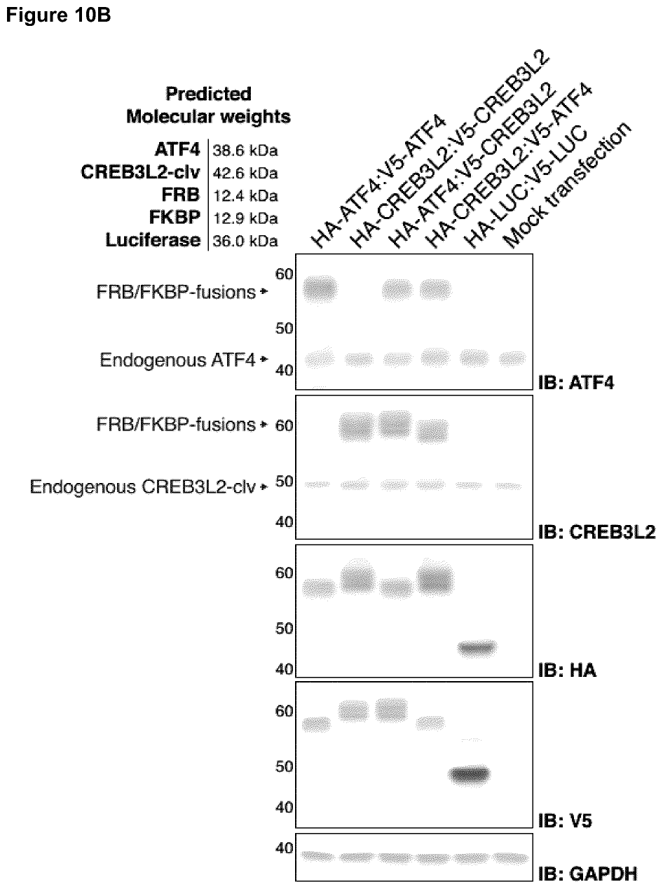

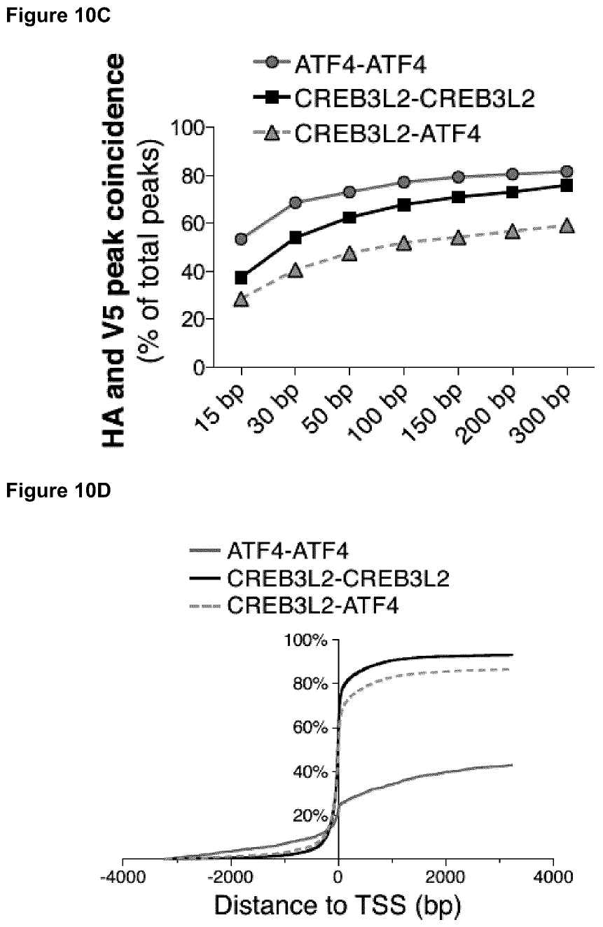

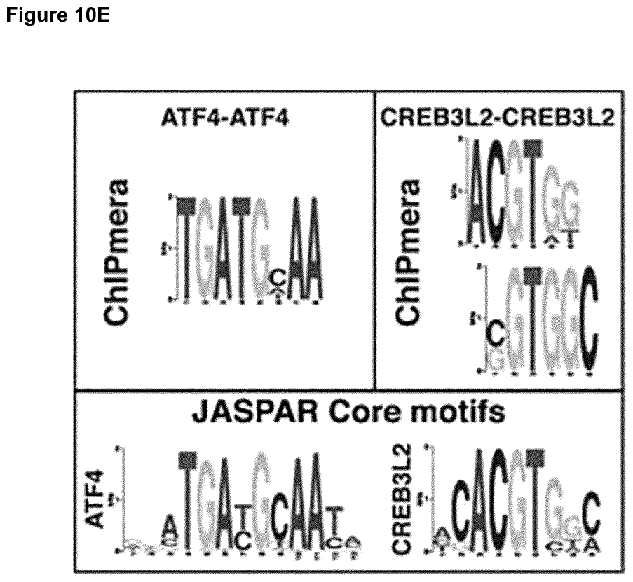

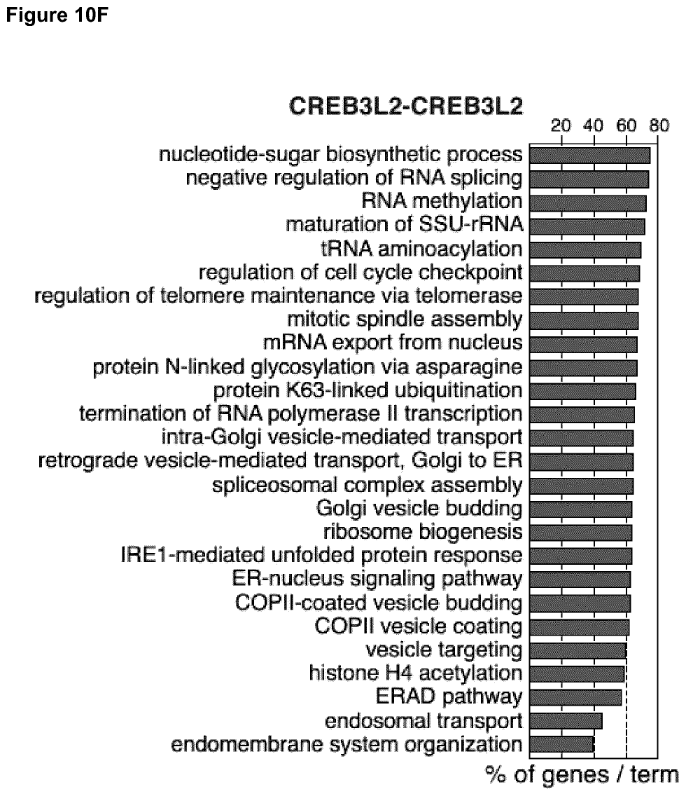

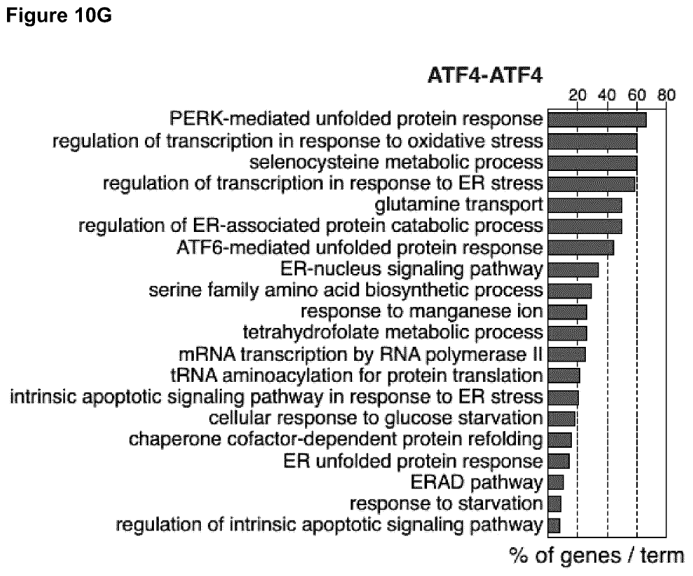

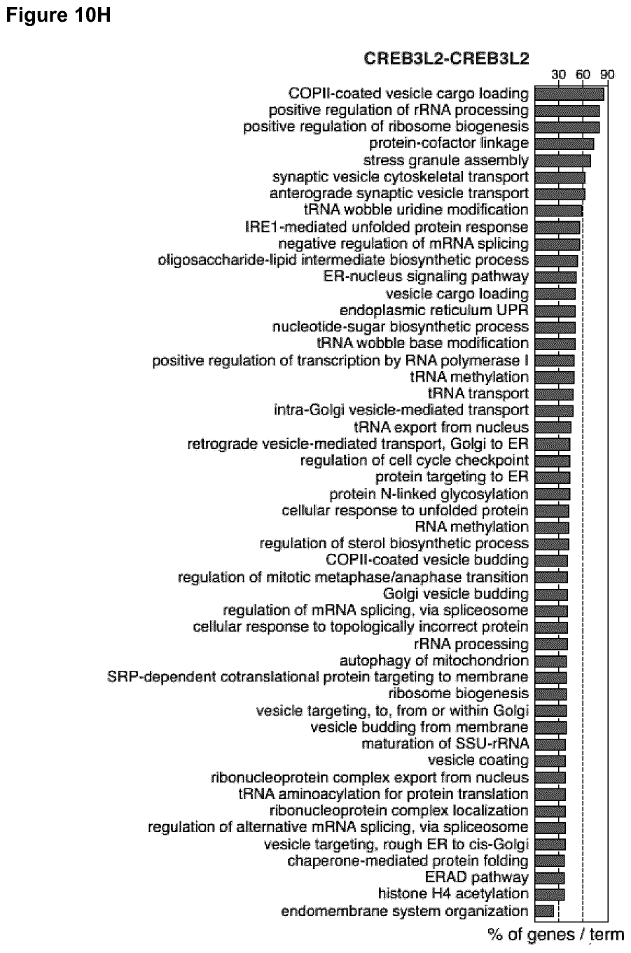

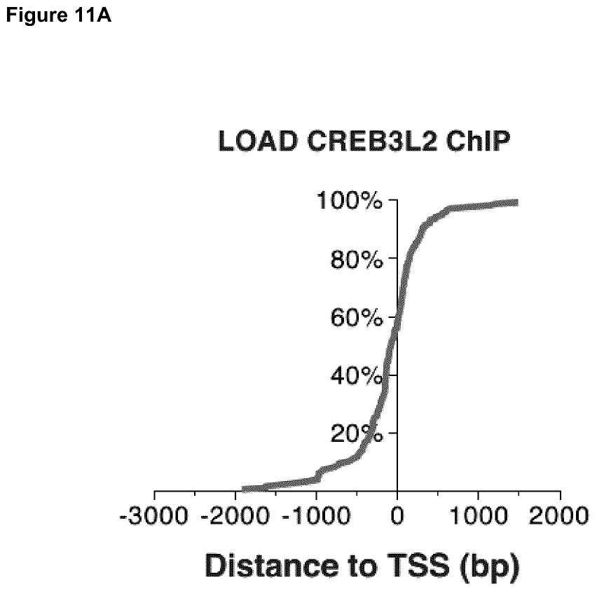

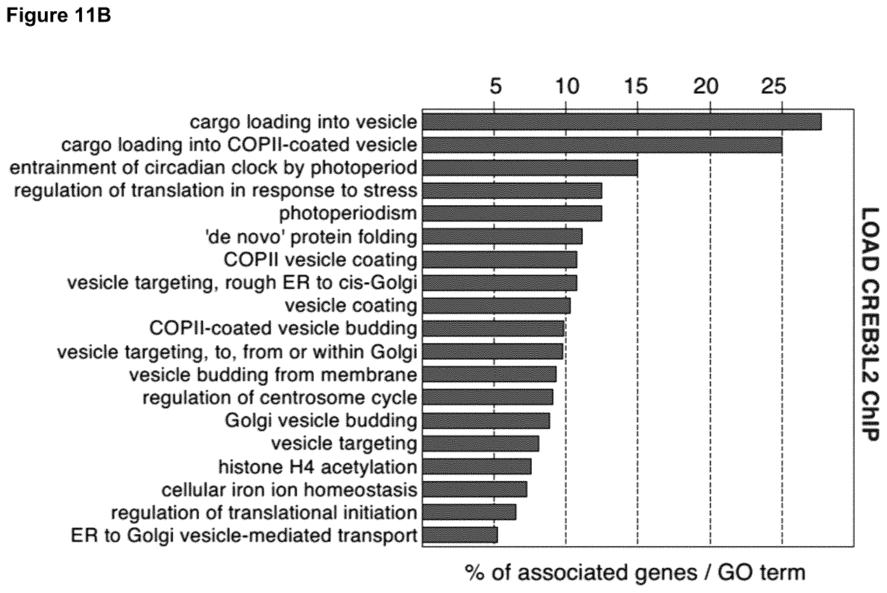

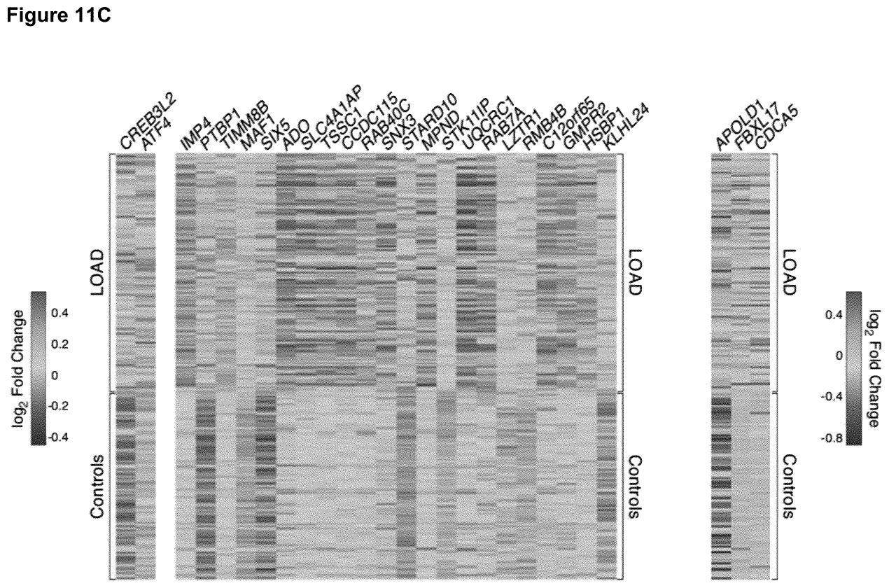

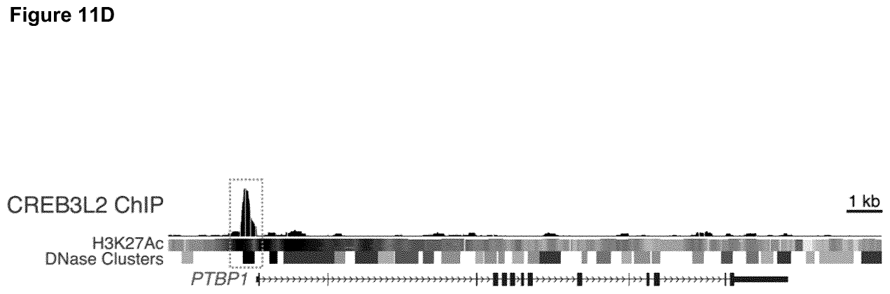

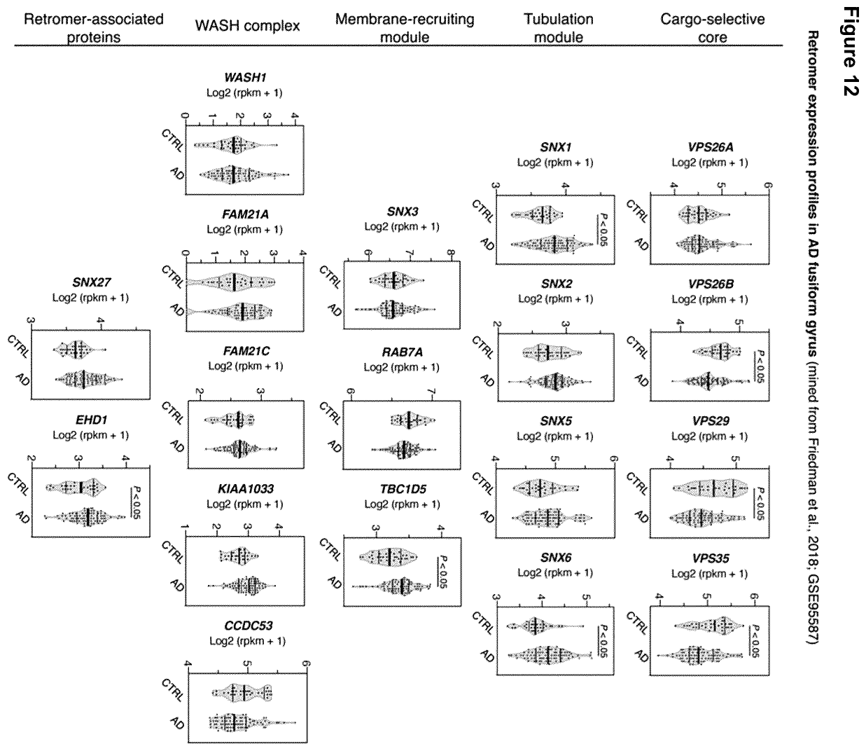

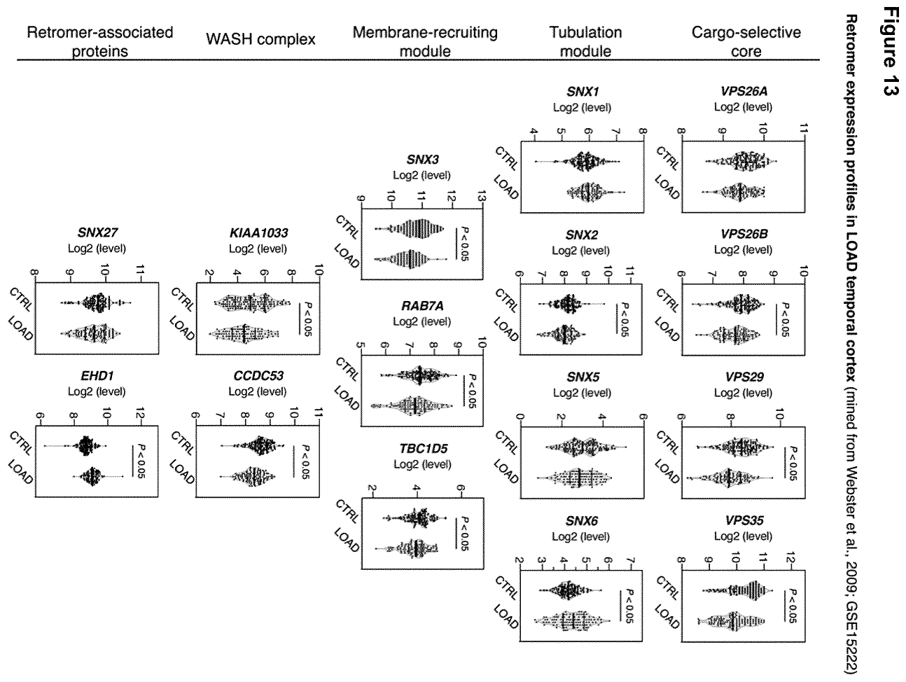

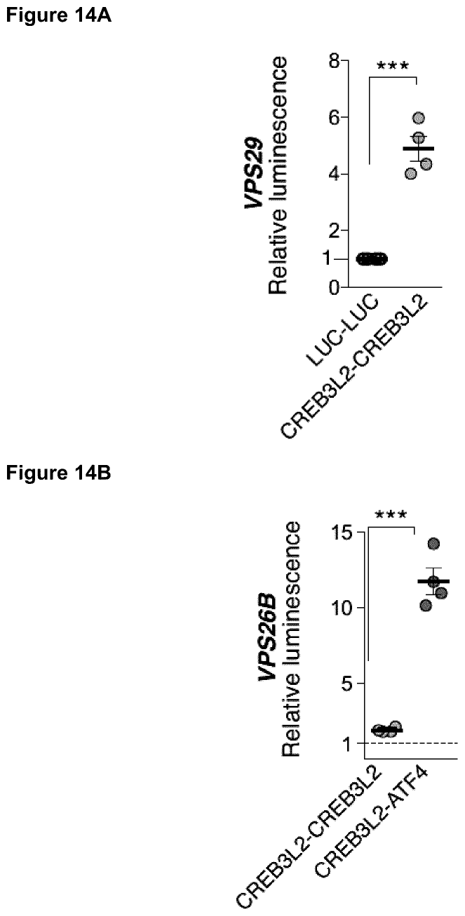

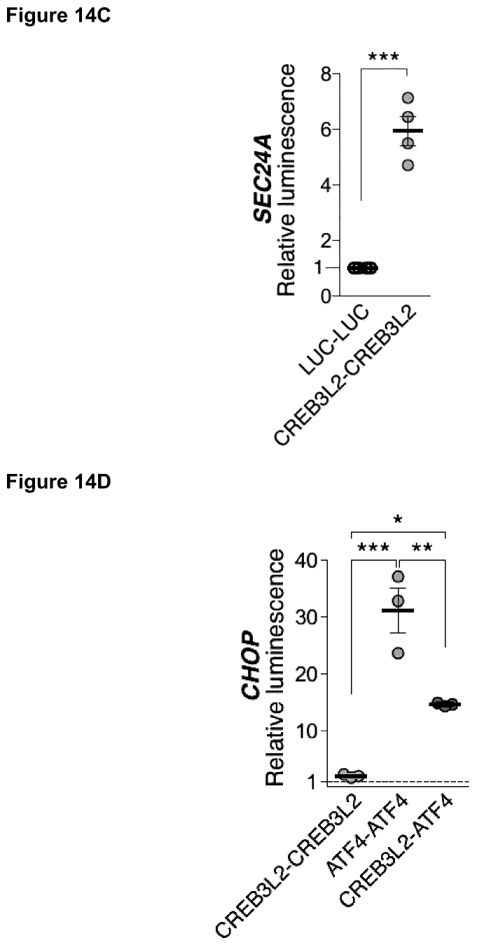

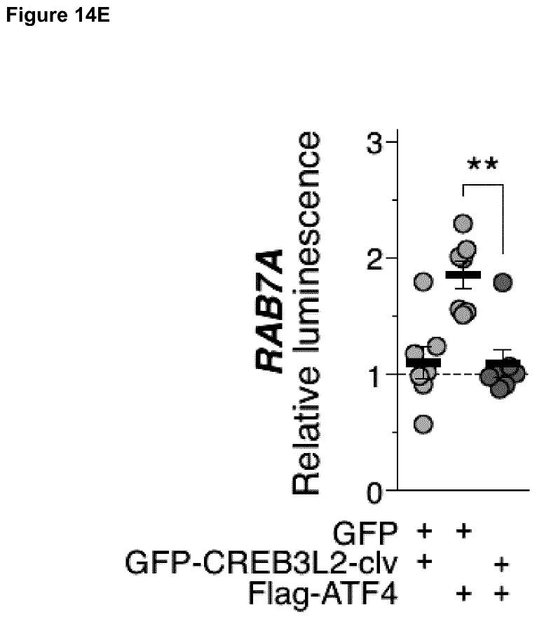

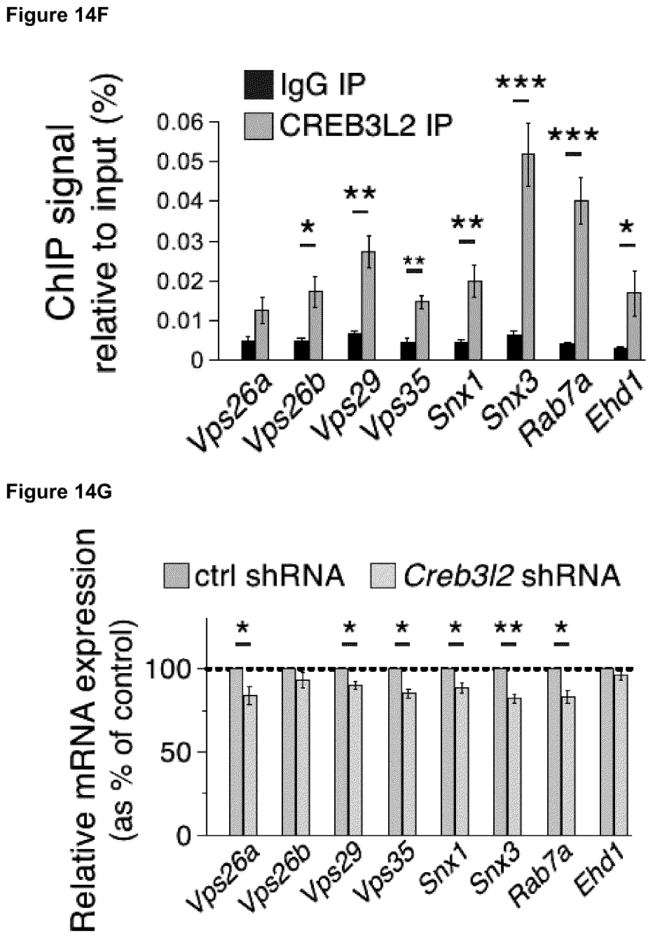

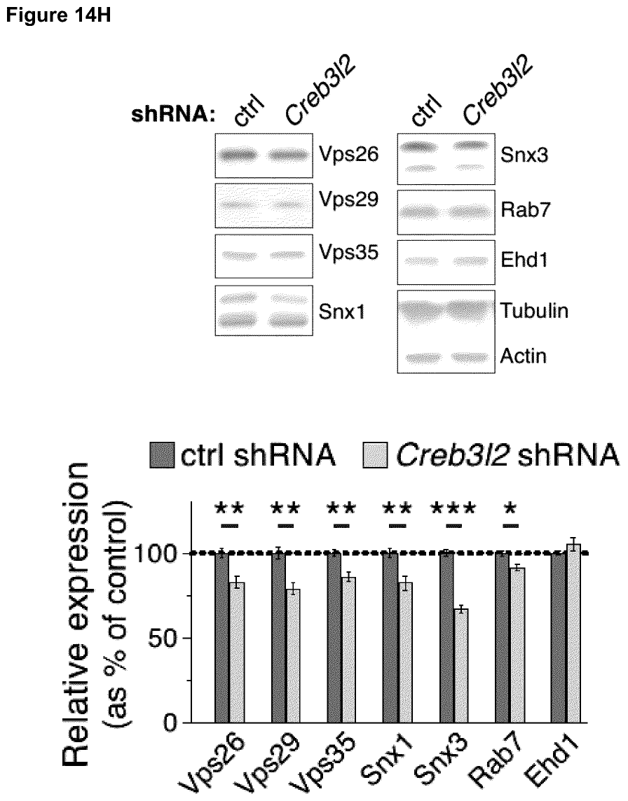

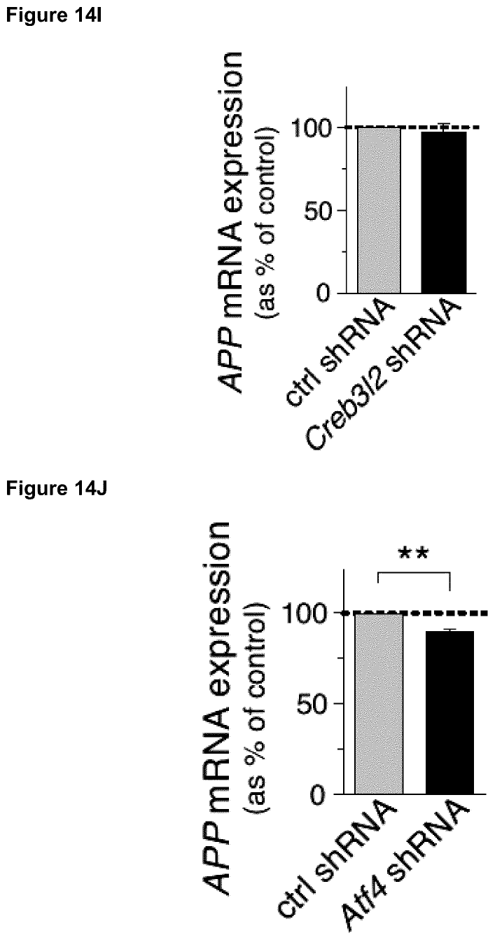

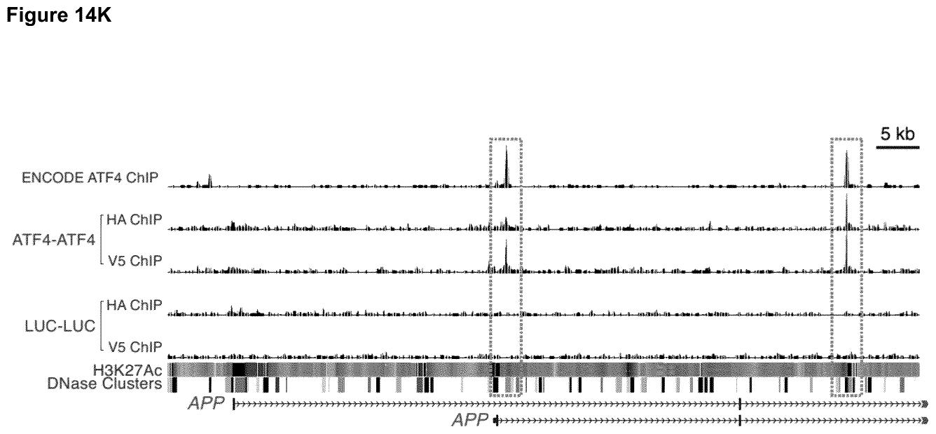

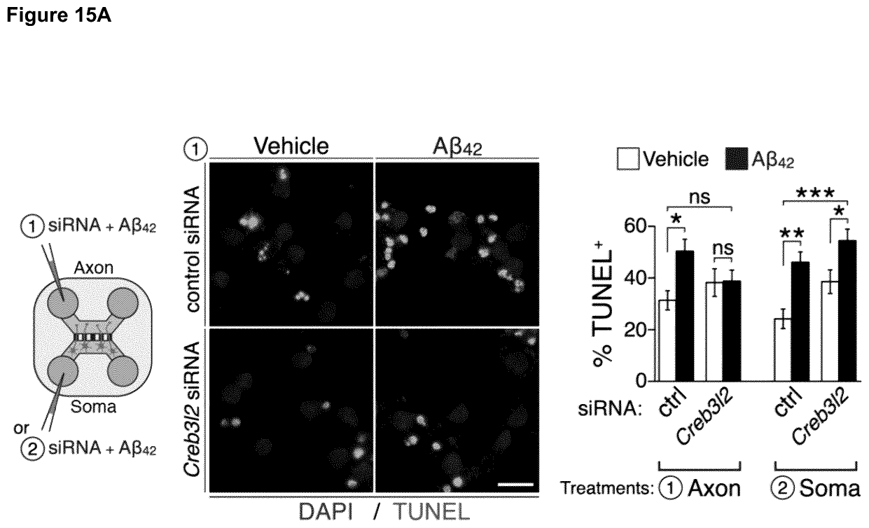

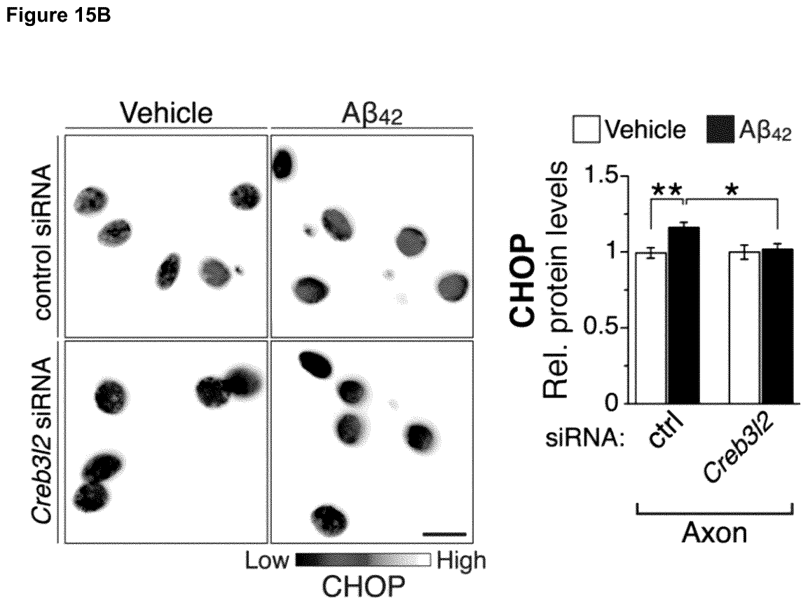

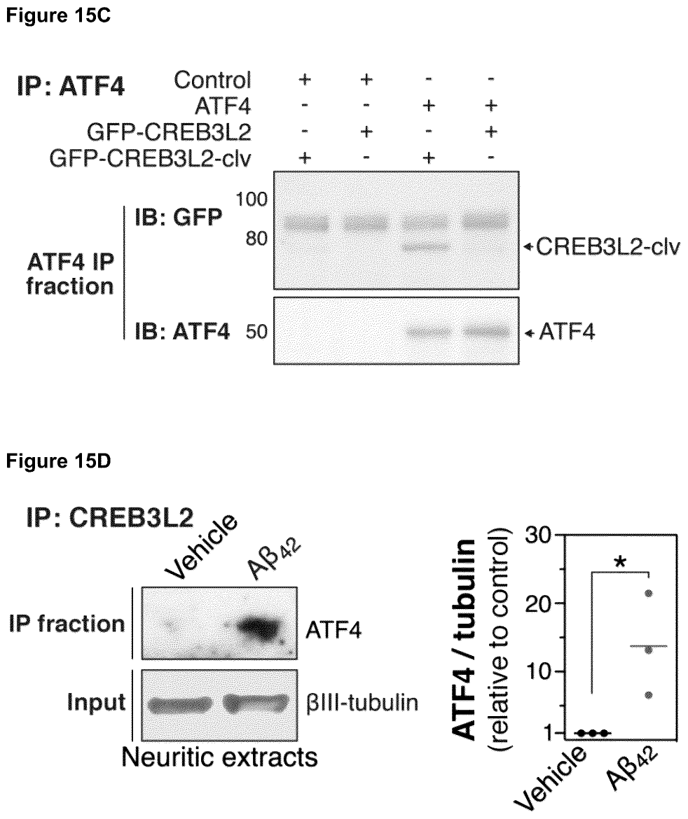

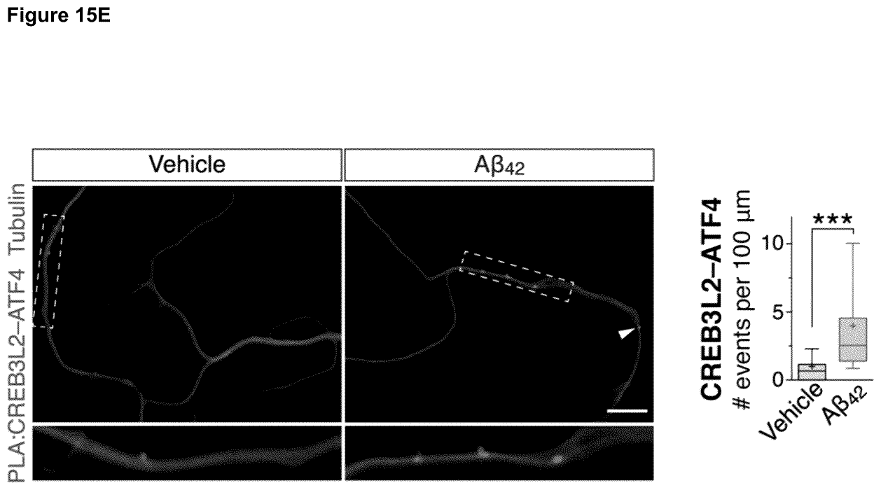

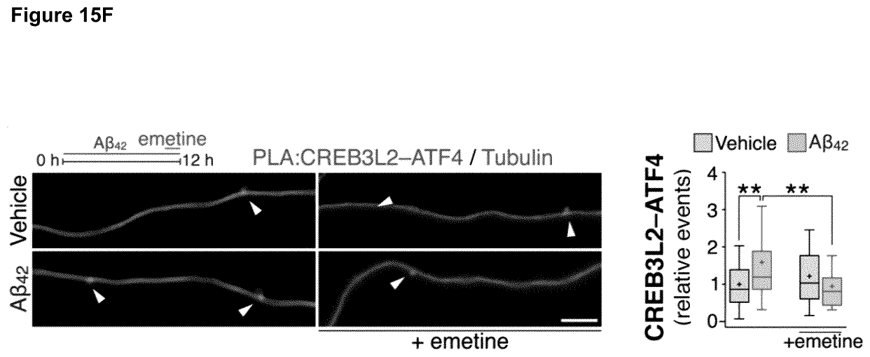

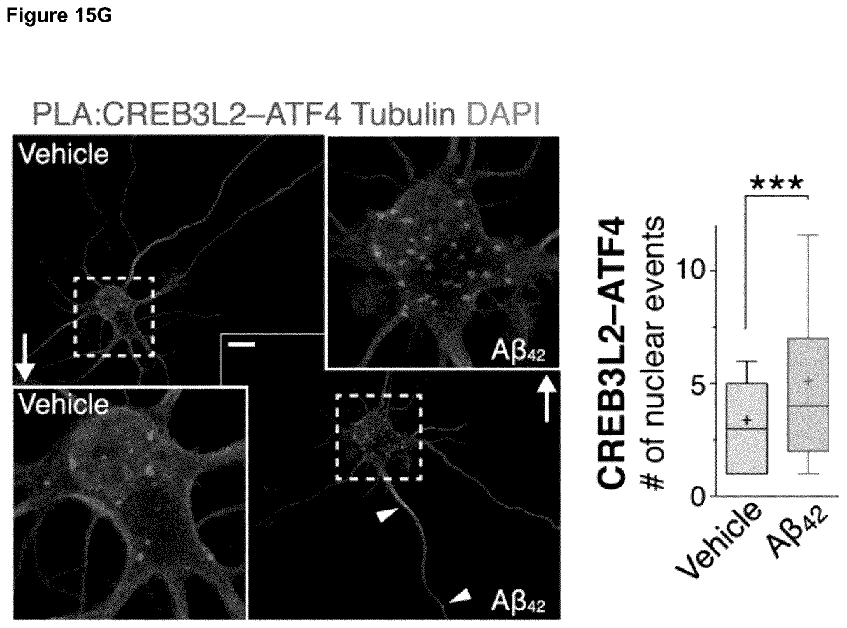

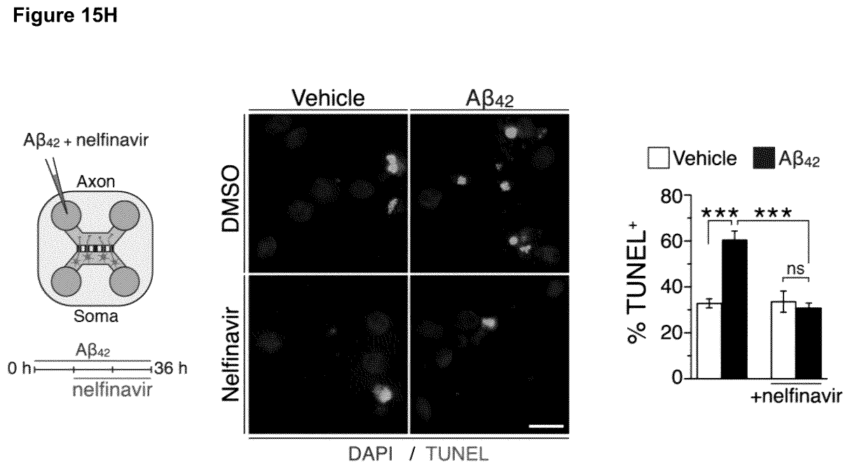

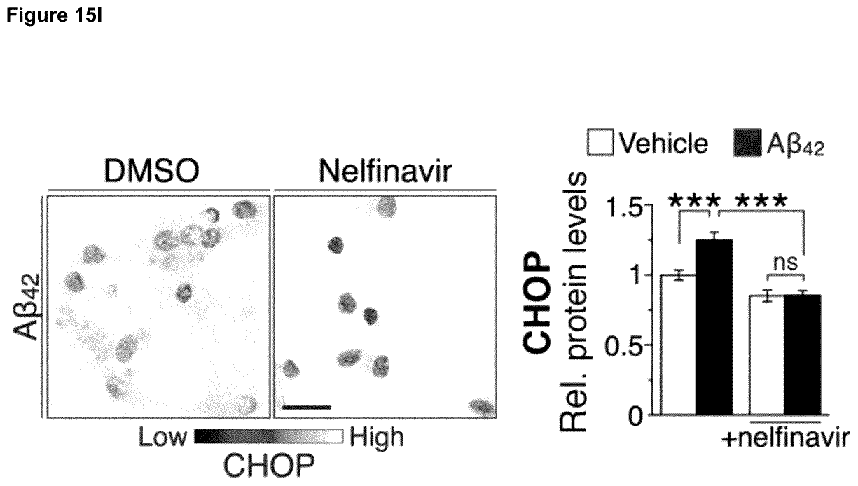

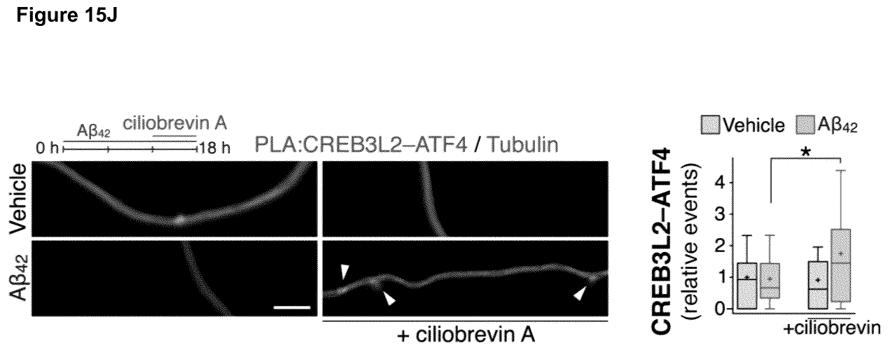

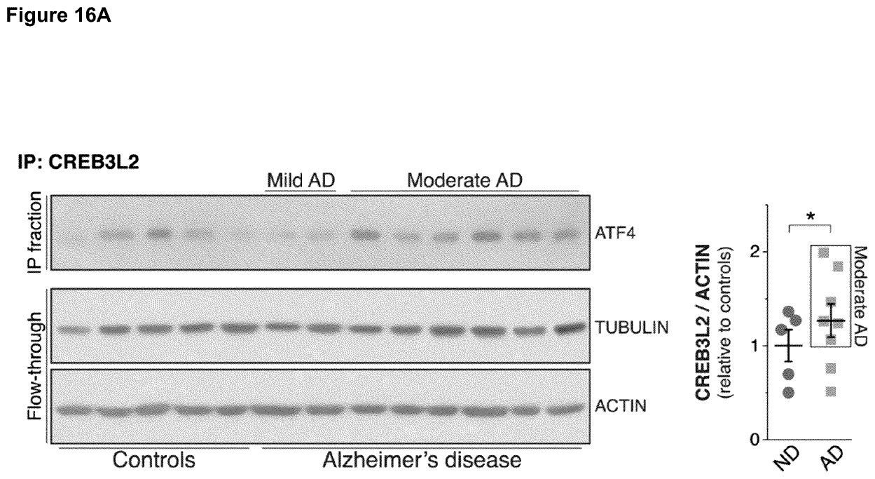

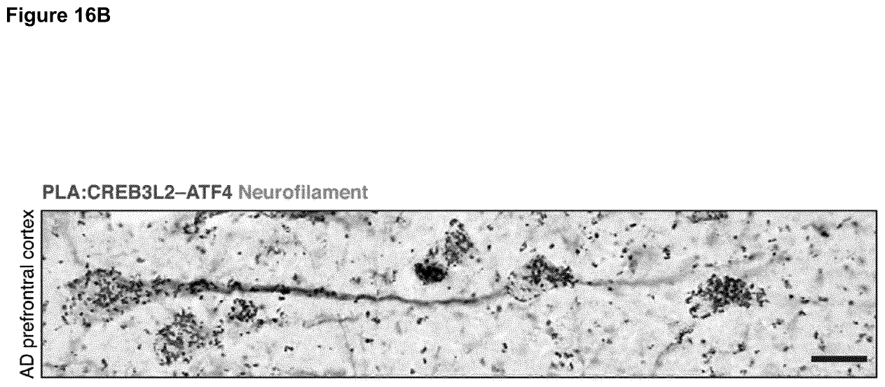

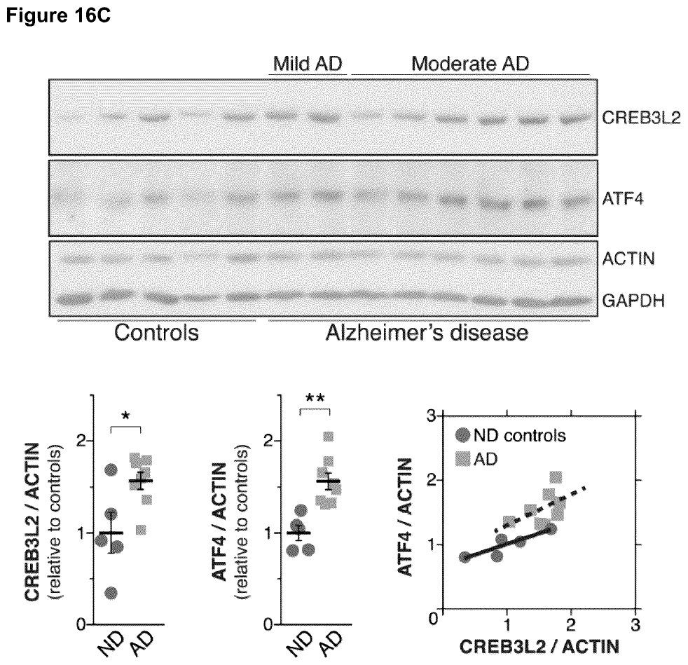

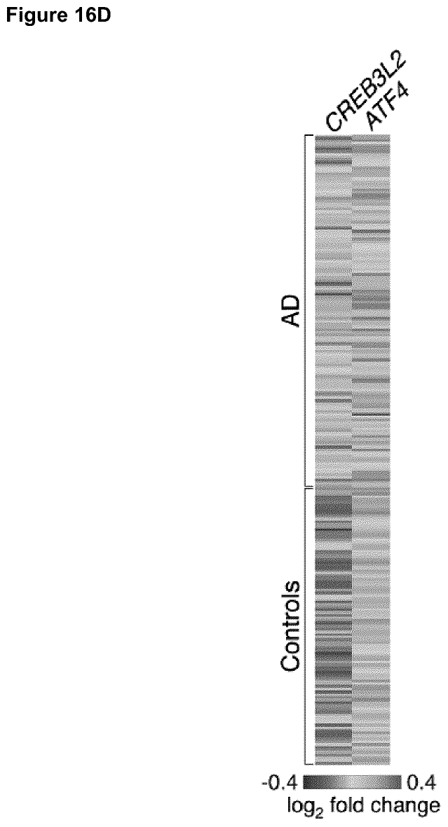

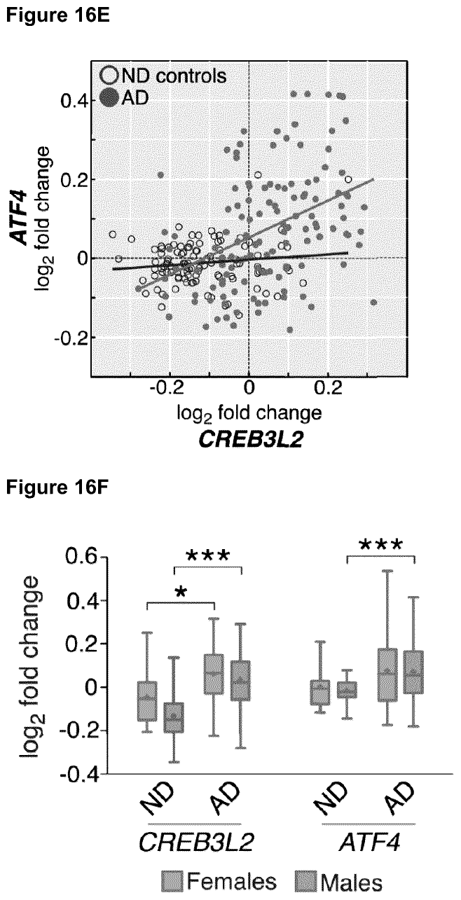

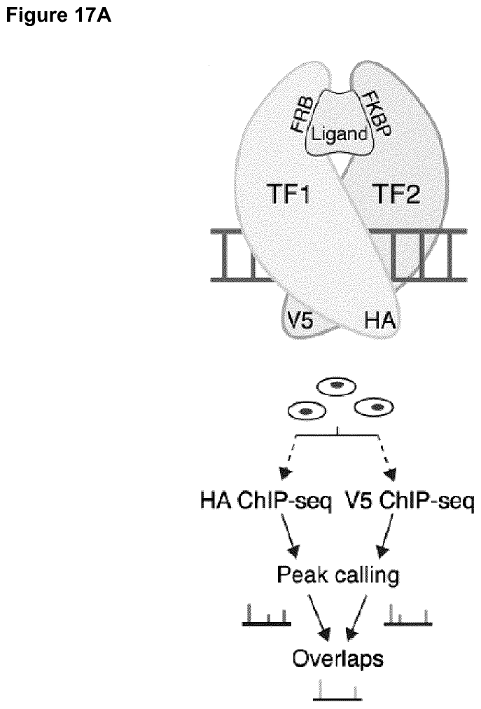

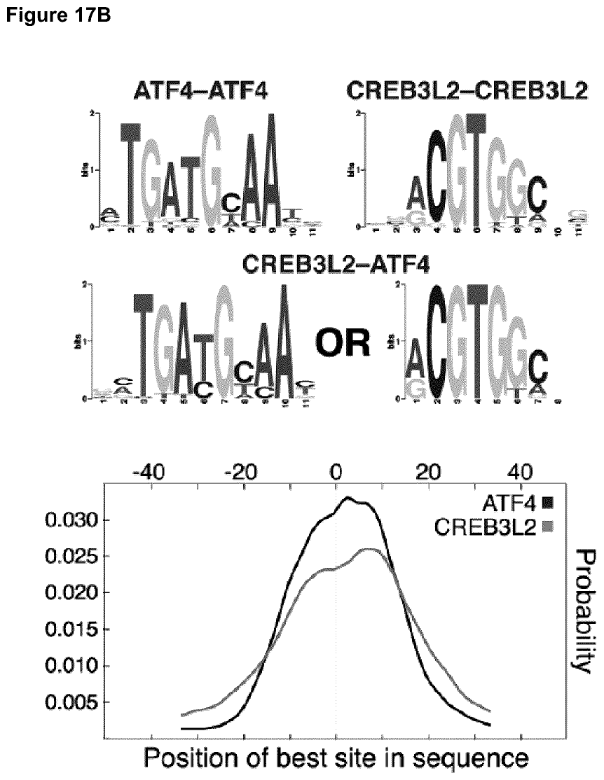

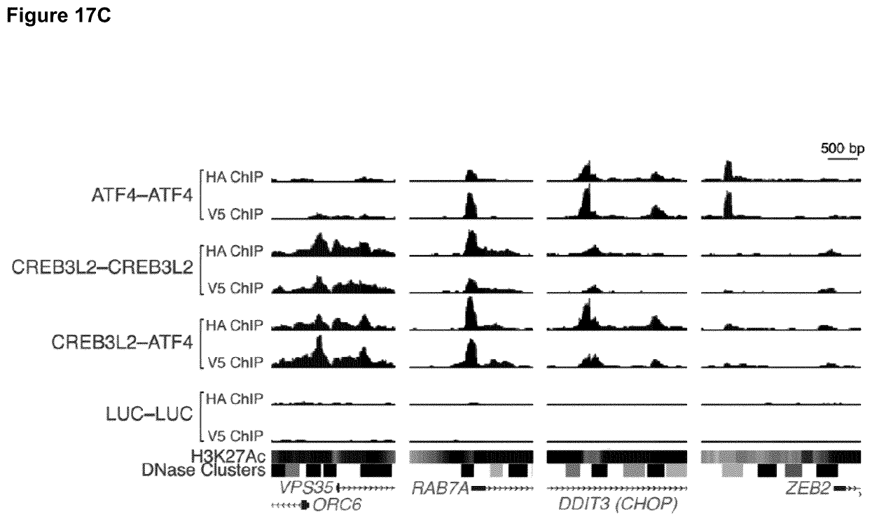

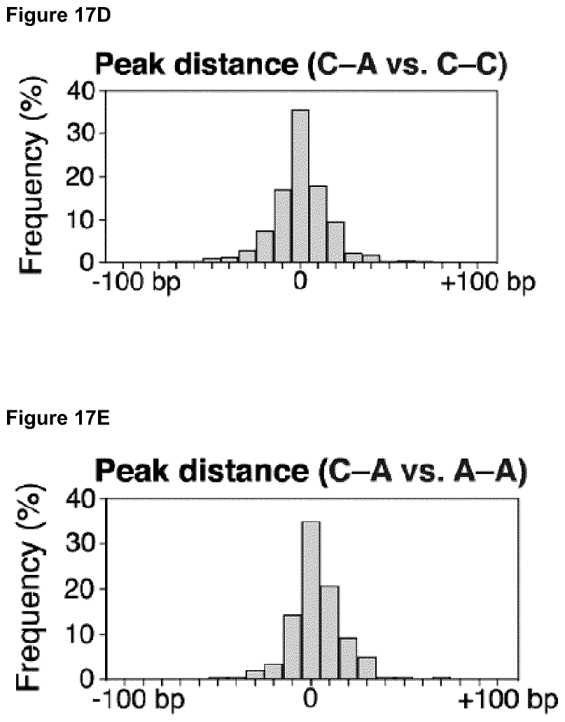

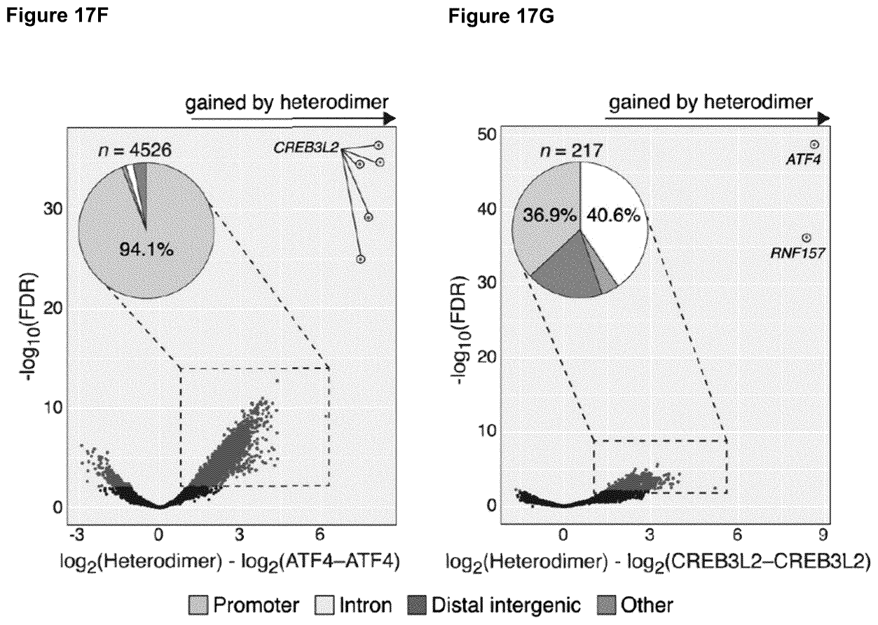

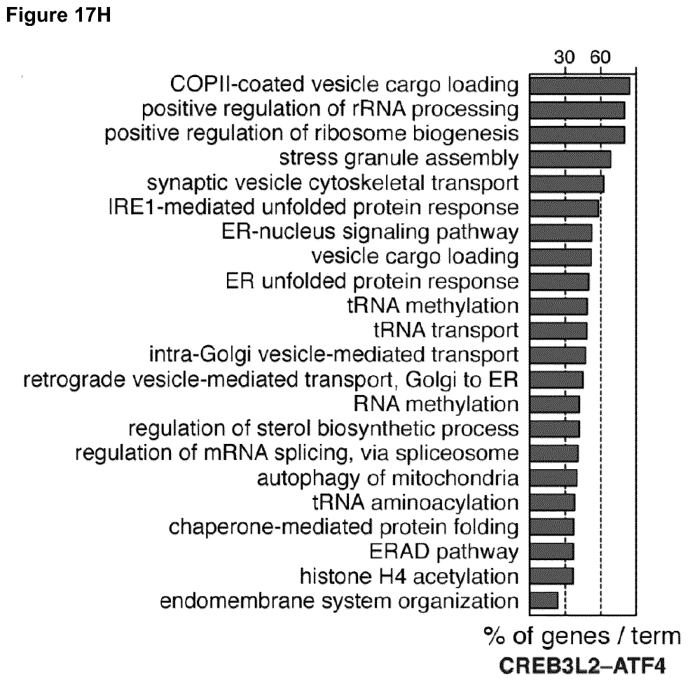

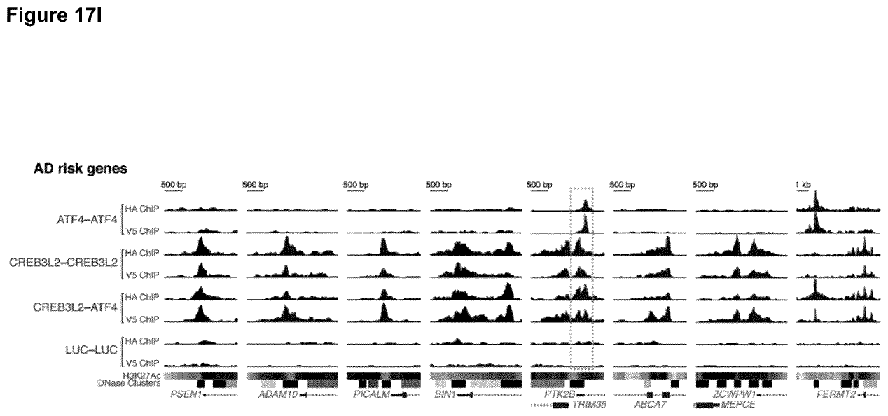

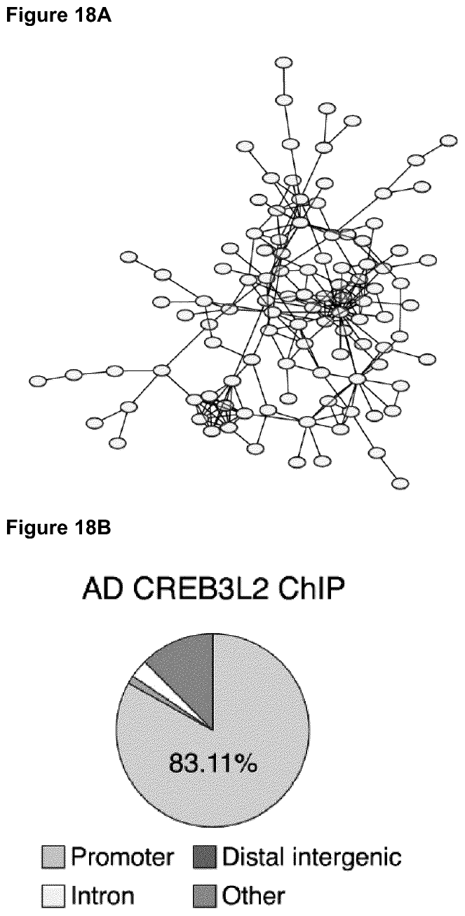

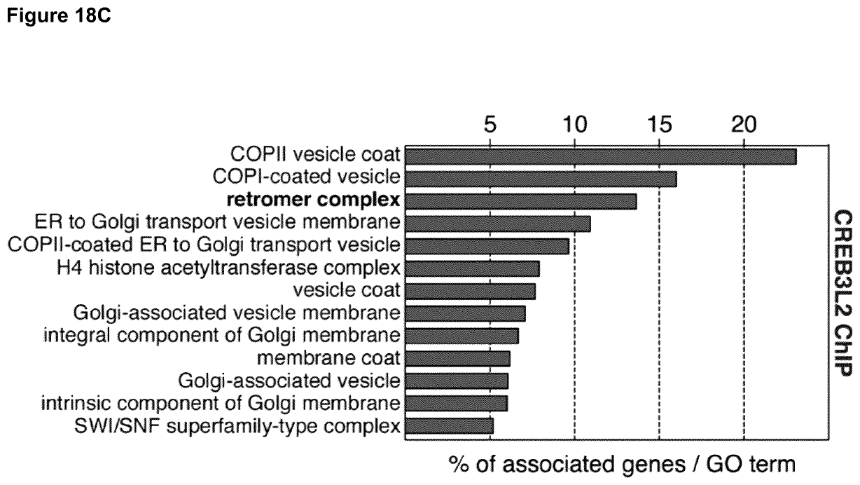

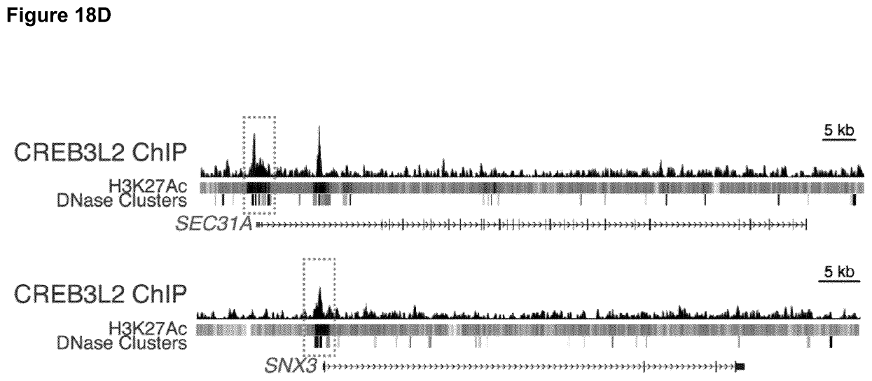

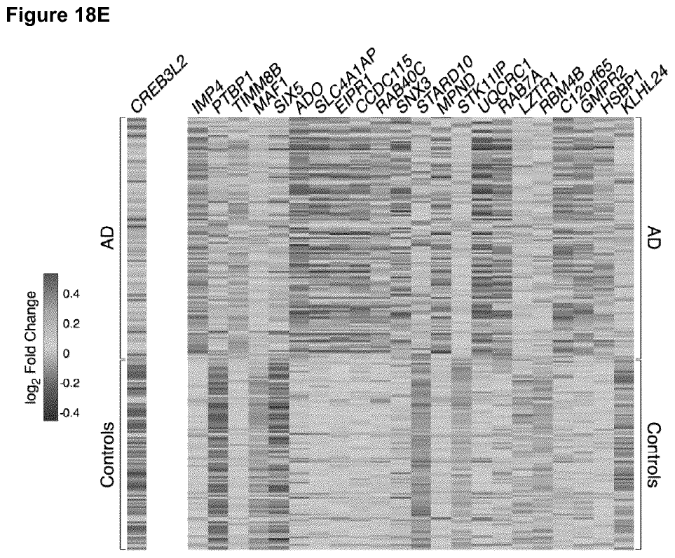

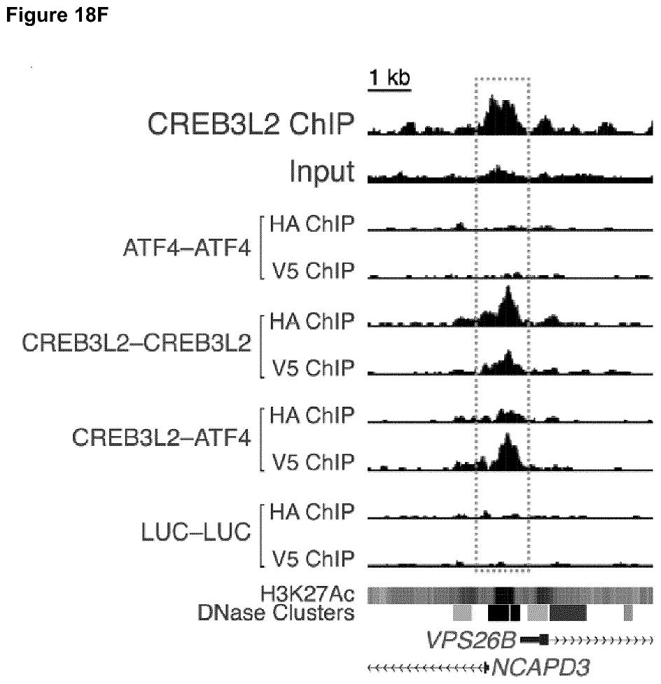

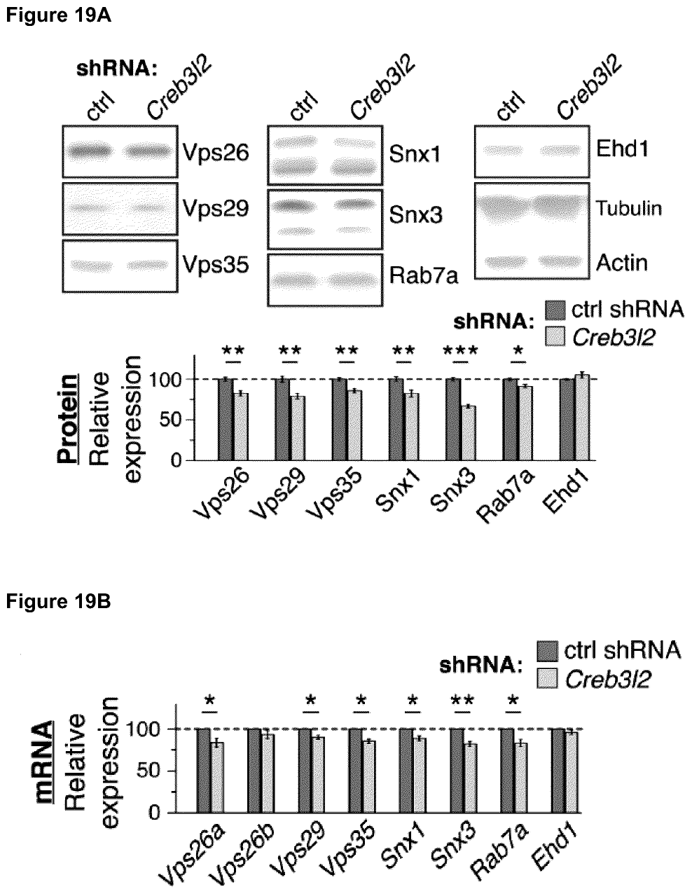

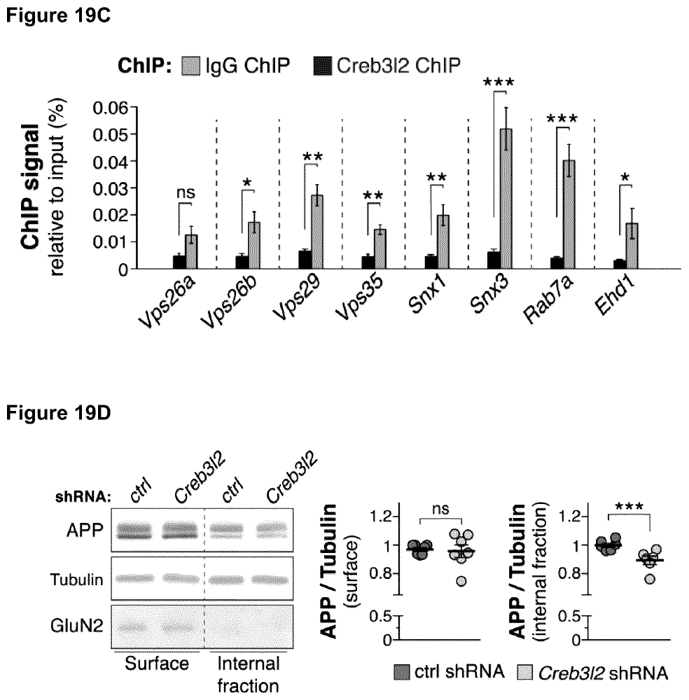

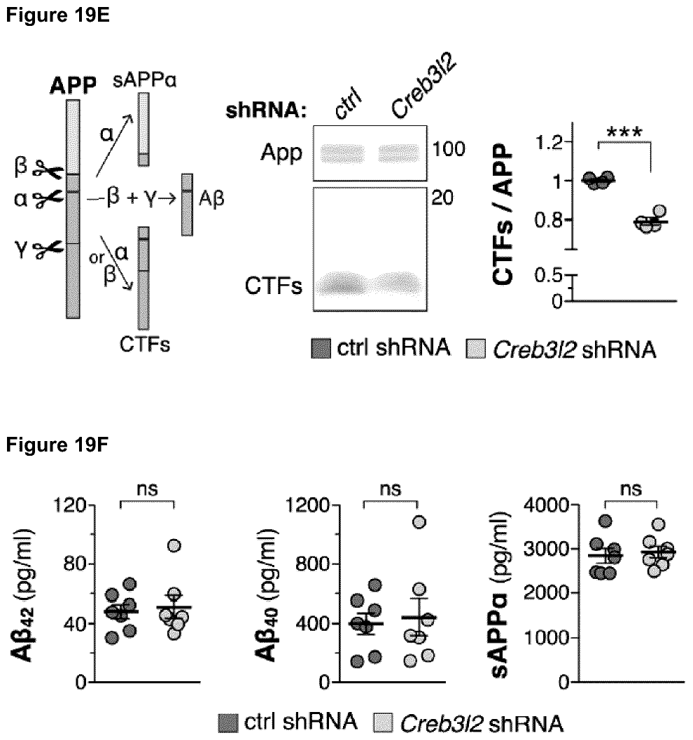

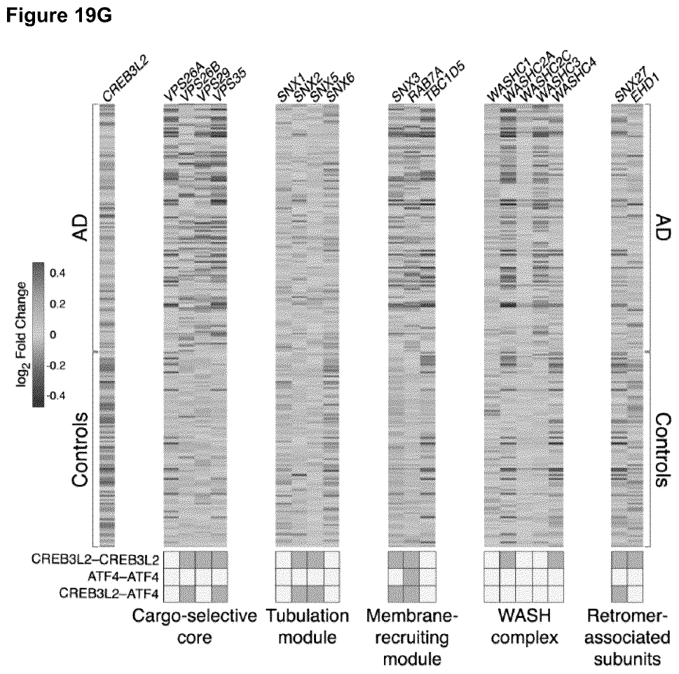

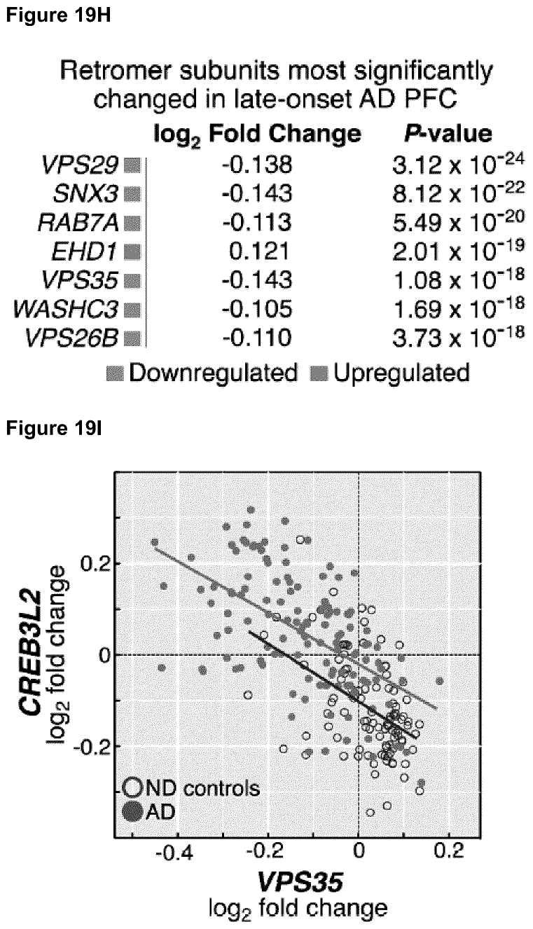

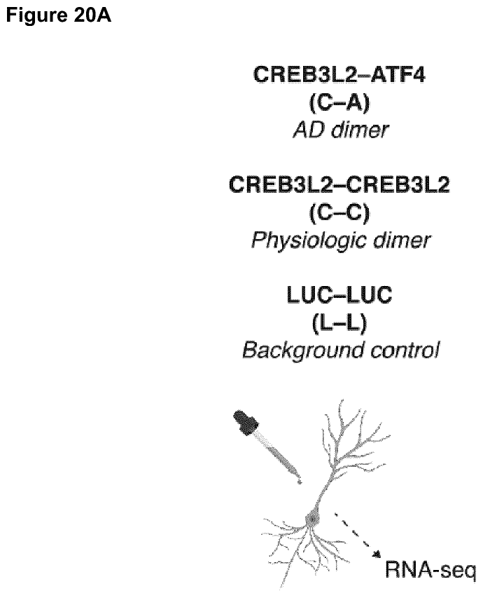

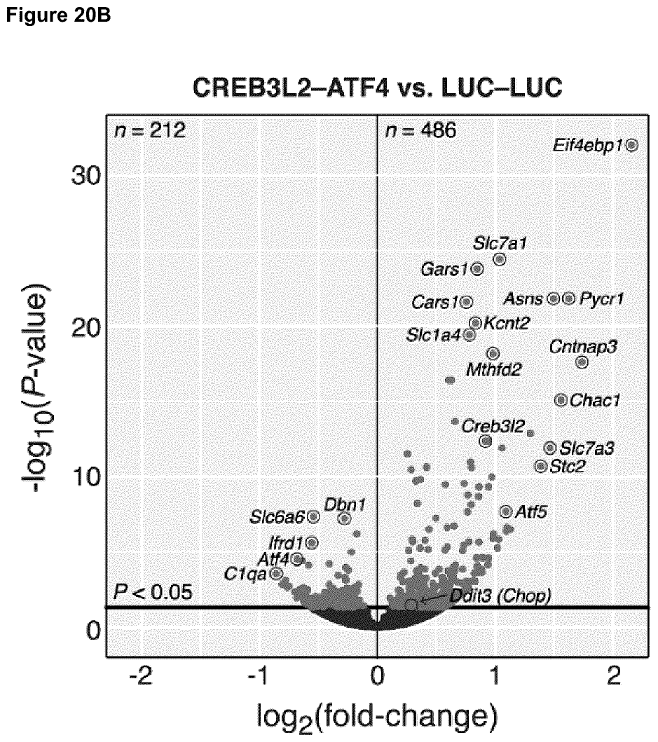

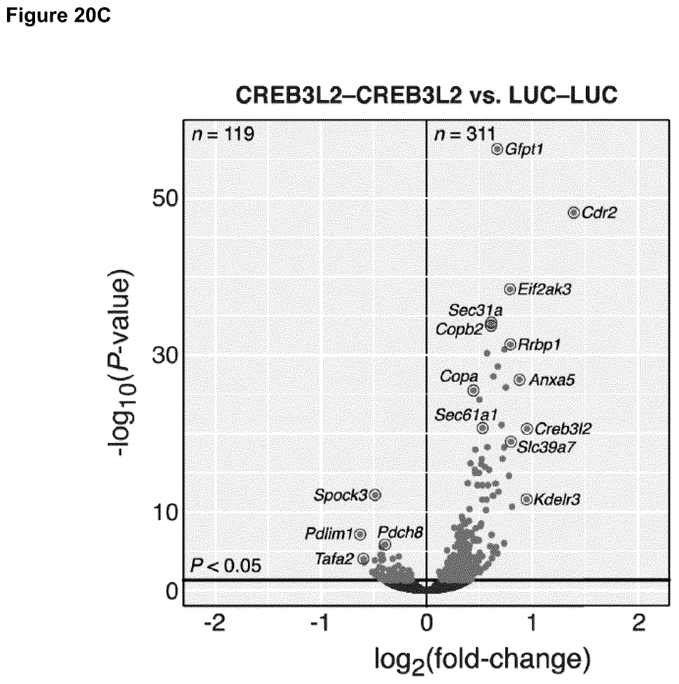

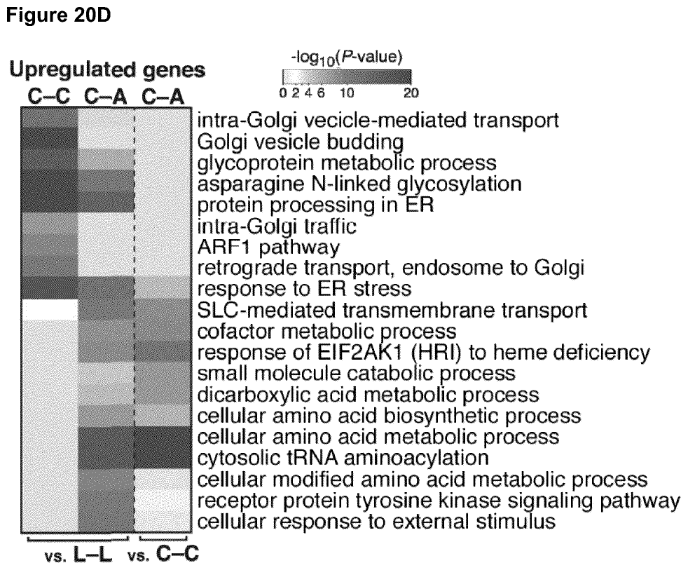

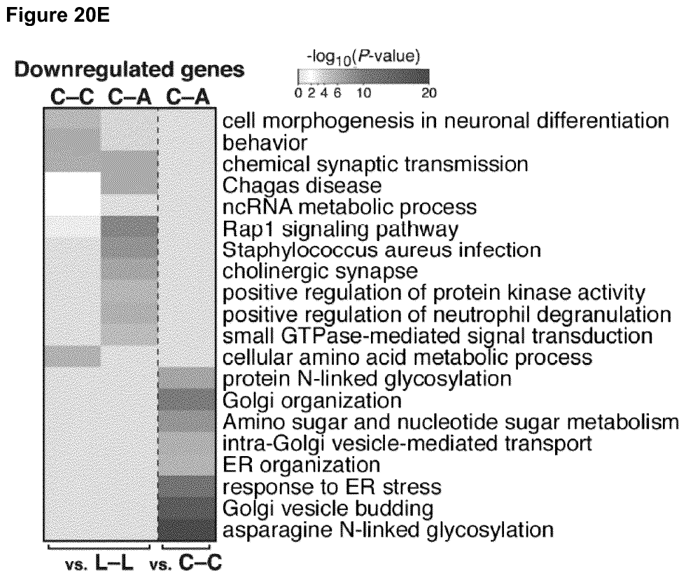

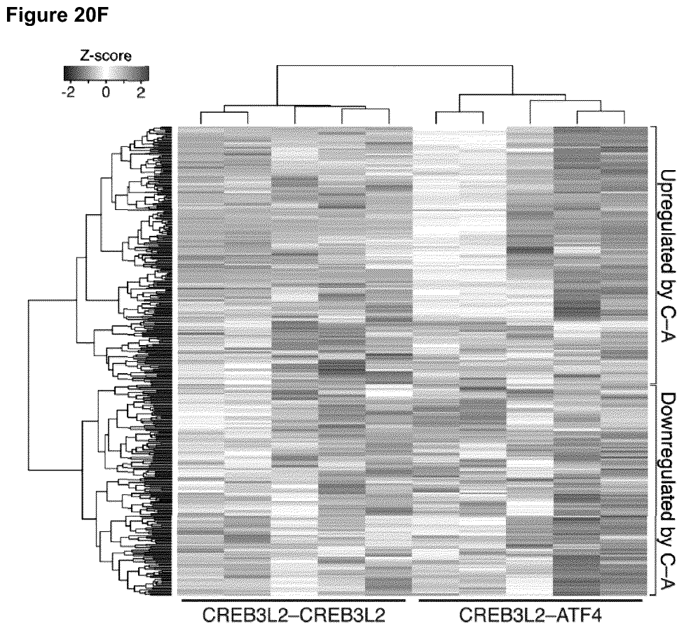

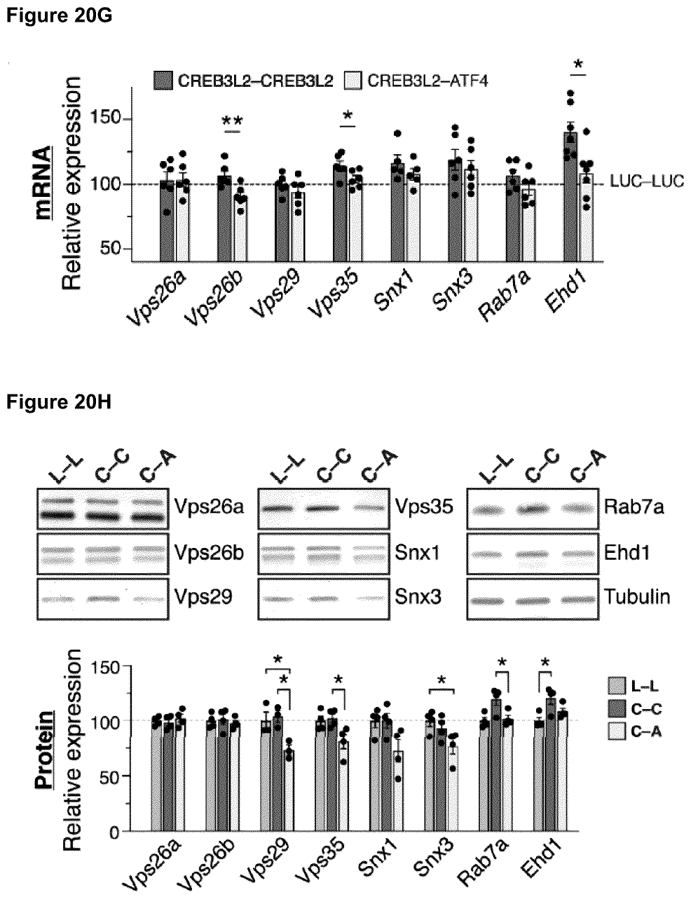

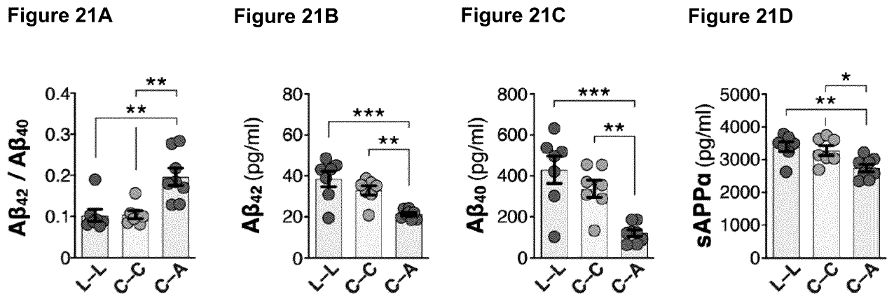

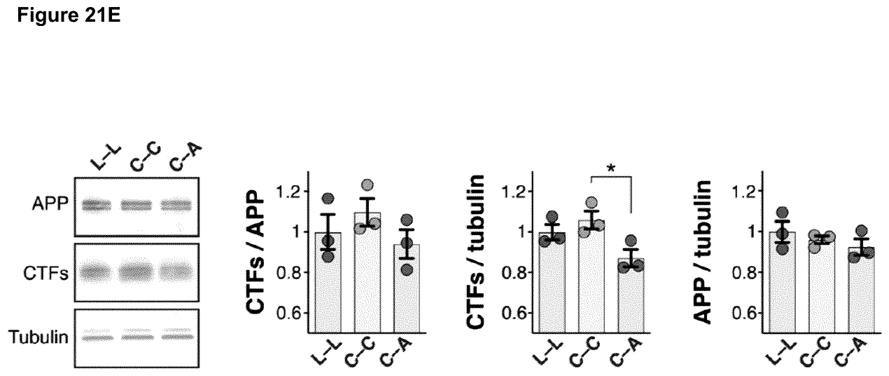

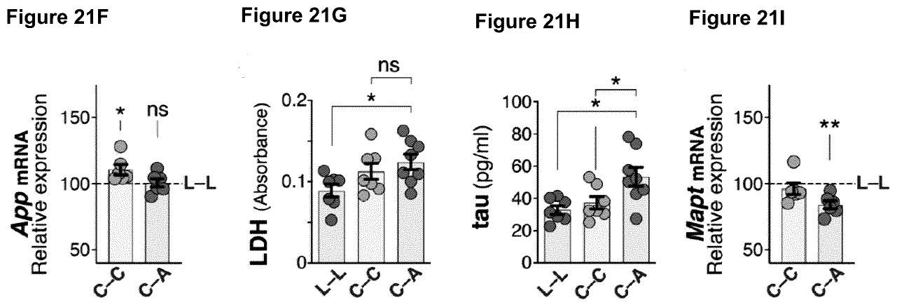

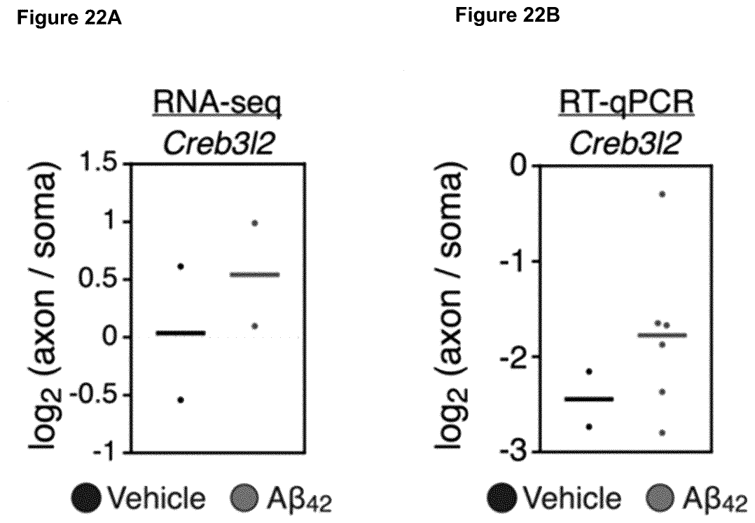

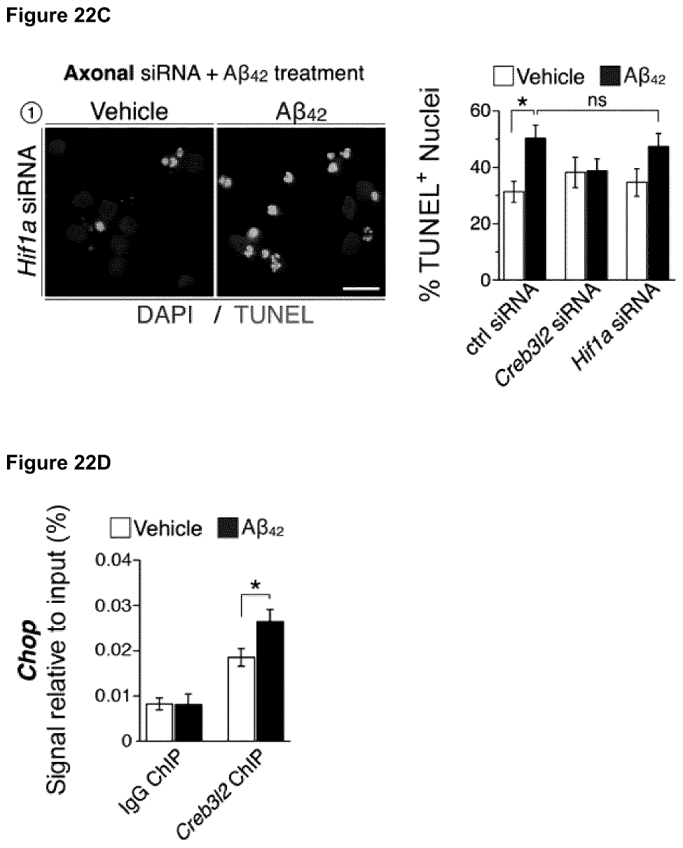

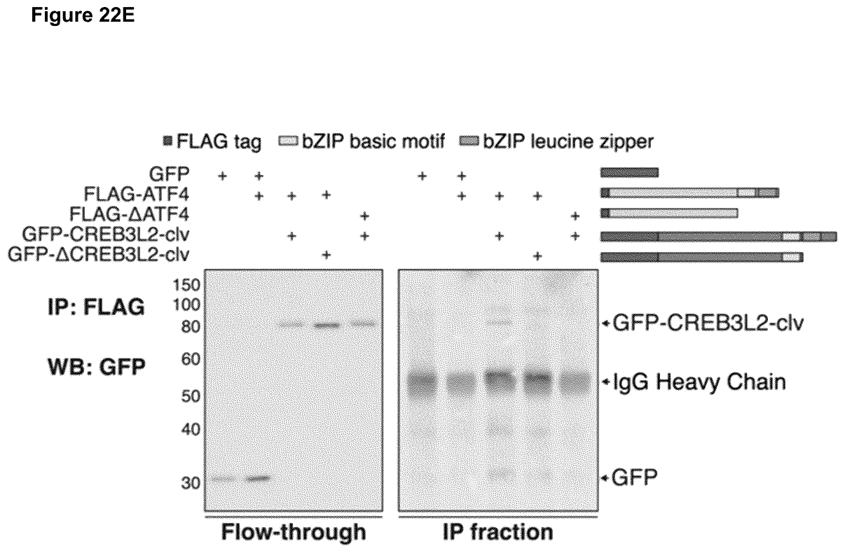

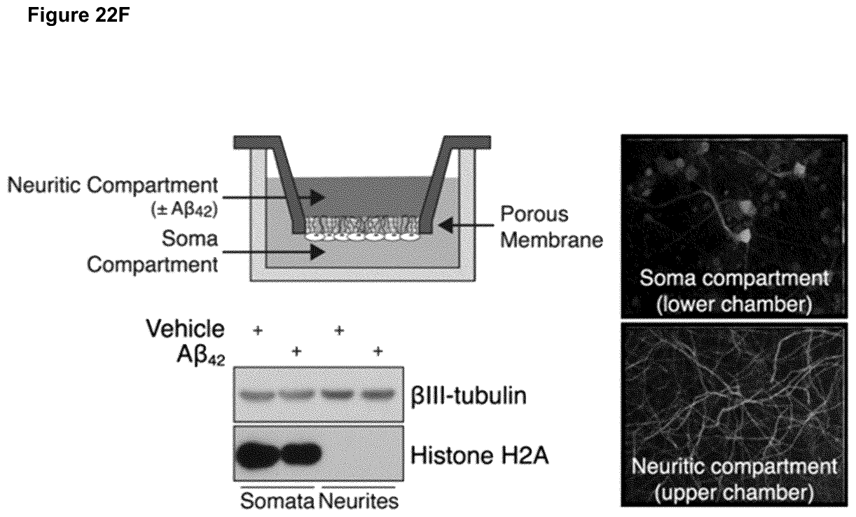

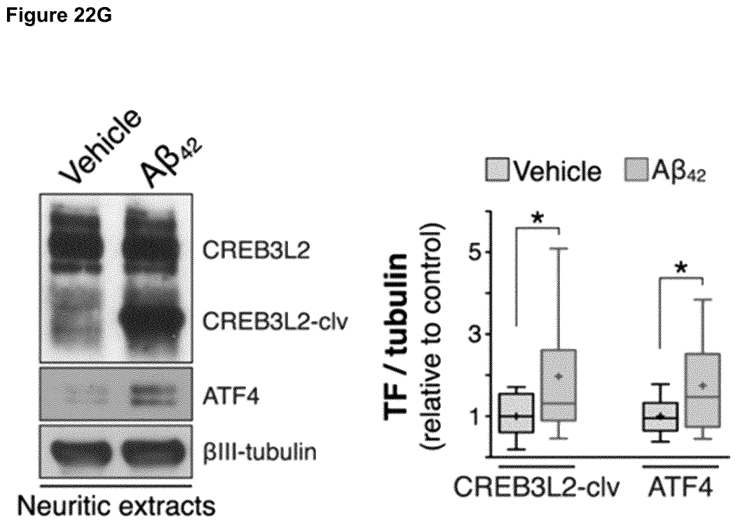

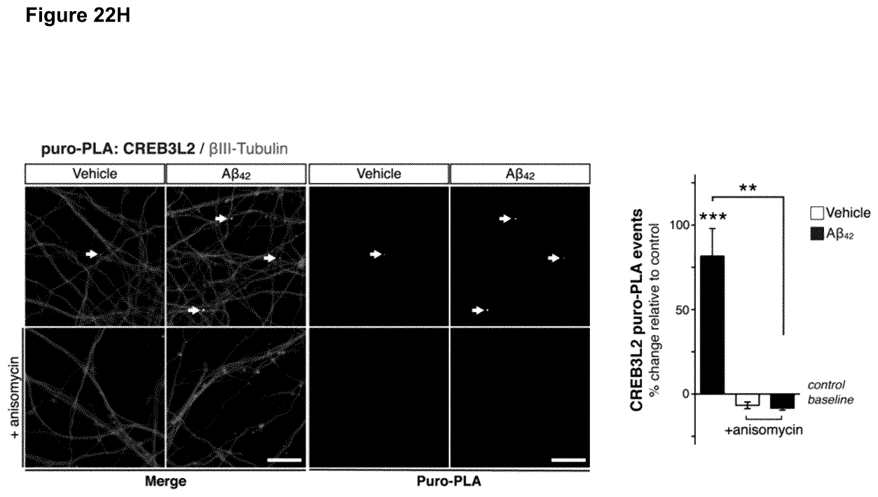

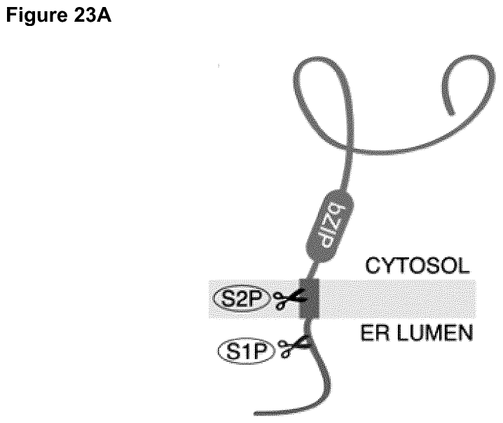

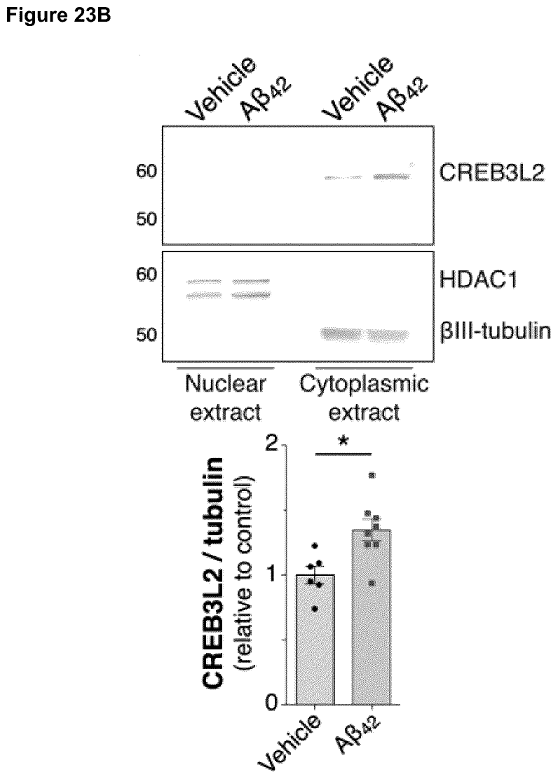

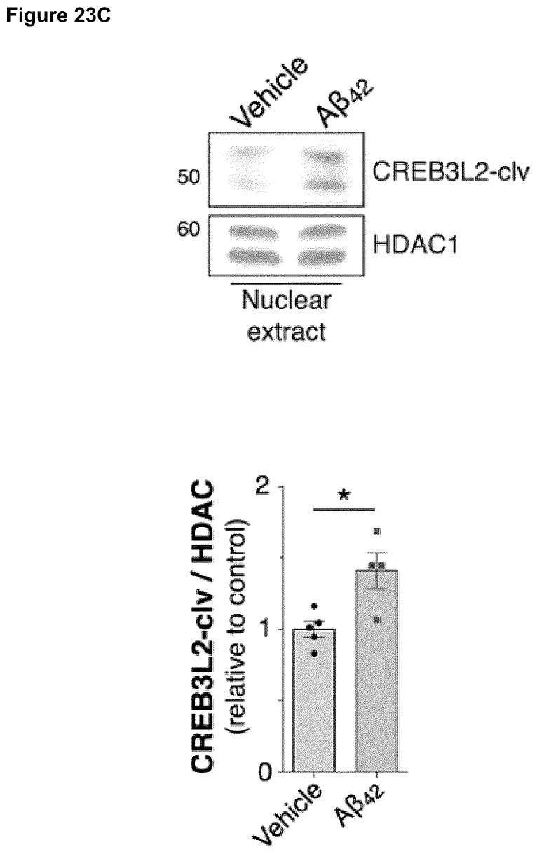

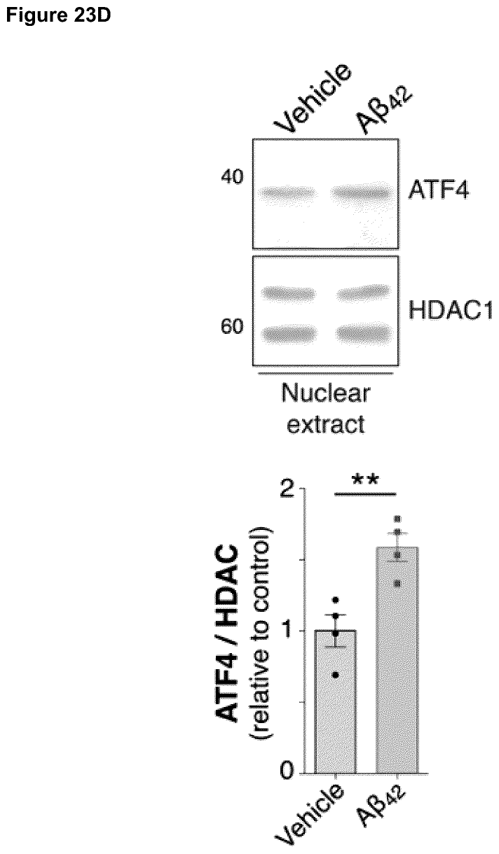

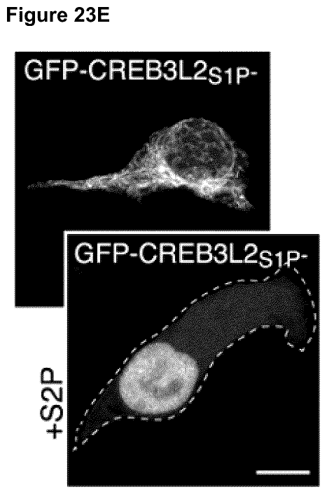

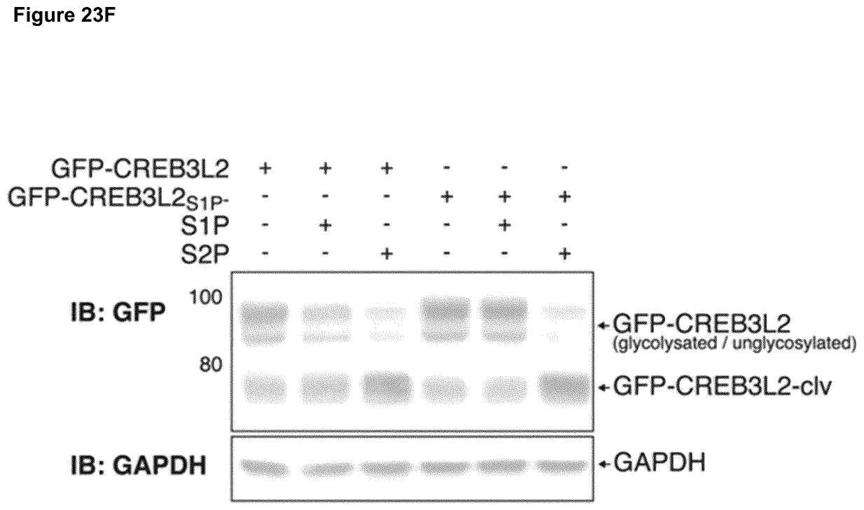

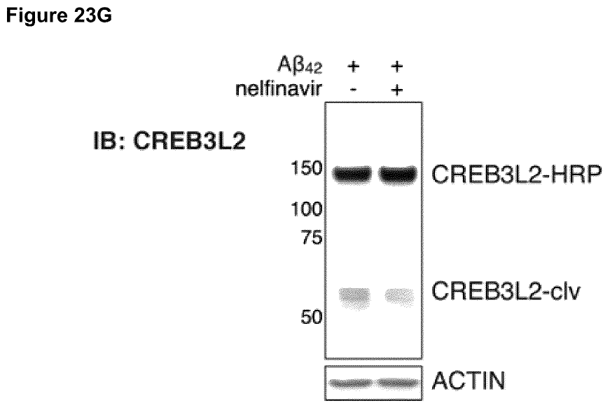

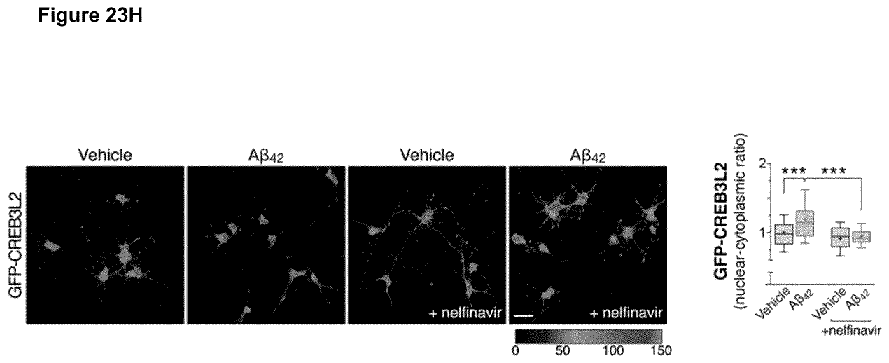

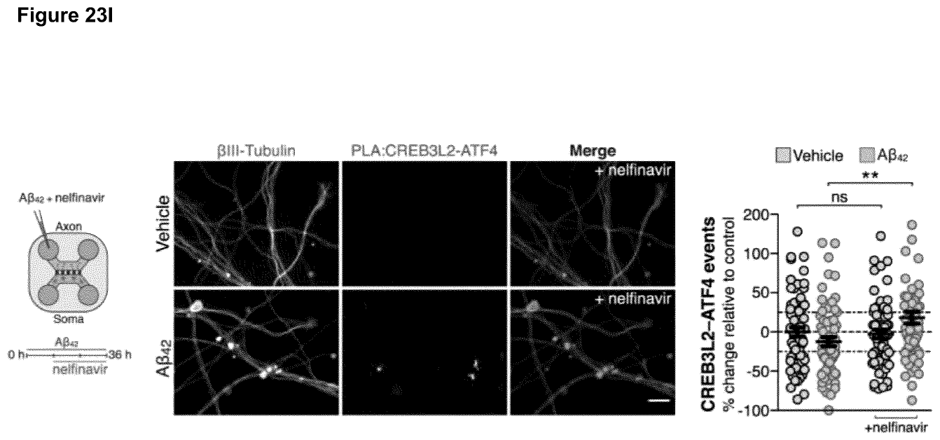

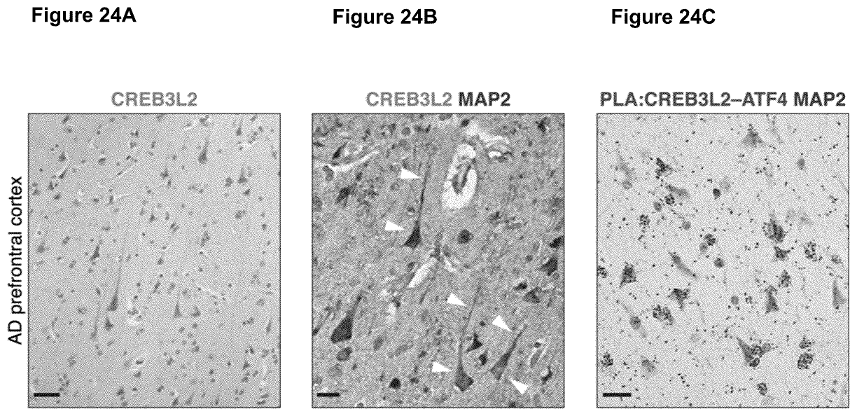

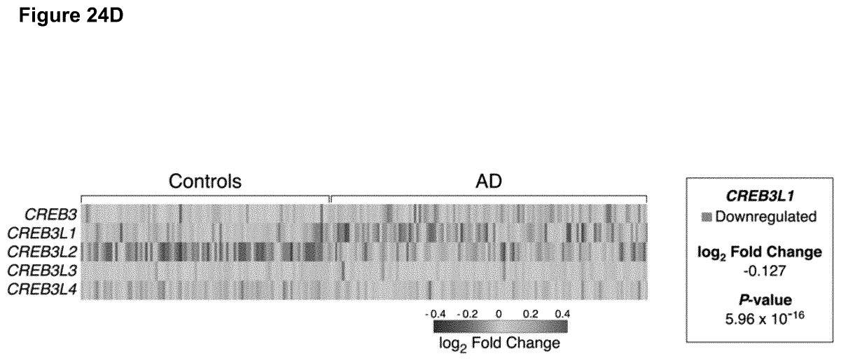

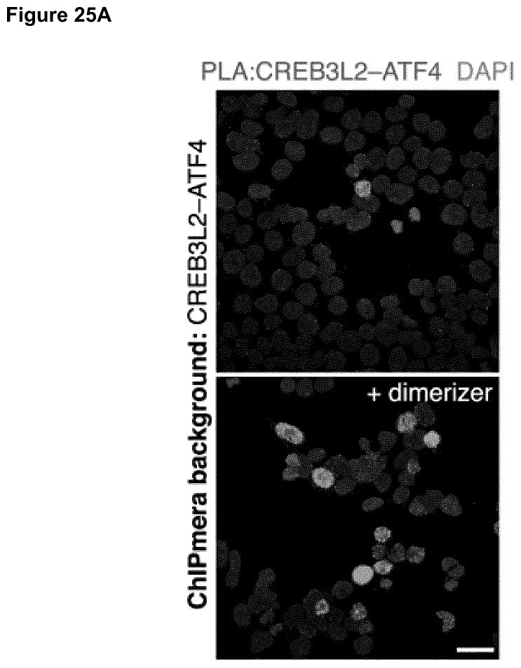

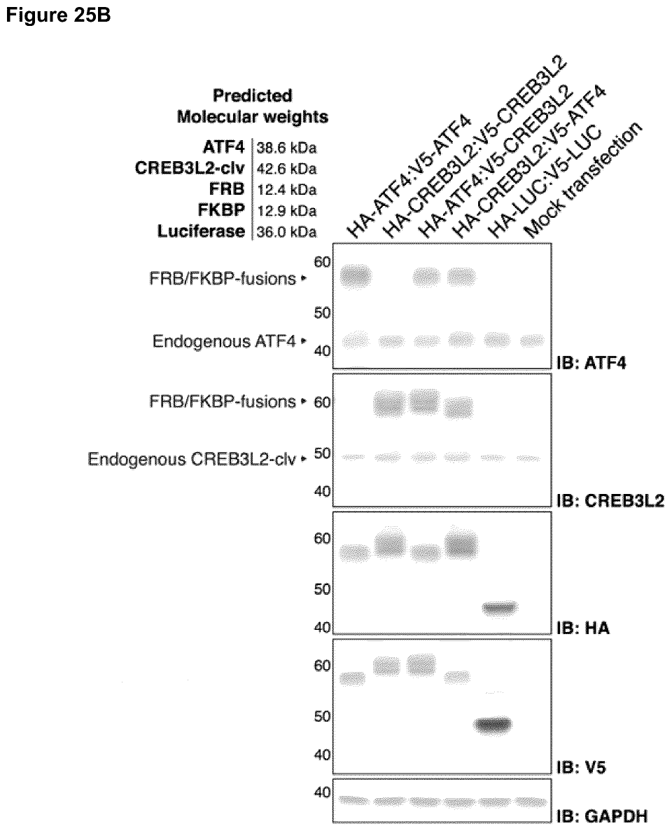

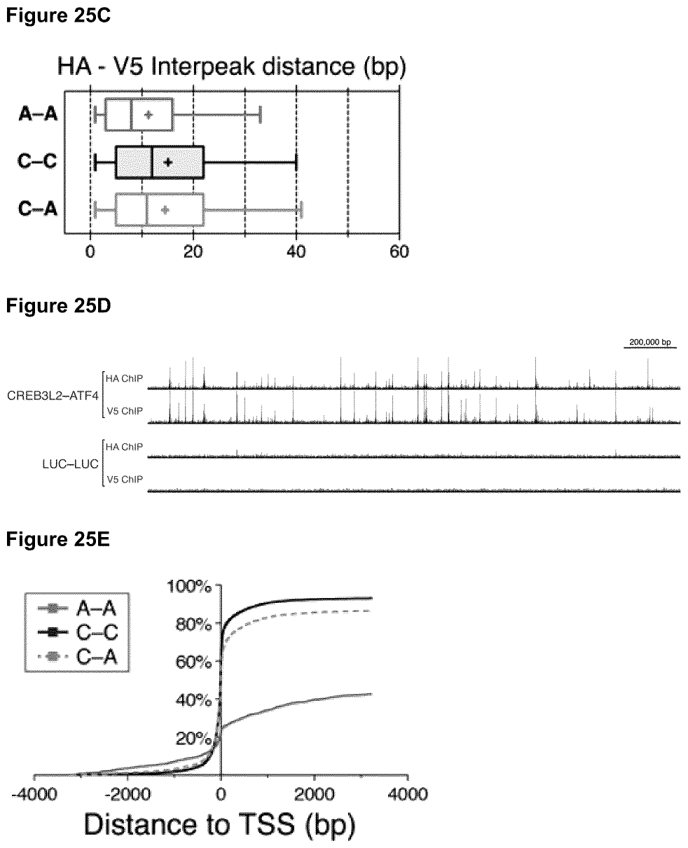

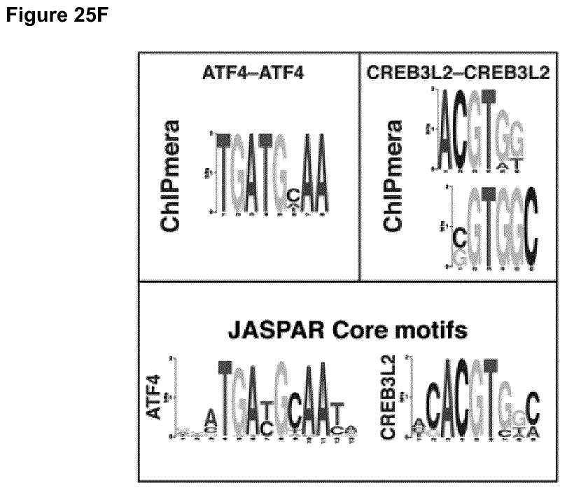

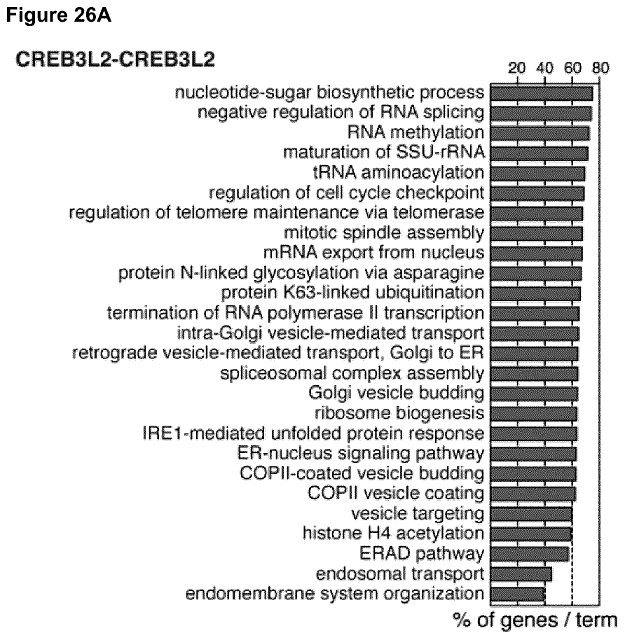

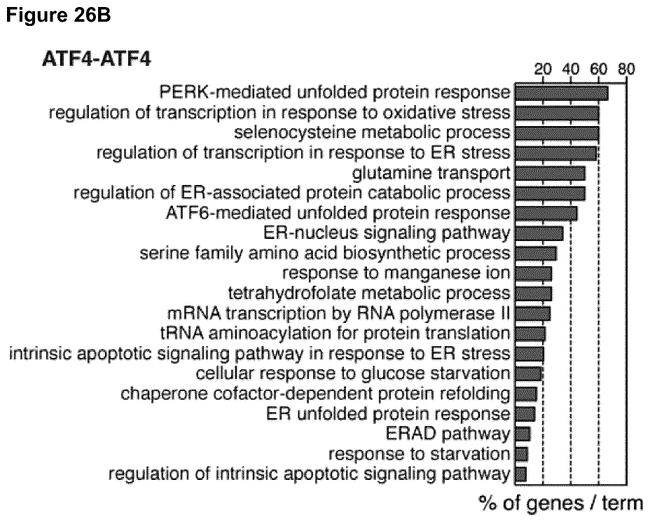

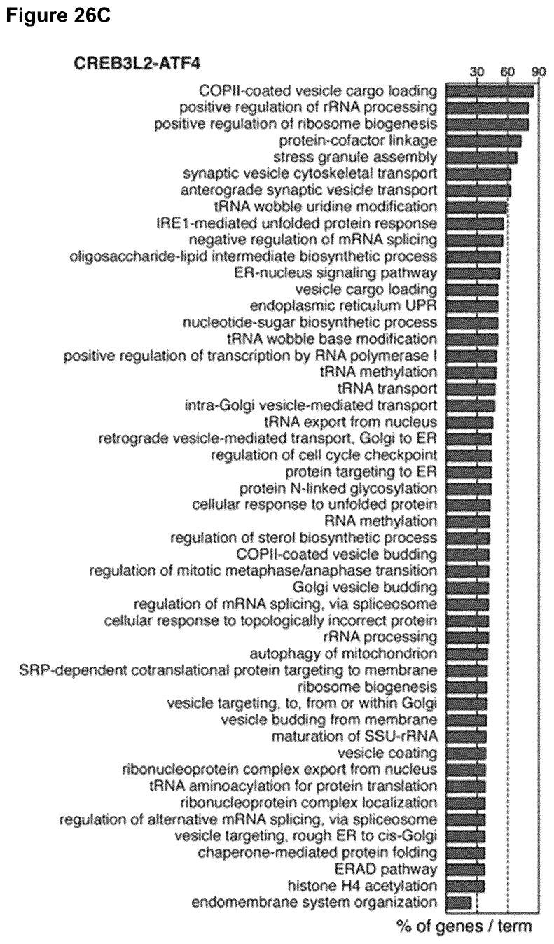

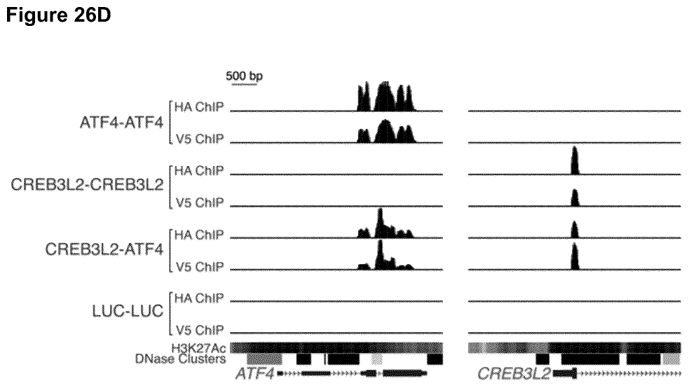

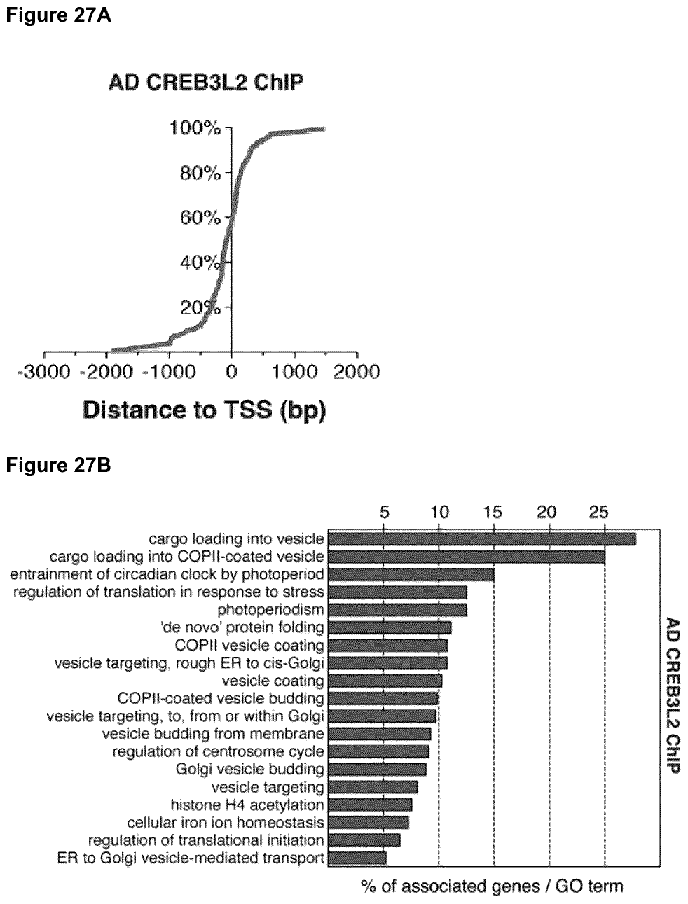

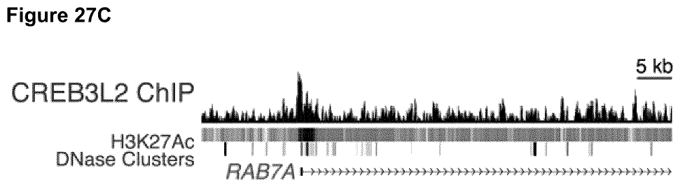

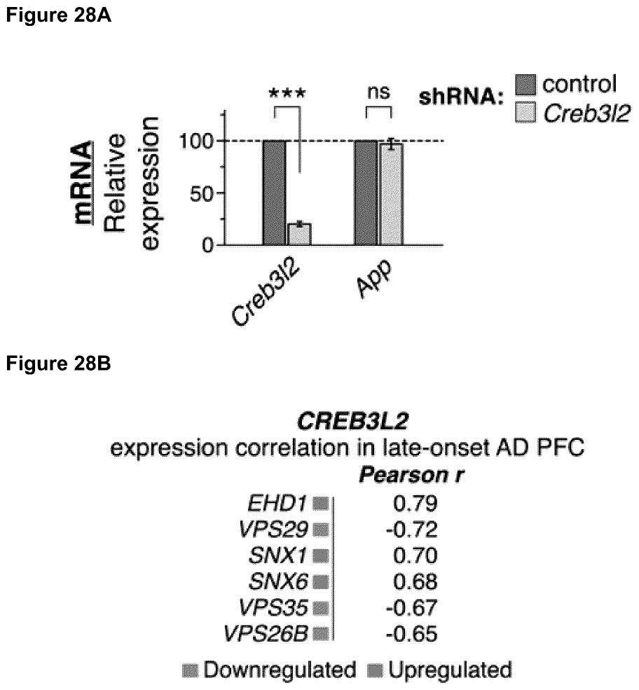

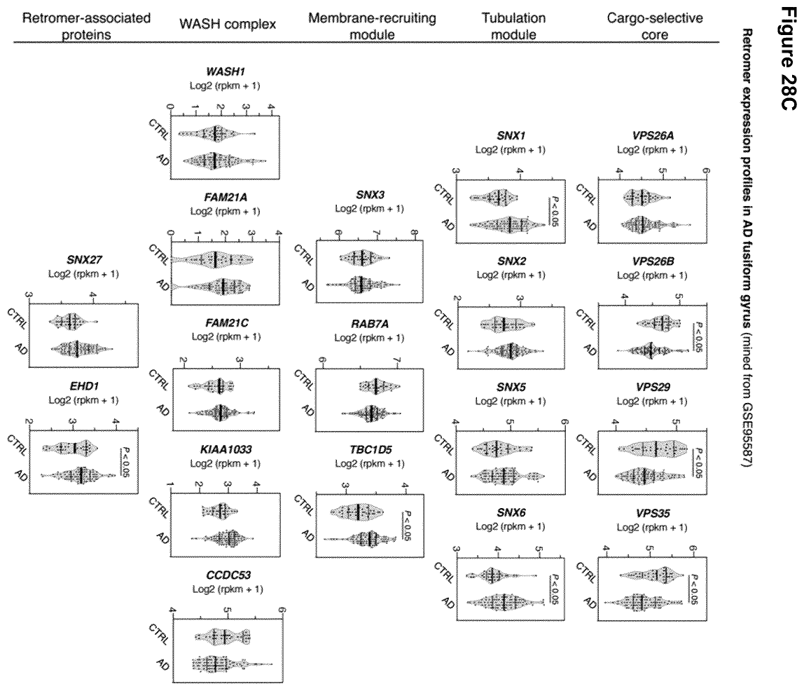

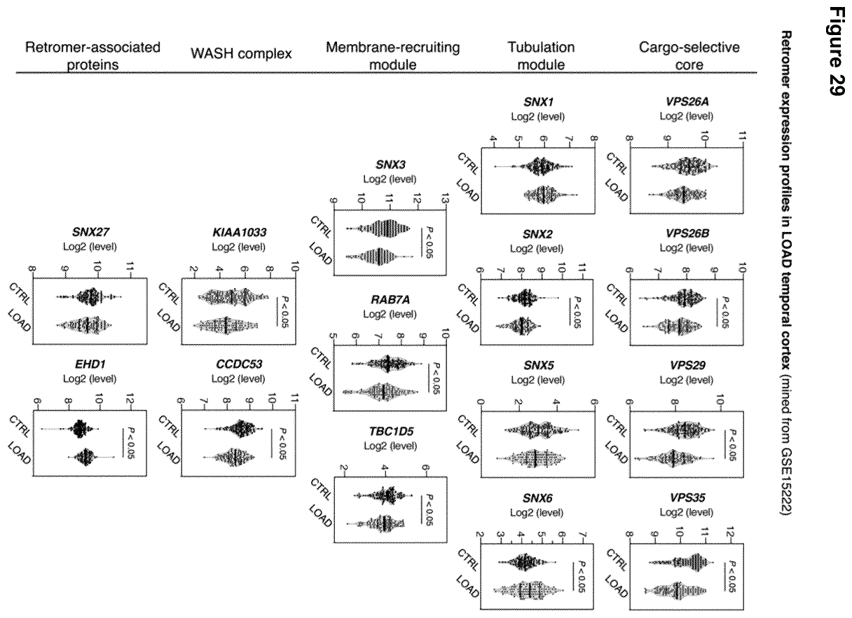

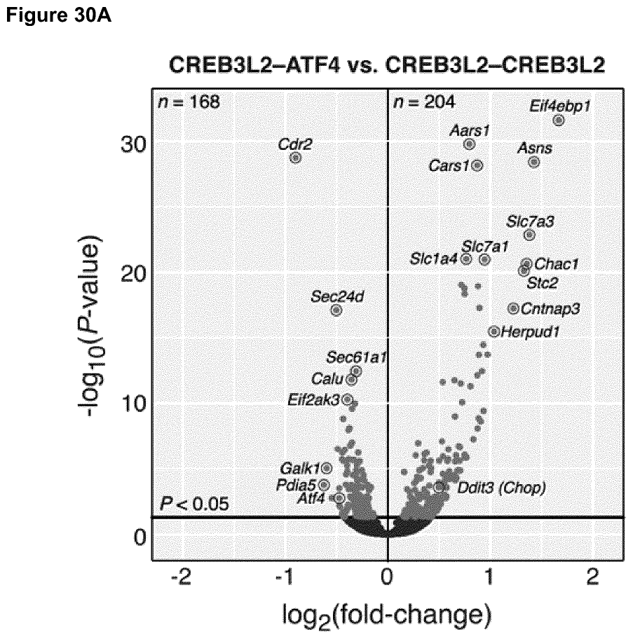

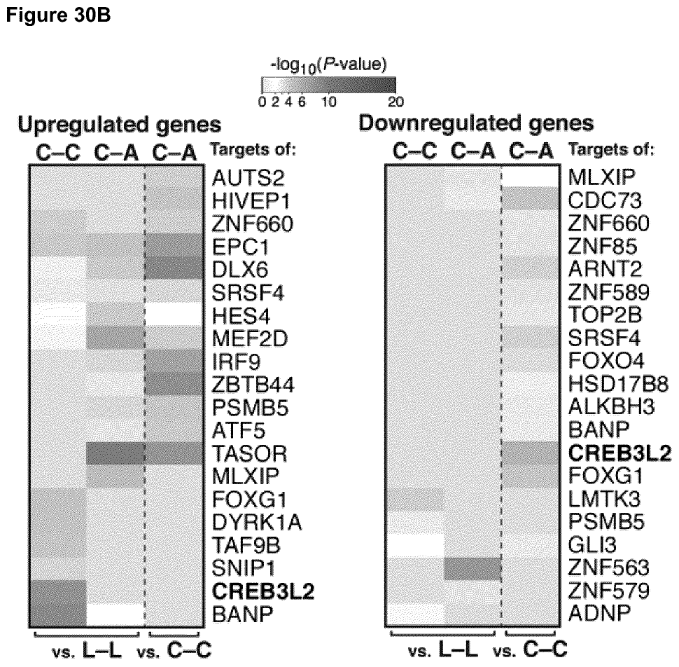

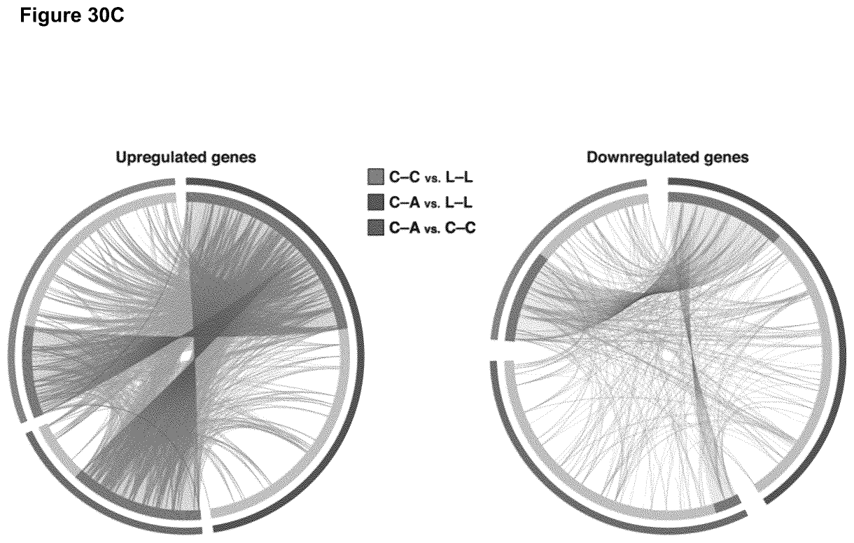

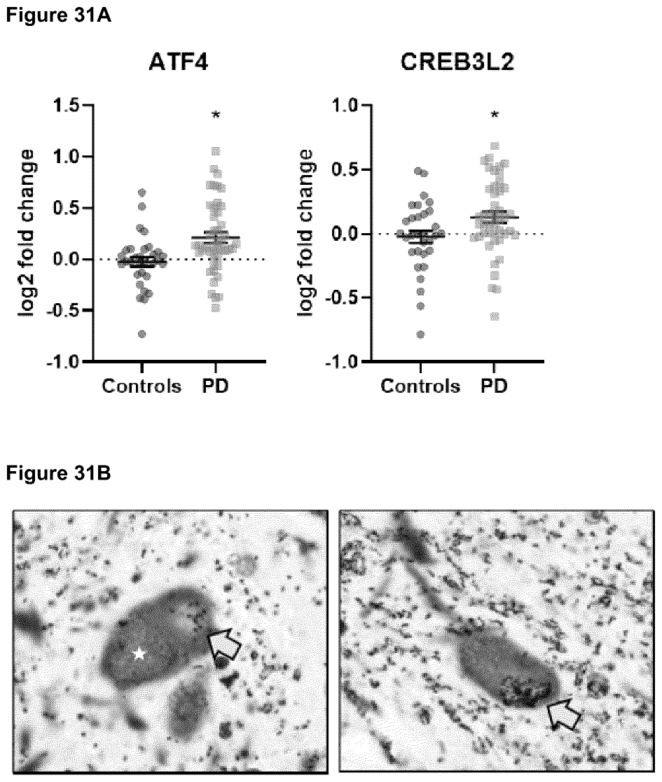

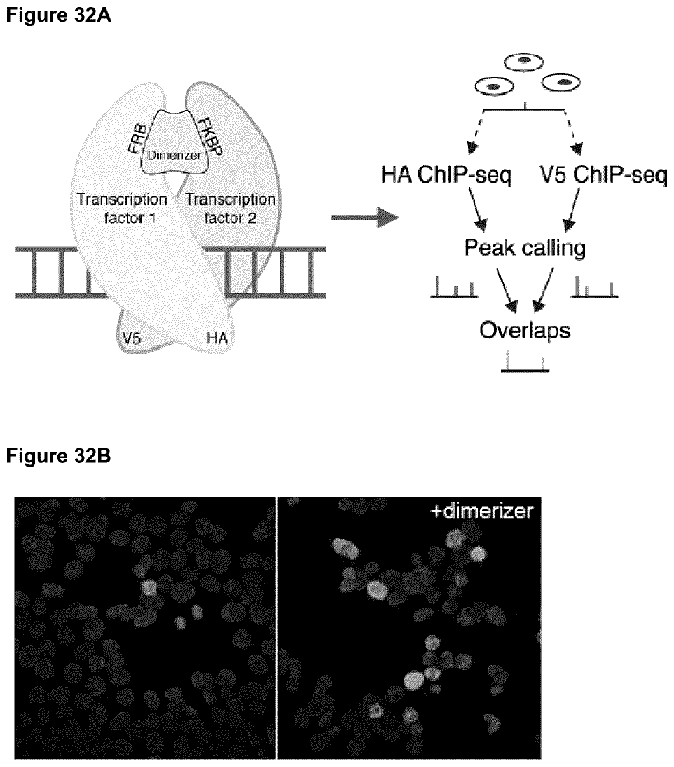

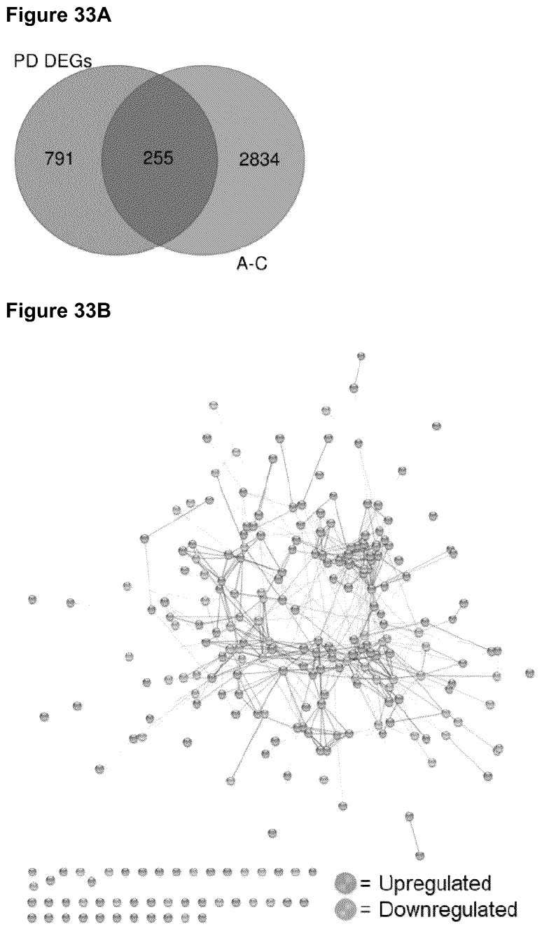

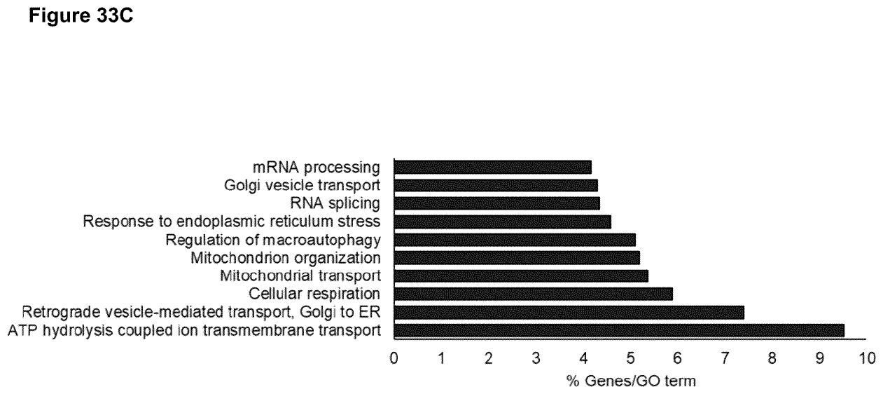

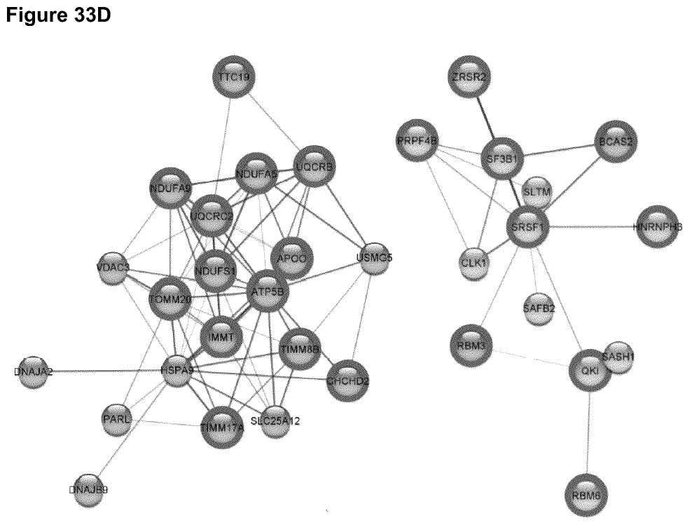

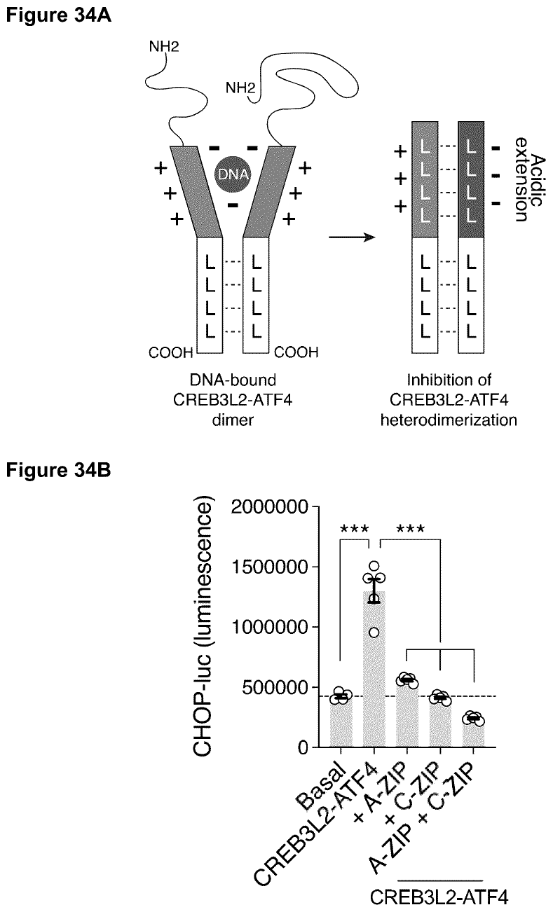

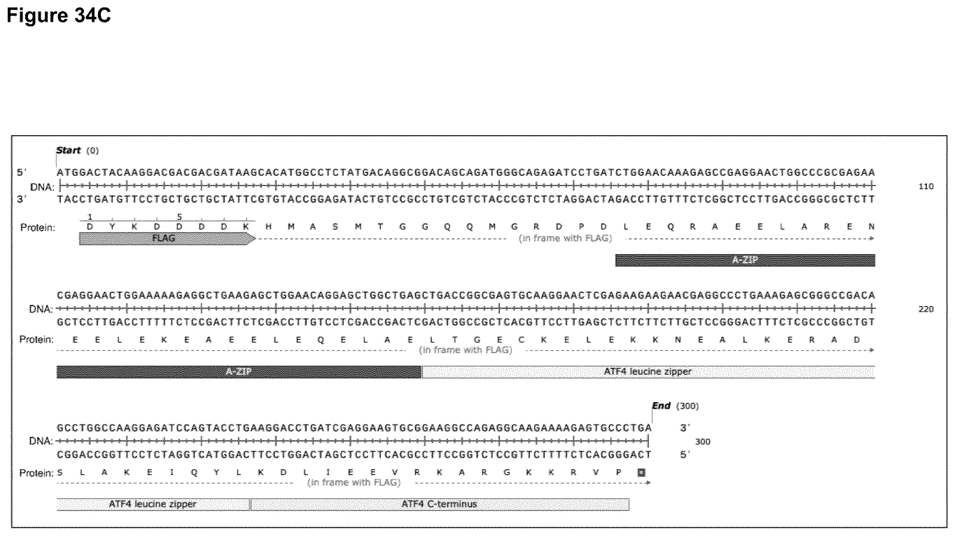

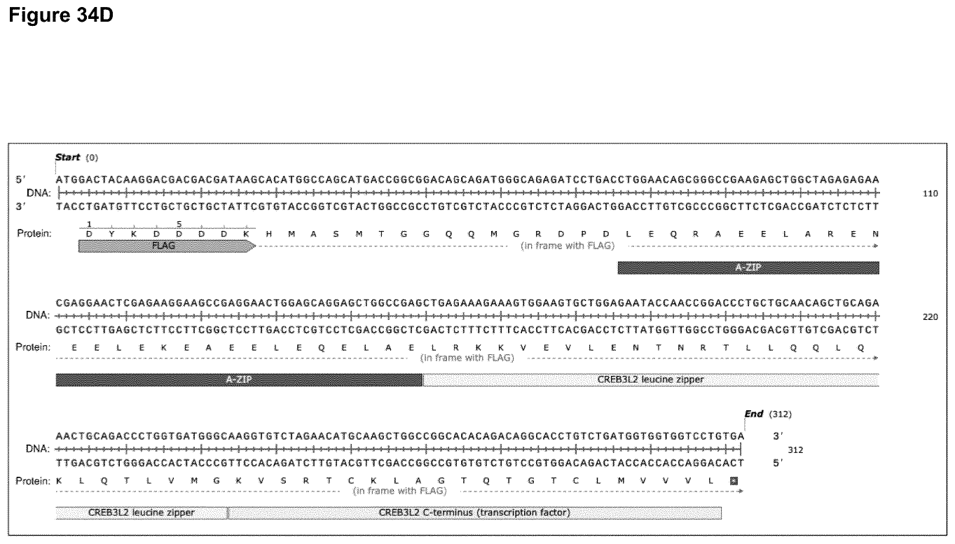

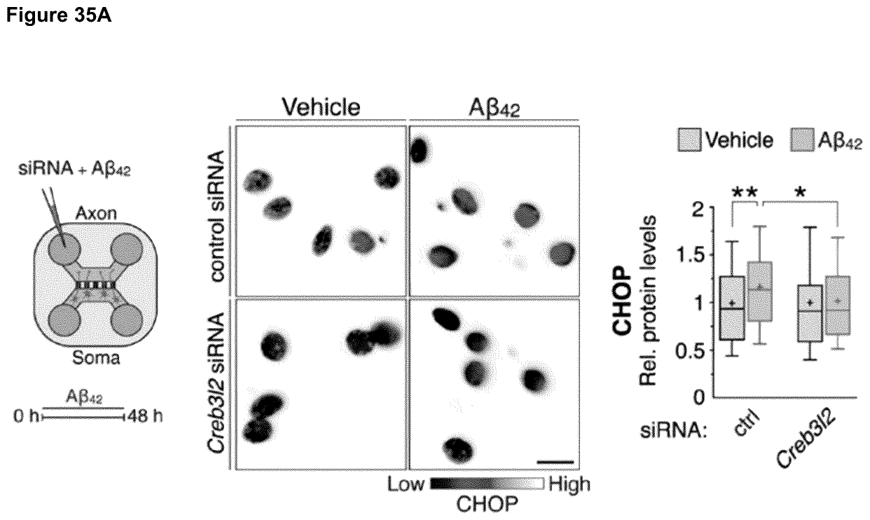

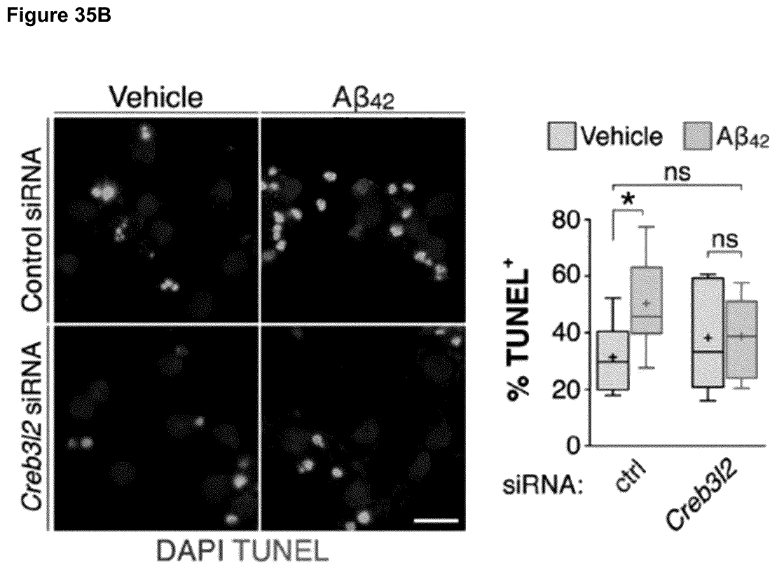

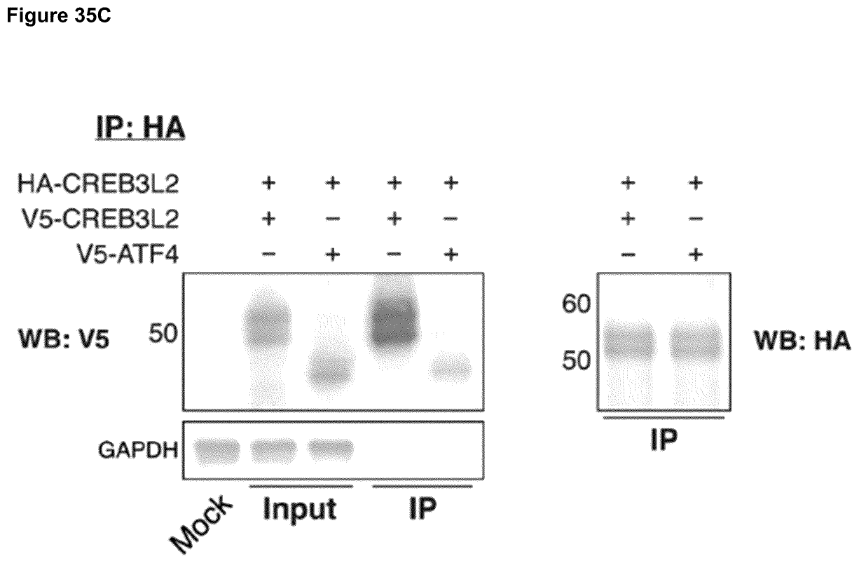

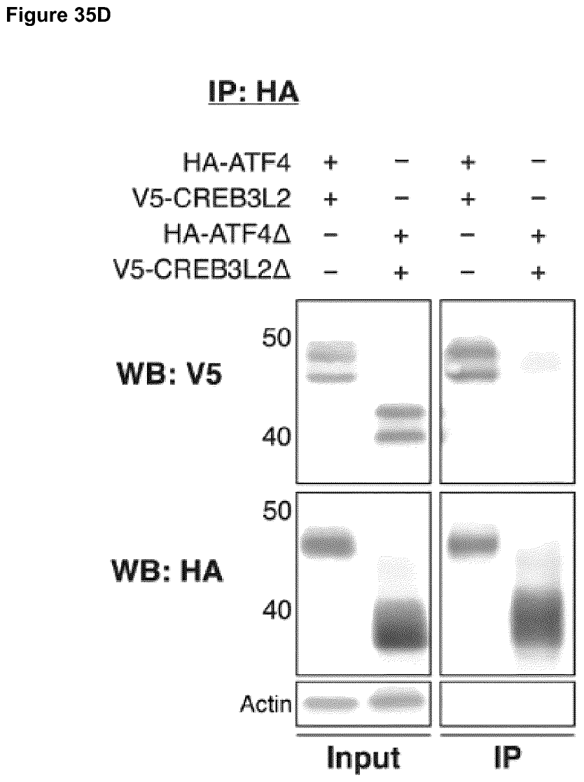

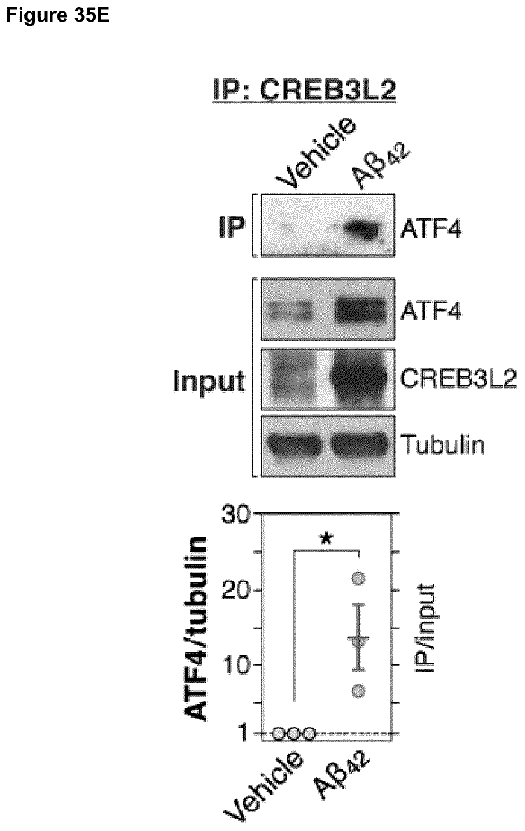

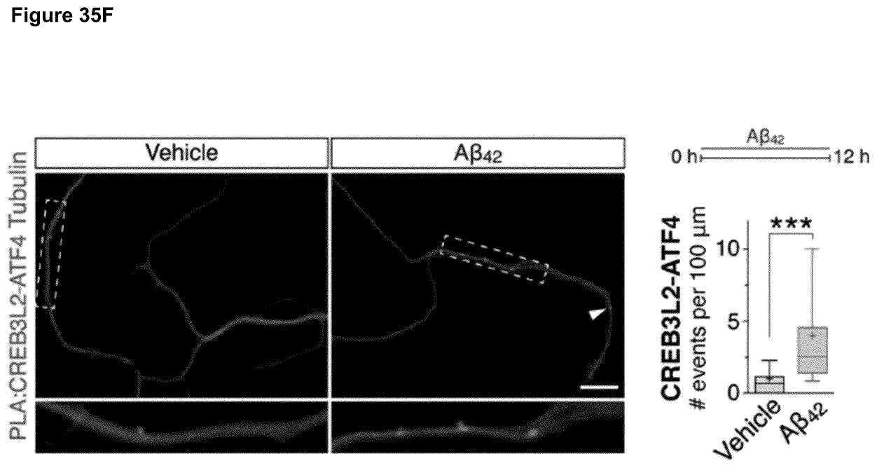

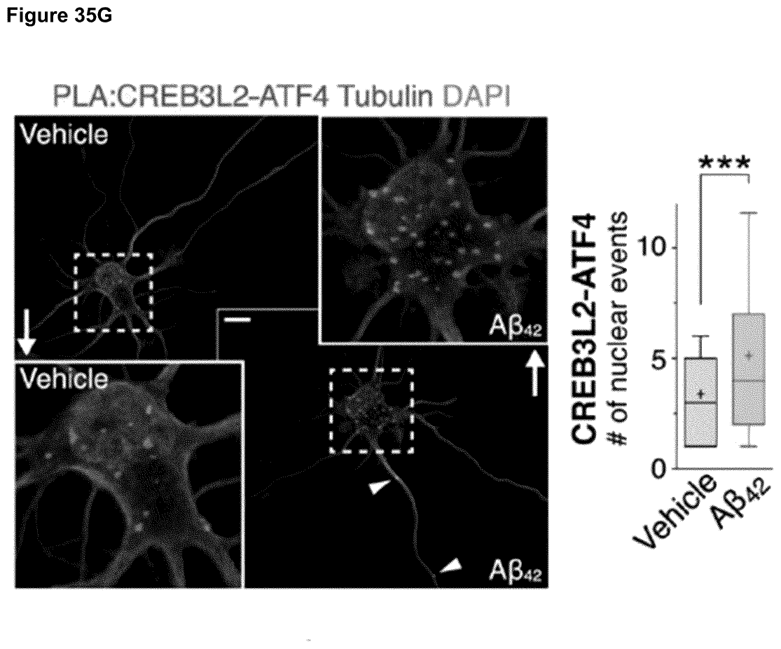

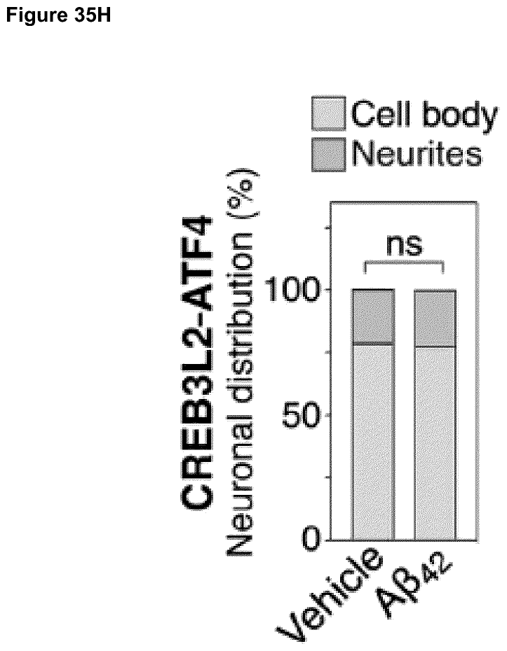

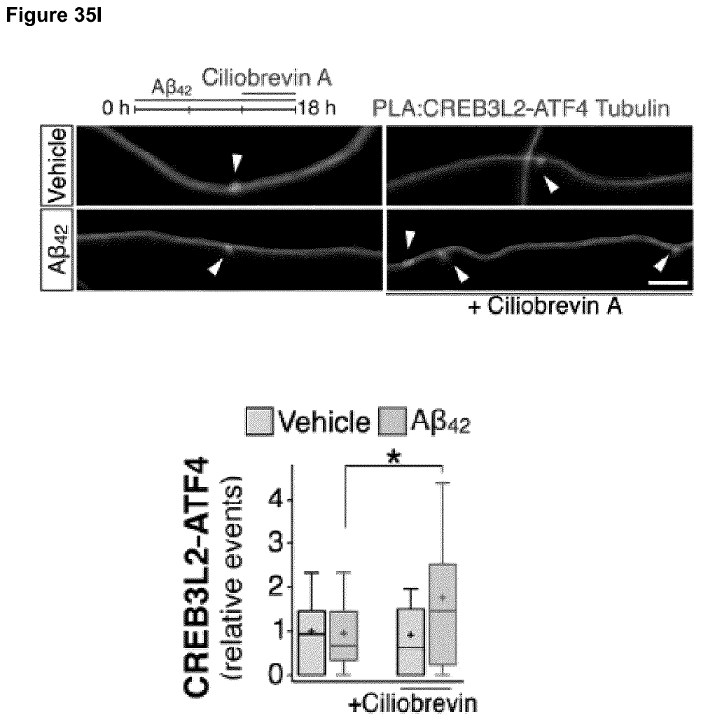

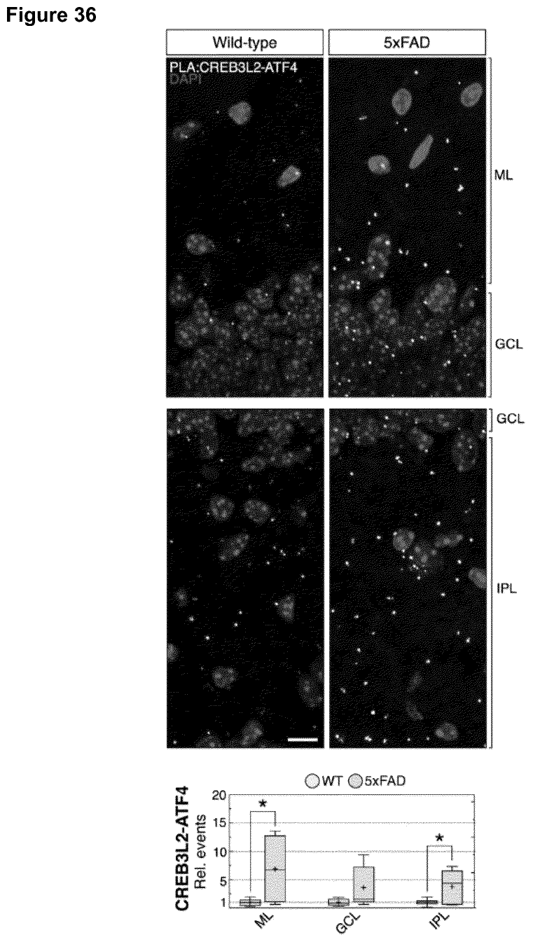

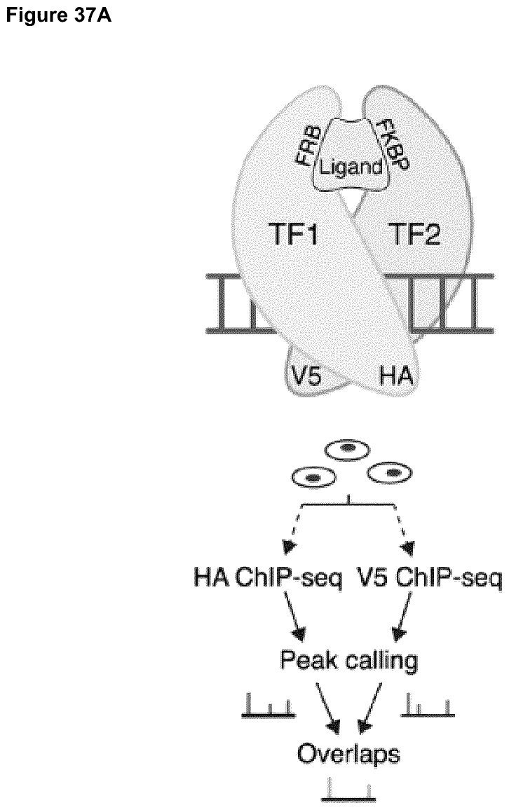

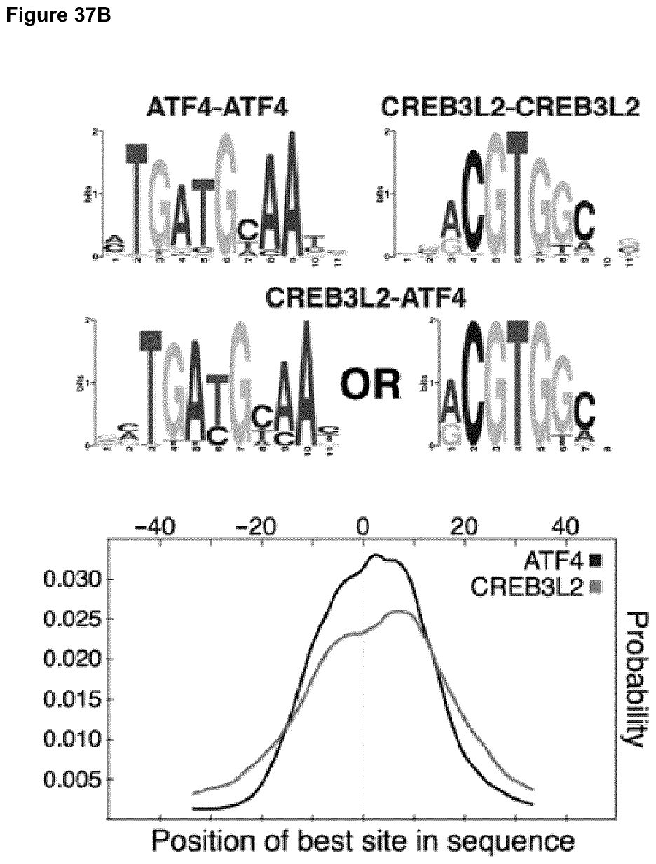

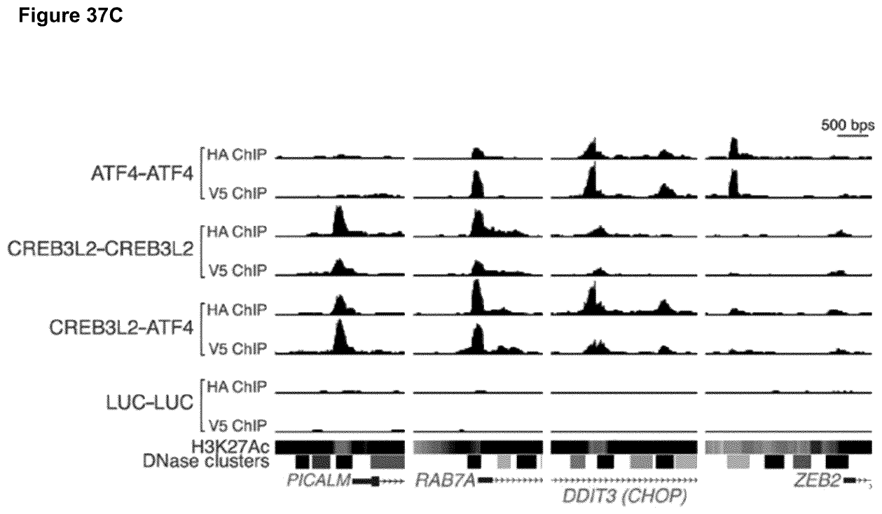

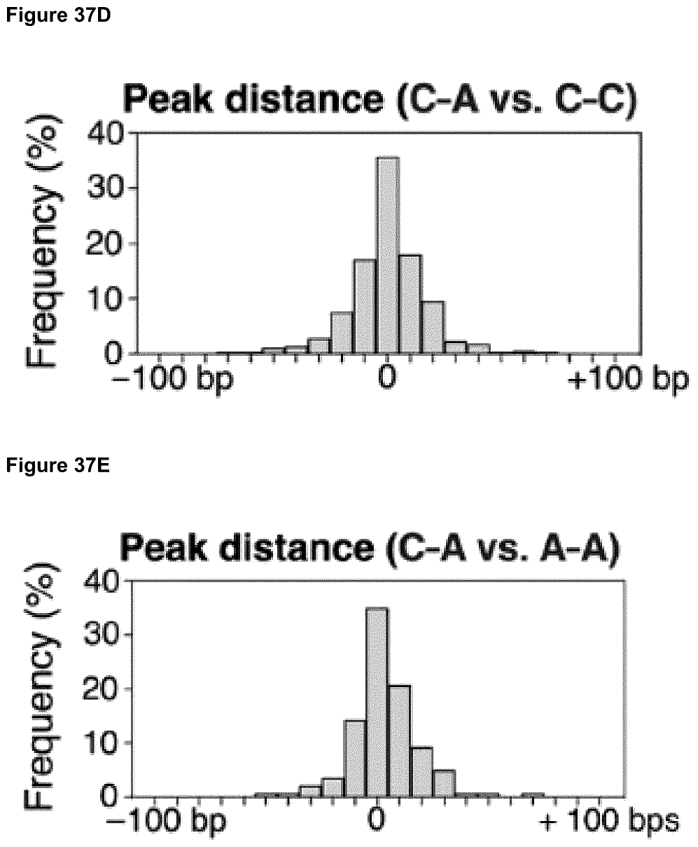

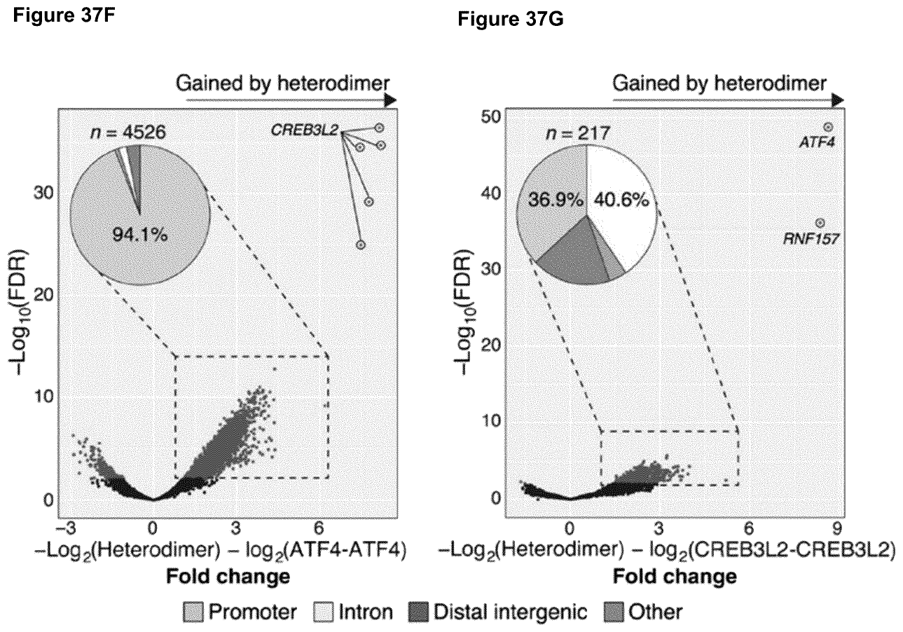

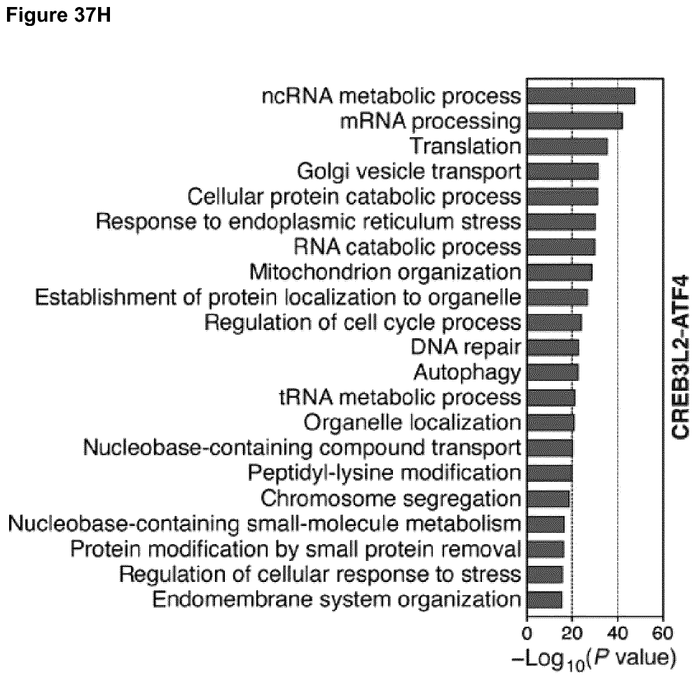

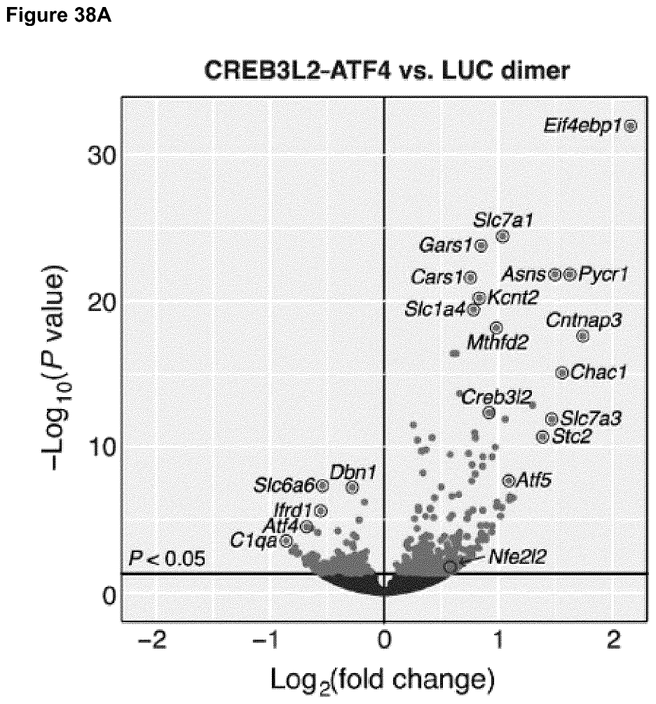

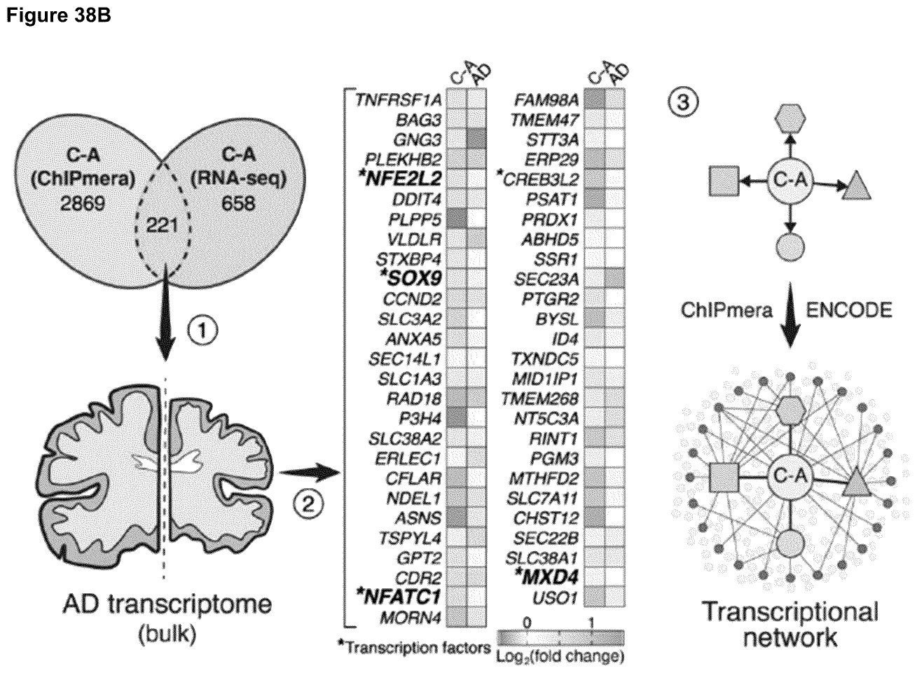

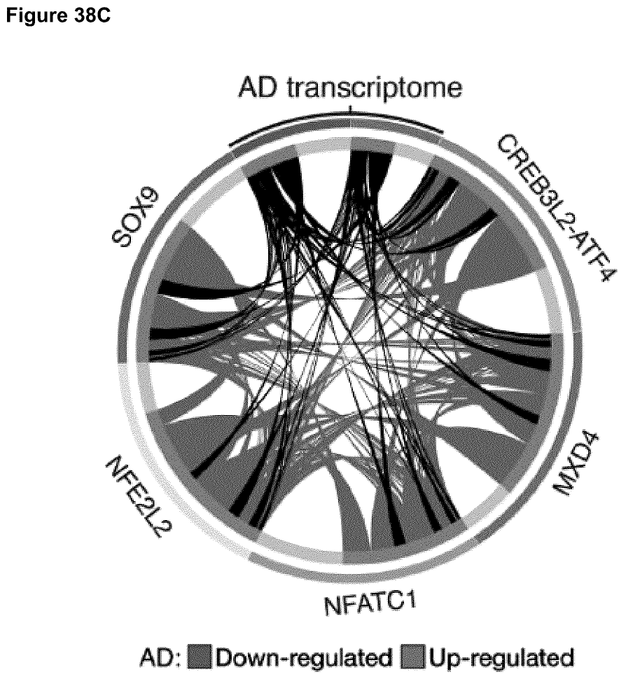

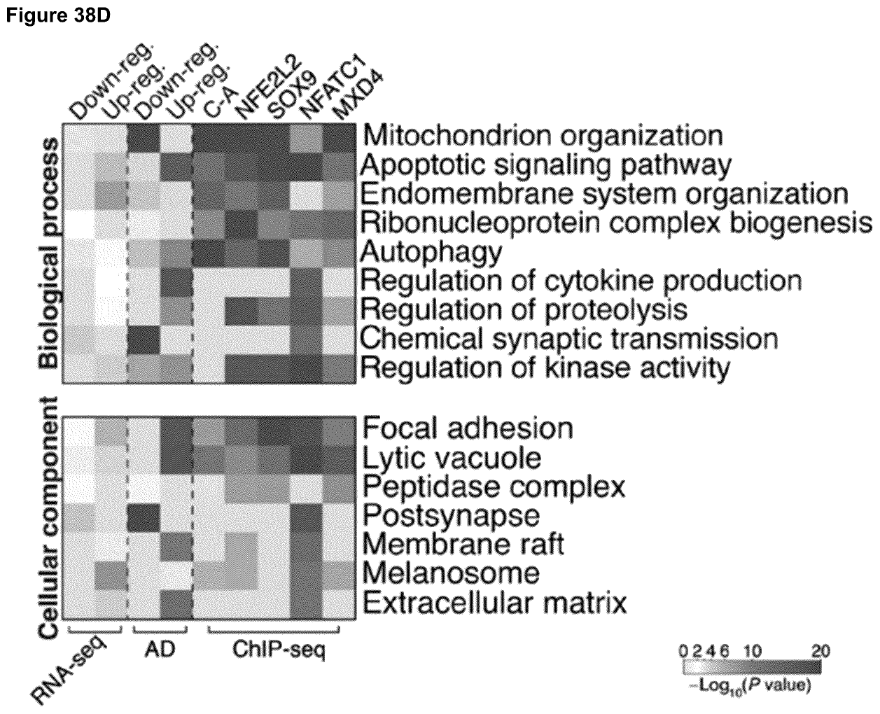

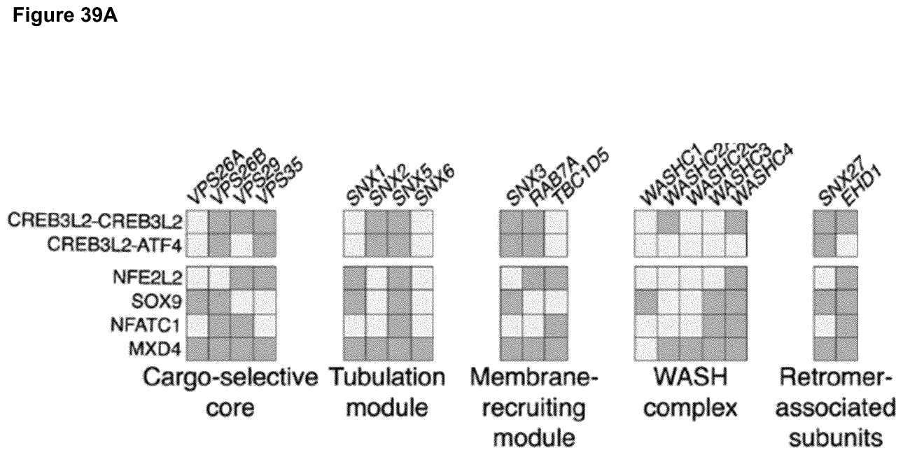

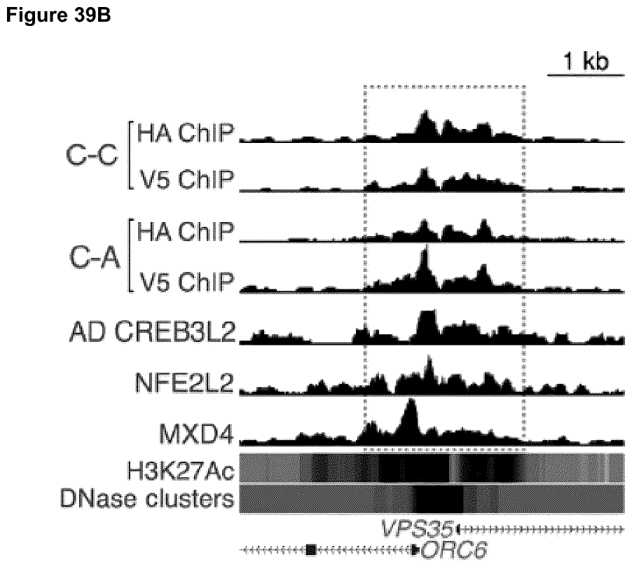

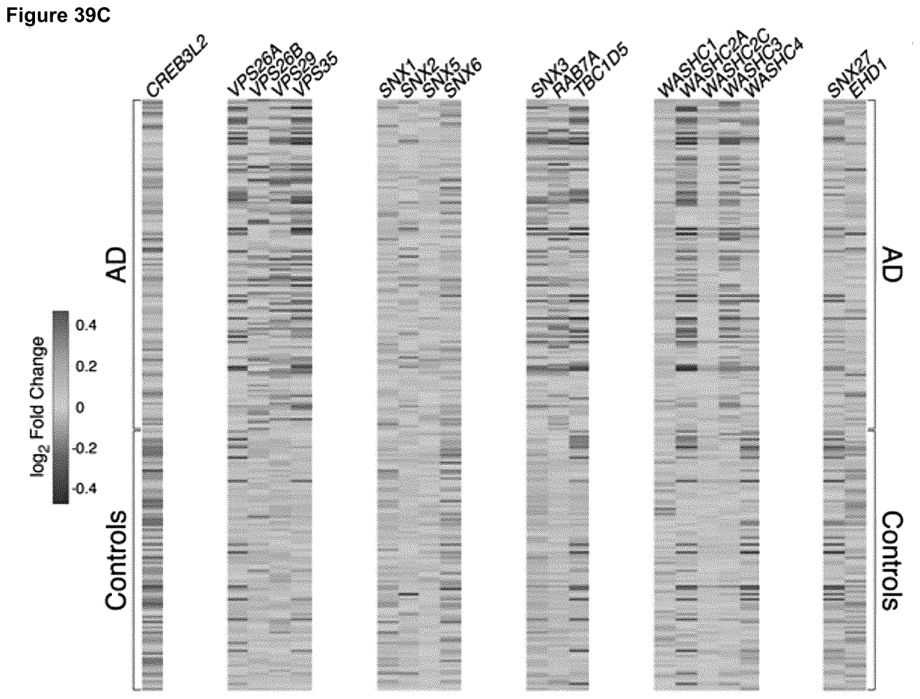

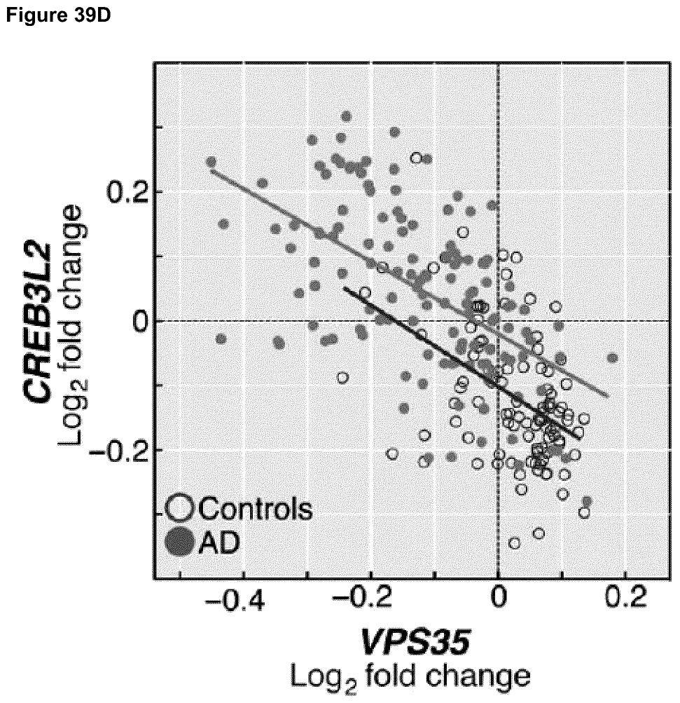

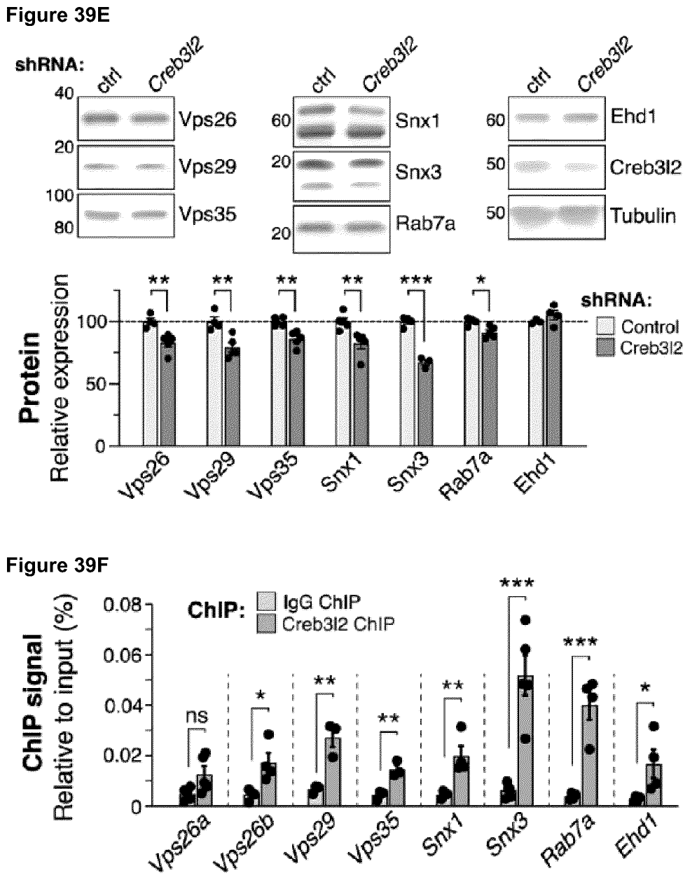

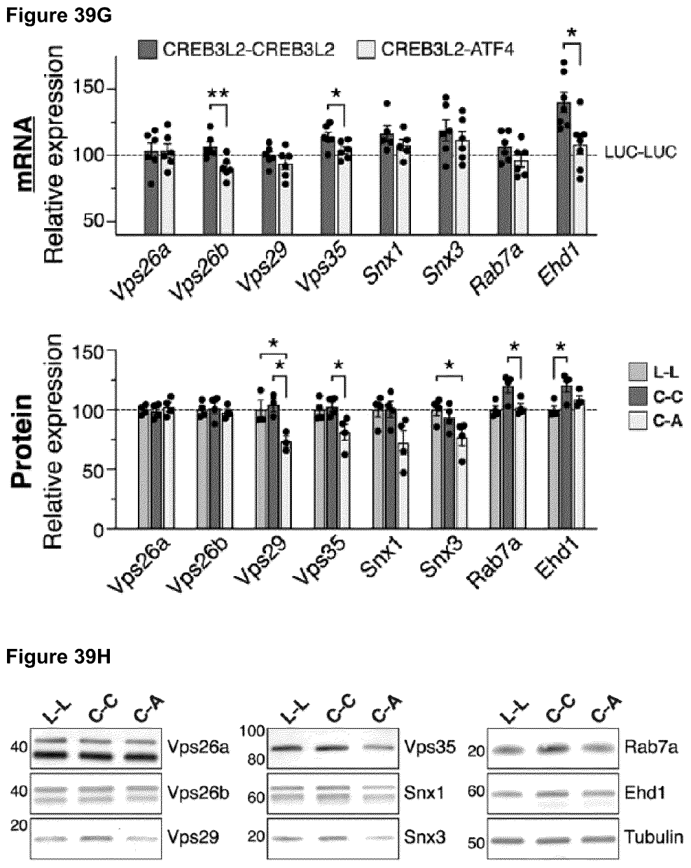

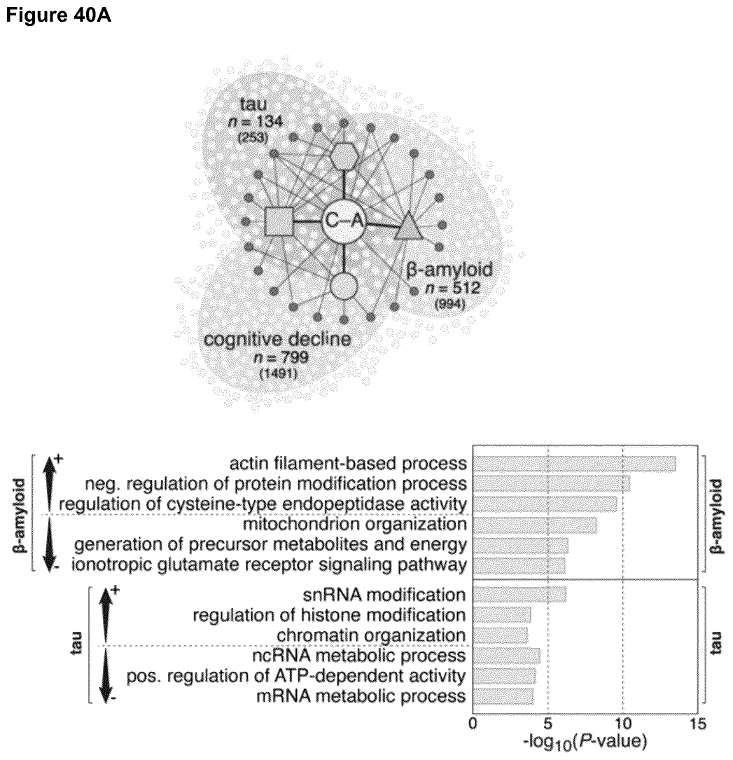

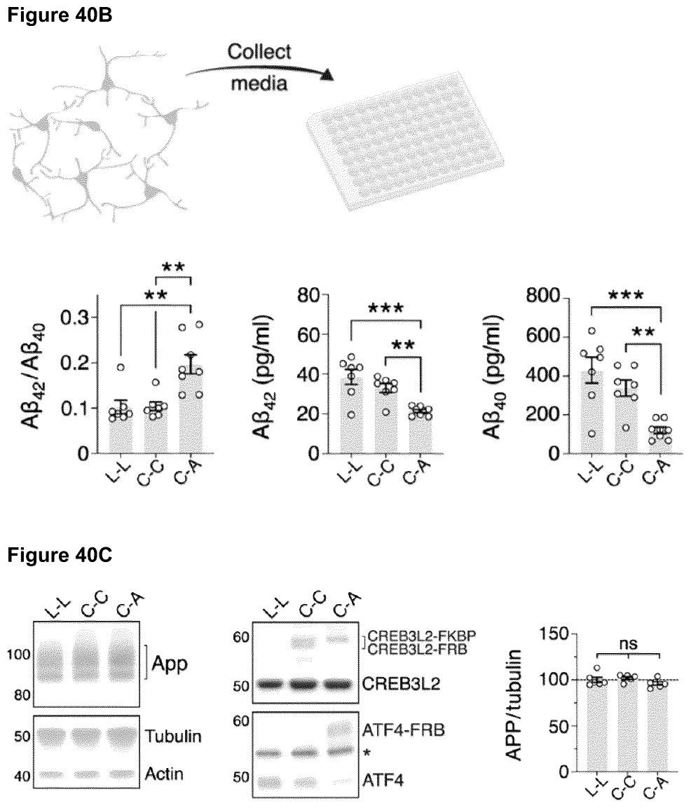

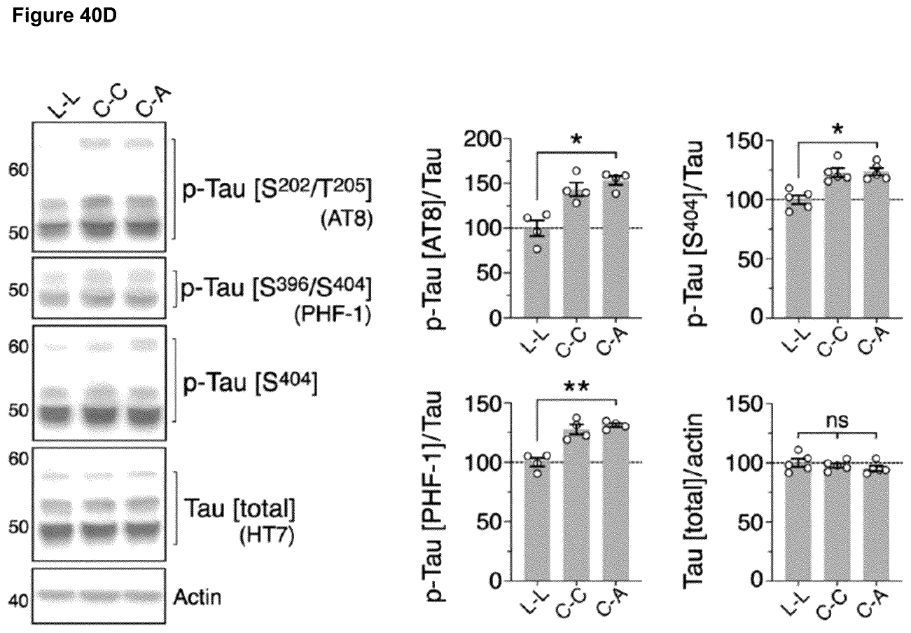

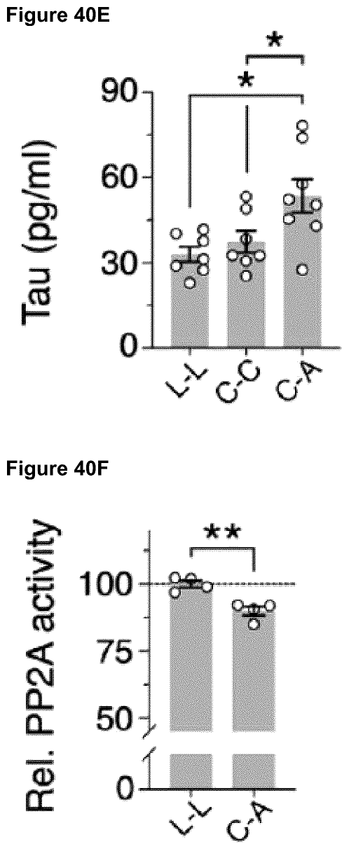

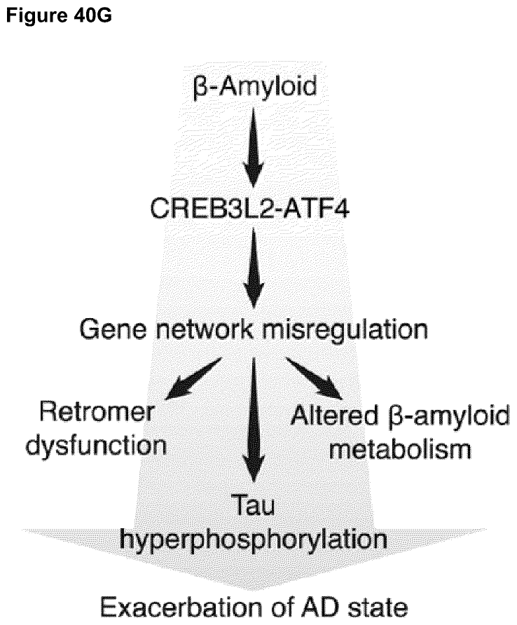

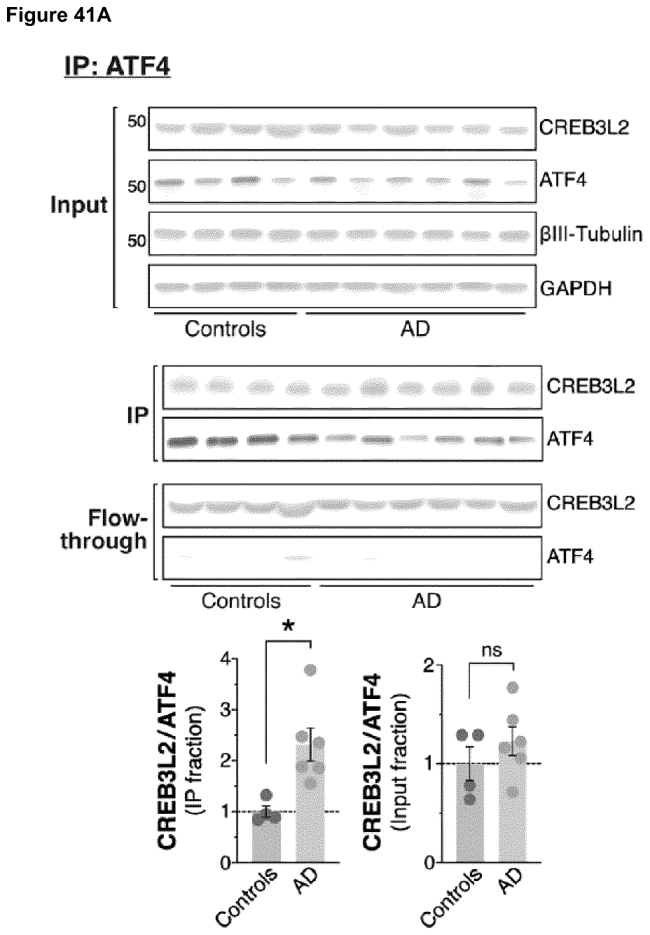

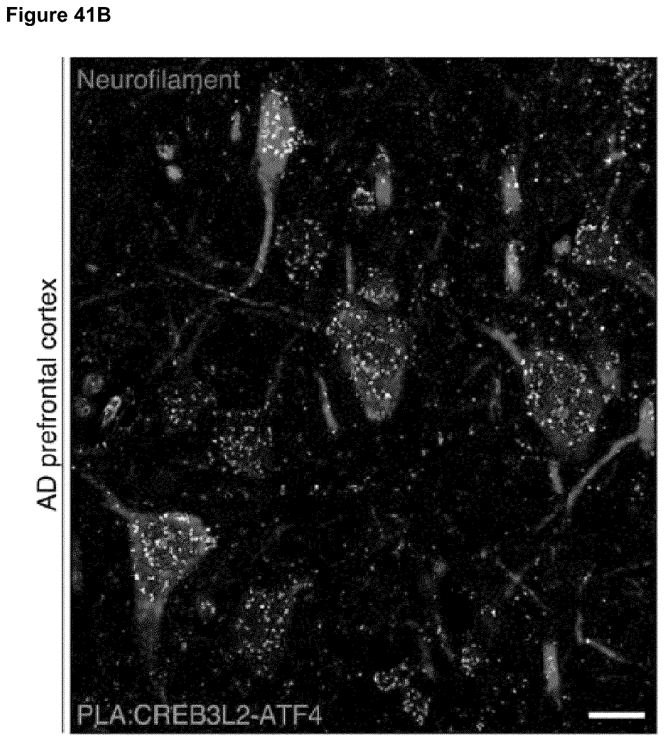

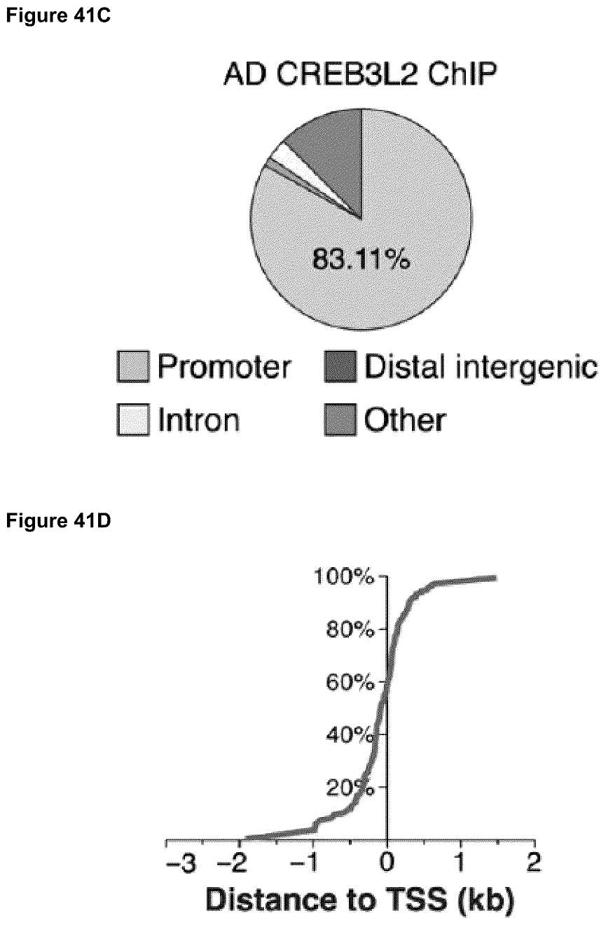

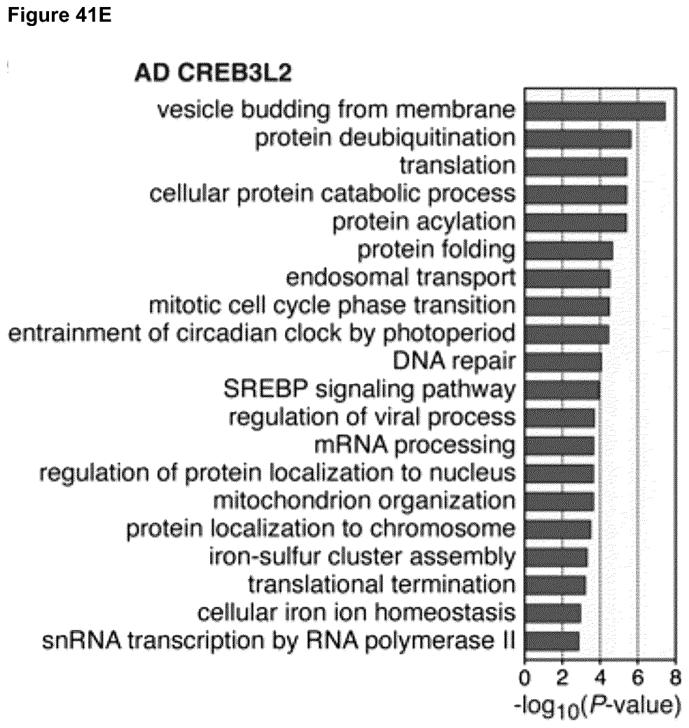

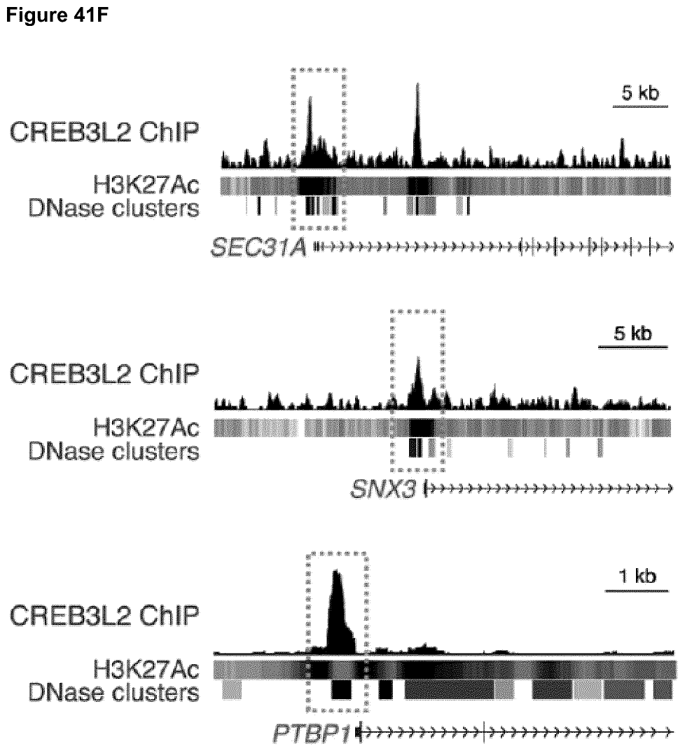

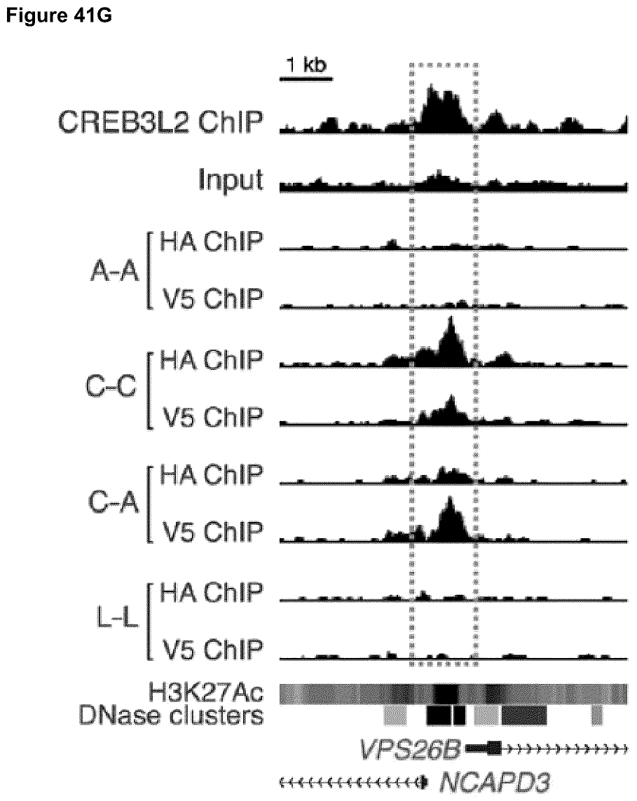

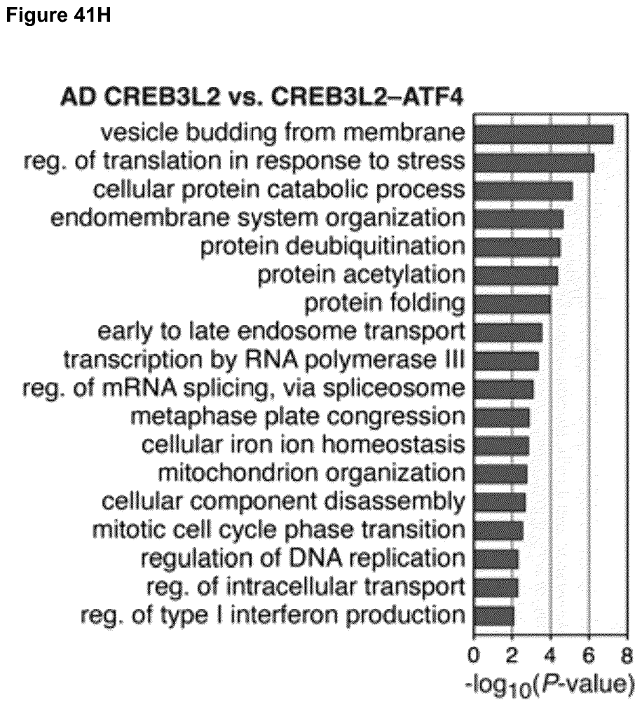

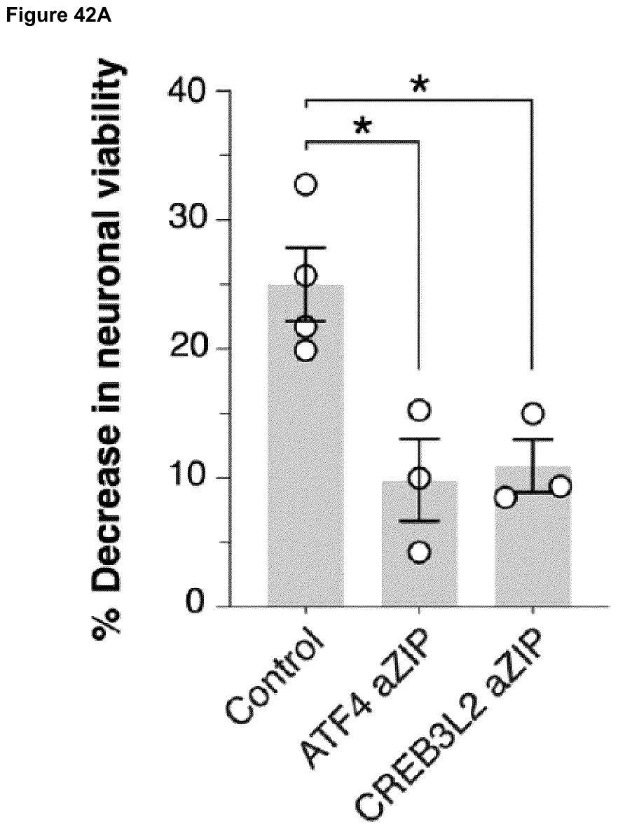

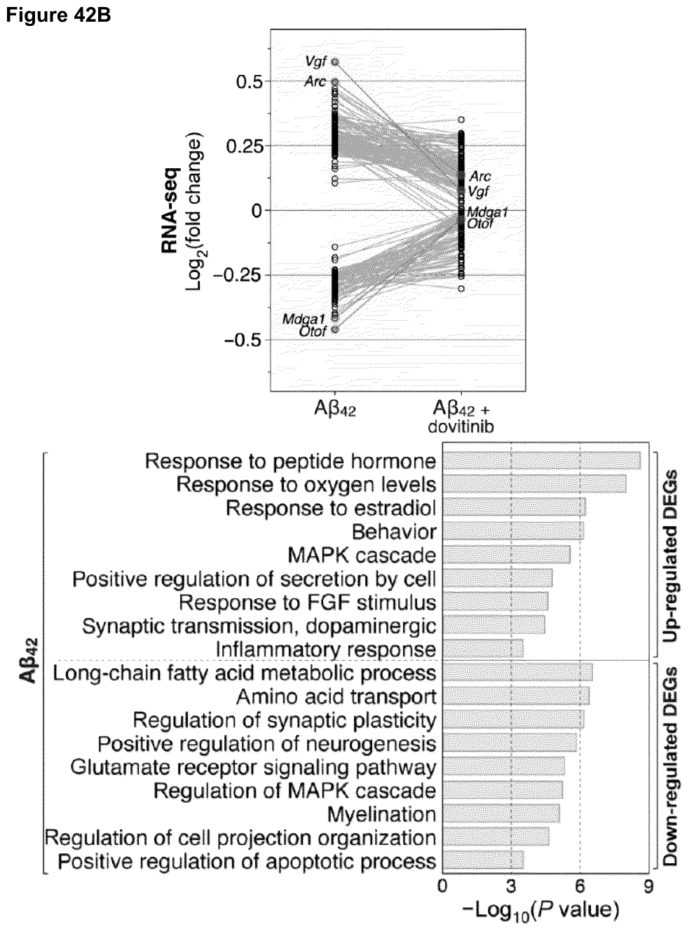

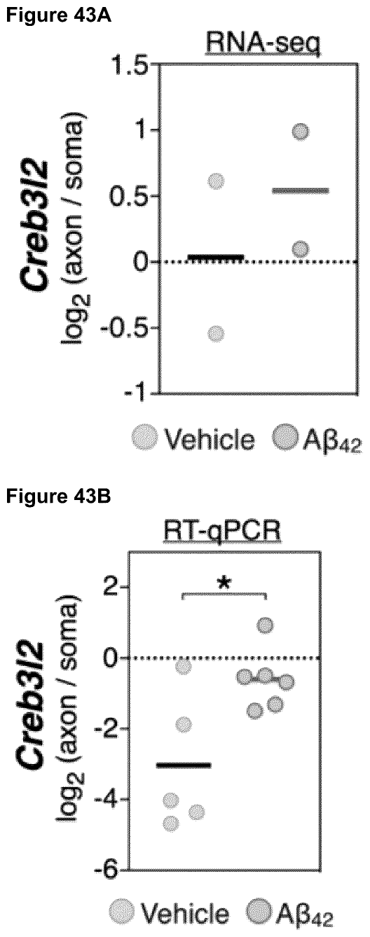

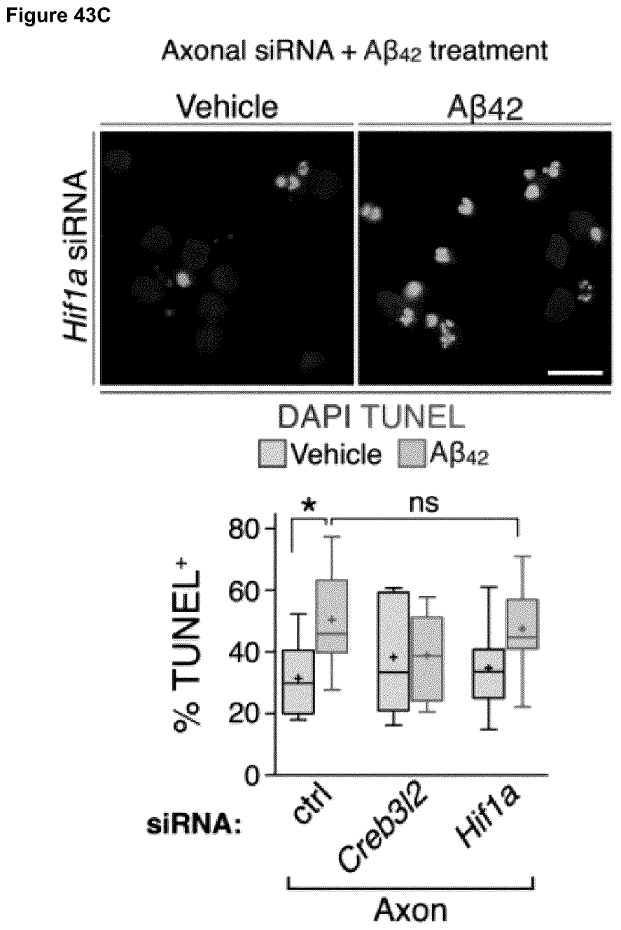

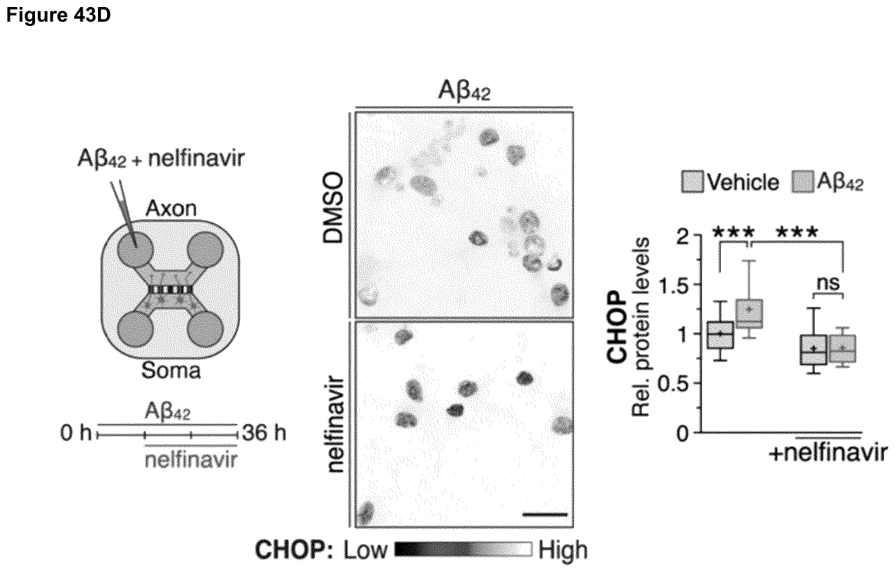

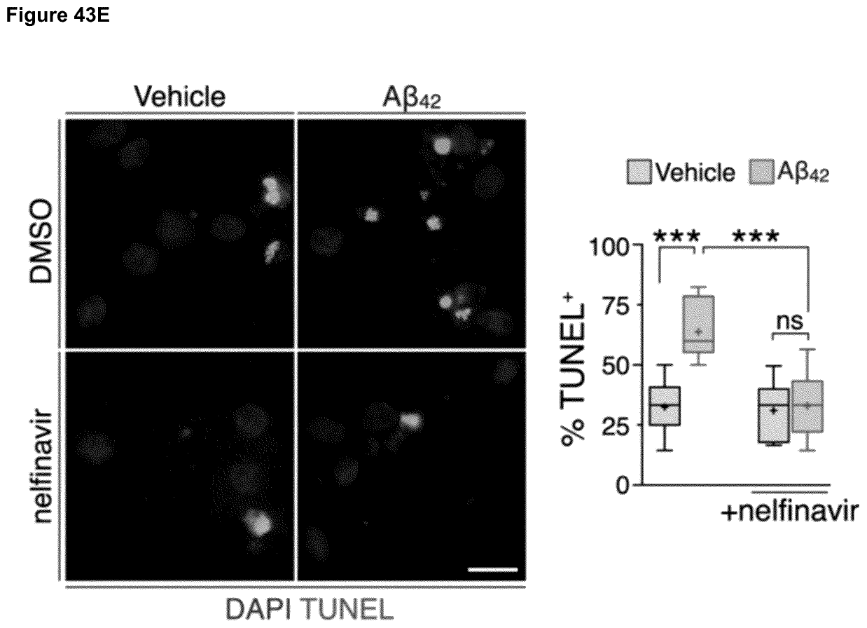

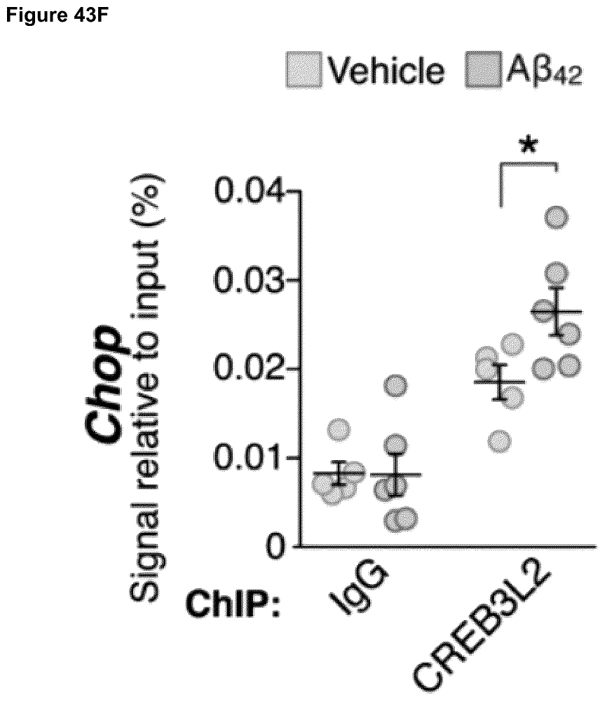

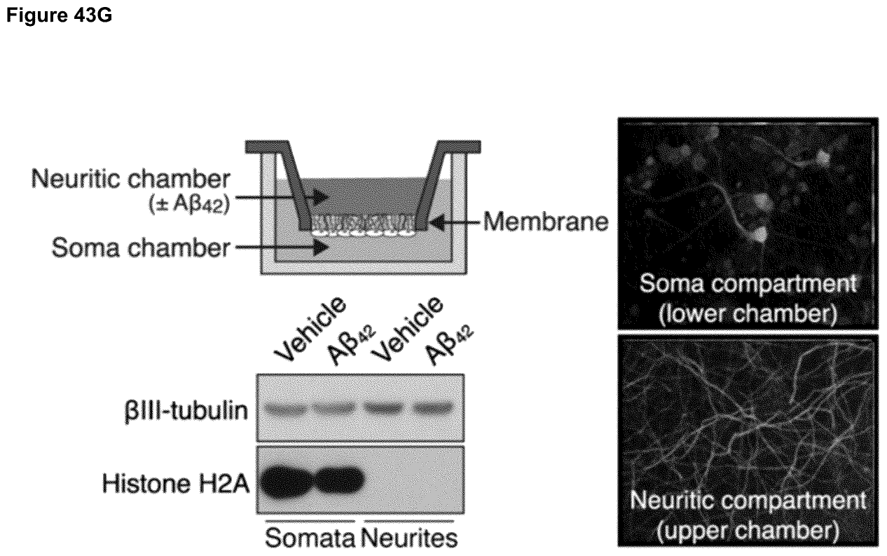

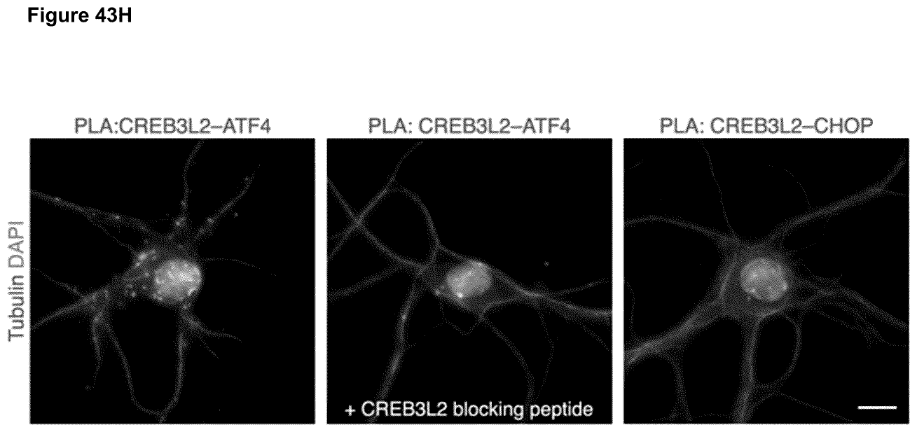

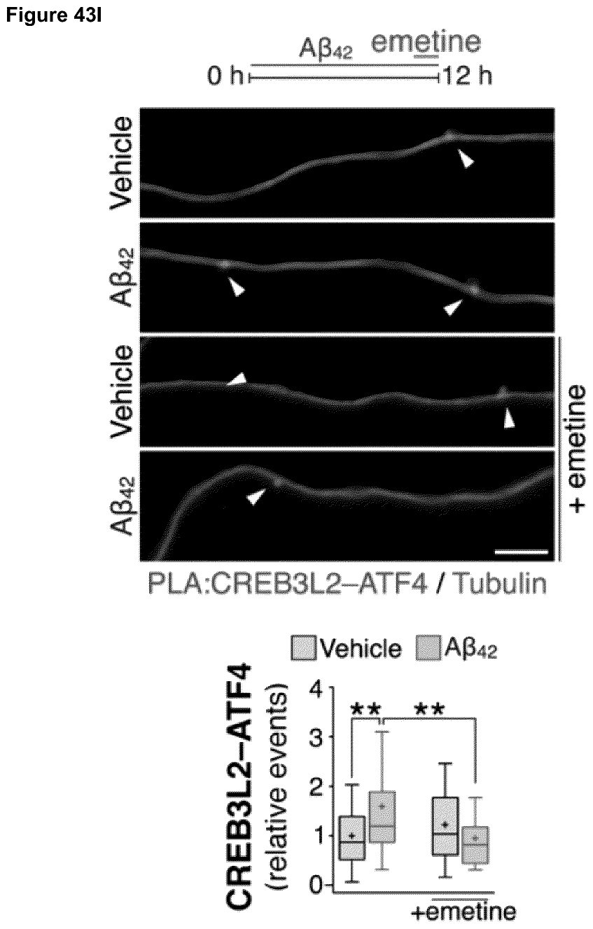

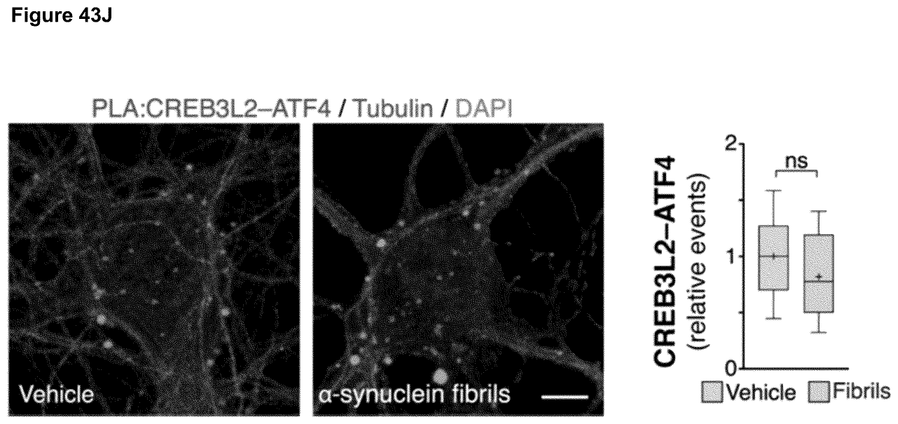

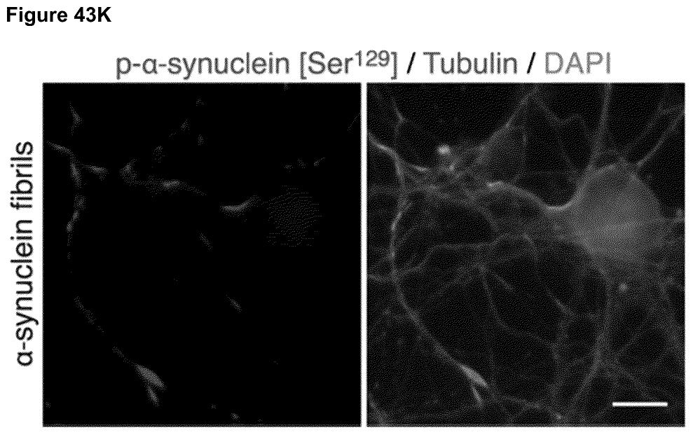

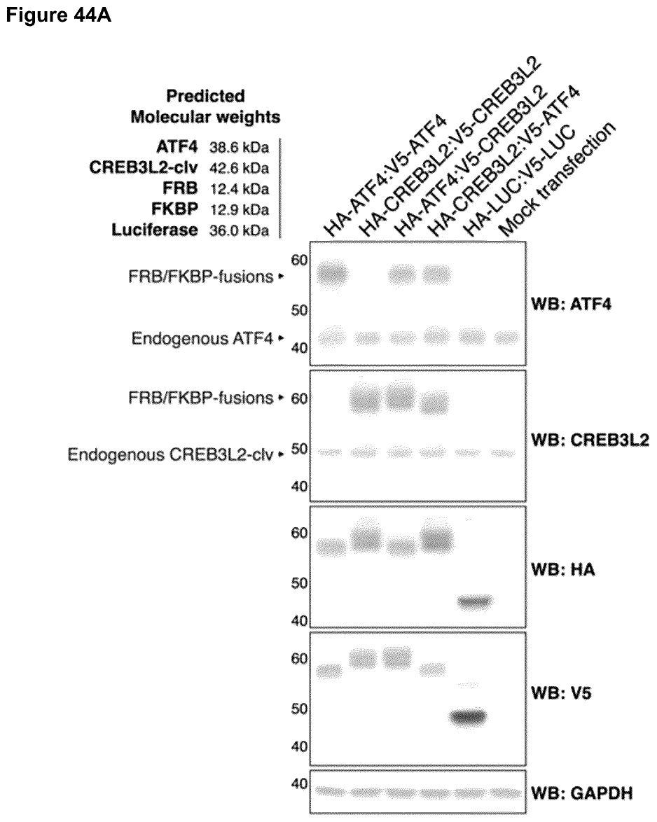

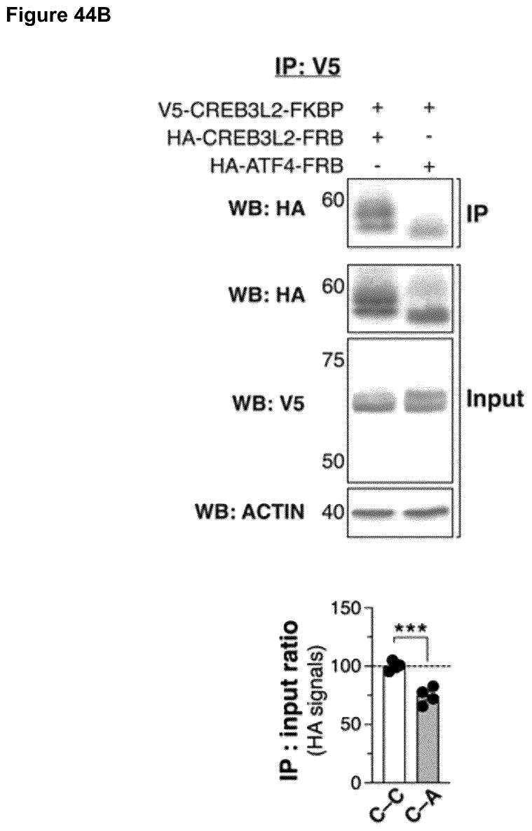

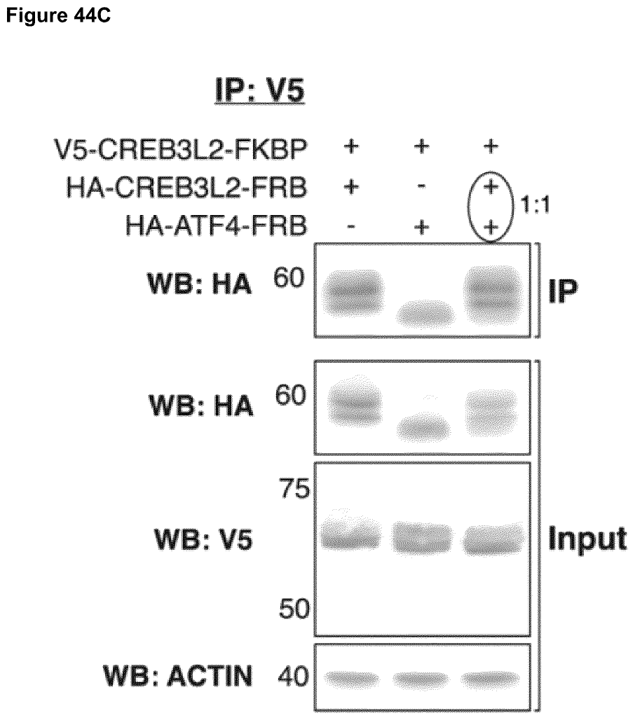

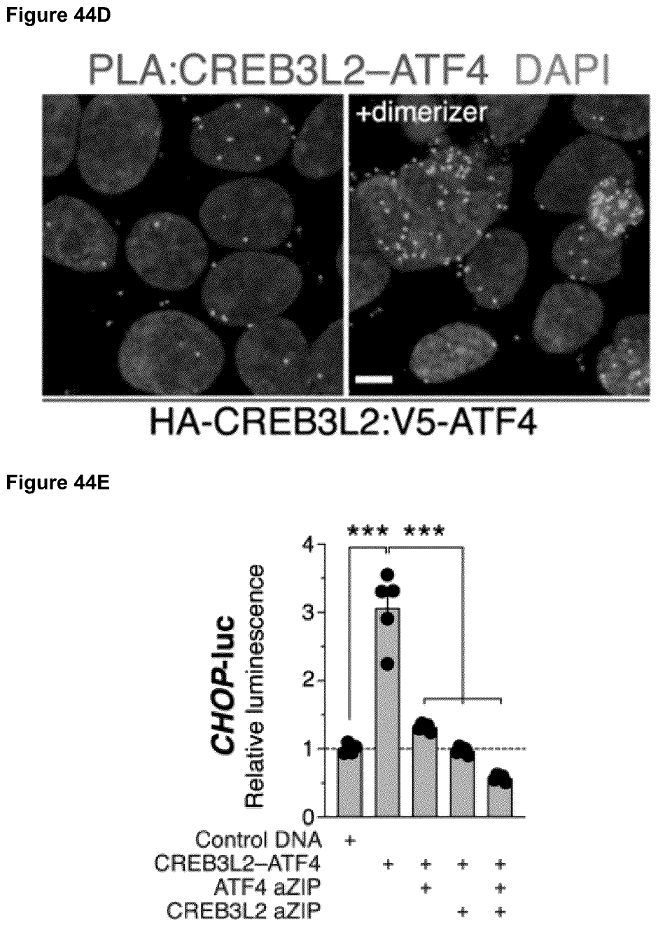

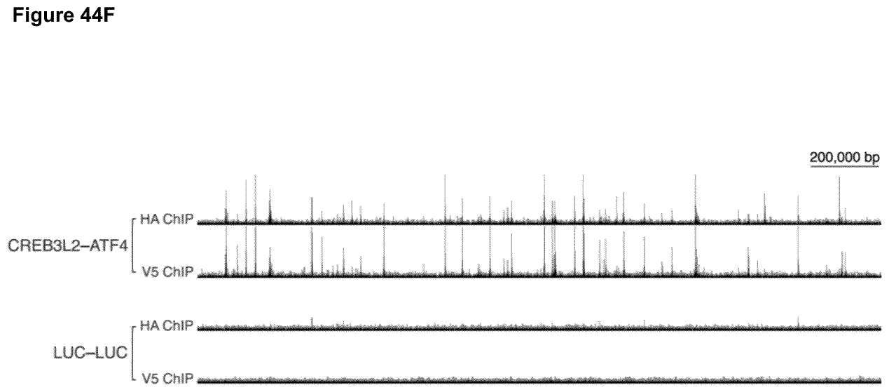

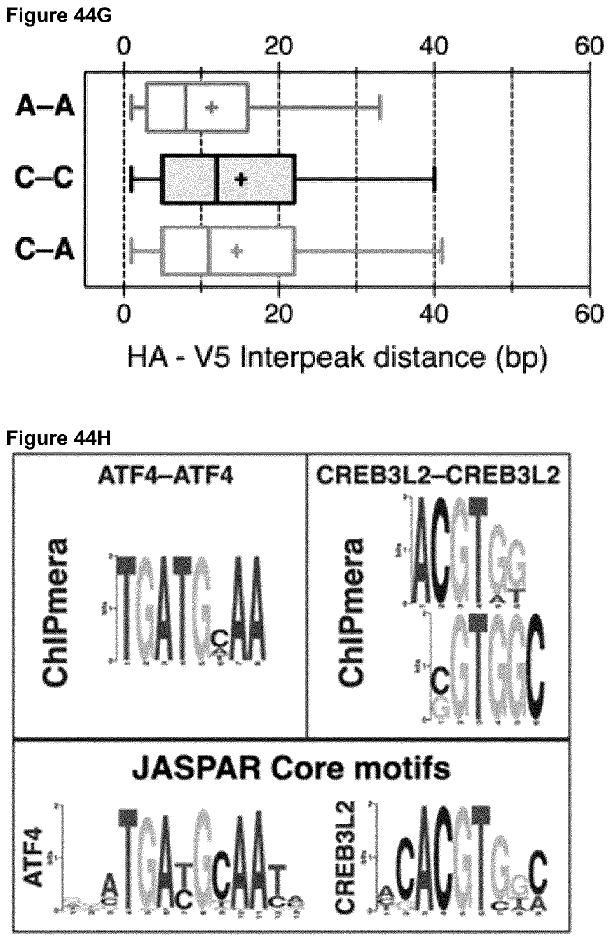

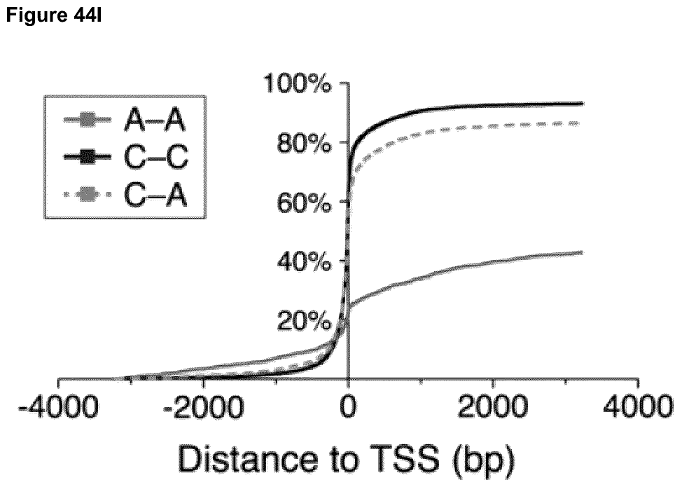

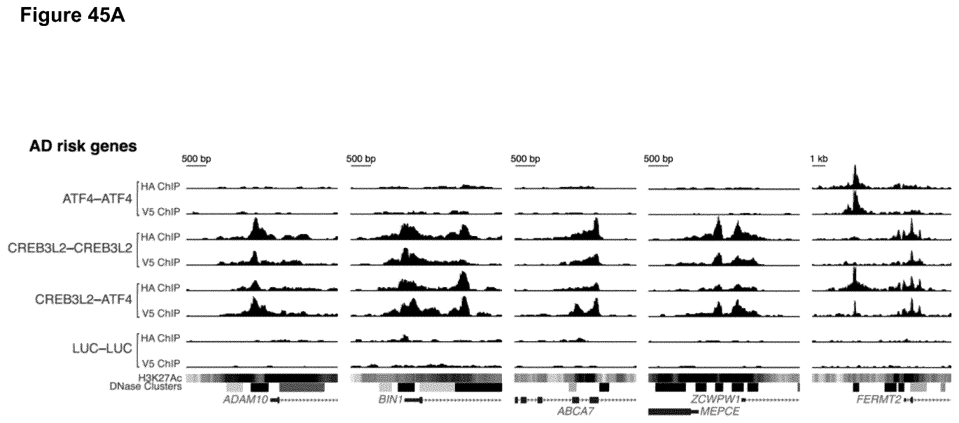

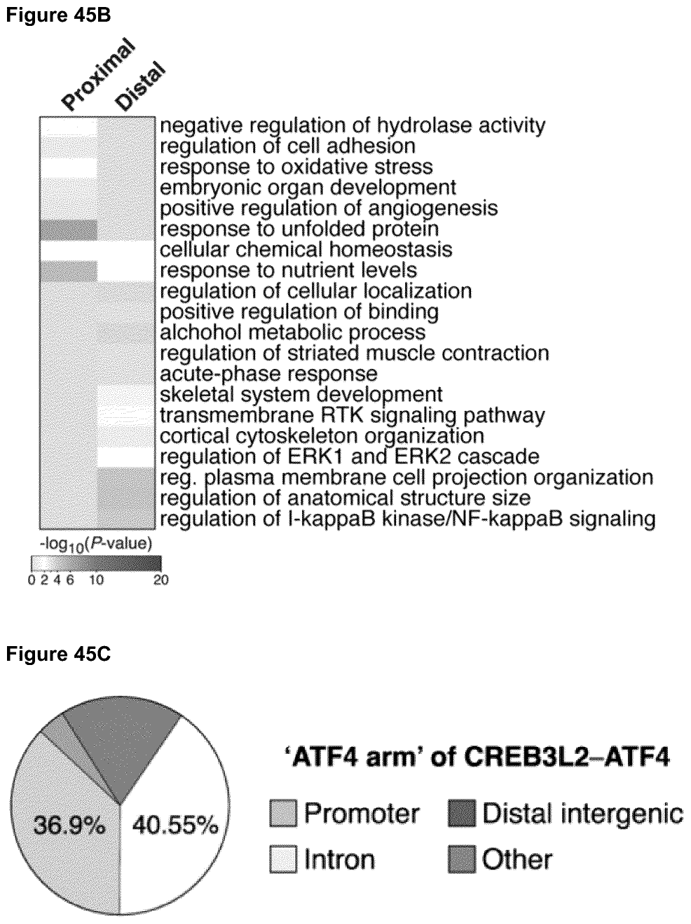

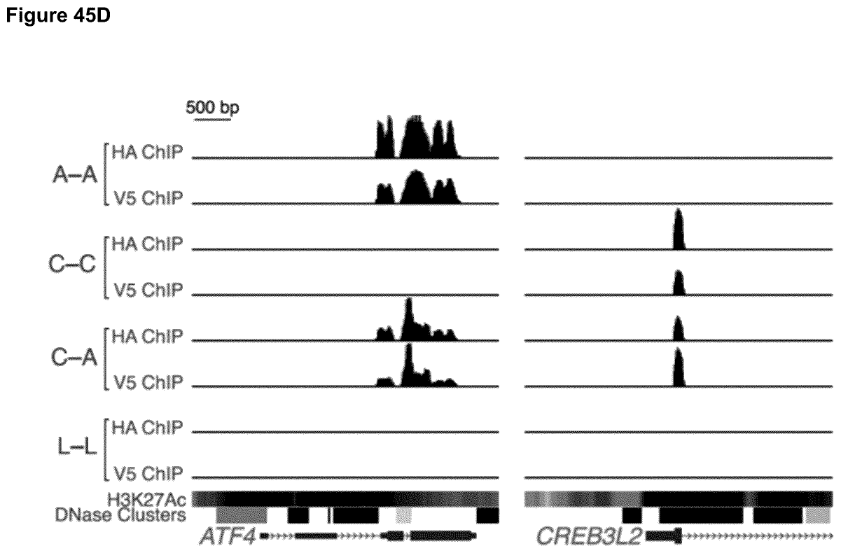

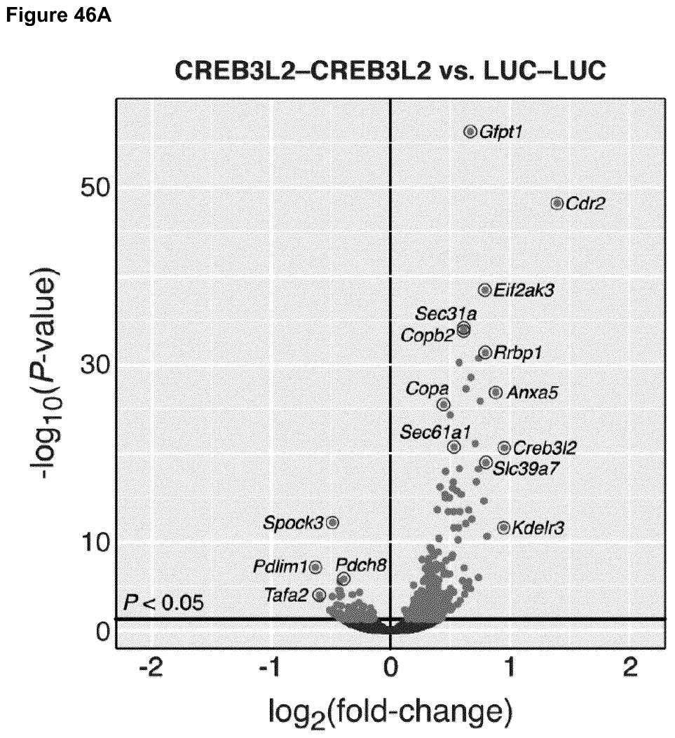

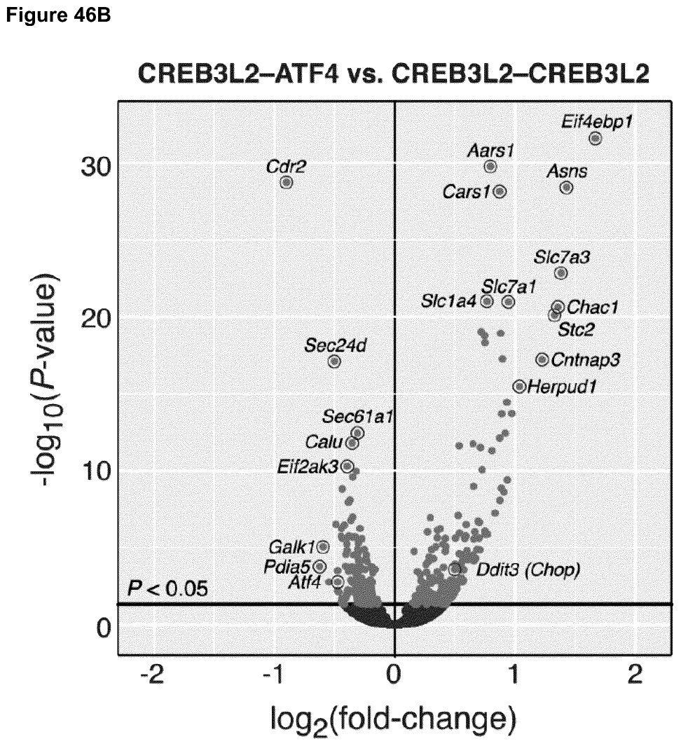

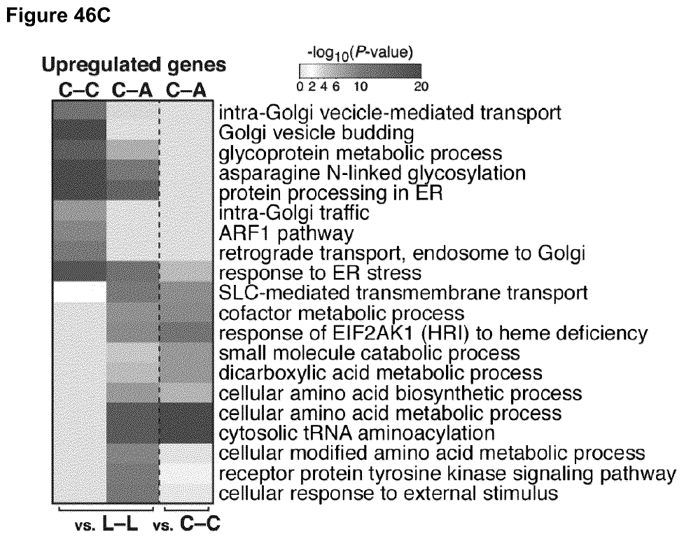

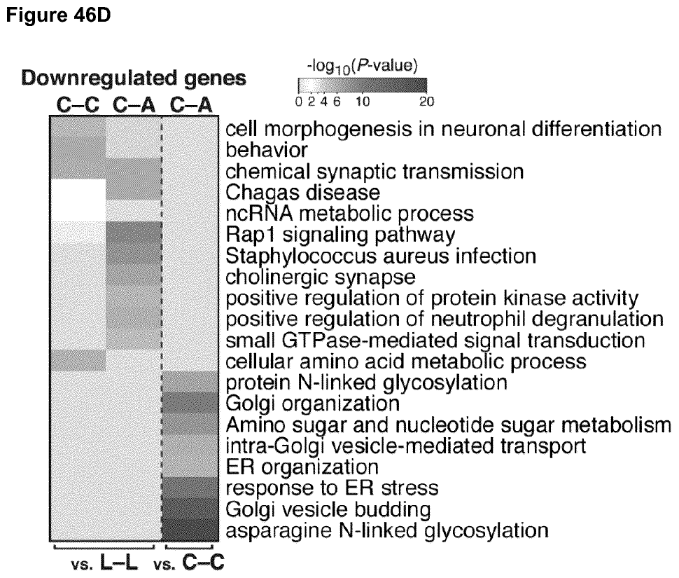

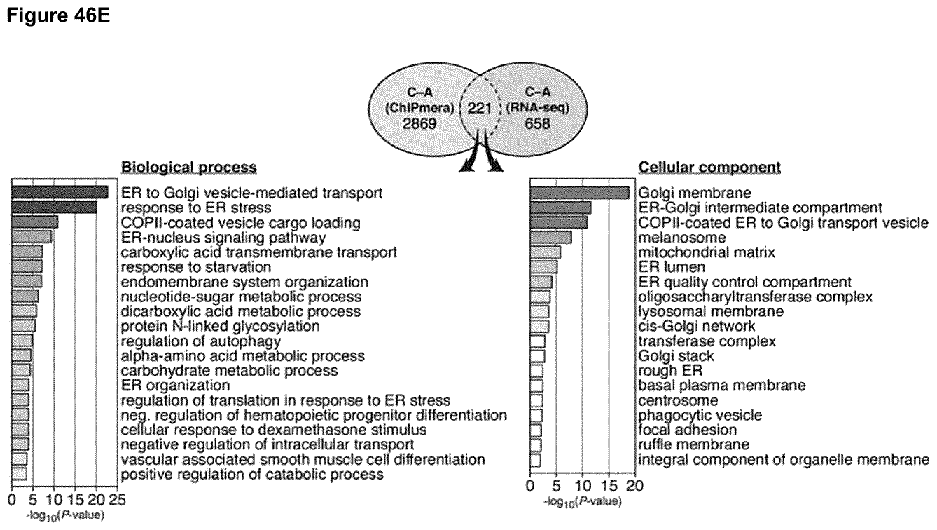

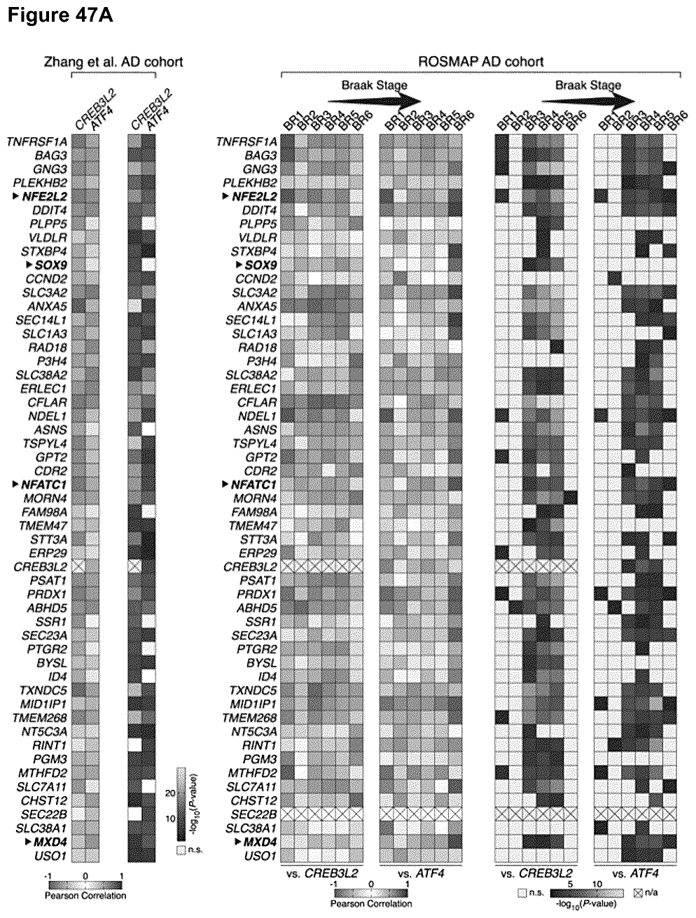

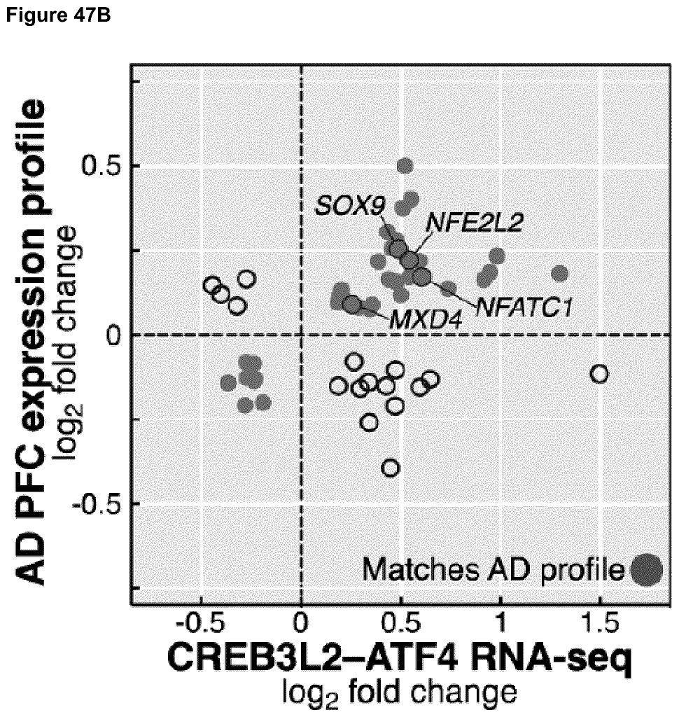

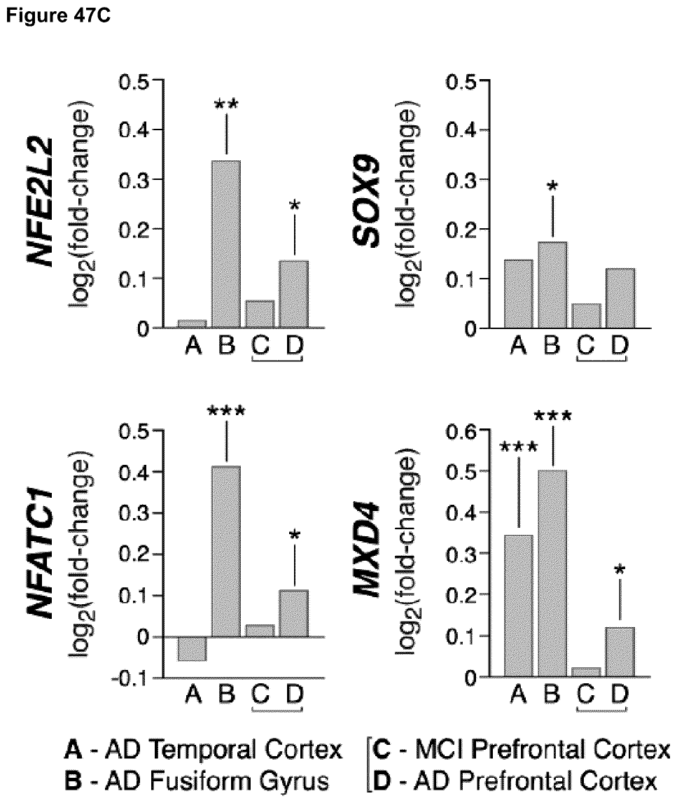

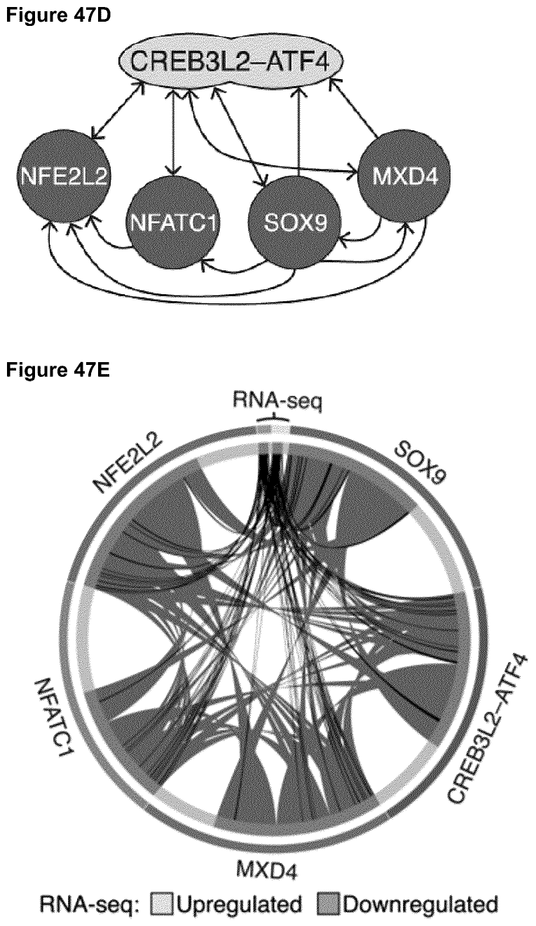

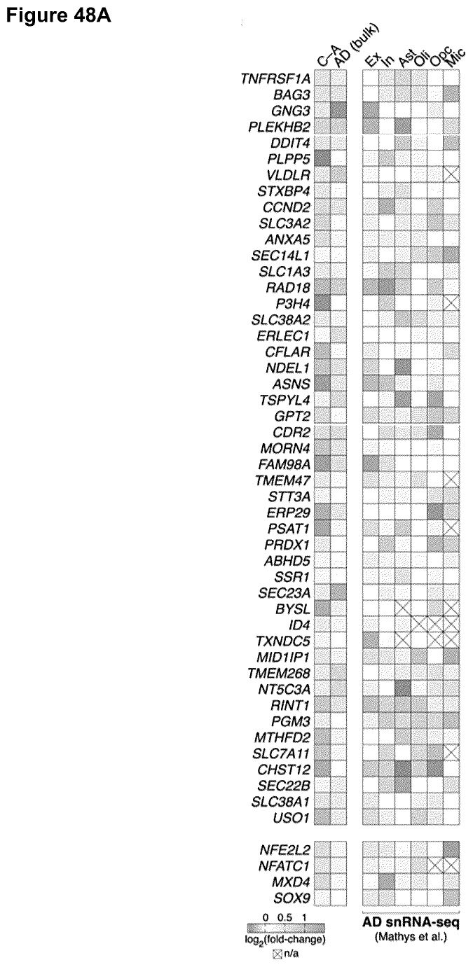

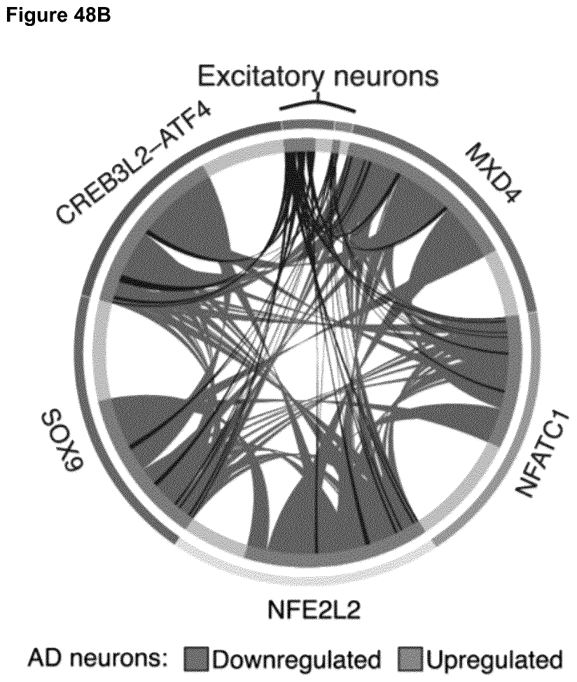

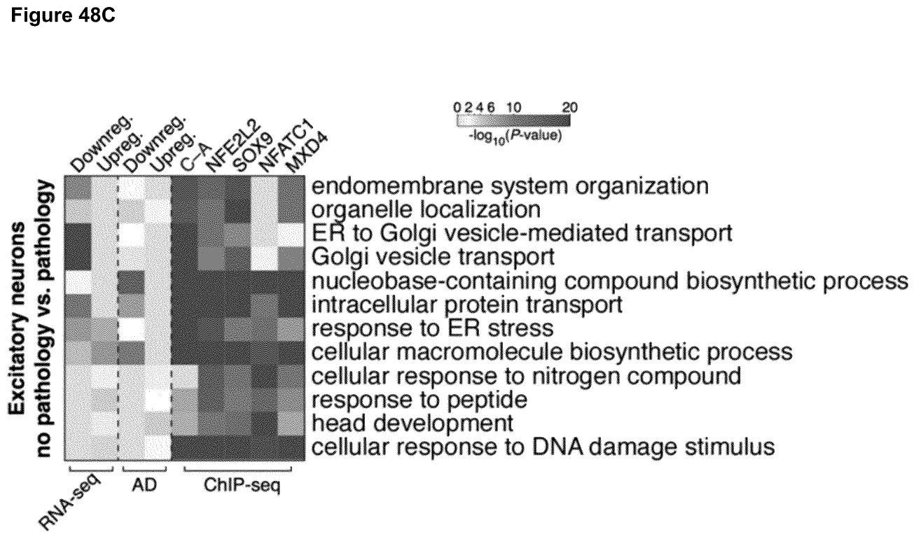

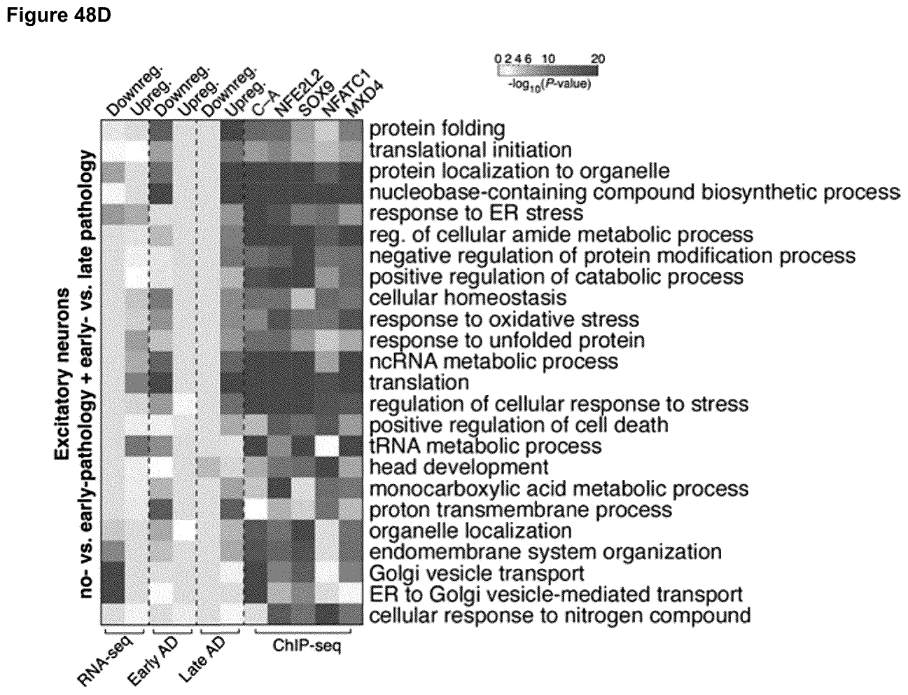

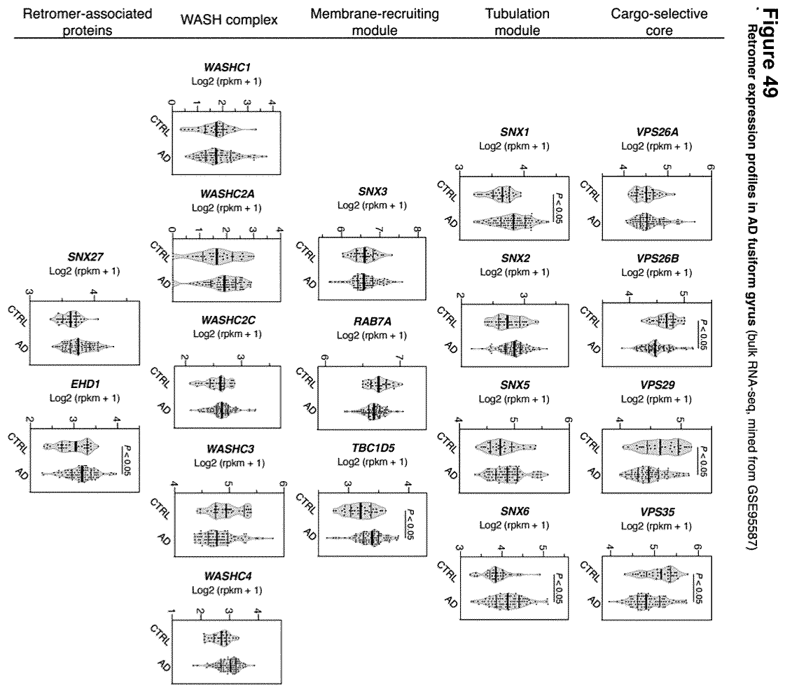

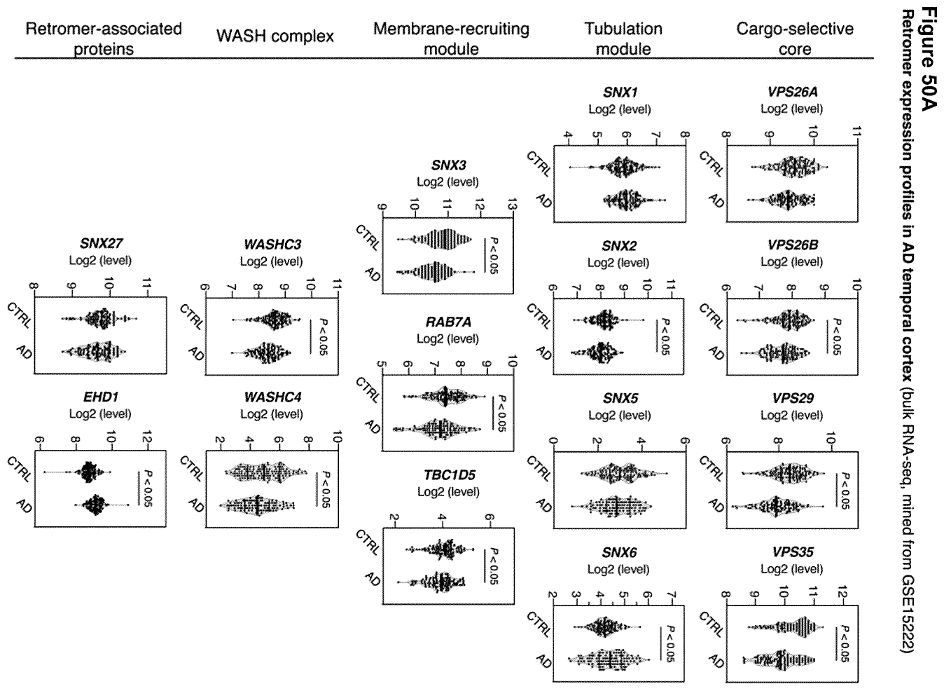

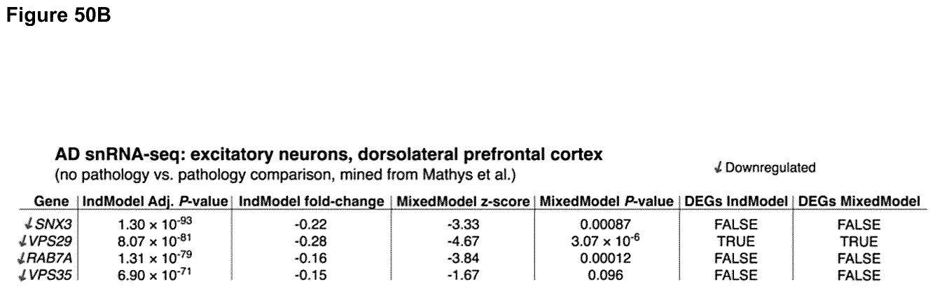

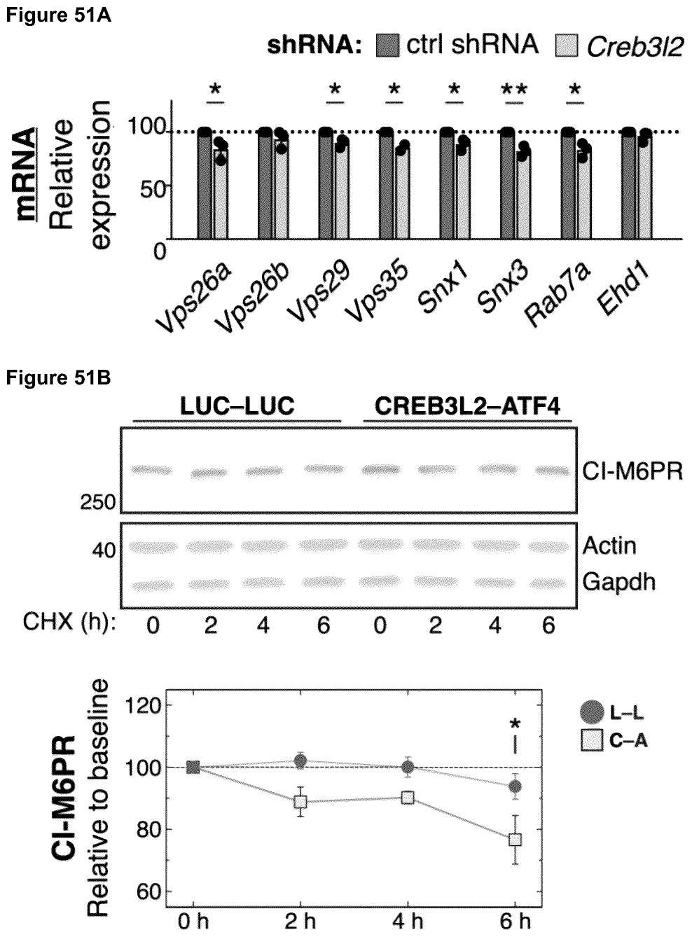

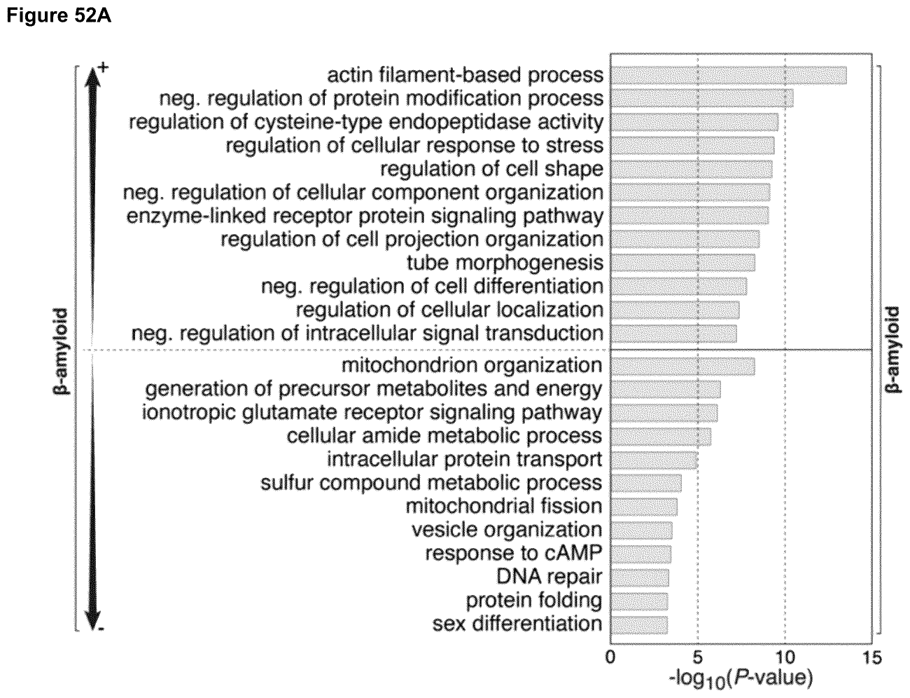

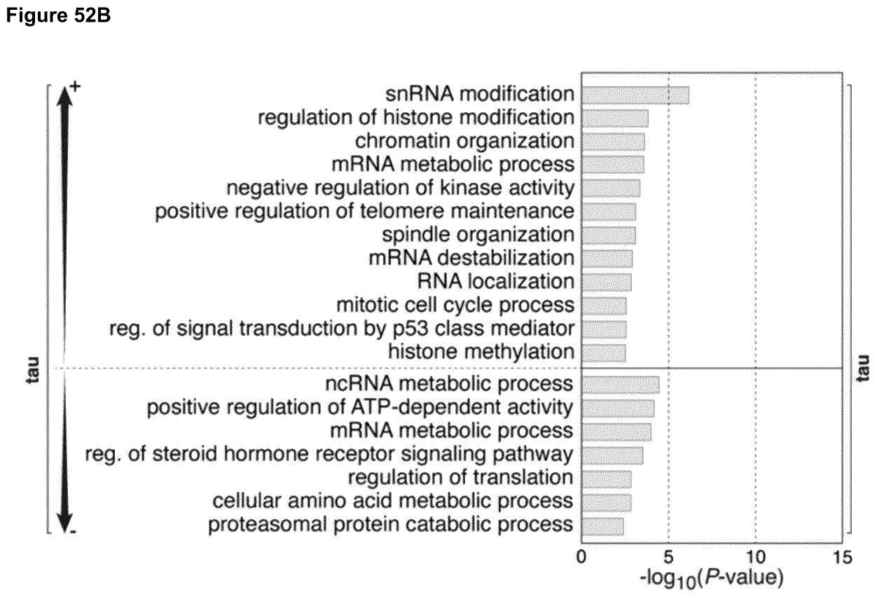

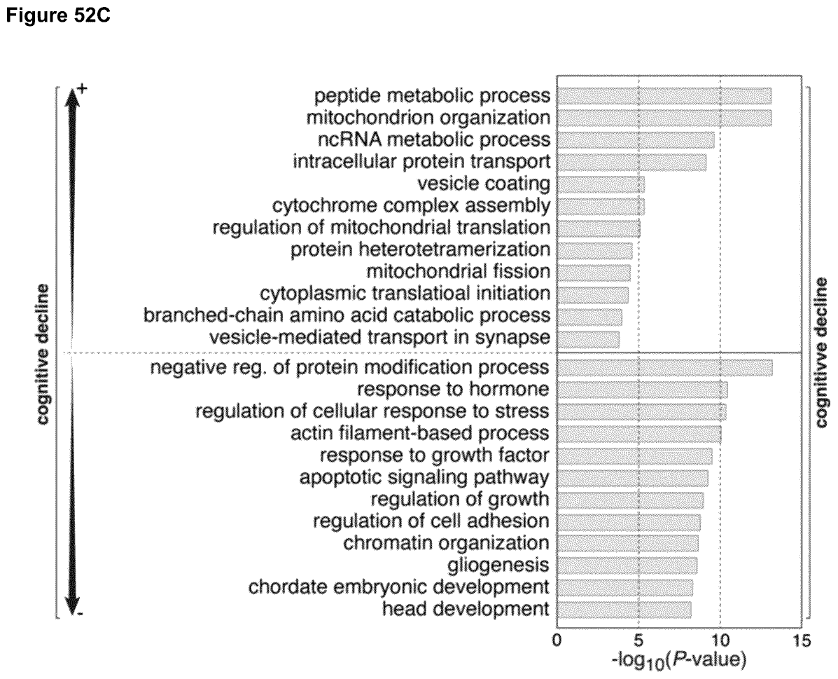

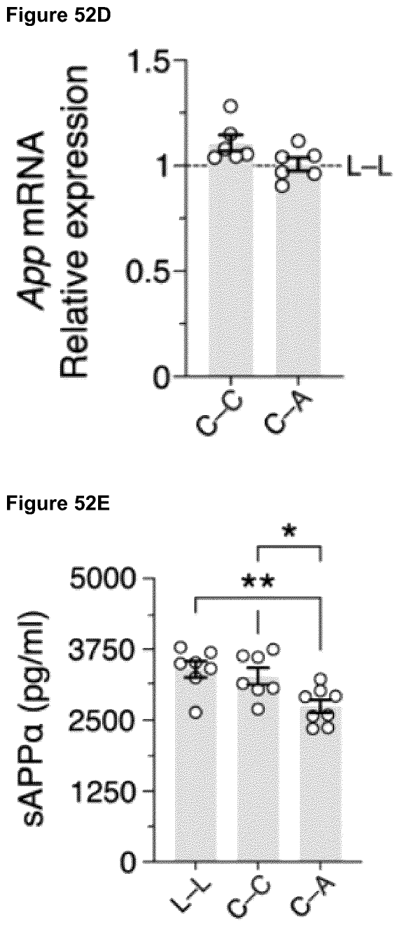

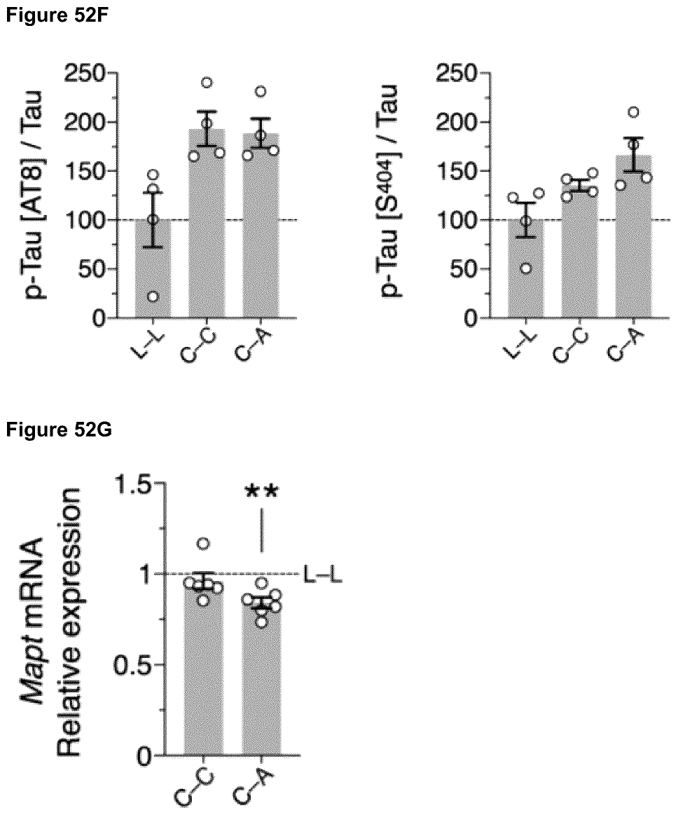

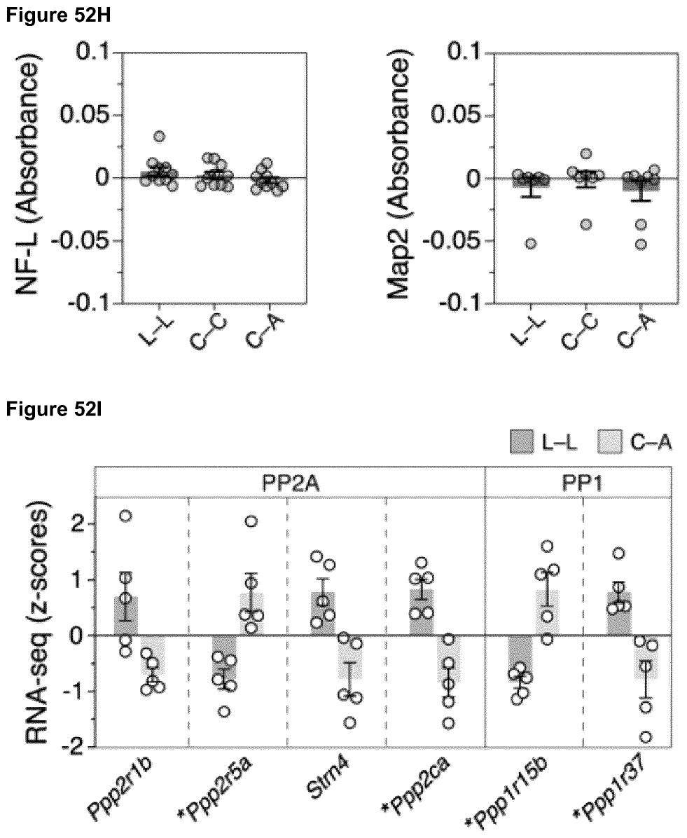

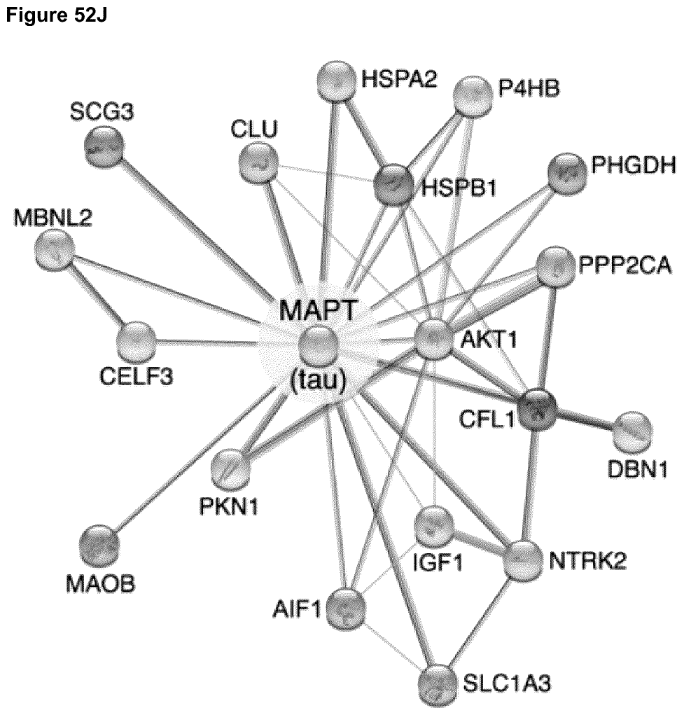

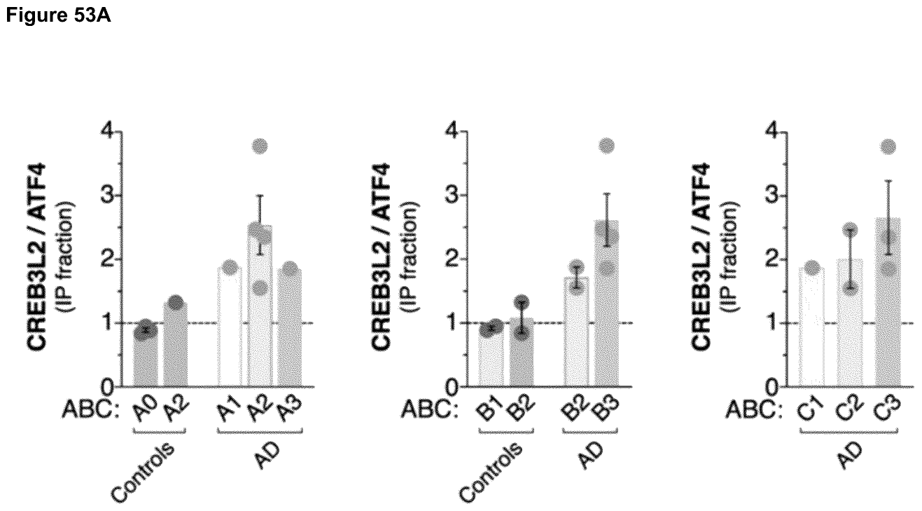

FIG. 6J shows that culture supernatants from control and Creb3/2-deficient rat cortical neurons were probed for Aβ₁₋₄₂ and Aβ₁₋₄₀ peptides using an antibody-capture plate assay. Box-plots display a summary of all measurements obtained (control n=11; Creb3/2 knockdown n=14; *P-value=0.0158, unpaired t-test); box-plot whiskers locate minimum and maximum values.

FIG. 6K shows that the extracellular levels of sAPPα, a non-amyloidogenic App cleavage product, were measured in culture supernatants from control and Creb3/2-deficient rat cortical neurons with an antibody-capture plate assay and normalized against total protein. Box-plots represent a summary of all measurements obtained (control n=11; Creb3/2 knockdown n=14). Group mean differences were statistically nonsignificant (ns; P-value=0.99, unpaired t-test). Plot whiskers correspond to minimum and maximum values.

FIG. 6L shows that APP carboxy terminal fragments (CTFs) were compared by western blot between control and Creb3/2 suppressed rat cortical neurons using an antibody against the C-terminal portion of APP. Four independent control and Creb3/2 knockdown samples were measured (***P-value <0.0001, unpaired t-test). Box-plot whiskers trace minimum and maximum values.

FIGS. 7A-7I show that CREB3L2 is a locally produced dimerization partner of ATF4 in Aβ₁₋₄₂-treated axons.

FIG. 7A shows the scheme of a microfluidic chamber used to isolate axons of hippocampal neurons according to one aspect of the present disclosure. Due to their extended morphology, only axons can cross through two 200 μm long microgroove barriers into the adjacent, ‘axonal’ compartments.

FIG. 7B shows a representative assessment of somatic degeneration by TUNEL staining of rat hippocampal neurons after Aβ₁₋₄₂ stimulation. Axons were transfected with control, Creb3/2, or Hif1a-targeting siRNAs before treatment with Aβ₁₋₄₂ for 48 hours. Hif1a is another TF-encoding message recruiting into axons following Aβ₁₋₄₂ stimuli. Unlike Creb3/2, silencing axonal Hif1a expression did not ameliorate Aβ₁₋₄₂-induced cell death. Data are presented as mean±SEM of n=10-12 biological replicates; *P-value=0.0357, one-way ANOVA test with post hoc Bonferroni multiple comparison correction. Scale bar, 15 μm.

FIG. 7C shows a ChIP-qPCR assay of CREB3L2 binding to CHOP promoter regions in vehicle or Aβ₁₋₄₂-treated rat cortical neurons. Neurons were grown without microfluidic compartmentalization (i.e., dissociated cultures), and an Aβ₁₋₄₂ bath-applied for 36 hours. ChIP signals in each sample were normalized against total input chromatin. Data are presented as mean±SEM of n=5 independent replicates; ***P-value <0.0001, one-way ANOVA test with post hoc Bonferroni multiple comparison correction.

FIG. 7D shows a coimmunoprecipitation analysis of CREB3L2 and ATF4 association in HEK293 cells (overexpression model). While wild type CREB3L2-clv and ATF4 can associate as part of a complex, mutant CREB3L2 and ATF4 lacking leucine zippers are unable to establish detectable interactions.

FIG. 7E shows a schematic representation of a transwell Boyden chamber system employed in one aspect of the present disclosure to isolate neurites from cell bodies. As a control of its applicability, we tested for the presence of a resident nuclear protein, Histone H2A, which was only found in somatic extracts.

FIG. 7F shows a RNA-seq analysis of Creb3/2 axonal expression following Aβ₁₋₄₂ stimulation of hippocampal neurons grown on microfluidic chambers, mined from Baleriola et al. 2014. Data points are presented normalized to somatic Creb3/2 levels. Values are log₂-transformed.

FIG. 7G shows a RT-qPCR analysis of Creb3/2 axonal expression following Aβ₁₋₄₂ stimulation of hippocampal neurons grown on microfluidic chambers. Data points are presented normalized to somatic Creb3/2 levels. Values are log₂-transformed.

FIG. 7H shows a Western blot analysis of CREB3L2 and ATF4 protein expression in neuritic extracts after Aβ₁₋₄₂ challenge. Cortico-hippocampal neurons were grown on transwell Boyden chambers and allowed to mature for 10-12 DIV before stimulation protocol. ATF4 and CREB3L2 levels were normalized against βIII-tubulin. Box-plots display a summary of n=6 independent experiments (CREB3L2: *P-value=0.0387; ATF4: *P-value=0.0378, unpaired t-tests); box-plot whiskers are drawn to the 10^{th} and 90^{th} percentiles.

FIG. 7I shows Puro-PLA detection of newly synthesized CREB3L2 protein in Aβ₁₋₄₂-treated axons. Hippocampal neurons were cultured in microfluidic chambers; a 10-minute puromycin pulse was applied at the end of a 12-hour Aβ₁₋₄₂ stimulation protocol (axonal treatments). Data are presented as mean±SEM of n=4 independently replicated experiments; per replicate, each condition was sampled from ca. 20 different optical fields; ***P-value <0.0001, one-way ANOVA test with post hoc Bonferroni multiple comparison correction. Anisomycin controls were performed twice. Scale bar, 20 μm.

FIGS. 8A-81 show that S2P mediates CREB3L2 activation in response to Δβ₁₋₄₂.

FIG. 8A shows a diagram depicting CREB3L2 and its activation by site 1 protease (S1P) and site 2 protease (S2P), which cleave CREB3L2 and liberate an N-terminal fragment that functions as a TF. CREB3L2 is a single-pass, type II transmembrane protein, i.e., it is oriented with its N-terminus facing the cytosol.

FIG. 8B shows a Western blot analysis of cytoplasmic (full-length) CREB3L2 protein levels in vehicle or Aβ₁₋₄₂-treated cortical neurons. Data are presented as mean±SEM of n=6-8 independent replicates, normalized to βIII-tubulin expression; *P-value=0.0103, unpaired t-test.

FIG. 8C shows a Western blot analysis of nuclear (cleaved) CREB3L2 protein levels in vehicle or Aβ₁₋₄₂-treated cortical neurons. Data are presented as mean±SEM of n=4-5 independent replicates, normalized to HDAC1 expression; *P-value=0.0153, unpaired t-test.

FIG. 8D shows a Western blot analysis of nuclear ATF4 protein levels in vehicle or Aβ₁₋₄₂-treated cortical neurons. Data are presented as mean±SEM of n=4 independent replicates, normalized to HDAC1 expression; **P-value=0.0082, unpaired t-test.

FIG. 8E shows the subcellular distribution of a GFP-CREB3L2 fusion carrying a mutated S1P cleavage site with or without S2P coexpression in HEK293. GFP signals were originally reticular in nature, but distinctively accumulate in the nucleus with S2P introduction. Scale bar, 25 μm.

FIG. 8F shows a Western blot study of CREB3L2 proteolytic cleavage. Coexpression of S2P, but not S1P, together with full-length GFP-CREB3L2 in HEK293 cells is sufficient to promote the activation of CREB3L2. Preventing S1P processing by eliminating its cleavage site does not impede S2P-mediated processing, indicating that CREB3L2 activation can occur independently of S1P.

FIG. 8G shows a Western blot analysis of CREB3L2 processing in HEK293 cells treated with S2P inhibitor nelfinavir. HRP-tagged CREB3L2 (full-length) was transiently transfected and cells co-treated with Aβ₁₋₄₂ oligomers and nelfinavir for 2 hours (Aβ₁₋₄₂: 1 μM; nelfinavir 15 μM).

FIG. 8H shows axonal CREB3L2-ATF4 heterodimers detected by PLA in hippocampal neurons cultured in microfluidic chambers. Axons were treated with vehicle or Aβ₁₋₄₂ oligomers for 36 hours, plus the S2P inhibitor nelfinavir. PLA signals were normalized to axon length and are reported as percentage fold change (±SEM) over vehicle control. Data were obtained from n=4 independent replicates, each including ca. 15 optical fields per condition; *P-value=0.0188, one-way ANOVA test with post hoc Bonferroni multiple comparison correction. Scale bar, 5 μm.

FIG. 8I shows cortical neurons expressing GFP-CREB3L2 (full-length) incubated with Aβ₁₋₄₂ and nelfinavir. Cells were pre-treated for 30 minutes with nelfinavir before Aβ₁₋₄₂ stimulation (2 hours). Data (nuclear-to-cytoplasmic GFP fluorescence ratios) are presented as mean±SEM of 138-161 neurons per condition, obtained over n=3 independent replicates; ***P-value <0.0001, one-way ANOVA test with post hoc Bonferroni multiple comparison correction. Scale bar, 25 μm.

FIGS. 9A-9H show that CREB3L2-ATF4 complexes are upregulated in LOAD.

FIGS. 9A and 9B show CREB3L2 and ATF4 mRNA expression profiles during pre- and postnatal development in the human dorsolateral prefrontal cortex (9A) and hippocampal formation (9B). Data were retrieved from BrainSpan.org, a gene expression atlas of the developing human brain (Miller et al., 2017). In total, 42 brain specimens, including both males and females, were surveyed using RNA-seq.

FIGS. 9C and 9D show CREB3L2 and ATF4 mRNA expression profiles in elder, non-demented (i.e., control) individuals. Data were obtained from the Aging, Dementia and Traumatic Brain Injury study and include regional gene expression analyses of the hippocampus (n=51) (9C) and cortical white matter (n=44) (9D) from both male and female individuals (Miller et al., 2017).

FIG. 9E shows a heatplot of a CREB3-like family of TFs in control and LOAD PFC. Rows denote individual cases. Controls, n=101; LOAD n=129. CREB3/LUMAN, P-value=9.07×10⁻⁵; CREB3L1/OASIS, P-value=8.60×10⁻¹7; CREB3L2, P-value=1.21×10⁻²⁰. CREB3L3/CREB-H, P-value=9.51×10⁻¹; CREB3L4/AIbZIP, P-value=1.65×10⁻⁸.

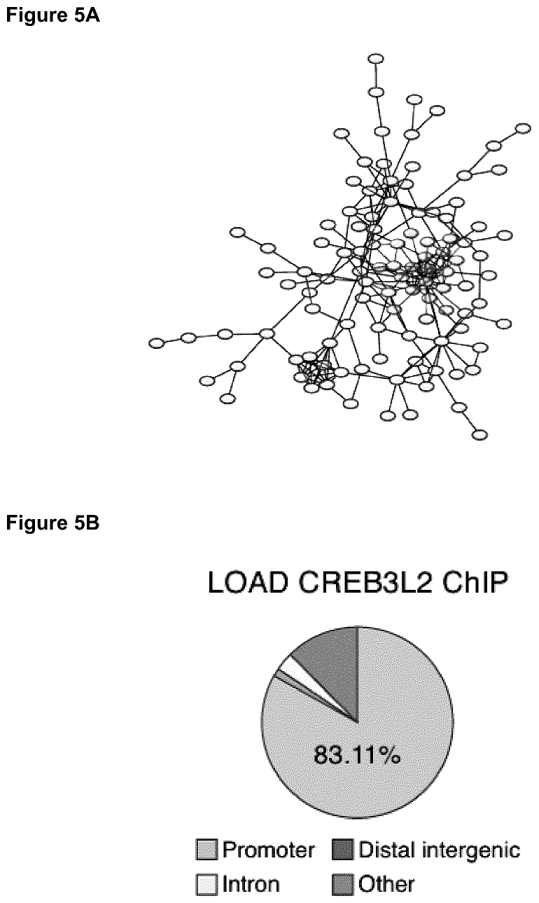

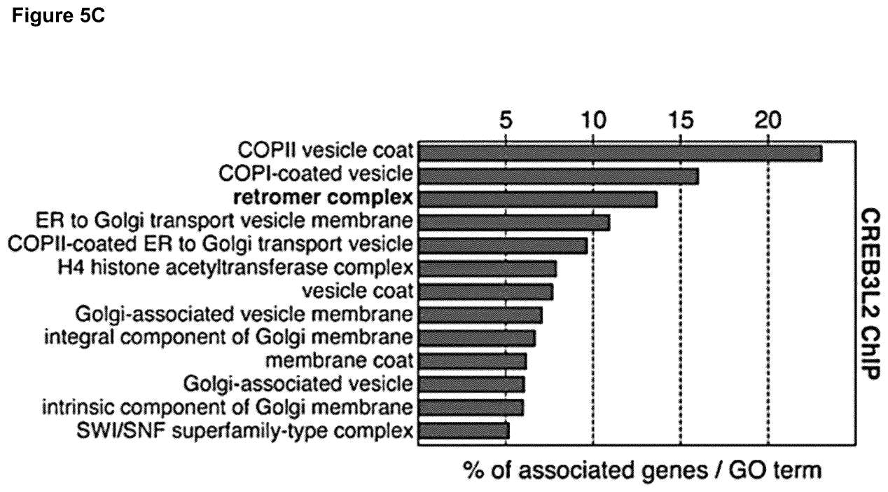

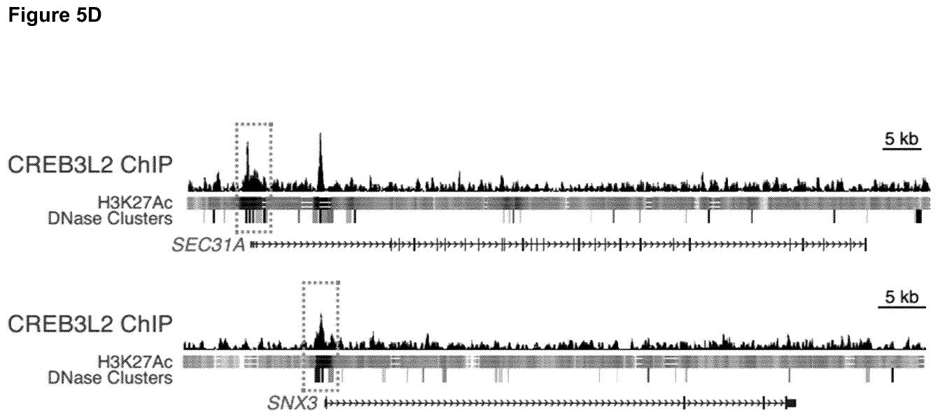

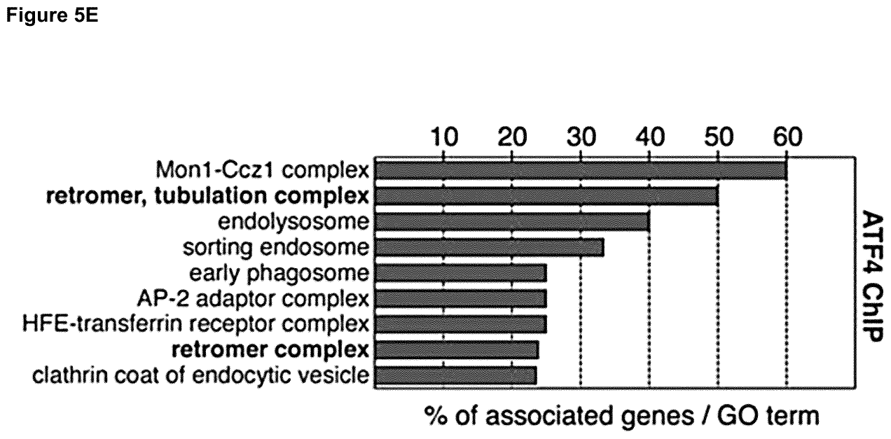

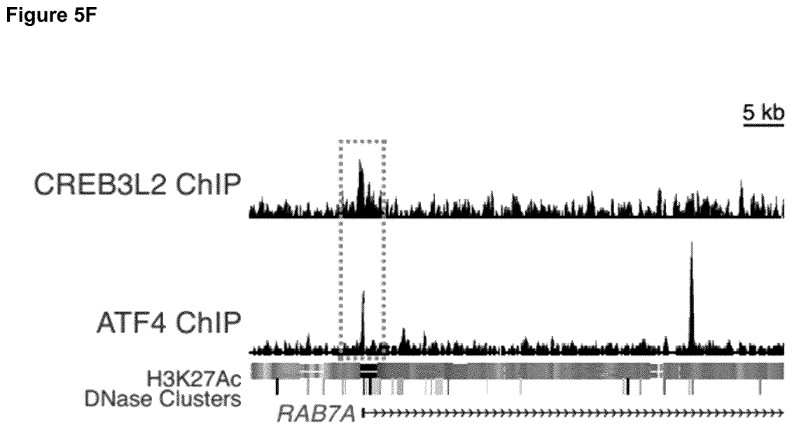

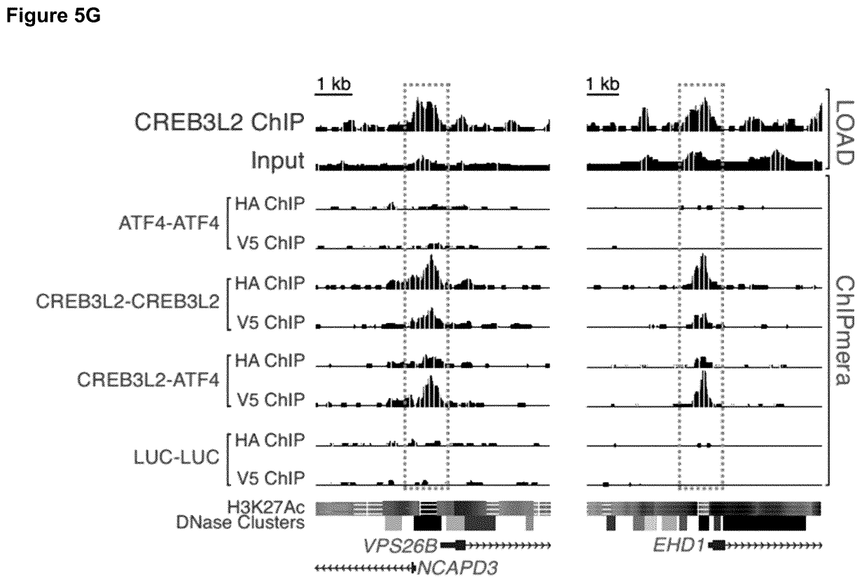

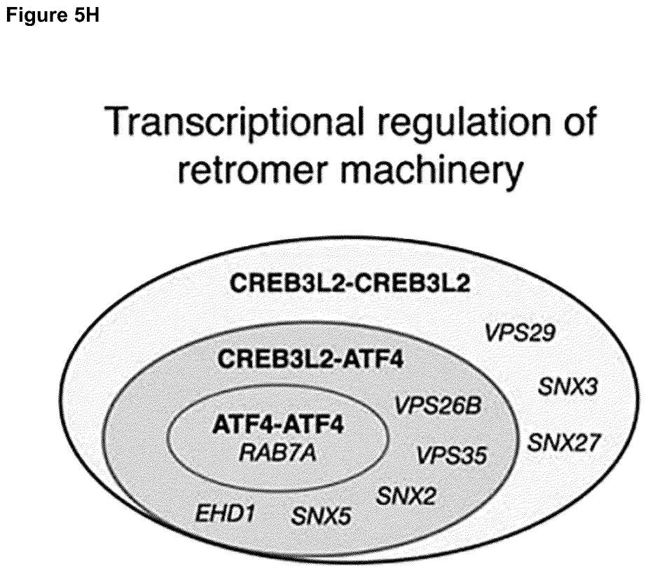

FIGS. 9F-9H show representative photomicrographs of CREB3L2 protein expression in LOAD prefrontal cortex (female, with mild pathology). Double immunohistochemical staining was achieved by combination of chromogen substrates with high color contrast (red/blue; dark brown signals, resulting from red+blue coincidence, denote antigen co-localization). CREB3L2 was stained red and cell-specific markers (MAP2 [neurons], GFAP [astrocytes], and IBA1 [microglia]) colored blue. Globally, evaluation of signals indicates that CREB3L2 is primarily expressed in neuronal cells in LOAD, in line with other reports. Some signal colocalization is observed with IBA1, indicating that CREB3L2 may be, to some extent, also expressed in microglia. This is the same LOAD case examined by ChIP-seq in FIGS. 5A-5H. Scale bar, 200 μm.

FIGS. 10A-10H show a representative CREB3L2-ATF4 transcriptional program according to one aspect of the present disclosure.

FIG. 10A shows PLA detection of CREB3L2-ATF4 heterodimers in HEK293 cells before and after addition of a bivalent dimerizer. Heterodimer detection employed anti-CREB3L2 and ATF4 antibodies. DAPI (nuclear counterstain), 4′,6-diamidino-2-phenylindole. Scale bar, 25 μm.

FIG. 10B shows a Western blot analysis of ChIPmera transgene expression in HEK293 cells. Different combinations of tagged TFs were co-expressed and probed 24 hours later with anti-CREB3L2, ATF4, HA or V5 antibodies, revealing overall comparable levels of expression between backgrounds.

FIG. 10C shows an analysis of HA and V5-ChIP-seq peak coincidence. Co-occurring hits are binned within defined genomic windows (e.g., peaks found no more that 50 bp apart) and shown as percentage of total peak number. The resolution of a ChIP-seq assay typically ranges from 200-500 bp.

FIG. 10D shows the cumulative frequency distribution of ChIPmera peaks proximal (<3 kb) to a TSS.

FIG. 10E shows an ATF4 and CREB3L2 homodimer motif similarity analysis using a Tomtom algorithm. JASPAR Core (2018 version) is a curated database of TF DNA-binding preferences. ATF4 homodimer versus JASPAR ATF4: P-value=7.08×10⁻⁸; CREB3L2 homodimer versus JASPAR CREB3L2: P-value=2.89×10⁻⁵.

FIGS. 10F-10H show a representative gene ontology functional analysis of targets bound by ATF4-ATF4, CREB3L2-CREB3L2 and CREB3L2-ATF4. These are not exhaustive lists; rather, in their elaboration, we aimed to provide a global perspective of enriched GO terms

FIGS. 11A-11D show a representative CREB3L2 transcriptional program in LOAD brain according to one aspect of the present disclosure.

FIG. 11A shows a cumulative frequency distribution of CREB3L2 ChIP-seq peaks proximal (<3 kb) to a TSS.

FIG. 11B shows a gene ontology term enrichment analysis of promoter-associated CREB3L2 targets (biological process).

FIG. 11C shows a heatplot visualization of PFC transcriptomic datasets from non-demented control and LOAD cases. It includes 25 significantly deregulated (P<1×10⁻²⁰) CREB3L2-targeted genes. Rows denote individual cases. Controls, n=101; LOAD cases, n=129 (Zhang et al., 2013).

FIG. 11D shows LOAD CREB3L2 ChIP-seq genome browser tracks in PTBP1 locus juxtaposed with ENCODE-produced H3K27Ac and DNaseI hypersensitivity profiles. PTBP1 encodes an RNA-binding protein involved in RNA splicing. For H3K27Ac and DNaseI hypersensitivity profiles, signal strength is indicated by increasingly darker shades.

FIG. 12 shows widespread retromer deregulation in LOAD brain (fusiform gyrus), using differential expression analysis of retromer-associated genes in the fusiform gyrus. Datasets comparing control and AD cohorts were mined from Friedman et al. 2018, and originate from bulk-extracted RNA examined by RNA-seq. Controls, n=33; LOAD cases, n=84.

FIG. 13 shows widespread retromer deregulation in LOAD brain (temporal cortex), using differential expression analysis of retromer-associated genes in the temporal cortex. Datasets comparing control and LOAD cohorts were mined from Webster et al. 2009, and originate from bulk-extracted RNA probed examined by microarray chip technology. Controls, n=135; LOAD cases, n=106.

FIGS. 14A-14K show a transcriptional mechanism of retromer deregulation acceding to one aspect of the present disclosure.

FIGS. 14A-14D show relative firefly luciferase reporter activities in homodimer- or heterodimer-expressing HEK293 cells. For each reporter, a Renilla luciferase homodimer, also induced using chemically induced proximity, was used to establish control background signals and replicate normalization (dashed lines). Individual data points (and mean±SEM summaries) of independently replicated experiments are shown: n=4 (SEC24A, VPS26B, VPS29), and n=3 (CHOP). Statistical significance was determined by one-way ANOVA test with post hoc Bonferroni multiple comparison correction.

FIG. 14E shows relative luminescence readings in HEK293 cells transiently expressing CREB3L2 and/or ATF4 together with a luciferase reporter (firefly) under control of the human RAB7A promoter/enhancer. In contrast to FIG. 6G, chemically induced proximity was not employed in this analysis; rather, ATF4 and CREB3L2 were simply overexpressed. Luminescence readings were normalized to total RNA levels and are presented relative to baseline signals measured from GFP-transfected cells. Individual data points (and mean±SEM summaries) of n=7 independently replicated experiments are shown; ***P-value <0.0001, one-way ANOVA test with post hoc Bonferroni multiple comparison correction.

FIG. 14F shows a ChIP-qPCR analysis of CREB3L2 binding to retromer gene promoters in rat cortical neurons grown in culture. ChIP signals were normalized against total input chromatin. Data are presented as mean±SEM of n=3-5 independent experiments; unpaired t-tests were used to determine statistical significance. Vps26b: *P-value=0.0209; Vps29: **P-value=0.0070; Vps35: **P-value=0.0085; Snx1: **P-value=0.0082; Snx3: ***P-value=0.0004; Rab7a: ***P-value=0.0009; Ehd1: *P-value=0.0495.

FIG. 14G shows a RT-qPCR analysis of retromer gene expression in Creb3/2-depleted rat cortical neurons. Measurements were normalized to Tubb3 and Pgk1, and are presented relative to control expression levels. Bar graphs show mean±SEM of n=3 independent experiments; unpaired t-tests were used to determine statistical significance. Vps26a: *P-value=0.0402; Vps29: *P-value=0.0113; Vps35: *P-value=0.0316; Snx1: *P-value=0.0186; Snx3: **P-value=0.0035; Rab7a: *P-value=0.0133.

FIG. 14H shows a Western blot analysis of retromer gene expression in rat cortical neurons infected with control or Creb3/2-targeting shRNAs. Measurements were normalized to βIII-tubulin levels and are presented relative to control baseline. Bar graphs show mean±SEM of n=3-4 independent experiments; unpaired t-tests were used to determine statistical significance. Vps26: **P-value=0.0063; Vps29: **P-value=0.0062; Vps35: **P-value=0.0060; Snx1: **P-value=0.0099; Snx3: ***P-value=0.0001; Rab7a: *P-value=0.0159.

FIG. 14I shows a RT-qPCR analysis of APP mRNA expression in rat cortical neurons infected with control or Creb3/2-targeting shRNAs. Data are presented as mean±SEM of n=4 independent experiments.

FIG. 14J shows a RT-qPCR analysis of APP mRNA expression in rat cortical neurons infected with control or Atf4-targeting shRNAs. Data are presented as mean±SEM of n=3 independent experiments; **P-value=0.0017, unpaired t-test.

FIG. 14K shows genome browser tracks centered on human APP locus from ENCODE-produced ATF4 ChIP-seq and ATF4 homodimer ChIPmera datasets, juxtaposed with H3K27Ac and DNaseI hypersensitivity profiles. For H3K27Ac and DNaseI hypersensitivity profiles, signal strength is indicated by increasingly darker shades. Two APP splice variants are shown, with Ensembl IDs ENST00000346798.8 (top) and ENST00000440126.7 (bottom).

FIGS. 15A-15J show that Aβ₄₂ promotes CREB3L2-ATF4 heterodimerization.

FIG. 15A shows the assessment of somatic degeneration by TUNEL staining of rat hippocampal neurons after Aβ₄₂ stimulation. Cells were grown until DIV 10-12 in microfluidic chambers, which allow for the fluidic isolation of axons from cell bodies and dendrites. Axons or cell bodies were transfected with control or Creb3/2-targeting siRNAs before axonal or somatic treatment with Aβ₄₂ for 48 hours, respectively. Data are presented as mean±SEM of n=10-12 biological replicates. Axonal dataset: *P-value=0.0357; somatic dataset: ***P-value=0.0003; statistical significance was determined by one-way ANOVA test with post hoc Bonferroni multiple comparison correction. Scale bar, 15 μm.

FIG. 15B shows experimental outline same as in FIG. 15A, except that nuclear CHOP protein expression levels were instead measured by quantitative immunofluorescence. Data are presented as mean±SEM of 150-184 optical fields per condition, obtained over n=8 independent replicates; ***P-value=0.0008, one-way ANOVA test with post hoc Bonferroni multiple comparison correction. Scale bar, 15 μm.

FIG. 15C shows coimmunoprecipitation of CREB3L2 with ATF4. Both proteins were produced by in vitro translation using lysates of rabbit reticulocytes, which are anucleated cells with all the cellular machinery necessary for protein synthesis but free of nuclear contaminants.

FIG. 15D shows coimmunoprecipitation analysis of CREB3L2 and ATF4 association in neuritic extracts after Aβ₄₂ challenge. Cortico-hippocampal neurons were grown on transwell inserts and matured for 10-12 DIV before stimulation protocol. ATF4 levels in CREB3L2 immunoprecipitates are normalized against input βIII-tubulin. Data are presented as mean±SEM of n=3 independent experiments (*P-value=0.0418, unpaired t-test).

FIG. 15E shows in situ visualization of axonal CREB3L2-ATF4 heterodimers detected by PLA in hippocampal neurons cultured in microfluidic chambers. Axons were treated with vehicle or Aβ₄₂ oligomers for 12 hours. Box-plots display a summary of relative interaction events obtained over n=3 independent experiments (***P-value <0.0001, unpaired t-test); box-plot whiskers are drawn to the 10^{th} and 90^{th} percentiles. PLA signals were normalized to 100-μm axon segments. Scale bar, 10 μm.

FIG. 15F shows axonal CREB3L2-ATF4 heterodimers detected by a proximity ligation assay. Emetine was delivered to hippocampal axons using a microfluidic chamber system. Approximately 60 axonal fields were analyzed per condition, over n=3 independent replicates; ***P-value <0.0001, one-way ANOVA test with post hoc Bonferroni multiple comparison correction. Scale bar, 10 μm.

FIG. 15G shows CREB3L2-ATF4 heterodimers visualized by PLA in vehicle- or Aβ₄₂-treated dissociated hippocampal neurons. Oligomeric Aβ₄₂ was bath-applied for 12 hours. βIII-tubulin and DAPI, a nuclear marker, were used as counterstains. Box-plots display a summary of relative interaction events obtained over n=3 independent experiments (nuclear: ***P-value=0.0007; somatic: **P-value=0.0016, unpaired t-tests); box-plot whiskers are drawn to the 10^{th} and 90^{th} percentiles. Scale bar, 10 μm.

FIG. 15H shows TUNEL staining of rat hippocampal neurons. Hippocampal neurons were cultured in microfluidic chambers for 10-12 DIV and Aβ₄₂ oligomers applied for 36 hours; nelfinavir, an S2P inhibitor, was applied in the last 24 hours of the Aβ₄₂ stimulus. Both treatments were delivered specifically to axons. Mean±SEM of n=3 independent experiments; per replicate, each condition was sampled from 10 different optical fields; ***P-value <0.0001, one-way ANOVA test with post hoc Bonferroni multiple comparison correction. Scale bar, 15 μm.

FIG. 15I shows quantitative immunofluorescence analysis of somatic CHOP protein expression levels. The experimental outline was the same as in (h). Mean±SEM of n=3 independent experiments; per replicate, each condition was sampled from 10 different optical fields; ***P-value <0.0001, one-way ANOVA test with post hoc Bonferroni multiple comparison correction. Scale bar, 15 μm.

FIG. 15J shows the detection of CREB3L2-ATF4 heterodimers by PLA in axons after local retrograde transport inhibition. Ciliobrevin A was delivered to axons in the last 6 hours of an 18-hour Aβ₄₂ protocol using a microfluidic chamber system. Box-plots display a summary of relative interaction events obtained over n=3 independent replicates (***P-value <0.0001, one-way ANOVA test with post hoc Bonferroni multiple comparison correction); box-plot whiskers are drawn to the 10^{th} and 90th percentiles. PLA signals were normalized to 100-μm axon segments. Scale bar, 10 μm.

FIGS. 16A-16F show that CREB3L2-ATF4 heterodimers are characteristic of AD.

FIG. 16A shows coimmunoprecipitation analysis of CREB3L2 and ATF4 association in control and late-onset AD PFC. Stratification by clinical stage reveals significant CREB3L2-ATF4 heterodimer upregulation in moderate pathology cases. Controls, n=5; AD n=8 (mild, n=2 and moderate, n=6); *P-value=0.0311, one-way ANOVA test; moderate AD vs. controls group comparison: *P-value=0.0312, Wilcoxon signed-rank test. ND, non-demented controls.

FIG. 16B shows PLA detection of CREB3L2-ATF4 heterodimers (red punctate signals) in AD PFC together with neurofilament staining (blue labelling), a neuronal marker particularly abundant in axons. Scale bar, 25 μm.

FIG. 16C shows western blot analysis of CREB3L2 and ATF4 protein levels in the PFC of control and late-onset AD cases. Controls, n=5; AD n=8; CREB3L2, *P-value=0.0195; ATF4, **P-value=0.0015, unpaired t-tests. Note that these are the same autopsy subjects examined in FIG. 16A.

FIG. 16D shows heatmap of CREB3L2 and ATF4 mRNA expression in control and AD PFC. Rows denote individual cases. Controls, n=101; AD n=129; CREB3L2, P-value=1.21×10⁻²⁰; ATF4, P-value=1.46×10⁻⁷.

FIG. 16E shows that the scatter plot of CREB3L2 (X-axis) and ATF4 (Y-axis) mRNA expression in control and AD PFC. AD cases showed a significant increase in CREB3L2 and ATF4 expression correlation. Controls, Pearson r=0.1325, P-value=0.19 (not significant), linear regression slope=0.07; AD, Pearson r=0.3972, ***P-value <0.0001, linear regression slope=0.47.

FIG. 16F shows CREB3L2 and ATF4 mRNA expression profiles stratified by sex and condition.

FIGS. 17A-17I show the CREB3L2-ATF4 transcriptional program.

FIG. 17A shows the experimental outline. ChIPmera exploits chemically induced proximity to induce the formation of specific TF pairs. Each monomer is engineered with two unique features: a specific dimerization domain (FRB or FKBP, C-terminally fused) and an N-terminal epitope tag (HA or V5). They can then be brought together in cells using a bivalent ligand that recognizes both the FRB and the FKBP domains with high affinity. Dimer-specific DNA-binding programs are elucidated from parallel HA and V5 ChIP-seq analyses. DNA transfection and dimerizer drug were delivered to HEK293T cells concurrently, and the ChIPmera system incubated for 24 hours before chromatin immunoprecipitation. ChIPmera background signals were established using a chemically induced Renilla luciferase homodimer.

FIG. 17B shows motif analysis of CREB3L2-CREB3L2, ATF4-ATF4, and CREB3L2-ATF4 binding sites. Heterodimer-bound sequences were centrally enriched in canonical CREB3L2 or ATF4 recognition motifs (CRE3L2 motif: present in 46% of input sequences [CentriMo E-value=4.7×10⁻³⁹]; ATF4 motif: present in 23% of input sequences [CentriMo E-value=4.0×10⁻³⁴]. Other statistically significant motifs found within ±50 bp ChIPmera peaks were not centrally distributed.

FIG. 17C shows representative binding behaviors displayed by CREB3L2-CREB3L2, ATF4-ATF4, and CREB3L2-ATF4 dimers. Also shown are ENCODE-generated histone H3 lysine 27 acetylation (H3K27Ac) and DNaseI hypersensitivity profiles, which mark important regulatory regions, such as gene promoters; in both cases, signal strength is indicated by increasingly darker shades.

FIG. 17D shows coincidence analysis of CREB3L2-ATF4 and CREB3L2-CREB3L2 DNA-binding sites across the genome. Genomic distances were computed within a ±2 kb window around CREB3L2-CREB3L2 peaks and are plotted as a frequency histogram.

FIG. 17E shows the same as in FIG. 17D, except that CREB3L2-ATF4 and ATF4-ATF4 dimers were analyzed instead.

FIG. 17F shows differential enrichment analysis of genomic regions bound by CREB3L2-ATF4 and ATF4-ATF4 dimers. Enrichment fold-change and statistical significance are plotted along the X- and Y-axes, respectively, from n=2 independent replicates. Magenta data points: false discovery rate (FDR) s 0.01; blue data points: FDR >0.01.

FIG. 17G shows differential enrichment analysis of genomic regions bound by CREB3L2-ATF4 and CREB3L2-CREB3L2 dimers. Axes layout and data point codification as in (d).

FIG. 17H shows GO functional analysis of CREB3L2-ATF4 targets (biological process).

FIG. 17I shows genome browser tracks in AD susceptibility loci juxtaposed with ENCODE-produced H3K27Ac and DNaseI hypersensitivity profiles.

FIGS. 18A-18F show that the retromer complex is a target of CREB3L2-ATF4.

FIG. 18A shows STRING protein interaction network of CREB3L2-bound genes identified by ChIP-seq on PFC AD chromatin. Nodes represent proteins, and edges denote predicted functional associations. STRING analysis identified 107 interconnected CREB3L2-regulated proteins.

FIG. 18B shows the distribution of CREB3L2 ChIP-seq signals across the genome. Cutoff for proximal promoter/enhancer regions was defined as ±3 kb from a TSS.

FIG. 18C shows GO term enrichment analysis of CREB3L2 targets (cellular component).

FIG. 18D shows AD CREB3L2 ChIP-seq genome browser tracks in SEC31A and SNX3 loci juxtaposed with ENCODE-produced H3K27Ac and DNaseI hypersensitivity profiles. SEC31A encodes a component of the COPII protein complex and participates in vesicle budding from the ER; SNX3 governs the interaction between the retromer and the early endosome.

FIG. 18E shows heatplot visualization of PFC transcriptomic profiles from non-demented control and AD individuals (Small and Petsko, 2015). Only the most significantly altered CREB3L2-targeted genes are included (cutoff defined as P<1×10⁻²⁰). Rows denote individual cases. Controls, n=101; AD cases, n=129.

FIG. 18F shows coincident CREB3L2 ChIP-seq peaks from AD PFC chromatin together with ChIPmera signals around VPS26B. For clarity, AD ChIP-seq and ChIPmera tracks are displayed using different viewing ranges.

FIGS. 19A-19I show that CREB3L2 is necessary for normal retromer expression and correlates with retromer misregulation in AD.

FIG. 19A shows western blot analysis of retromer gene expression in rat cortical neurons infected with control or Creb3/2-targeting shRNAs. Measurements were normalized to βIII-tubulin levels and are presented relative to control baseline. Bar graphs show mean±SEM of n=3-4 independent experiments; unpaired t-tests were used to determine statistical significance. Vps26: **P-value=0.0063; Vps29: **P-value=0.0062; Vps35: **P-value=0.0060; Snx1: **P-value=0.0099; Snx3: ***P-value=0.0001; Rab7a: *P-value=0.0159.

FIG. 19B shows RT-qPCR analysis of retromer gene expression in Creb3/2-depleted rat cortical neurons. Measurements were normalized to Tubb3 and Pgk1, and are presented relative to control expression levels. Bar graphs show mean±SEM of n=3 independent experiments; unpaired t-tests were used to determine statistical significance. Vps26a: *P-value=0.0402; Vps29: *P-value=0.0113; Vps35: *P-value=0.0316; Snx1: *P-value=0.0186; Snx3: **P-value=0.0035; Rab7a: *P-value=0.0133.

FIG. 19C shows ChIP-qPCR analysis of CREB3L2 binding to retromer DNA regulatory regions in dissociated rat cortical neurons. ChIP signals were normalized against total input chromatin. Data are presented as mean±SEM of n=3-5 independent experiments; unpaired t-tests were used to determine statistical significance. Vps26b: *P-value=0.0209; Vps29: **P-value=0.0070; Vps35: **P-value=0.0085; Snx1: **P-value=0.0082; Snx3: ***P-value=0.0004; Rab7a: ***P-value=0.0009.

FIG. 19D shows that membrane and cytosolic APP levels were analyzed by western blot in Creb3/2-suppressed rat cortical neurons. Surface proteomes were marked using a cell-impermeable biotin label. Efficient surface proteome capture is shown by glutamate receptor subunit staining (GluN2 A/B). βIII-tubulin levels were used for normalization between conditions (Wolff, 2009). Scatter plots display individual measurements and mean±SEM of n=6 independent replicates (***P-value=0.0086, unpaired t-test).

FIG. 19E shows that APP carboxy terminal fragments (CTFs) were compared by western blot between control and Creb3/2-suppressed rat cortical neurons using an antibody against the C-terminal portion of APP (Y188). Scatter plot displays individual measurements and mean±SEM of n=4 independent replicates (***P-value <0.0001, unpaired t-test). α, β, and □ denote α-secretase, β-secretase, and □-secretase, respectively.

FIG. 19F shows that culture supernatants from control and Creb3/2-deficient rat cortical neurons were probed for Aβ₄₂, Aβ₄₀, and sAPPα using antibody-capture plate assays (Meso Scale immunoassay [Aβ₄₂ and Aβ₄₀] and ELISA [sAPPα]). Scatter plots display individual measurements and mean±SEM of n=7 independent replicates. Differences between groups were statistically nonsignificant (ns; unpaired t-test).

FIG. 19G shows heatplot of retromer mRNA expression in PFC areas of non-demented control and AD human cohorts contrasted against CREB3L2. Data are log₂-transformed. Rows denote individual cases (Controls, n=101; AD n=129).

FIG. 19H shows retromer subunits with most significant differential expression in AD PFC.

FIG. 19I shows scatter plot of VPS35 (X-axis) and CREB3L2 (Y-axis) mRNA expression in control and AD PFC. Controls, Pearson r=−0.4481, ***P-value <0.0001, linear regression slope=−0.63; AD, Pearson r=−0.5595, ***P-value <0.0001, linear regression slope=−0.56.

FIGS. 20A-20H show that Retromer and other trafficking pathways are disrupted by CREB3L2-ATF4 signaling.

FIG. 20A shows that primary rat hippocampal neurons were infected with lentiviral particles carrying FK-domain-tagged transgenes and specific dimer configurations (CREB3L2-CREB3L2 [C-C], CREB3L2-ATF4 [C-A], and control luciferase-luciferase [L-L]) promoted by chemically induced proximity. Transcriptional profiles were analyzed by RNA-seq.

FIG. 20B shows volcano plot depicting changes in gene expression triggered by CREB3L2-ATF4 in hippocampal neurons in relation to control (Renilla luciferase homodimer) background levels. Fold-changes over baseline (log₂-transformed) and adjusted P-values (−log₁₀-transformed), as calculated by DESeq2 from n=5 independent replicates, are plotted along the X- and Y-axes, respectively. Magenta data points: adjusted P-value <0.05; blue data points: adjusted P-value >0.05.

FIG. 20C shows the same as in FIG. 20B, except that here CREB3L2-CREB3L2 homodimer-expressing neurons are compared against controls.

FIG. 20D shows meta-enriched ontology clusters (top 20) across CREB3L2-CREB3L2, CREB3L2-ATF4, and control backgrounds, colored by P-values (−log₁₀-transformed). Only significantly upregulated genes were taken part in this analysis. The first two columns denote enrichments over control, while the third column shows a direct comparison between CREB3L2-ATF4 and CREB3L2-CREB3L2 neurons.

FIG. 20E shows the same as in FIG. 20D, except that in this case, downregulated genes amongst all conditions were analyzed.

FIG. 20F shows the expression signatures (transformed as Z-score) for differentially expressed genes found in a head-to-head comparison between CREB3L2-ATF4 and CREB3L2-CREB3L2 neurons. Columns represent biological replicates; rows indicate individual genes.

FIG. 20G shows RT-qPCR analysis of retromer gene expression in CREB3L2-ATF4, CREB3L2-CREB3L2, and control neurons. Measurements were normalized to Tubb3 (βIII-tubulin) and are presented relative to baseline levels (luciferase dimer background), indicated by the dashed line. Scatter plots show individual measurements and mean±SEM of n=5-7 independent experiments; one-way ANOVA tests with Tukey's multiple comparison correction were used to determine statistical significance. Vps26b: **ANOVA P-value=0.0042, **Tukey's P-value (C-C vs. C-A)=0.0032. Vps35: **ANOVA P-value=0.0042, **Tukey's P-value (L-L vs. C-C)=0.0040, *Tukey's P-value (C-C vs. C-A)=0.0325. Ehd1: ***ANOVA P-value=0.0005, **Tukey's P-value (L-L vs. C-C)=0.0005, *Tukey's P-value (C-C vs. C-A)=0.0044.

FIG. 20H shows western blot analysis of retromer gene expression in CREB3L2-ATF4 and CREB3L2-CREB3L2 neurons. Measurements were normalized to βIII-tubulin levels and are shown relative to control baseline (luciferase dimer background). Scatter plots show individual measurements and mean±SEM of n=4 independent experiments (Vps29, n=3); one-way ANOVA tests with Sidak's multiple comparison correction were used to determine statistical significance. Vps29: *ANOVA P-value=0.0254, *Sidak's P-value (L-L vs. C-A)=0.0455, *Sidak's P-value (C-C vs. C-A)=0.0256. Vps35: *ANOVA P-value=0.0321, *Sidak's P-value (C-C vs. C-A)=0.0327. Snx3: *Sidak's P-value (L-L vs. C-A)=0.0434. Rab7a: *ANOVA P-value=0.0165, *Sidak's P-value (C-C vs. C-A)=0.0298. Ehd1: *ANOVA P-value=0.0199, *Sidak's P-value (L-L vs. C-C)=0.0195.

FIGS. 21A-21I show that P-amyloid and tau are downstream of CREB3L2-ATF4.

FIGS. 21A-21C show analysis of extracellular Aβ₄₂, Aβ₄₀, and Aβ₄₂/Aβ₄₀ ratios in culture supernatants collected from primary rat hippocampal neurons expressing CREB3L2-ATF4 (C-A), CREB3L2-CREB3L2 (C-C), or Renilla luciferase (L-L) dimers. Scatter dot plots show individual measurements and mean±SEM of n=7 independent replicates; one-way ANOVA tests with Tukey's multiple comparison correction were used to determine statistical significance. Aβ₄₂/Aβ₄₀: ***ANOVA P-value=0.0007, **Tukey's P-value (L-L vs. C-A)=0.0018, **Tukey's P-value (C-C vs. C-A)=0.0022. Aβ₄₂: ***ANOVA P-value=0.0003, ***Tukey's P-value (L-L vs. C-A)=0.0003, **Tukey's P-value (C-C vs. C-A)=0.0094. Aβ₄₀: ***ANOVA P-value=0.0003, ***Tukey's P-value (L-L vs. C-A)=0.0003, **Tukey's P-value (C-C vs. C-A)=0.0069. Here and henceforth, non-significant differences are not detailed.

FIG. 21D shows analysis of extracellular sAPPα levels by ELISA. Scatter dot plots show individual measurements and mean±SEM of n=7 independent replicates; statistical significance was determined by one-way ANOVA test with Tukey's multiple comparison correction. **ANOVA P-value=0.0052, **Tukey's P-value (L-L vs. C-A)=0.0066, *Tukey's P-value (C-C vs. C-A)=0.0269.

FIG. 21E shows that CTFs/APP ratios, total CTFs, and total APP were determined by western blot analysis using an antibody against the C-terminal portion of APP (Y188). Total CTFs and APP measurements were normalized to βIII-tubulin. Scatter plots display individual measurements and mean±SEM of n=3 independent replicates; statistical significance was evaluated by one-way ANOVA test with Tukey's multiple comparison correction. Total CTFs: *ANOVA P-value=0.0485, *Tukey's P-value (C-C vs. C-A)=0.0445.

FIG. 21F shows RT-qPCR analysis of App mRNA expression in CREB3L2-ATF4, CREB3L2-CREB3L2, and control neurons. Measurements were normalized to Tubb3 (pIII-tubulin) and are presented relative to baseline levels (Renilla luciferase dimer background), indicated by the dashed line. Scatter plot shows individual measurements and mean±SEM of n=6 independent experiments; statistical significance was evaluated by one-way ANOVA test with Tukey's multiple comparison correction: *ANOVA P-value=0.0308, *Tukey's P-value (C-C vs. L-L)=0.0448.

FIG. 21G shows the detection of LDH release by CREB3L2-ATF4, CREB3L2-CREB3L2, and control neurons using a bioluminescence protocol. Scatter plot shows individual measurements and mean±SEM of n=7 independent experiments; statistical significance was evaluated by one-way ANOVA test with Tukey's multiple comparison correction: *ANOVA P-value=0.0425, *Tukey's P-value (C-A vs. L-L)=0.0356.

FIG. 21H shows the extracellular tau protein levels. Scatter dot plots show individual measurements and mean±SEM of n=7 independent replicates; one-way ANOVA tests with Tukey's multiple comparison correction were used to determine statistical significance. **ANOVA P-value=0.0095, *Tukey's P-value (L-L vs. C-A)=0.0109, *Tukey's P-value (C-C vs. C-A)=0.0486.

FIG. 21I shows RT-qPCR analysis of Mapt mRNA expression. Measurements were normalized to Tubb3 (βIII-tubulin) and are presented relative to baseline levels (Renilla luciferase dimer background), indicated by the dashed line. Scatter plot shows individual measurements and mean±SEM of n=6 independent experiments; statistical significance was evaluated by one-way ANOVA test with Tukey's multiple comparison correction: *ANOVA P-value=0.0059, **Tukey's P-value (C-A vs. L-L)=0.0060, *Tukey's P-value (C-C vs. L-L)=0.0351.

FIGS. 22A-22H show that Aβ₄₂ promotes CREB3L2-ATF4 heterodimer formation.

FIG. 22A shows RNA-seq analysis of axonal Creb3/2 expression following Aβ₄₂ stimulation of hippocampal neurons grown under microfluidic isolation. This dataset was originally obtained by Baleriola et al., 2014. Log₂-transformed data points are presented normalized to somatic Creb3/2 levels.

FIG. 22B shows RT-qPCR analysis of axonal Creb3/2 expression following Aβ₄₂ stimulation of hippocampal neurons grown under microfluidic isolation. Log₂-transformed data points are presented normalized to somatic Creb3/2 levels.

FIG. 22C shows assessment of somatic degeneration by TUNEL staining of rat hippocampal neurons after Aβ₄₂ stimulation. Axons were transfected with control, Creb3/2, or Hif1a-targeting siRNAs before treatment with Aβ₄₂ for 48 hours. Hif1a is another TF-encoding message recruited into axons following Aβ₄₂ stimuli⁵. Unlike Creb3/2, silencing axonal Hif1a expression did not ameliorate Aβ₄₂-induced cell death. Data are presented as mean±SEM of n=10-12 biological replicates; *P-value=0.0357, one-way ANOVA test with post hoc Bonferroni multiple comparison correction. Scale bar, 15 μm.

FIG. 22D shows ChIP-qPCR assay of CREB3L2 binding to Chop regulatory DNA in vehicle- or Aβ₄₂-treated rat cortical neurons. Neurons were grown without microfluidic compartmentalization (i.e., dissociated cultures), and Aβ₄₂ bath-applied for 36 hours. ChIP signals in each sample were normalized against total input chromatin. Data are presented as mean±SEM of n=5 independent replicates; ***P-value <0.0001, one-way ANOVA test with post hoc Bonferroni multiple comparison correction.

FIG. 22E shows coimmunoprecipitation analysis of CREB3L2 and ATF4 binding in HEK293 cells (overexpression model). While wild-type CREB3L2-clv and ATF4 can heterodimerize, mutant CREB3L2 and ATF4 lacking leucine zippers do not establish detectable interactions.

FIG. 22F shows a schematic representation of the transwell Boyden chamber system employed to isolate neurites from cell bodies for biochemical analyses. To validate its applicability, we tested for the presence of a nuclear protein, Histone H2A, which was exclusively found in somatic extracts.

FIG. 22G shows western blot analysis of CREB3L2 and ATF4 protein expression in neuritic extracts after Aβ₄₂ challenge. Cortico-hippocampal neurons were grown on transwell Boyden chambers and matured for 10-12 DIV before stimulation protocol. ATF4 and CREB3L2 levels were normalized against βIII-tubulin. Box-plots display a summary of n=6 independent experiments (CREB3L2: *P-value=0.0387; ATF4: *P-value=0.0378, unpaired t-tests); box-plot whiskers are drawn to the 10^{th} and 90^{th} percentiles.

FIG. 22H shows Puro-PLA detection of newly synthesized CREB3L2 protein in Aβ₄₂-treated axons. Hippocampal neurons were cultured in microfluidic chambers; a 10-minute puromycin pulse was applied right at the end of a 12-hour Aβ₄₂ stimulus (both treatments were applied exclusively to axons). Data are presented as mean±SEM of n=4 independently replicated experiments; per replicate, each condition was sampled from ca. 20 different optical fields; ***P-value <0.0001, one-way ANOVA test with post hoc Bonferroni multiple comparison correction. Anisomycin controls were performed twice. Scale bar, 20 μm.

FIGS. 23A-23I show that S2P cleavage mediates CREB3L2-ATF4 heterodimer activation.

FIG. 23A shows a diagram depicting CREB3L2 and its activation by site 1 protease (S1P) and site 2 protease (S2P), which cleave CREB3L2 and liberate an N-terminal fragment that functions as a TF. CREB3L2 is a single-pass, type II transmembrane protein—i.e., its N-terminus is oriented facing the cytosol.

FIG. 23B shows western blot analysis of cytoplasmic (full-length) CREB3L2 protein levels in vehicle- or Aβ₄₂-treated cortical neurons. Data are presented as mean±SEM of n=6-8 independent replicates, normalized to βIII-tubulin expression; *P-value=0.0103, unpaired t-test.

FIG. 23C shows western blot analysis of nuclear (cleaved) CREB3L2 protein levels in vehicle- or Aβ₄₂-treated cortical neurons. Data are presented as mean±SEM of n=4-5 independent replicates, normalized to HDAC1 expression; *P-value=0.0153, unpaired t-test.

FIG. 23D shows western blot analysis of nuclear ATF4 protein levels in vehicle- or Aβ₄₂-treated cortical neurons. Data are presented as mean±SEM of n=4 independent replicates, normalized to HDAC1 expression; **P-value=0.0082, unpaired t-test.

FIG. 23E shows subcellular distribution of a GFP-CREB3L2 fusion carrying a mutated S1P cleavage site with or without S2P coexpression in HEK293. GFP signals were originally reticular in nature (membrane-bound, full-length CREB3L2) but distinctively accumulated in the nucleus with S2P co-expression, indicating that cleavage and activation can occur independently of S1P. Scale bar, 25 μm.

FIG. 23F shows western blot study of CREB3L2 activation. Expression of S2P, but not S1P, in HEK293 cells is sufficient to promote the cleavage of CREB3L2. Also, eliminating S1P processing altogether by mutating its cleavage site does not impede S2P-mediated processing, indicating that CREB3L2 activation can occur independently of S1P.

FIG. 23G shows western blot analysis of CREB3L2 processing in HEK293 cells treated with the S2P inhibitor nelfinavir. HRP-tagged CREB3L2 (full-length) was transiently transfected and cells co-treated with Aβ₄₂ oligomers and nelfinavir for 2 hours (Aβ₄₂: 1 μM; nelfinavir 15 μM).

FIG. 23H shows cortical neurons expressing GFP-CREB3L2 (full-length) incubated with Aβ₄₂ and nelfinavir. Cells were pre-treated for 30 minutes with nelfinavir before Aβ₄₂ stimulation (2 hours). Data (nuclear-to-cytoplasmic GFP fluorescence ratios) are presented as mean±SEM of 138-161 neurons per condition, obtained over n=3 independent replicates; ***P-value <0.0001, one-way ANOVA test with post hoc Bonferroni multiple comparison correction. Scale bar, 25 μm.

FIG. 23I shows axonal CREB3L2-ATF4 heterodimers detected by PLA in hippocampal neurons cultured in microfluidic chambers. Axons were treated with vehicle or Aβ₄₂ oligomer plus nelfinavir. PLA signals were normalized to axon length and are reported as percentage fold change (±SEM) over vehicle control. Data were obtained from n=4 independent replicates, each including ca. 15 optical fields per condition; *P-value=0.0188, one-way ANOVA test with post hoc Bonferroni multiple comparison correction. Scale bar, 5 μm.

FIGS. 24A-24D show that CREB3L2-ATF4 heterodimers are characteristic of AD.

FIG. 24A shows immunohistochemical labeling of CREB3L2 protein in AD PFC (female, mild pathology). Scale bar, 50 μm.

FIG. 24B shows immunohistochemical labeling of CREB3L2 and MAP2 (a neuronal marker) protein in AD PFC. Double immunohistochemical staining was achieved by the combination of chromogen substrates with high color contrast; CREB3L2 was stained red and MAP2 colored blue. Scale bar, 50 μm.

FIG. 24C shows PLA detection of CREB3L2-ATF4 heterodimers (red punctate signals) in AD PFC together with MAP2 staining (blue labeling). Scale bar, 50 μm.

FIG. 24D shows heatplot of CREB3-like family of TFs in control and AD PFC⁵. Rows denote individual cases. Controls, n=101; AD n=129. CREB31LUMAN, P-value=9.07×10⁻⁵; CREB3L1/OASIS, P-value=8.60×10⁻¹⁷; CREB3L2, P-value=1.21×10⁻²⁰; CREB3L31CREB-H, P-value=9.51×10⁻¹; CREB3L4/AIbZIP, P-value=1.65×10⁻⁸.

FIGS. 25A-25F show that ChIPmera identifies DNA-binding patterns of dimeric TFs across the genome.

FIG. 25A shows PLA detection of CREB3L2-ATF4 heterodimers in HEK293 cells with anti-CREB3L2 and anti-ATF4 antibodies before and after addition of dimerizer. DAPI (nuclear counterstain), 4′,6-diamidino-2-phenylindole. Scale bar, 25 μm.

FIG. 25B shows western blot analysis of ChIPmera transgene expression in HEK293 cells. Different permutations of tagged TFs were co-expressed and probed 24 hours later with anti-CREB3L2, ATF4, HA, or V5 antibodies, revealing overall comparable expression levels between backgrounds.

FIG. 25C shows the analysis of HA and V5-ChIP-seq peak coincidence for each dimer pair. A-A, ATF4-ATF4; C-C, CREB3L2-CREB3L2; C-A, CREB3L2-ATF4.

FIG. 25D shows genome browser tracks for CREB3L2-ATF4 and control Renilla luciferase LUC-LUC dimers. This representative 2,000,000 bp genomic window on chromosome 1 (chr1:150,151,864-152,151,863) in the vicinity of SNX27 shows various strong and coincident peaks for CREB3L2-ATF4. By contrast, LUC-LUC dimers produce neglectable ChIP-seq signals. Indeed, the MACS peak calling algorithm predicted, on average, just 12 significant peaks across the whole genome between the four LUC-LUC ChIP-seq experiments analyzed.

FIG. 25E shows cumulative frequency distribution of ChIPmera peaks proximal (<3 kb) to a transcription start site (TSS). A-A, ATF4-ATF4; C-C, CREB3L2-CREB3L2; C-A, CREB3L2-ATF4.

FIG. 25F shows motif similarity analysis using the Tomtom algorithm. JASPAR Core (2018 version) is a curated database of TF DNA-binding preferences (http://jaspar.genereg.net/). ATF4-ATF4 versus JASPAR ATF4: P-value=7.08×10⁻⁸; CREB3L2-CREB3L2 versus JASPAR CREB3L2: P-value=2.89×10⁻⁵.

FIGS. 26A-26D show the CREB3L2-ATF4 transcriptional program.

FIGS. 26A-26C show GO functional analyses of targets bound by ATF4-ATF4, CREB3L2-CREB3L2, and CREB3L2-ATF4. These are not exhaustive lists; rather, in their elaboration, we aimed to provide a global perspective of enriched terms.

FIG. 26D shows ChIPmera genome browser tracks in the vicinity of CREB3L2 and ATF4 loci juxtaposed with ENCODE-produced H3K27Ac and DNaseI hypersensitivity profiles.

FIGS. 27A-27C show the CREB3L2 transcriptional program in AD brain.

FIG. 27A shows cumulative frequency distribution of CREB3L2 ChIP-seq peaks proximal (<3 kb) to a TSS.

FIG. 27B shows GO term enrichment analysis of CREB3L2 targets (biological process).

FIG. 27C shows AD CREB3L2 ChIP-seq genome browser tracks in RAB7A locus juxtaposed with ENCODE-produced H3K27Ac and DNaseI hypersensitivity profiles. For H3K27Ac and DNaseI hypersensitivity profiles, signal strength is indicated by increasingly darker shades.

FIGS. 28A-28C show the widespread retromer misregulation in AD brain.

FIG. 28A shows RT-qPCR analysis of retromer gene expression in Creb3/2-depleted rat cortical neurons. Measurements were normalized to Tubb3 and are presented relative to control expression levels (ctrl shRNA). Bar graphs show mean±SEM of n=3-4 independent experiments; unpaired t-tests were used to determine statistical significance. Creb3/2: ***P-value <0.0001; ns, non-significant statistical differences.

FIG. 28B shows expression correlation analysis between CREB3L2 and individual retromer subunits in dorsolateral PFC.

FIG. 28C shows differential expression analysis of retromer-associated genes in the fusiform gyrus. Datasets comparing control and AD cohorts originate from bulk-extracted RNA examined by RNA-seq and were retrieved from Friedman et al. 2018. Controls, n=33; AD cases, n=84.

FIG. 29 shows the widespread retromer misregulation in AD brain (temporal cortex). Differential expression analysis of retromer-associated genes in the temporal cortex. Datasets comparing control and AD cohorts originate from bulk-extracted RNA probed examined by microarray chip technology and were retrieved from Webster et al. 2009. Controls, n=135; AD cases, n=106.

FIGS. 30A-30C show that CREB3L2-ATF4 heterodimerization alters retromer regulation.

FIG. 30A shows the volcano plot depicting differentially expressed genes in CREB3L2-ATF4 and CREB3L2-CREB3L2 neurons. CREB3L2-ATF4-induced transcriptional changes were determined against CREB3L2-CREB3L2 baseline expression levels. Fold-changes (log₂-transformed) and adjusted P-values (−log₁₀-transformed), as calculated by DESeq2 from n=5 independent replicates, are plotted along the X- and Y-axes, respectively. Magenta data points: adjusted P-value <0.05; blue data points: adjusted P-value >0.05.

FIG. 30B shows metascape output showing enriched TF-target interactions within differentially expressed genes.

FIG. 30C shows circos plot illustrating the overlap between differentially expressed genes in CREB3L2-ATF4, CREB3L2-CREB3L2, and control neurons. Outer arcs indicate the different backgrounds. On the inside, dark orange represents genes that appear in more than one list (e.g., a gene upregulated by CREB3L2-ATF4 and CREB3L2-CREB3L2 signaling), whereas light orange denotes genes that are unique to a specific background. Purple lines link a shared gene, while blue lines connect genes within the same GO term.

FIGS. 31A-31B show the increased expression and formation of the ATF4-CREB3L2 TF dimer in PD.

FIG. 31A shows a meta-analysis of 7 published microarray expression datasets (Moran et al. 2006; Zheng et al. 2010; Lesnick et al. 2007) of substantia nigra (SN) from PD and control patients was performed to assess for ATF4 and CREB3L2 expression.

FIG. 31B shows proximity ligation assay (PLA) for the ATF4-CREB3L2 complex in PD and control SN. Red-brown puncta indicate an interaction event. Nuclei containing puncta are highlighted by the arrow. Tyrosine hydroxylase is stained in blue, with nuclei in purple. Lighter brown areas inside neurons (*) are neuromelanin. Sections obtained from The New York Brain Bank at Columbia University. *=p<0.05.

FIGS. 32A-32B show ChIPmera (Chemically induced proximity+chromatin immunoprecipitation).

FIG. 32A shows that tagged TFs are induced to dimerize by addition of a chemical dimerizer (Stanton et al. 2018). ChIP-seq is performed for each tagged TF and resulting peaks compared for overlaps, which represent likely targets of the complex.

FIG. 32B shows PLA for ATF4 and CREB3L2 in the ChIPmera system before (left) and after (right) addition of the dimerizer. Green puncta represent interaction events within DAPI-stained nuclei.

FIGS. 33A-33D show the characterizing the transcriptional effect of the ATF4-CREB32 complex in PD.

FIG. 33A shows Venn diagram of the intersection between a published dataset of PD-associated differentially expressed genes (DEGs) (Kelly et al. 2019) and ATF4-CREB3L2 ChIPmera (labelled “A-C”).

FIG. 33B shows STRING network analysis of the 255 overlapping genes (Szklarczyk et al. 2018).

FIG. 33C shows functional enrichment analysis (GO process) of the overlapping gene dataset.

FIG. 33D shows that the network graph was further analyzed with a Markov Clustering algorithm (Brohee and Helden, 2006) to identify clusters of the most tightly related genes. The two clusters associated with the GO terms “mitochondrion organization” (left) and “mRNA processing” (right) are shown. Blue circles highlight the genes in each cluster associated with each term.

FIG. 34A shows a schematic view of dominant-negative inhibition of CREB3L2-ATF4 heterodimerization. bZIP transcription factors such as CREB3L2 and ATF4 heterodimerize by forming a parallel coiled-coil (the leucine zipper’) and bind DNA using a region rich in basic (i.e., positively charged) amino acids. Two proof-of-concept inhibitors were designed, each individually targeting CREB3L2 or ATF4. The strategy involved substituting the bZIP basic region of CREB3L2 or ATF4 with negatively charged residues. This so-called an ‘acidic extension’ electrostatically mimics DNA and provides an alternative interaction surface for the target bZIP basic region. Hence, by forming an extended coilded-coil structure, these dominant-negative ‘acidic-ZIP’ peptides can simultaneously prevent bZIP dimerization and DNA binding.

FIG. 34B shows CHOP luciferase reporter activities in CREB3L2-ATF4-expressing HEK293 cells with or without A-ZIP inhibitors. CHOP is a major target gene downstream of CREB3L2-ATF4 in Abeta42-mediated neurodegeneration, encoding for a pro-death transcription factor. A-ZIP peptides are shown to be extremely effective in preventing the increase of CHOP reporter activity promoted by CREB3L2-ATF4, indicating their applicability in a cellular model. Plot shows mean±SEM; “‘P-value <0.0001, one-way ANOVA with Dunnett's multiple comparison test.

FIG. 34C shows codon-optimized DNA sequence encoding for the ATF4 A-ZIP peptide. The amino acid sequence of the peptide is also shown.

FIG. 34D shows codon-optimized DNA sequence encoding for the CREB3L2 A-ZIP peptide. As in FIG. 34C, the amino acid sequence of the encoded peptide is shown.

FIG. 35A shows the analysis of nuclear CHOP protein by quantitative immunofluorescence. Axons were transfected with control or Creb3/2-targeting siRNAs before Aβ₄₂ treatment. Box-plots summarize n=8 replicates; ***P-value=0.0008, one-way ANOVA with Bonferroni correction. Here and henceforth, whiskers denote 10-90 percentile; ‘+’ sign marks sample mean. Creb3/2 knockdown ranged between 77.3-86.5% in dissociated neurons. Scale bar, 15 μm.

FIG. 35B shows the TUNEL assay in hippocampal neurons. Experimental outline as in (a). Box-plots summarize n=10-12 replicates; *P-value=0.0357, one-way ANOVA with Bonferroni correction. Scale bar, 15 μm.

FIG. 35C shows CREB3L2-ATF4 co-immunoprecipitation using in vitro translated proteins.

FIG. 35D shows CREB3L2-ATF4 co-immunoprecipitation analysis in HEK293 cells.

FIG. 35E shows co-immunoprecipitation of endogenous CREB3L2-ATF4 in neuritic extracts treated with Aβ₄₂. Mixed cortical and hippocampal neurons were grown on transwell inserts. ATF4 levels are normalized against input βIII-tubulin. Plot shows mean of n=3 replicates; *P-value=0.0418, unpaired t-test.

FIG. 35F shows the visualization of axonal CREB3L2-ATF4 by PLA. Hippocampal neurons were cultured in microfluidic chambers and axons treated with Aβ₄₂ for 12 hours. Box-plots summarize n=3 replicates; ***P-value <0.0001, unpaired t-test. Scale bar, 10 μm.

FIG. 35G shows PLA visualization of CREB3L2-ATF4 in dissociated hippocampal neurons. Aβ₄₂ was bath-applied for 12 hours. Box-plots summarize n=3 replicates. Nuclear CREB3L2-ATF4 events: ***P-value=0.0007; somatic CREB3L2-ATF4 events: **P-value=0.0016 (unpaired t-tests). Scale bar, 10 μm.

FIG. 35H shows the subcellular distribution of CREB3L2-ATF4.

FIG. 35I shows PLA visualization of CREB3L2-ATF4 after inhibition of axonal retrograde transport. Ciliobrevin A was delivered in the last 6 hours of an 18-hour Aβ₄₂ protocol. Box-plots summarize n=3 replicates; ***P-value <0.0001, one-way ANOVA test with Bonferroni correction. PLA signals were normalized to axon length. Scale bar, 10 μm.

FIG. 36 shows the in vivo detection and quantification of CREB3L2-ATF4 dimers by PLA in the dentate gyrus of 10-weeks-old 5×FAD or wild-type hippocampus. ML, molecular layer; IPL, inner polymorphic layer; GCL, granule cell layer. Wild-type, n=6; 5×FAD, n=5; ML, *P-value=0.0182; GCL, P-value=0.0574; IPL, *P-value=0.0285; unpaired one-tailed t-tests. Whiskers extend to the smallest and largest data values and sample means are indicated by ‘+’ sign. Scale bar, 10 μm.

FIG. 37A shows the experimental outline. ChIPmera uses chemically induced proximity to promote the formation of specific TF pairs. Each monomer is engineered with two unique features: a specific dimerization domain (FRB or FKBP, C-terminally fused) and an N-terminal epitope tag (HA or V5).

FIG. 37B shows the motif analysis of CREB3L2-CREB3L2, ATF4-ATF4, and CREB3L2-ATF4 binding sites. Heterodimer-bound sequences were centrally enriched in canonical CREB3L2 or ATF4 recognition motifs. CREB3L2: CentriMo E-value=4.7×10⁻³⁹; ATF4: CentriMo E-value=4.0×10⁻³. Other motifs identified were not centrally distributed.

FIG. 37C shows the representative binding behaviors displayed by CREB3L2-CREB3L2, ATF4-ATF4, and CREB3L2-ATF4 dimers. Also shown are ENCODE-generated histone H3 lysine 27 acetylation (H3K27Ac) and DNaseI hypersensitivity profiles.

FIG. 37D shows the coincidence analysis of CREB3L2-ATF4 and CREB3L2-CREB3L2 DNA-binding sites across the genome. Genomic distances were computed within a ±2 kb window around CREB3L2-CREB3L2 peaks and are plotted as a frequency histogram using a 10-bp bin size.

FIG. 37E is same as in FIG. 37D, except that here CREB3L2-ATF4 and ATF4-ATF4 dimers are compared.

FIG. 37F shows differential enrichment analysis of genomic regions bound by CREB3L2-ATF4 and ATF4-ATF4 dimers. Enrichment fold-change and statistical significance are plotted along the X- and Y-axes, respectively, from n=2 independent replicates (each replicate includes two parallel ChIP-seq runs). Magenta data points: false discovery rate (FDR) s 0.01; blue data points: FDR >0.01.

FIG. 37G shows the differential enrichment analysis of genomic regions bound by CREB3L2-ATF4 and CREB3L2-CREB3L2 dimers. Axes layout and data point codification are the same as in FIG. 37F. The strong signal ascribed to RNF157 coincides with a ATF4 pseudogene found within this locus.

FIG. 37H shows GO functional analysis of the CREB3L2-ATF4 transcriptional program (biological process).

FIG. 38A shows gene expression changes triggered by CREB3L2-ATF4 in rat hippocampal neurons analyzed by RNA-seq. Basal expression levels were measured in cells expressing Renilla luciferase homodimers. Fold-changes over baseline (log₂-transformed) and adjusted P-values (−log₁₀-transformed), as calculated by DESeq2 from n=5 independent replicates, are plotted along the X- and Y-axes, respectively. Magenta data points: adjusted P-value <0.05; blue data points: adjusted P-value >0.05.

FIG. 38B shows the strategy used to characterize the AD-associated transcription network regulated by CREB3L2-ATF4. First, we asked which of the differentially expressed genes in our RNA-seq dataset were direct DNA-binding targets of CREB3L2-ATF4 as identified by ChIPmera. Second, we evaluated the transcriptional signatures of these common hits in AD prefrontal cortex in order to understand which CREB3L2-ATF4-regulated targets had relevant disease-associated expression profiles (significance cutoff: P<1×10⁻¹⁵). We found that this subset included four upregulated TFs, NFE2L2, SOX9, NFATC1, and MXD4, as well as CREB3L2. Third, we explored the regulatory connections and functional relationships within this extended transcription network, described in FIG. 38C and FIG. 38D.

FIG. 38C shows the circos plot illustrating the regulatory relationships within the wider CREB3L2-ATF4 transcription network and their interaction with the AD transcriptome (bulk tissue-level). Genes within each category (e.g., CREB3L2-ATF4 targets) are arranged along the ideogram's arc. Inner lines link genes shared by two datasets (e.g., SOX9 and NFATC1); black-colored lines additionally identify genes with AD-associated transcriptional profiles.

FIG. 38D shows the representative GO terms enriched across input gene lists, colored by P-values (−log₁₀-transformed). This comparative analysis integrates the DNA-binding program of each TF, AD-associated gene expression changes, and the neuronal transcriptional profile promoted by CREB3L2-ATF4 measured in FIG. 38A.

FIG. 39A shows the retromer regulatory inputs within the CREB3L2-ATF4 transcription network. Pink-colored squares denote significant enrichments.

FIG. 39B shows the VPS35 ChIP-seq tracks alongside ENCODE-produced H3K27Ac marks and DNaseI hypersensitivity profiles.

FIG. 39C shows the retromer transcriptomic profiles (mined from 4) in non-demented control and AD individuals (bulk prefrontal cortex). Data are log₂-transformed. Rows denote individual cases (controls, n=101; AD n=129). EHD1: Person r=0.79; VPS29: Person r=−0.72; SNX1: Person r=0.70; SNX6: Person r: 0.68; VPS35: Person r: −0.67; VPS26B: Person r=−0.65.

FIG. 39D shows the VPS35 (X-axis) and CREB3L2 (Y-axis) mRNA expression prefrontal cortex. Controls, Pearson r=−0.4481, ***P-value <0.0001; AD, Pearson r=−0.5595, ***P-value <0.0001.

FIG. 39E shows the retromer protein levels in hippocampal neurons infected with control or Creb3/2-targeting shRNAs. Measurements were normalized to βIII-tubulin and are presented relative to control baseline. Mean±SEM of n=3-4 replicates; unpaired t-tests.

FIG. 39F shows ChIP-qPCR analysis of CREB3L2 normalized to total input chromatin. Mean±SEM of n=3-5 replicates; unpaired t-tests.

FIG. 39G shows RT-qPCR analysis of retromer gene expression in CREB3L2-ATF4 (C-A), CREB3L2-CREB3L2 (C-C), and control (L-L) hippocampal neurons. Measurements were normalized to Tubb3 (βIII-tubulin) and are presented relative to control (L-L background). Plots show individual measurements and mean±SEM of n=5-7 replicates; one-way ANOVA with Tukey's multiple comparison tests.

FIG. 39H shows the Western blot analysis of retromer protein levels in CREB3L2-ATF4 and CREB3L2-CREB3L2 neurons. Measurements were normalized to βIII-tubulin levels and are shown relative to control (L-L) baseline. Plots show individual measurements and mean±SEM of n=4 replicates (Vps29, n=3); one-way ANOVA tests with Sidak's multiple comparison correction.

FIG. 40A shows the top 3 enriched GO terms for CREB3L2-ATF4-interacting genes positively and negatively associated with β-amyloid or tau neuropathologies (−3≤signed −log₁₀ P-value ≥3). β-amyloid pathology overlap: representation factor=1.4, P<7.54×10⁻²³, hypergeometric test; tau pathology overlap: representation factor=1.4, P<8.15×10⁻⁸, hypergeometric test); cognitive decline overlap: representation factor=1.5, P<3.36×10⁻⁴³, hypergeometric test).

FIG. 40B shows the analysis of extracellular Aβ₄₂, Aβ₄₀, and Aβ₄₂/Aβ₄₀ ratios in culture supernatants from hippocampal neurons expressing CREB3L2-ATF4 (C-A), CREB3L2-CREB3L2 (C-C), or Renilla luciferase (L-L) dimers. Plots show individual measurements and mean±SEM of n=7 replicates; one-way ANOVA tests with Tukey's correction.

FIG. 40C shows the Western blot analysis of full-length APP. Plots display individual measurements and mean±SEM of n=6 replicates.

FIG. 40D shows the Western blot quantification of tau phosphorylation in rat hippocampal neurons normalized against total tau levels. Plots show individual measurements and mean±SEM of n=4 replicates; one-way ANOVA tests with Tukey's correction. Measurements shown here pertain only to tau signals around 50 kDa—see also FIG. 52F.

FIG. 40E shows the extracellular tau protein levels. Plots show individual measurements and mean±SEM of n=7 replicates; one-way ANOVA tests with Tukey's correction.

FIG. 40F shows the analysis of PP2A phosphatase activity in purified neuronal extracts. Plot show mean±SEM of relative cell number-normalized absorbance measurements from n=4 replicates; **P-value=0.0031, unpaired t-test.

FIG. 40G is a schematic showing that the findings support a model whereby CREB3L2-ATF4 promotes AD-linked gene expression changes and contributes to characteristic features of AD pathophysiology, including retromer dysfunction, altered β-amyloid metabolism, and tau hyperphosphorylation.

FIG. 41A shows the co-immunoprecipitation analysis of CREB3L2-ATF4 heterodimers in control and late-onset AD prefrontal cortex (immunoprecipitation with anti-ATF4 antibody). Plots show individual measurements and mean±SEM of CREB3L2/ATF4 ratios from n=4 controls and n=6 AD cases; *P-value=0.0133, unpaired t-test.

FIG. 41B shows PLA detection of CREB3L2-ATF4 heterodimers (green punctate signals) in AD dorsolateral prefrontal cortex co-stained for neurofilament (magenta labelling), a neuronal marker. This pseudocolored representative micrograph was produced using chromogenic detection methods. See FIG. 53D for quantification and technical controls. Scale bar, 25 μm.

FIG. 41C shows the genomic distribution of AD CREB3L2 ChIP-seq signals. Cutoff for proximal promoter/enhancer regions was defined as ±3 kb from a transcription start site.

FIG. 41D shows the cumulative frequency distribution of CREB3L2 ChIP-seq peaks relative to known transcription start sites.

FIG. 41E shows GO functional analysis of AD CREB3L2 transcriptional program (biological process).

FIG. 41F shows the representative CREB3L2 ChIP-seq tracks juxtaposed with ENCODE-produced H3K27Ac and DnaseI hypersensitivity profiles. SEC31A encodes a component of the COPII protein complex and participates in vesicle budding from the ER; SNX3 governs the interaction between the retromer and the early endosome; PTBP1 is a splicing regulator.

FIG. 41G shows the AD CREB3L2 ChIP-seq genome browser tracks in VPS26B locus juxtaposed with ChIPmera datasets and ENCODE-produced H3K27Ac and DNaseI hypersensitivity profiles. For clarity, AD ChIP-seq and ChIPmera tracks are displayed using different viewing ranges, as the ChIPmera signals are consistently stronger.

FIG. 41H shows GO term enrichment analysis (biological process) of targets common to both CREB3L2-ATF4 ChIPmera and AD CREB3L2 ChIP-seq datasets.

FIG. 42A shows the cell viability analysis of dissociated rat hippocampal neurons after 48-hour Aβ₄₂ stimulation protocol. Plots show individual measurements and mean±SEM of n=3 biological replicates. For each background, data are presented as Aβ₄₂/vehicle ratios. ATF4 aZIP: *P-value=0.0167; CREB3L2 aZIP: *P-value=0.0137; unpaired t-tests.

FIG. 42B shows gene expression changes triggered by Aβ₄₂ in rat hippocampal neurons analyzed by RNA-seq. Basal expression levels were measured in vehicle-treated cells. DEGs identified in Aβ₄₂ condition are compared against their expression values in neurons co-treated with dovinitib. Individual datapoints show fold-changes over baseline (log₂-transformed) of n=2 biological replicates. Aβ₄₂ and dovitinib were applied for 24 hours before RNA collection. A GO enrichment analysis of DEGs (n=203; 128 upregulated, 75 downregulated) in Aβ₄₂-treated neurons is also shown. FGF, fibroblast growth factor.

FIG. 43A shows axonal Creb3/2 expression following Aβ₄₂ stimulation of hippocampal neurons grown under microfluidic isolation. This analysis was performed using an RNA-seq dataset (GSE70860) previously published by our laboratory (Baleriola et al., 2014). log₂-transformed data points are normalized to somatic Creb3/2 levels.

FIG. 43B shows RT-qPCR analysis of axonal Creb3/2 expression following Aβ₄₂ stimulation of hippocampal neurons grown under microfluidic isolation. Log₂-transformed data points are normalized to somatic Creb3/2 levels. Summary of n=4 replicates; *P-value=0.0199, unpaired t-test.

FIG. 43C shows TUNEL assay of rat hippocampal neurons after Aβ₄₂ stimulation. Axons were transfected with control, Creb3/2, or Hif1a-targeting siRNAs before treatment with Aβ₄₂ for 48 hours. Hif1a is another TF-encoding gene whose mRNA is recruited into axons following Aβ₄₂ stimuli (Baleriola et al., 2014). Unlike Creb3/2, silencing axonal Hif1a expression did not ameliorate Aβ₄₂-induced cell death. Box-plots display summary of n=10-12 replicates; *P-value=0.0357, one-way ANOVA test with post hoc Bonferroni multiple comparison correction. Scale bar, 15 μm.

FIG. 43D shows the analysis of nuclear CHOP protein levels by quantitative immunofluorescence. Cells were cultured in microfluidic chambers for 10-12 DIV and Aβ₄₂ oligomers applied for 36 hours; nelfinavir, an S2P inhibitor, was added in the last 24 hours of the Aβ₄₂ protocol. Both treatments were delivered specifically to axons. Box-plot summary of n=3 experiments; per replicate, each condition was sampled from 10 different optical fields; ***P-value <0.0001, one-way ANOVA test with post hoc Bonferroni multiple comparison correction. Scale bar, 15 μm.

FIG. 43E shows the cell death analysis by TUNEL assay of rat hippocampal neurons. The experimental outline was the same as in FIG. 43D. Box-plot summary of n=3 independent experiments; per replicate, each condition was sampled from 10 different optical fields; ***P-value <0.0001, one-way ANOVA test with post hoc Bonferroni multiple comparison correction. Scale bar, 15 μm.

FIG. 43F shows ChIP-qPCR assay of CREB3L2 binding to Chop regulatory DNA in vehicle- or Aβ₄₂-treated rat cortical neurons. Neurons were grown without microfluidic compartmentalization (i.e., dissociated cultures), and Aβ₄₂ was bath-applied for 36 hours. ChIP signals in each sample were normalized against total input chromatin. Summary of n=5 independent replicates; ***P-value <0.0001, one-way ANOVA test with post hoc Bonferroni multiple comparison correction.

FIG. 43G shows a schematic representation of the transwell Boyden chamber system employed to isolate neurites from cell bodies for biochemical analyses. To validate its applicability, we tested for the presence of a nuclear protein, Histone H2A, which was exclusively found in somatic extracts.

FIG. 43H shows representative control reactions for CREB3L2-ATF4 PLA signals in dissociated hippocampal neurons counterstained with βIII-tubulin and DAPI. CREB3L2 antibody and blocking peptide were co-incubated before proceeding with the assay. CHOP is not known to interact with CREB3L2 and, in line with this, a PLA targeting both transcription factors produces negligible signals. Scale bar, 10 μm.

FIG. 43I shows the detection of axonal CREB3L2-ATF4 heterodimers by PLA after inhibition of local protein synthesis. Emetine was delivered to axons in the last 60 minutes of a 12-hour Aβ₄₂ stimulation protocol. Hippocampal neurons were cultured using a microfluidic chamber system to allow for axon-specific manipulations. Approximately 60 axonal fields were analyzed per condition, over n=3 independent replicates; ***P-value <0.0001, one-way ANOVA test with post hoc Bonferroni multiple comparison correction. Scale bar, 10 μm.

FIG. 43J shows the detection of neuronal CREB3L2-ATF4 heterodimers by PLA in dissociated hippocampal neurons. a-synuclein pre-formed fibrils were bath-applied for 10 days. βIII-tubulin and DAPI were used as counterstains. Box-plots display a summary of relative interaction events; 10 neurons were analyzed per condition in each experiment, over n=3 independent replicates. Scale bar, 5 μm.

FIG. 43K shows immunofluorescence analysis of intraneuronal a-synuclein accumulation after 10-day incubation protocol. Scale bar, 10 μm.

FIG. 44A shows the Western blot analysis of ChIPmera transgene expression in HEK293 cells. Different permutations of tagged TFs were co-expressed and probed 24 hours later with anti-CREB3L2, ATF4, HA, or V5 antibodies, revealing overall comparable expression levels between backgrounds.

FIG. 44B shows the co-immunoprecipitation analysis of chemically induced CREB3L2-CREB3L2 (C-C) and CREB3L2-ATF4 (C-A) dimers in HEK293 cells. Mean±SEM of n=4 independent replicates, normalized to input HA signals; ***P-value=0.0009, unpaired t-test.

FIG. 44C shows the co-immunoprecipitation analysis of chemically induced CREB3L2-CREB3L2 and CREB3L2-ATF4 dimers in HEK293 cells. Despite CREB3L2-ATF4 heterodimers forming a less efficient association (and/or being more prone to degradation) than CREB3L2-CREB3L2 homodimers, ATF4 is still able to compete with CREB3L2 for binding, as indicated by its appreciable presence in V5-CREB3L2 immunoprecipitates when HA-ATF4 and HA-CREB3L2 are co-expressed.

FIG. 44D shows PLA detection of chemically induced CREB3L2-ATF4 heterodimers in HEK293 cells with anti-CREB3L2 and anti-ATF4 antibodies before and after addition of dimerizer. DAPI (nuclear counterstain), 4′,6-diamidino-2-phenylindole. Scale bar, 10 μm.

FIG. 44E shows CHOP luciferase reporter activities in CREB3L2-ATF4-expressing HEK293 cells. Plot shows mean±SEM; ***P-value <0.0001, one-way ANOVA test with Dunnett's multiple comparison test.

FIG. 44F shows the genome browser tracks for CREB3L2-ATF4 and control Renilla luciferase LUC-LUC dimers. This representative 2,000,000 bp genomic window on chromosome 1 (chr1:150,151,864-152,151,863) in the vicinity of SNX27 shows various strong and coincident peaks for CREB3L2-ATF4. By contrast, LUC-LUC dimers produce neglectable ChIP-seq signals. Indeed, the MACS peak calling algorithm predicted, on average, just 12 significant peaks across the whole genome between the four LUC-LUC ChIP-seq experiments analyzed.

FIG. 44G shows the analysis of HA and V5-ChIP-seq peak coincidence for each dimer pair. A-A, ATF4-ATF4; C-C, CREB3L2-CREB3L2; C-A, CREB3L2-ATF4.

FIG. 44H shows the motif similarity analysis using the Tomtom algorithm. JASPAR Core (2018 version) is a curated database of TF DNA-binding preferences (http://jaspar.genereg.net/). ATF4-ATF4 versus JASPAR ATF4: P-value=7.08×10⁻⁸; CREB3L2-CREB3L2 versus JASPAR CREB3L2: P-value=2.89×10⁻⁵

FIG. 44I shows the cumulative frequency distribution of ChIPmera peaks proximal (<3 kb) to a transcription start site (TSS). A-A, ATF4-ATF4; C-C, CREB3L2-CREB3L2; C-A, CREB3L2-ATF4.

FIG. 45A shows ChIPmera genome browser tracks centered in AD susceptibility loci. bp, base pair; kb, kilobase.

FIG. 45B shows GO functional analysis (biological process) of the CREB3L2-ATF4-specific genes (i.e., not shared with CREB3L2-CREB3L2). Genes were classified as ‘proximal’ or ‘distal’ depending on the proximity of its corresponding peak to the nearest transcription start site.

FIG. 45C shows the genomic distribution of CREB3L2-ATF4 ChIPmera peaks not associated with CREB3L2-CREB3L2. The large majority of these (91.7%) coincide with ChIP-seq ATF4 signals described by the ENCODE Consortium. Cutoff for proximal promoter/enhancer regions was defined as ±3 kb from a transcription start site.

FIG. 45D shows ChIPmera genome browser tracks in the vicinity of CREB3L2 and ATF4 loci juxtaposed with ENCODE-produced H3K27Ac and DNaseI hypersensitivity profiles. A-A, ATF4-ATF4; C-C, CREB3L2-CREB3L2; C-A, CREB3L2-ATF4; L-L, LUC-LUC.

FIG. 46A shows Volcano plot depicting changes in gene expression triggered by CREB3L2-CREB3L2 homodimers in hippocampal neurons. Basal expression levels were measured in cells expressing Renilla luciferase homodimers. Fold-changes (log₂-transformed) and adjusted P-values (−log₁₀-transformed), as calculated by DESeq2 from n=5 independent replicates, are plotted along the X- and Y-axes, respectively. Magenta data points: adjusted P-value <0.05; blue data points: adjusted P-value >0.05.

FIG. 46B is the same as in FIG. 46A, except that here a head-to-head comparison between neurons expressing CREB3L2-ATF4 or CREB3L2-CREB3L2 dimers is shown.

FIG. 46C shows the meta-enriched ontology clusters (top 20) across CREB3L2-CREB3L2, CREB3L2-ATF4, and control backgrounds, colored by P-values (−log₁₀-transformed). Only significantly upregulated genes were included in this analysis. The first two columns denote enrichments over control, while the third column shows a direct comparison between CREB3L2-ATF4 and CREB3L2-CREB3L2 neurons.

FIG. 46D is the same as in FIG. 46C, except that downregulated genes were analyzed.

FIG. 46E shows GO functional analyses of the 221 DEGs downstream of CREB3L2-ATF4 activation in neurons previously identified as direct DNA-binding targets of the heterodimer in our ChIPmera study.

FIG. 47A shows the expression correlation between CREB3L2-ATF4 targets with AD-associated transcriptional profiles and CREB3L2 or ATF4 in diseased individuals. These analyses were performed in two independent cohorts (ROSMAP, Religious Order Study/Memory and Aging Project) (Zhang et al., 2013; Bennett et al., 2018). P-values indicate correlation significances. It is noteworthy that the number of cases assigned to each Braak stage varies considerably, with most individuals in the ROSMAP cohort being stratified between BR3 and BR5. n/a, not available (SEC22B) or not applicable (CREB3L2).

FIG. 47B shows the head-to-head comparison of gene expression profiles in CREB3L2-ATF4 neurons and AD prefrontal cortex (Zhang et al., 2013). These are the same 53 genes directly targeted by the heterodimer and misregulated in AD.

FIG. 47C shows NFE2L2, SOX9, NFATC1, and MXD4 AD gene expression profiles across various disease-relevant brain regions (Webster et al., 2009; Friedman et al., 2018). Prefrontal cortex samples shown here were mined from the ROSMAP cohort (Bennett et al., 2018).

FIG. 47D shows the regulatory interconnections within the wider CREB3L2-ATF4-activated NRF2-SOX9-NFATC1-MXD4 network.

FIG. 47E shows the circos plot illustrating the regulatory relationships within the wider CREB3L2-ATF4 transcription network and their interaction with CREB3L2-ATF4-promoted transcriptional changes in neurons. Inner lines link genes shared by two datasets (e.g., SOX9 and NFATC1); black-colored lines additionally identify genes with altered transcriptional profiles downstream of CREB3L2-ATF4 activation in neurons.

FIG. 48A shows cell-type-specific transcriptomic analysis using snRNA-seq datasets produced by Mathys et al., 2019. These are the same 53 DEGs examined in FIG. 38B. Controls, n=24 (individuals with no or very low β-amyloid burden; AD-pathology, n=24 (cases with high levels of β-amyloid and other neuropathological hallmarks of AD). Ex, excitatory neurons; In, inhibitory neurons; Ast, astrocytes; Oli, oligodendrocytes; Opc, oligodendrocyte precursor cells; Mic, microglia.

FIG. 48B shows the regulatory relationships within the CREB3L2-ATF4 transcription network and their interaction with DEGs in AD excitatory neurons. Black-colored lines identify genes with AD-associated transcriptional profiles.

FIGS. 48C and 48D show the representative GO terms enriched across input gene lists, colored by P-values (−log₁₀-transformed). This comparative analysis integrates the DNA-binding program of each TF within the CREB3L2-ATF4 network, neuronal gene expression changes detected in AD excitatory neurons (Mathys et al., 2019), and the transcriptional profile promoted by CREB3L2-ATF4 in cultured neurons described in FIG. 38A. In FIG. 48D, progressive gene expression changes were considered by subgrouping individuals according to clinicopathological traits. ‘Early pathology’, as defined by Mathys and colleagues (Mathys et al., 2019), included individuals with some amyloid burden but modest neurofibrillary tangles/cognitive impairment; cases presenting with higher amyloid loads, increased neurofibrillary tangles, and cognitive decline were classified as ‘late pathology’.

FIG. 49 shows the differential expression analysis of retromer-associated genes in the fusiform gyrus. Datasets comparing control and AD cohorts were produced by Friedman et al. from bulk-extracted RNA (Friedman et al., 2018). Controls, n=33; AD cases, n=84.

FIG. 50A shows the differential expression analysis of retromer-associated genes in the temporal cortex. Datasets comparing control and AD cohorts were produced by Webster et al. from bulk-extracted RNA (Webster et al., 2009). Controls, n=135; AD cases, n=106.

FIG. 50B shows the expression profiles for top-ranked retromer subunits in AD excitatory neurons derived from dorsolateral prefrontal cortex snRNA-seq datasets (Mathys et al., 2019). DEGs IndModel: logical indication of whether a gene meets ‘false discovery rate (FDR)-adjusted P-value’<0.01 and ‘absolute log₂(foldchange)’>0.25 conditions; DEGs MixedModel: logical indication of whether a gene meets ‘FDR-adjusted MixedModel P-value’<0.05 condition.

FIG. 51A shows the RT-qPCR analysis of retromer gene expression in Creb3/2-depleted rat cortical neurons. Measurements were normalized to Tubb3 and Pgk1 and are presented relative to control expression levels. Mean±SEM of n=3 independent experiments. Vps26a: *P-value=0.0402; Vps29: *P-value=0.0113; Vps35: *P-value=0.0316; Snx1: *P-value=0.0186; Snx3: **P-value=0.0035; Rab7a: *P-value=0.0133; unpaired t-tests.

FIG. 51B shows the Western blot analysis of CI-M6PR degradation kinetics in control (LUC-LUC homodimer) and CREB3L2-ATF4-expressing hippocampal neurons after treatment with 40 μg/ml cycloheximide for 2, 4, or 6 hours. CI-M6PR levels are presented relative to t=0 timepoint. Plot shows mean±SEM of n=4 independent experiments; two-way ANOVA with Sidak's multiple comparison test. Column factor (Background): **ANOVA P-value=0.0014; Row factor (Time): **ANOVA P-value=0.0078; *Sidak's P-value (t=6 h)=0.0203.

FIGS. 52A-52C show the enriched GO terms for CREB3L2-ATF4-interacting genes positively and negatively associated (−3 s signed −log₁₀ P-value >3) with β-amyloid or tau neuropathologies. Pathology-interacting molecular networks were originally elucidated by De Jager and colleagues (Mostafavi et al., 2018). Cognition, unlike β-amyloid and tau neuropathologies, declines over time; hence, a GO term negatively correlated with cognition indicates a deleterious association.

FIG. 52D shows the RT-qPCR analysis of App mRNA expression in CREB3L2-ATF4, CREB3L2-CREB3L2, and control neurons. Measurements were normalized to Tubb3 (βIII-tubulin) and are presented relative to baseline levels (Renilla luciferase dimer background), indicated by the dashed line. Plot shows individual measurements and mean±SEM of n=6 independent experiments; one-way ANOVA test with Tukey's multiple comparison correction: *ANOVA P-value=0.0308, *Tukey's P-value (C-C vs. L-L)=0.0448.

FIG. 52E shows the analysis of extracellular sAPPα levels by ELISA. Plots show individual measurements and mean±SEM of n=7 independent replicates; one-way ANOVA test with Tukey's multiple comparison correction. **ANOVA P-value=0.0052, **Tukey's P-value (L-L vs. C-A)=0.0066, *Tukey's P-value (C-C vs. C-A)=0.0269.

FIG. 52F shows the Western blot quantification of tau phosphorylation in rat hippocampal neurons normalized against total tau levels. Plots show individual measurements and mean±SEM of n=4 independent replicates; sample differences are not statistically significant due to overall higher variability in control replicates. Measurements shown here pertain only to tau signals of 60 kDa isoform—see immunoblots in FIG. 40D.

FIG. 52G shows the RT-qPCR analysis of Mapt mRNA expression. Measurements were normalized to Tubb3 (βIII-tubulin) and are presented relative to baseline levels (Renilla luciferase dimer background), indicated by the dashed line. Plot shows individual measurements and mean±SEM of n=6 independent experiments; one-way ANOVA test with Tukey's multiple comparison correction: *ANOVA P-value=0.0059, **Tukey's P-value (C-A vs. L-L)=0.0060, *Tukey's P-value (C-C vs. L-L)=0.0351.

FIG. 52H shows ELISA quantification of extracellular Neurofilament-light (NF-L) and MAP2 levels. Plots show individual measurements and mean±SEM. NF-L: n=10 independent replicates; MAP2: n=8 independent replicates.

FIG. 52I shows the differentially expressed PP2A and PP1 subunits after CREB3L2-ATF4 activation in primary hippocampal neurons, as determined by RNA-seq. Ppp2r1b: log fold-change=−0.40, P-value=0.017; Ppp2r5a: log fold-change=+0.29, P-value=0.014; Strn4: log fold-change=−0.17, P-value=0.034; Ppp2ca: log fold-change=−0.15, P-value=0.016; Ppp1r15b: log fold-change=+0.37, P-value=0.00012; Ppp1r37: log fold-change=−0.20, P-value=0.034. Asterisks denote DEGs found within the CREB3L2-ATF4 transcription network.

FIG. 52J shows the STRING protein interaction network of CREB3L2-ATF4-induced DEGs with links to tau (average local clustering coefficient=0.787; network enrichment: P-value=1.14×10⁷). Nodes represent proteins, and edges denote predicted functional associations. Purple edges represent experimentally determined protein-protein associations.

FIG. 53A shows CREB3L2-ATF4 co-immunoprecipitation dataset shown in FIG. 41A stratified according to ABC scores (A, amyloid; B, Braak; C, CERAD [neuritic plaques]). Plots show individual measurements and mean±SEM of CREB3L2/ATF4 ratios from n=4 controls and n=6 AD cases.

FIG. 53B shows additional quantifications of co-immunoprecipitation reactions of CREB3L2-ATF4 heterodimers in control and late-onset AD prefrontal cortex shown in FIG. 41A. Plots show mean±SEM of n=4 controls and n=6 AD cases; unpaired t-tests were performed for statistical comparisons; **P-value=0.0022. Decreased overall levels of ATF4 may indicate prolonged CREB3L2-ATF4 activation (see, for example, FIGS. 38A, 40C and 45D).

FIG. 53C shows the immunoprecipitation reaction with control rabbit IgG in control and AD prefrontal cortex shows no enrichment of ATF4. These are the same cases analyzed in FIGS. 41A, 53A and 53B.

FIG. 53D shows the quantification of dorsolateral prefrontal CREB3L2-ATF4 heterodimers (red punctate signals) in control and AD cases. Brains were co-stained for neurofilament (blue labelling), a neuronal marker. PART, primary age-related tauopathy. Plots show, for each case, average neuronal CREB3L2-ATF4 signals across non-contiguous layer III-V segments (mean±SEM). Controls, n=3; AD, n=4; PART, n=1; *P-value=0.0234, unpaired t-test. Technical control 1: CREB3L2 PLA probe and CREB3L2 blocking peptide were co-incubated before proceeding with the assay; Technical control 2: CREB3L2 PLA probe was substituted with rabbit IgG PLA probe. Scale bar, 5 μm.

FIG. 53E shows the GO term enrichment analysis of AD CREB3L2 targets (cellular component).

FIG. 53F shows AD CREB3L2 ChIP-seq genome browser tracks in RAB7A locus juxtaposed with ENCODE-produced H3K27Ac and DNaseI hypersensitivity profiles. For H3K27Ac and DnaseI hypersensitivity profiles, signal strength is indicated by increasingly darker shades.

FIG. 53G shows the representative GO terms enriched across input gene lists, colored by P-values (−log₁₀-transformed). This comparative analysis integrates the DNA-binding program of each TF within the CREB3L2-ATF4-activated NRF2-SOX9-NFATC1-MXD4 transcription network, as well as the AD CREB3L2 ChIP-seq readout.

### DETAILED DESCRIPTION OF THE DISCLOSURE

In the present disclosure, a pathological TF complex associated with LOAD and CREB3L2-ATF4, was identified and characterized. This association was discovered in a screen for axon-derived modulators of neurodegenerative responses set off by peripheral β-amyloid and confirmed its pathological significance in LOAD brain tissue. To dissect its role in disease, a new methodology, ChIPmera, was developed, which resolves the DNA-binding profile of dimeric TFs in vivo. In combination with other analyses, the retromer machinery was identified as a disease-relevant transcriptional target of CREB3L2-ATF4, and the heterodimer was linked to disrupted retromer gene expression.

Accordingly, one embodiment of the present disclosure is a method for treating or ameliorating the effects of a neurodegenerative disease in a subject, comprising: (a) determining the level of CREB3L2-ATF4 transcription factor (TF) complex in a sample obtained from the subject; and (b) administering to the subject an effective amount of an agent that modulates the association between CREB3L2 and ATF4, if the level of CREB3L2-ATF4 complex determined in step (a) is significantly different from that of a control subject.

As used herein, “significantly different” refers to statistical significance, which means a result is unlikely due to chance. It can be determined by comparing p-value, which is the probability of obtaining a result at least as extreme, with an arbitrary pre-set significance level (a). In the context of this disclosure, if a is selected to be 0.05, the level of CREB3L2-ATF4 complex in a sample is significantly different from that of a control subject if the calculated p-value is less than 0.05.

In some embodiments, the neurodegenerative disease is associated with retromer complex dysfunction, altered β-amyloid metabolism, tau hyperphosphorylation, or combinations thereof. In some embodiments, the neurodegenerative disease is selected from the group consisting of Alzheimer's Disease, Parkinson's Disease, Frontotemporal Lobar Degeneration, Down's Syndrome, Hereditary Spastic Paraplegia, Neuronal Ceroid Lipofuscinoses, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, and Progressive supranuclear palsy. In some embodiments, the neurodegenerative disease is Alzheimer's disease or Parkinson's disease. In some embodiments, the neurodegenerative disease is late-onset Alzheimer's disease (LOAD).

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments of the present disclosure, the phrase “a subject” means a subject having a neurodegenerative disease such as, e.g., Alzheimer's disease.

In some embodiments, the agent that modulates the association between CREB3L2 and ATF4 does not ablate CREB3L2 signaling. An exemplary agent may include those that reduce ATF4 activity such as, e.g., ursolic acid and tomatidine. In some embodiments, the agent is dovitinib.

In some embodiments, the determining step (i.e., step (a)), may be carried out using any conventional method for determining the level of CREB3L2-ATF4 TF complex in a biological sample. In certain aspects of this embodiment, the determining step is as set forth in, e.g., Examples 1 and 4 of the present disclosure.

As used herein, a “biological sample” means a biological specimen, which may be a bodily fluid or a tissue. Biological samples include, for example, whole blood, serum, plasma, cerebro-spinal fluid, leukocytes or leukocyte subtype cells (e.g. neutrophils, basophils, and eosinophils, lymphocytes, monocytes, macrophages), fibroblast sample, olfactory neuron sample, and tissues from the central nervous system, such as the cortex (e.g., dorsolateral PFC) and hippocampus, and cells previously exposed to the CNS environment, such as dendritic cells trafficked from the brain, or other immune or other cell types (Mohamed-M G et al., 2014). Examples of preferred biological samples include, e.g., a blood sample, a biopsy sample, a plasma sample, a saliva sample, a tissue sample, a serum sample, a tear sample, a sweat sample, a skin sample, a cell sample, a hair sample, an excretion sample, a waste sample, a bodily fluid sample, a nail sample, a cheek swab, a cheek cell sample, or a mucous sample. In some embodiments, the biological sample can be a tissue section or a biopsy from dorsolateral PFC, blood, or other appropriate bodily fluid.

Another embodiment of the present disclosure is a method for restoring retromer complex function in a subject, comprising administering to the subject an effective amount of an agent that modulates CREB3L2 expression.

In some embodiments, the modulation of CREB3L2 expression comprises modulating the association between CREB3L2 and ATF4.

In some embodiments, the modulation of CREB3L2 expression results in at least one retromer-associated gene in the subject being deregulated by CREB3L2. In some embodiments, the at least one retromer-associated gene is selected from the group consisting of SNX3, SNX27, RAB7A, SNX1, VPS29, SNX5, VPS26B, SNX2, EHD1, SNX6, VPS26A, VPS35, and combinations thereof. In some embodiments, the at least one retromer-associated gene is selected from the group consisting of VPS26B, VPS35, SNX2, SNX5, SNX3, RAB7A, EHD1, and combinations thereof.

As defined above, in the context of the present disclosure, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments of the present disclosure, the phrase “a subject” means a subject having retromer complex(es) dysfunction.

Another embodiment of the present disclosure is a method for determining the progression of a neurodegenerative disease in a subject, comprising: (a) determining the level of CREB3L2-ATF4 transcription factor (TF) complex in a sample obtained from the subject; and (b) concluding that the neurodegenerative disease in the subject is progressing, if the level of CREB3L2-ATF4 complex determined in step (a) is significantly increased from that of a control subject.

In the context of this disclosure, the term “significantly increased”, as defined above, means that the increase of the level of CREB3L2-ATF4 complex in the sample over that of a control subject is statistically significant (e.g., with a p-value <0.05).

In some embodiments, the neurodegenerative disease is associated with retromer complex dysfunction, altered β-amyloid metabolism, tau hyperphosphorylation, or combinations thereof. In some embodiments, the neurodegenerative disease is selected from the group consisting of Alzheimer's Disease, Parkinson's Disease, Frontotemporal Lobar Degeneration, Down's Syndrome, Hereditary Spastic Paraplegia, Neuronal Ceroid Lipofuscinoses, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, and Progressive supranuclear palsy. In some embodiments, the neurodegenerative disease is Alzheimer's disease or Parkinson's disease. In some embodiments, the neurodegenerative disease is late-onset Alzheimer's disease (LOAD).

As defined above, in the context of the present disclosure, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments of the present disclosure, the phrase “a subject” means a subject having a neurodegenerative disease such as, e.g., Alzheimer's disease.

In some embodiments, the determining step (i.e., step (a)), may be carried out using any conventional method for determining the level CREB3L2-ATF4 TF complex in a biological sample. In certain aspects of this embodiment, the determining step is as set forth in, e.g., Examples 1 and 4 of the present disclosure.

In some embodiments, if the level of CREB3L2-ATF4 complex determined in step (a) is significantly increased from that of a control subject, then the subject may optionally be treated with an agent that modulates the association between CREB3L2 and ATF4. An exemplary agent may include those that reduce ATF4 activity such as, e.g., ursolic acid and tomatidine.

In the context of this disclosure, a “sample” or “biological sample” is as defined above. In some embodiments, the sample is obtained from the dorsolateral prefrontal cortex (PFC) of the subject.

Another embodiment of the present disclosure is method for identifying the DNA-binding profile of a dimeric transcription factor complex in vivo, comprising: (a) generating a DNA construct of a first transcription factor comprising: (i) fusing a specific first dimerization domain to the C-terminal of the first transcription factor; and (ii) adding a first N-terminal epitope tag to the first transcription factor; (b) generating a DNA construct of a second transcription factor comprising: (i) fusing a specific second dimerization domain to the C-terminal of the second transcription factor, wherein the second dimerization domain is different from the first dimerization domain; and (ii) adding a second N-terminal epitope tag to the second transcription factor, wherein the second N-terminal epitope tag is different from the first N-terminal epitope tag; (c) identifying a bivalent ligand that recognizes both dimerization domains from steps (a) and (b); (d) co-transfecting a host cell with the DNA constructs generated in steps (a)-(b) and co-expressing polypeptides encoded by the DNA constructs in the presence of the bivalent ligand identified in step (c) to form the dimeric transcription factor complex; and (e) identifying the DNA-binding profile of the dimeric transcription factor complex by determining the complex's binding sites to the genome using ChIP-sequencing (ChIP-seq).

In some embodiments, the dimerization domain is selected from the group consisting of FKBP, Calcineurin A (CNA), CyP-Fas, FRB, GyrB, GAI, GID1, SNAP-tag, HaloTag, eDHFR, Bcl-xL, and Fab(AZ1).

In some embodiments, the bivalent ligand is selected from the group consisting of FK1012, FK506, FKCsA, Rapamycin, Coumermycin, Gibberellin, HaXS, TMP-HTag, and ABT-737.

In some embodiments, the co-transfection in step (d) is a transient co-transfection. In some embodiments, the transient co-transfection can be carried out by a liposome-mediated method, a non-liposomal method, a viral delivery method, or electroporation.

In some embodiments, the DNA construct generation step (i.e., steps (a) and (b)), identification step (i.e., steps (c) and (e)) and co-transfection step (i.e., step (d)) may be carried out using any conventional method in the art. In certain aspects of this embodiment, such steps are as set forth in, e.g., Examples 1 and 5 of the present disclosure.

In some embodiments, the host cell is selected from the group consisting of HEK293, COS, CHO, and BHK cells. In some embodiments, the host cell is HEK293 cell. In some embodiments, the methods described above can be used in animals such as, e.g., a mammal including a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

A further embodiment of the present disclosure is a method for restoring amyloid precursor protein (APP) homeostasis in a subject in need thereof, comprising: (a) determining the Aβ₁₋₄₂/Aβ₁₋₄₀ ratio in a sample obtained from the subject; and (b) administering to the subject an effective amount of an agent that increases the expression level of CREB3L2 or prevents the dimerization of CREB3L2 with ATF4, if the Aβ₁₋₄₂/Aβ₁₋₄₀ ratio determined in step (a) is significantly higher that a predetermined reference.

As used herein, “amyloid precursor protein (APP) homeostasis” means an optimal condition of amyloid precursor protein (APP) in a subject. APP has been implicated as a regulator of synapse formation, neural plasticity, antimicrobial activity, and iron export. Either up- or down-regulation of APP expression may contribute to physiological deficits that are disease-associated. Proteolytic processing of APP results in the release of Aβ peptides of different length including Aβ₁₋₄₂ and Aβ₁₋₄₀. The longer Aβ₁₋₄₂ peptide exhibits stronger neurotoxic properties, and has been identified as the major component in senile plaques, which presumably contributes to Alzheimer's disease. In the context of this disclosure, β-amyloid precursor protein (APP) homeostasis can be assessed by measuring the concentrations of Aβ₁₋₄₂, Aβ₁₋₄₀, and/or calculating the Aβ₁₋₄₂/Aβ₁₋₄₀ ratio in a biological sample from a subject. The concentrations or calculated ratio cutoff can vary in different types of biological samples as defined above.

In the context of this disclosure, the term “significantly higher”, as defined above, means that the increase of the Aβ₁₋₄₂/Aβ₁₋₄₀ ratio determined in the sample over that of a control subject is statistically significant (e.g., with a p-value <0.05).

An additional embodiment of the present disclosure is a method for restoring tau metabolism in a subject in need thereof, comprising administering to the subject an effective amount of an agent that modulates the association between CREB3L2 and ATF4. In some embodiments, the modulation is to reduce level of CREB3L2-ATF4 heterodimer in the subject. In some embodiments, the modulation of CREB3L2-ATF4 association restores expression level of holoenzyme protein phosphatase 2A (PP2A). In some embodiments, the modulation of CREB3L2-ATF4 association restores expression levels of genes selected from the group consisting of SCG3, CLU, HSPA2, P4HB, HSPB1, PHGDH, MBNL2, PPP2CA, CELF3, AKT1, PKN1, CFL1, DBN1, MAOB, IGF1, NTRK2, AIF1, SLC1A3, and cominations thereof. In some embodiments, the restoration of tau metabolism comprises reduction of phosphorylation at Ser404.

Another embodiment of the present disclosure is a composition comprising a nucleotide of SEQ ID NO: 68 or SEQ ID NO: 69. The nucleotide of SEQ ID NO: 68 in the present disclosure is named “CREB3L2 aZIP”, comprising: 5′-GGATCCGCCACCATGGACTACAAAGATGATGACGACAAGCACATGGCCAGCATGA CCGGGGGCCAGCAGATGGGAAGAGACCCTGATTTGGAACAAAGGGCAGAGGAGC TGGCCCGGGAGAACGAAGAACTGGAGAAGGAAGCTGAGGAACTTGAGCAGGAGC TCGCTGAACTTCGGAAGAAGGTGGAGGTGCTGGAGAACACCAACAGGACTCTCCT TCAGCAACTTCAGAAGCTTCAGACTTTGGTGATGGGGAAGGTCTCTCGAACCTGCA AGTTAGCTGGTACACAGACTGGCACCTGCCTCATGGTCGTTGTGCTTTAAGAATTC-3′. The nucleotide of SEQ ID NO: 69 in the present disclosure is named “ATF4 aZIP”, comprising: 5′-GGATCCGCCACCATGGACTACAAAGATGATGACGACAAGCACATGGCCAGCATGA CCGGGGGCCAGCAGATGGGAAGAGACCCTGATTTGGAACAAAGGGCAGAGGAGC TGGCCCGGGAGAACGAAGAACTGGAGAAGGAAGCTGAGGAACTTGAGCAGGAGC TCGCTGAACTCACTGGCGAGTGTAAAGAGCTAGAAAAGAAGAACGAGGCTCTGAA AGAGAAGGCAGATTCTCTCGCCAAAGAGATTCAGTATCTAAAAGACCTGATAGAAG AGGTCCGTAAGGCAAGGGGGAAGAAGAGAGTTCCTTAAGAATTC-3′.

Another embodiment of the present disclosure is a method for preventing CREB3L2-ATF4 heterodimerization in a subject, comprising administering to the subject an effective amount of the composition comprising a nucleotide of SEQ ID NO: 68 or SEQ ID NO: 69.

Another embodiment of the present disclosure is a method for rescuing Aβ₄₂-induced neuronal cell death in a subject, comprising administering to the subject an effective amount of the composition comprising a nucleotide of SEQ ID NO: 68 or SEQ ID NO: 69.

An agent of the present disclosure (e.g., an agent that increases the expression level of CREB3L2) may be administered to a subject via oral, parenteral or other administration in any appropriate manner such as, e.g., intraperitoneal, subcutaneous, topical, intradermal, sublingual, intramuscular, intravenous, intraarterial, intrathecal, or intralymphatic. An agent of the present disclosure may be encapsulated or otherwise protected against gastric or other secretions, if desired. Further, such agent may be administered in conjunction with other treatments.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In the context of the present disclosure, the phrase “a subject in need thereof” means a subject in need of restoration for β-amyloid precursor protein (APP) homeostasis.

As used herein, the terms “modulate”, “modulating”, “modulator” and grammatical variations thereof mean to change, such as increasing or decreasing the expression of CREB3L2, or alternatively increasing or decreasing the association between CREB3L2 and ATF4.

In the present disclosure, an “effective amount” of an agent is an amount of such an agent that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of an agent according to the disclosure will be that amount of the agent, which is the lowest dose effective to produce the desired effect with no or minimal side effects. The effective dose of an agent according to the present disclosure may be administered as two, three, four, five, six or more sub-doses, administered separately at appropriate intervals throughout the day.

The following examples are provided to further illustrate the methods of the present disclosure. These examples are illustrative only and are not intended to limit the scope of the disclosure in any way.

### EXAMPLES

### Example 1

### Methods

### Neuronal Culture

Hippocampi were dissected from embryonic day (E) 16-18 rat embryos obtained from pregnant Sprague-Dawley dams (Envigo). All animal procedures were approved by the Institutional Animal Care and Use Committee at Columbia University. Cell dissociation was performed using TripLE Express. Neurons (50,000-60,000 per microfluidic chamber) were plated on 0.1 mg ml⁻¹ poly-D-lysine (Millipore Sigma) and 2 μg ml⁻¹ laminin (Bio-Techne) coated substrates and grown in Neurobasal supplemented with 10% fetal bovine serum, 2 mM L-glutamine, 1 mM sodium pyruvate, and antibiotics (50 U ml⁻¹ penicillin-streptomycin). Tripartite microfluidic chambers were produced with Dow Corning Sylgard 184 Silicone Encapsulant Clear Kit (10:1 mix ratio; Ellsworth Adhesives) cured at −70° C. for at least 4 hours following published protocols (Park et al. 2006; Desai et al. 2009). Chamber design incorporated two sets of 200-μm-long microgroove barriers to exclude crossing of cell bodies and dendrites (Taylor et al. 2005; Hengst et al. 2009). After 24 hours, medium was changed to Neurobasal containing 1×B27 and 2 mM L-glutamine. Subsequent medium changes (half volumes) were performed at DIV4 and thereafter every 3-5 days. To prevent glial cell proliferation, medium changes included 5-flurodeoxyuridine and uridine (final concentration 10 mM; Millipore Sigma) after DIV4. Neuronal cultures were grown in a 37° C., 5% CO₂ humidified atmosphere until DIV12-14. Whenever stated, axonal or cell body compartments were treated with 100 nM emetine (Millipore Sigma), 30 μM ciliobrevin A (R&D Systems), or 10/15 μM nelfinavir (Millipore Sigma). Unless otherwise specified, reagents were purchased from Thermo Fisher.

Aβ₁₋₄₂ peptide oligomerization and treatment

Lyophilized synthetic Aβ₁₋₄₂ peptides (Bachem) were dissolved to 1 mM in ice-cold 100% 1,1,1,3,3,3-hexafluoro-2-propanol (HFIP; Millipore Sigma) under a chemical fume hood by multiple rounds of pipetting, aliquoted and spun in a SpeedVac (Savant Instruments) for 30 minutes. The resulting peptide films were then resuspended in dimethylsulfoxide (DMSO; Millipore Sigma) to 5 mM, further diluted to 100 μM with HAM's F12 medium (Thermo Fisher), and incubated overnight at 4° C. Immediately before use, peptide concentration was adjusted with Neurobasal medium to a suitable working dilution, and added to axonal compartments at 3 μM, as previously described (Baleriola et al. 2014). Oligomerized Aβ₁-42 peptides were delivered to dissociated, non-compartmentalized cultures at concentrations in the range of 250-500 nM (reflecting batch-to-batch variations). Vehicle controls consisted of a DMSO/F12 mixture, similarly incubated overnight.

Axonal siRNA Transfection

siRNAs were delivered at a final concentration of 50 nM using NeuroPORTER transfection reagent (Genlantis) 24 hours prior to Aβ₁₋₄₂ treatment, as previously described (Baleriola et al. 2014). siRNA/NeuroPORTER mixture was prepared in serum- and antibiotic-free Neurobasal medium. Two hours post-transfection, axonal compartments were supplemented with an equal volume of Neurobasal medium containing 2×B27 and 4 mM L-glutamine. Stealth RNAi pre-designed siRNAs were used (Thermo Fisher); Creb3/2: 5′-CGAGGGCUAUCCCAUUCCAACCAAA-3′ (RSS324942; SEQ ID NO: 1); Hif1a: 5′-GCUAACAGAUGAUGGUGACAUGAUU-3′ (RSS310066; SEQ ID NO: 2). A control siRNA was similarly purchased from Thermo Fisher (Stealth RNAi siRNA Negative Control Med GC Duplex #3).

### Cell Death Assay (TUNEL)

Labeling of apoptotic cells was performed in accordance with manufacturer's instructions (DeadEnd Fluorometric TUNEL System, Promega). Cells were fixed in pre-warmed 4% paraformaldehyde, 4% sucrose phosphate-buffered saline (PBS) solution for 20 minutes at room temperature. After washing with PBS, a 3 mg ml⁻¹ bovine serum albumin, 100 mM glycine, 0.25% Triton X-100, PBS-based solution was incubated for 30 minutes at room temperature to permeabilize cells and block non-specific binding. Nuclei were counterstained with DAPI and samples mounted in ProLong Diamond Antifade Mountant (Thermo Fisher). Per replicate/condition, TUNEL-positive nuclei were scored against total cell number in no fewer than 10 fields situated in the vicinity of microgroove barriers.

### CHOP Immunocytochemistry and Analysis

Neurons grown in microfluidic chambers were fixed for 20 minutes at room temperature with a PBS-based 4% paraformaldehyde, 4% sucrose solution and thoroughly washed with PBS. Permeabilization and blocking was performed with 3 mg ml⁻¹ bovine serum albumin, 100 mM glycine and 0.25% Triton X-100 in PBS. Samples were incubated overnight with a primary antibody against CHOP (1:1,000; #2895, Cell Signaling Technology) diluted in permeabilization/blocking buffer. Following multiple PBS washes, a fluorophore-conjugated Alexa secondary antibody (1:1,000, Thermo Fisher) was applied for 1 hour. Samples were preserved in ProLong Diamond Antifade Mountant reagent (Thermo Fisher) and imaged using an EC Plan-Neofluar 40×/1.3 objective on an Axio-Observer.Z1 microscope equipped with an AxioCam MRm Rev. 3 camera (Zeiss). Image acquisition settings were selected so to avoid pixel saturation using AxioVision software (Zeiss). Acquisition parameters were kept constant between samples in any given experiment. Mean pixel intensity values were computed after background fluorescence subtraction.

### Chromatin Immunoprecipitation and Quantitative PCR (ChIP-qPCR)

For each immunoprecipitation/condition, approximately 15×10⁶ rat cortical neurons obtained from E16 Sprague-Dawley pups were cultured for a week on 0.1 mg ml⁻¹ poly-D-lysine (Millipore Sigma), 2 μg ml⁻¹ laminin (Bio-Techne) pre-coated flasks and maintained in Neurobasal medium supplemented with B27 and L-glutamine; Aβ₃₁₋₄₂ oligomers, prepared as described above, were bath-applied 36 hours prior to immunoprecipitation protocol. Protein-DNA cross-links were promoted using formaldehyde at a final concentration of 1% (v/v), and crosslinking reaction allowed to proceed for 10 minutes at room temperature, as per manufacturer's instructions (SimpleChIP Plus Kit [#9005], Cell Signaling Technology). After glycine quenching, cells were washed twice with ice-cold PBS, harvested in protease inhibitor-containing PBS by scrapping, and centrifuged at 2,000 g and 4° C. Chromatin fragments (150-900 base pair-long) were obtained by partial digestion with micrococcal nuclease (MNase) incubated for 11 minutes at 37° C. in a Thermomixer R (Eppendorf) with frequent mix cycles. Nuclear membranes were broken up by three rounds of 20-second pulses on a low setting (15% amplitude) using a Sonic Dismembrator (Model 500, Fisher Scientific) and lysates subsequently clarified by centrifugation. Adequate digestion was confirmed by agarose gel electrophoresis. Per immunoprecipitation/condition, 10 μg of digested, cross-linked chromatin were used and 2% input samples reserved. Antibodies were incubated overnight at 4° C. with end-over-end rotation: anti-CREB3L2 (2 μg; HPA015068, Atlas Antibodies) or, as negative control, rabbit normal serum IgG (#2729). Immunoprecipitates were captured using Protein G magnetic beads (#9006, Cell Signaling Technology) and washed with low- and high-salt buffers. Elution was performed at 65° C. and 1,200 rpm for 30 minutes using a thermomixer. Protein-DNA cross-links were then reversed by treatment with Proteinase K for 2 hours at 65° C., and DNA column-purified. ChIP signals were measured by quantitative PCR with QuantiTect SYBR Green PCR master mixes from Qiagen (Table 1). CREB3L2 enrichment was computed using the percent input method, whereby ChIP signals are normalized to input.

TABLE 1
List of qPCR primers used in ChIP analysis.

| Gene | Primer sequence |
|---|---|
| target | (5′ to 3′ direction) |
| Chop | ACTTCCGGGTCCGAGATAAC (SEQ ID NO: 3) |
| | GTGTCCAGGAGCCTACCAATC (SEQ ID NO: 4) |
| Vps26a | GAGGAAGCAAGGATTTGTGC (SEQ ID NO: 5) |
| | GTGAGATCAGGTGCGAAGGT (SEQ ID NO: 6) |
| | ACTCGGCTTTCCCTTAGGAG (SEQ ID NO: 7) |
| | GAGGAAAAGGAGTGGTCACG (SEQ ID NO: 8) |
| Vps26b | CGCACTCACTGAACTGCCTA (SEQ ID NO: 9) |
| | GTGGAGAGGGAGAAGACGTG (SEQ ID NO: 10) |
| | GGCACAGTACCTCCCCAGAT (SEQ ID NO: 11) |
| | CGGGAGTCTAGGCAGTTCAG (SEQ ID NO: 12) |
| Vps29 | CAATGAGACGACGAGTTTGC (SEQ ID NO: 13) |
| | GAGGAATTTCTCGCAGCAC (SEQ ID NO: 14) |
| Vps35 | GACTTTATGTGGGCCAATCG (SEQ ID NO: 15) |
| | ACAAGCAGCAGCGCCTAC (SEQ ID NO: 16) |
| Snx1 | GGCGCCAGTGAAAATATCCT (SEQ ID NO: 17) |
| | GGGAGGTGGTGGCTGTAG (SEQ ID NO: 18) |
| | GACTCCGGATCCATTCCAG (SEQ ID NO: 19) |
| | GCTCGCGGCACTGTCTATAA (SEQ ID NO: 20) |
| Snx3 | TCACAGTGAGGCACTGGACT (SEQ ID NO: 21) |
| | ACCCCGGAAATGATTTTAGC (SEQ ID NO: 22) |
| Rab7a | GCCTTGGGCTTTATGGTCA (SEQ ID NO: 23) |
| | GTCTCGTGACAGGCACTTCC (SEQ ID NO: 24) |
| | CACCATATTGGGCCAAGAAC (SEQ ID NO: 25) |
| | GTTCCAAAGGGGGACACTCT (SEQ ID NO: 26) |
| Ehd1 | GTCTGTACGCCGGTCCTTG (SEQ ID NO: 27) |
| | GGAGACAGAGCTGGCTGCTA (SEQ ID NO: 28) |

### Generation of GFP-Tagged CREB3L2

cDNA containing H. sapiens CREB3L2 open reading frame was acquired from Genecopoeia (NCBI Reference sequence: NM_194071; product #EX-H2495-M01) and subcloned into pEGFP-C1 (Clontech). EcoRI and BamHI restriction sites were introduced by PCR with CloneAmp HiFi polymerase (Takara; Table 2) and end products validated by Sanger sequencing. In-fusion cloning was used to mutate the S1P cleavage site (In-Fusion HD EcoDry Cloning Kit, Takara).

TABLE 2
Primers utilized in the generation
of GFP-CREB3L2 transgenes.

| | Primer sequence |
|---|---|
| Target | (5′ to 3′ direction) |
| CREB3L2 | ATTAGTGAATTCTGAGG |
| (full-length | TGCTGGAGAGCGGGGAG |
| form) | C (SEQ ID NO: 29) |
| | CGGACCGGATCCTTAGA |
| | AAGTGGTGTTCACTCTT |
| | (SEQ ID NO: 30) |
| CREB3L2-clv | ATTAGTGAATTCTGAGG |
| (activated | TGCTGGAGAGCGGGGAG |
| form) | C (SEQ ID NO: 31) |
| | CAATCGGGATCCTTACA |
| | GCACCACAACCATGAGG |
| | CAG (SEQ ID NO: 32) |
| CREB3L2-S1P- | GCAAACCTGGCGATCTA |
| (full-length | CGAGGAACATTCTCCCC |
| form | CAGAG |
| with mutated | (SEQ ID NO: 33) |
| S1P | GATCGCCAGGTTTGCGG |
| site) | ATCTCACCACGGAGGCT |
| | GTGTA |
| | (SEQ ID NO: 34) |

*Underlined sequences denote gene-specific sequences;
**15-base pair sequence overlap for In-Fusion cloning is highlighted in bold.

### Rabbit Reticulocyte Lysate Translation System and Immunoprecipitation

Capped, poly(A)-tailed H. sapiens CREB3L2 (GFP-tagged, full-length and cleaved forms) and ATF4 (FLAG-tagged; kind gift from Yihong Ye, Addgene plasmid #26114) transcripts were synthesized from Xbal-linearized pRK5 plasmids using the mMESSAGE mMACHINE SP6 and Poly(A) Tailing kits from Thermo Fisher. RNAs were column-purified in accordance to manufacturer's instructions (RNeasy Mini Kit, Qiagen) and efficient poly(A) tailing evaluated by agarose gel electrophoresis. CREB3L2 and ATF4 translation reactions were incubated separately for 90 minutes at 30° C. following vendor's guidelines (Rabbit Reticulocyte Lysate System, Promega). CREB3L2 and ATF4 protein products were confirmed by immunoblotting during pilot experiments. Upon completion of the translation protocol, lysates (45 μl) containing CREB3L2 and ATF4 were mixed and incubated at 37° C. for 30 minutes with gentle agitation (300 rpm for 5 seconds every minute) before immunoprecipitation, as previously described (Hai and Curran, 1991). Immunoprecipitation was carried out overnight at 4° C. with gentle rotation in PBS saline supplemented with 0.1% NP-40 and protease inhibitors (cOmplete, EDTA-free Protease Inhibitor Cocktail (Roche), using an anti-ATF4 antibody (1:200; #11815, Cell Signaling Technology). Immunoprecipitates were captured with M-280 Sheep Anti-Rabbit IgG Dynabeads (70 μl per immunoprecipitation; Thermo Fisher) pre-blocked with 0.1% BSA, with beads being incubated at 4° C. for 3 hours under rotary agitation. Washing cycles were repeated five times with immunoprecipitation buffer and the supernatant from the first wash put aside (‘flow-through fraction’). Complex elution was performed in 2×Laemmli buffer (130 mM Tris-Cl pH 6.8, 0.1 mM dithiothreitol, 20% (v/v) glycerol and 4% sodium dodecyl sulfate diluted in water) by boiling at 95° C. for 5 minutes. Immunoblot detection of GFP-tagged CREB3L2 in ATF4 immunoprecipitates: anti-GFP (1:2,000; ab290, Abcam), in conjugation with TrueBlot anti-rabbit IgG HRP (1:1,000; Rockland).

### Western Blot Analysis

Unless otherwise specified, protein extracts were prepared in ice-cold RIPA buffer (150 mM NaCl, 1% NP-40, 0.5% sodium deoxycholate, 0.1% SDS, 50 mM Tris [pH 8.0]) and resolved by SDS-PAGE using the NuPAGE electrophoresis system (Thermo Fisher). Both ‘semi-dry’ and ‘wet’ electroblotting methods were employed, depending on protein size. Nitrocellulose membranes (Amersham Protran 0.45 μm NC, GE Healthcare) were blotted using the following primary antibodies: anti-CREB3L2 (1:1000; HPA015068, Atlas Antibodies), anti-ATF4 (1:1,000; #11815, Cell Signaling Technology), anti-ATF4 (1:1,000, WH0000468M1, Millipore Sigma) or anti-ATF4 (ab50546, Abcam, at 1:1,000 [product discontinued], anti-GFP (1:2,000; ab290, Abcam), anti-VPS26 (1:1,000, ab23892, Abcam); anti-VPS29 (1:500; ab98929, Abcam); anti-VPS35 (1:2,500; ab10099, Abcam); anti-SNX1 (1:10,000, ab134126, Abcam); anti-SNX3 (1:500; ab56078, Abcam), anti-Rab7a (1:1,000; #9367, Cell Signaling Technology), anti-EHD1 (1:2,500; ab109311, Abcam), anti-APP (1:20,000; ab32136, Abcam), anti-N-Cadherin (1:1,000; #13116, Cell Signaling Technology), anti-FLAG (1:1,000; F1804, Millipore Sigma), anti-βIII-tubulin (1:10,000-1:20,000, #MA1-118, Thermo Fisher); anti-p-actin (1:10,000; #3700, Cell Signaling Technology) or anti-p-actin (1:10,000; MAB1501, Millipore Sigma); anti-GAPDH (1:10,000; 60004-1-Ig, Proteintech); anti-HDAC1 (ab109411; 1:10,000). Signals were visualized on a KwikQuant Imager (Kindle Biosciences) and quantified using Fiji/ImageJ.

### Proximity Ligation Assay

Cells grown in glass bottom dishes (P50G-1.5-30-F, MatTek Corporation) were fixed in pre-warmed 4% paraformaldehyde, 4% sucrose in PBS (pH 7.4) for 20 minutes at room temperature. After repeated wash cycles, microfluidic chambers were carefully removed (as applicable) and 0.25% Triton X-100 saline was added for 10 minutes to permeabilize membranes. Antigen retrieval with steaming 0.01 M sodium citrate (0.05% Tween 20, pH 6.0) was then carried out for 1 minute. Blocking was performed for 1 hour with 5% heat-inactivated goat serum diluted in PBS, and primary antibodies, prepared in blocking solution, incubated overnight at 4° C. (anti-CREB3L2: 1:100; HPA015068, Atlas Antibodies; anti-ATF4 (ab50546, Abcam, at 1:1,000 [product discontinued], or WH0000468M1, Millipore Sigma, at 1:100, or 60035-1-Ig, Proteintech, at 1:100). Proximity ligation assay protocol was performed using Duolink In situ Red Detection reagents (DU092008; Millipore Sigma) as per manufacturer's guidelines. Plus (DU092002) and minus (DU092004) probes were diluted 1:5 in blocking buffer and incubated at 37° C. for 1 hour in an hybridization oven (Hoefer Red Roller II). After the last wash step of the Duolink protocol, neurons were counterstained with Alexa 488-conjugated βIII-tubulin antibody (1:500; #801203, Biolegend) diluted in a water-based 2 mM Tris, 1 mM NaCl solution (pH 7.5) for 1 hour, and preserved in Duolink In situ Mounting Medium with DAPI (DU082040, Millipore Sigma). All incubations were performed in a humidity chamber. Samples were imaged using an Axio-Observer.Z1 microscope (Zeiss) equipped with an AxioCam MRm Rev. 3 camera through an EC Plan-Neofluar 40×/1.3 oil objective; alternatively, a LSM800 confocal microscope (Zeiss) with a Plan Apo 63×/1.4 oil objective was employed for analysis of nuclear interactions. Imaging settings were kept constant between conditions. Signals were counted manually and, when applicable, normalized to axon length.

### In Situ Visualization of Newly Synthesized Proteins (Puro-PLA)

Hippocampal neurons were cultured in microfluidic chambers until DIV12 and treated with Aβ₁₋₄₂ as described above. Puromycin (final concentration 1.8 μM; A1113802, Thermo Fisher) was supplemented into axonal media for 10 minutes at the end of the Aβ₁₋₄₂ stimulation. Puromycin labeling was combined with a proximity ligation assay protocol to detect newly synthesized CREB3L2 and signal specificity controlled for using the protein synthesis inhibitor anisomycin (final concentration: 10 μM; 176880, Thermo Fisher). Primary antibodies were incubated overnight at 4° C.; mouse anti-puromycin antibody (1:250; S9684, Millipore), rabbit anti-CREB3L2 (1:100; ARP34673_T100, Aviva Systems Biology).

### Transwell Neuronal Culture and Co-Immunoprecipitation

Millicell transwell inserts (1 μm pore size; Millipore [MCRP06H48]) were mounted on 6-well plates and both sides of the membrane sequentially coated with 0.1 mg ml⁻¹ poly-D-lysine (Millipore Sigma) and 2 μg ml⁻¹ laminin (Bio-Techne). A mix of E18 rat cortical and hippocampal neurons (800,000 per well) were plated on the bottom side of the insert (flipped up during this part of the protocol), and allowed to settle for 20-30 minutes. Transwell inserts were then carefully placed back in the plates, now reverted to their original orientation. With this set-up, neurites grow upward towards the top side of the membrane, which is more accessible to treatment. Cells were maintained for 10-12 DIV in Neurobasal containing 1×B27 and 2 mM L-glutamine, after the first 24 hours on Neurobasal supplemented with 10% fetal bovine serum, 2 mM L-glutamine, 1 mM sodium pyruvate, and antibiotics (50 U ml⁻¹ penicillin-streptomycin). Following a PBS wash (ice-cold), cell bodies were gently scraped off with a cotton swab and neuritic fractions (comprised of at least two pooled transwells, ca. 200 μg of protein) collected in CHAPS buffer (150 mM KCl, 50 mM HEPES, 0.1% CHAPS, adjusted to pH 7.4, plus protease inhibitors). Co-immunoprecipitations were performed overnight at 4° C. with rotation using antibody-bound M-280 Dynabeads (Thermo Fisher); antibodies: mouse anti-CREB3L2 (1:200; MABE1018, Millipore) or, as negative control, normal mouse IgG serum. Finally, beads were washed five times with PBS, resuspended in 25 μl of Laemmli buffer containing 5% P-mercaptoethanol and boiled before western blot analysis.

### Human Brain Sample Procurement and Processing

Post-mortem human material was obtained through the New York Brain Bank at Columbia University in accordance to institutional guidelines governed by approved protocols. Autopsy cases underwent a uniform neuropathological evaluation (Table 3 and Table 4), including assignment of CERAD (Mirra et al. 1991), Braak (Braak and Braak, 1991; Braak and Braak, 1993), and NIA-Reagan scores (Consensus recommendations for the postmortem diagnosis of Alzheimer's disease, 1997). Cerebral amyloid angiopathy was assessed according to the Vonsattel criteria (Greenberg and Vonsattel, 1997). Frozen dorsolateral prefrontal cortex tissue specimens were derived from Brodmann area (BA) 9/10. Protein extracts were prepared from 30-50 mg of tissue in ice-cold RIPA buffer supplemented with protease and phosphatase inhibitors (cOmplete cocktail tablets, Roche) using a Dounce homogenizer; RIPA buffer volumes were adjusted taking tissue weigh CREB3L2-ATF4 heterodimers were visualized t in consideration (300 μl per ˜5 mg of tissue). Samples were incubated in RIPA buffer for 2 hours at 4° C. under constant rotation. Halfway through this step, a 10-minute ice-cold sonication bath was run to improve tissue lysis (Branson 1510). Extract aliquots (500 μl) were centrifuged for 20 minutes at 12,000 rpm and 4° C., and supernatants mixed with 2×Laemmli buffer. Extracts were then denatured at 95° C. for 5 minutes before western blot analysis.

TABLE 3
Case history.

| Classification | Sample | Age | Gender | Cold PMI^{a} | Frozen PMI^{b} |
|---|---|---|---|---|---|
| Control | 346 | 84 | M | 600 | 850 |
| Control | 336 | 84 | F | 400 | 1251 |
| Control | 5382 | 62 | M | na | 324 |
| Control | 148 | 66 | M | 625 | 1315 |
| Control | 3799 | 89+ | F | 175 | 849 |
| Mild AD | 4051 | 89+ | M | 190 | 1015 |
| Mild AD | 4857 | 89+ | M | Na | 760 |
| Moderate AD | 4871 | 84 | F | 110 | 1425 |
| Moderate AD | 4897 | 89+ | F | 15 | 1115 |
| Moderate AD | 4856 | 89+ | F | 45 | 990 |
| Moderate AD | 5450 | 89+ | F | 85 | 1210 |
| Moderate AD | 4389 | 86 | M | 130 | 585 |
| Moderate AD | 5226 | 89 | F | 160 | 650 |

Cold PMI: Post mortem interval calculated from the reported time of death to the time the patient was brought into the cold room
Frozen PMI: Post mortem interval calculated from the reported time of death to the mean time the brain was processed

TABLE 4
Neuropathological evaluation of autopsy material.

| | | CERAD | Braak | Braak | NIA- | Cerebral |
|---|---|---|---|---|---|---|
| | | Plaque | NFT | Lewy | Reagan | amyloid |
| Classification | Sample | Score | Stage | Body | consensus | angiopathy |
| Control | 346 | A | II | 0 | Low | 2 |
| Control | 336 | A | | 0 | Low | None |
| Control | 5382 | Not | I | 0 | Not eligible | None |
| | | eligible | | | | |
| Control | 148 | Not | 0 | 0 | Not eligible | None |
| | | eligible | | | | |
| Control | 3799 | Not | III | 0 | Not eligible | None |
| | | eligible | | | | |
| Mild AD | 4051 | B | V | 0 | Intermediate | 1 |
| Mild AD | 4857 | B | III | 0 | Intermediate | 1 |
| Moderate AD | 4871 | B | V | 0 | Intermediate | None |
| Moderate AD | 4897 | C | V | 0 | Intermediate | None |
| Moderate AD | 4856 | C | V | 0 | High | 2 |
| Moderate AD | 5450 | A | V | * | Intermediate | 2 |
| Moderate AD | 4389 | C | V | 0 | Intermediate | 1 |
| Moderate AD | 5226 | C | VI | 2 | Intermediate | None |

*Outsider-not assignable, seemingly not following Braak staging

### Analysis of LOAD-Associated and Related Transcriptional Profiles

LOAD mRNA expression measurements and significance P-values were obtained from publicly available datasets (E-GEOD-44770 [ArrayExpress identifier]; GSE95587 [GEO] and GSE15222 [GEO]) (Zhang et al. 2013; Webster et al. 2009; Friedman et al, 2018). Developmental and aging transcriptome profiles were retrieved from BrainSpan and the Aging, Dementia and Traumatic Brain Injury atlases (Miller et al. 2017; Miller et al. 2014). Heatmaps were generated using Plotly Chart Studio.

### Immunohistochemical Analysis of CREB3L2 in LOAD Prefrontal Cortex

Paraffin-embedded sections (5-μm-thick) were obtained from LOAD dorsolateral prefrontal cortex through the New York Brain Bank at Columbia University. Before deparaffinization with xylene, slides were placed in a 60° C. oven for 1 hour; we proceeded by rehydrating slides using a graded ethanol series (100%>95%>70%>50%>water), plus two 10-minute PBS-T (0.1% Tween 20) washes. Epitope unmasking was done for 20 minutes in steaming 0.01 M sodium citrate buffer (0.05% Tween 20, pH 6.0), followed by three 5-minute PBS-T rinses. For blocking, a PBS-based 10% heat-inactivated goat serum (v/v), 1% BSA (w/v), 0.1% Triton X-100 (v/v), 0.1% sodium azide (w/v) solution was applied for at least 1 hour. Primary antibodies were incubated overnight at 4° C.: anti-MAP2 (1:2,000; ab5392, Abcam), anti-GFAP (1:250; ab10062, Abcam), anti-IBA1 (1:1,000; ab5076, Abcam); antibodies were prepared in PBS supplemented with 1% BSA (w/v), 0.1% Triton X-100 (v/v), 0.1% sodium azide (w/v) (henceforth termed ‘antibody diluent’). MAP2, GFAP or IBA1 detection utilized alkaline phosphatase-conjugated secondary antibodies (incubated for 2 hours at room temperature) coupled with Vector Blue substrate precipitation (SK-5300, Vector Laboratories); Vector blue substrate working solution was prepared in 150 mM Tris-Cl buffer (pH 8.2) supplemented with levamisole (SP-5000, Vector Laboratories), as per manufacturer's instructions. We subsequently quenched endogenous peroxidases slides with 1% hydrogen peroxide for 30 minutes and continued by staining for CREB3L2 (1:100 dilution, overnight at 4° C.; HPA015068, Atlas Antibodies). For visualization, slides were incubated with a biotinylated secondary antibody for at least 1 hour at room temperature; next, we made use of the Vectastain ABC system (PK-6101, Vector Laboratories) to increase detection sensitivity, and finally developed signals employing the Vector NovaRED substrate kit (SK-4800, Vector Laboratories), all in accordance with manufacturer's guidelines. Sections were dehydrated with ethanol (50%>70%>95%>100%), cleared with Histo-Clear (64110-01, Electron Microscopy Sciences), and mounted in Vectamount (H-5000, Vector Laboratories). Slides were imaged on an Axioplan 2 microscope equipped with an AxioCam HRc and Plan-Apochromat 20×/0.75 objective (Zeiss); panoramas were created in Photoshop CSS (Adobe) from multiple, overlapping fields.

### Complex Co-Immunoprecipitation in Human Brain Tissue

Protein A magnetic beads (#S1425S, New England Biolabs) were washed in PBS containing 0.1% BSA and incubated at 4° C. for 1 hour with rotation. Following two rinses with PBS, beads were resuspended in lysis buffer, mixed for 4 hours with anti-CREB3L2 antibody (1 μg per immunoprecipitation; HPA015068, Atlas Antibodies), and washed three times with lysis buffer. At this point, we proceeded by covalently cross-linking the immobilized anti-CREB3L2 antibody to Protein A beads using bis(sulfosuccinimidyl)suberate (BS3; #21586, Thermo Fisher) following manufacturer's guidelines.

Dorsolateral prefrontal cortex tissue (approximately 20-30 mg) was processed in ice-cold lysis buffer (20 mM Tris-Cl pH 8, 137 mM NaCl, 1% Nonidet P-40 (NP-40), 2 mM EDTA, supplemented with protease and phosphatase inhibitors [cOmplete cocktail tablets, Roche]; sample volumes were weight-adjusted in a sample-by-sample manner using a Dounce homogenizer; tissue extracts were then incubated for 2 hours at 4° C. with end-over-end rotation. During this incubation, a 10-minute bath sonication step was performed to improve extraction efficiency. After centrifugation at 12,000 rpm and 4° C., pellets were discarded and supernatants transferred to new tubes. Equal amounts of antibody-bead conjugates were mixed with lysates overnight at 4° C. with constant rotation, and washed a total of four times with ice-cold PBS containing 0.1% NP-40 and protease inhibitors (cOmplete cocktail, Roche); the supernatant resulting from the first wash step was saved for further analysis (‘flow-through’ fraction). CREB3L2 immunoprecipitates were eluted in 50 μl of 0.2 M glycine buffer (pH 2.5) allowed to react for 5 minutes at 4° C. with rotation after a short vortexing step. Eluates were transferred to a new tube, and the elution protocol repeated. Pooled eluates were neutralized by the addition of 20 μl of 1 M Tris-Cl (pH 9.0), and Laemmli buffer-treated sampled heated at 80° C. for 5 minutes. ATF4 signals were visualized using anti-ATF4 serum (sc390063, Santa Cruz Biotechnology) and a light chain-specific monoclonal secondary antibody (211-032-171, Jackson ImmunoResearch).

### CREB3L2 ChIP-Sequencing in LOAD Prefrontal Cortex

Autopsy cases #4856 and #5450 (Tables 3 and 4), both females with moderate LOAD pathology were chosen for ChIP-seq analysis based on: 1) high CREB3L2 and ATF4 expression level, 2) CREB3L2-ATF4 complex accumulation, and 3) reduced postmortem processing intervals. Frozen minced brain tissue (approximately 150 mg per immunoprecipitation and a total of 300 mg per case) was transferred to a conical tube containing 6 ml of PBS supplemented with protease inhibitors (PBS+PI), and protein-DNA cross-linking allowed to develop for 20 minutes at room temperature using formaldehyde at a final concentration of 1.5% (v/v). Cross-linking reaction was then quenched with glycine (5 minutes at room temperature), as per manufacturer's instructions (SimpleChIP Plus Kit [#9005], Cell Signaling Technology). After rinsing in ice-cold PBS+PI, further proceeded by disaggregating tissue using an ice-cold Dounce homogenizer (7 ml total capacity) until a single-cell suspension was obtained. A 2,000 g and 4° C. centrifugation step followed, and supernatants discarded. Chromatin fragments (mainly 1-3 nucleosomes in size) were obtained by partial digestion with micrococcal nuclease (MNase; 2 μl in 500 μl) incubated for 13 minutes at 37° C. in a Thermomixer R (Eppendorf) programmed for frequent mix cycles. Nuclear membranes were broken up by three rounds of 20-second, 15% amplitude pulses using a Sonic Dismembrator Model 500 (Fisher Scientific), and lysates subsequently clarified by centrifugation. Adequate digestion was confirmed by agarose gel electrophoresis. Approximately 6 μg of chromatin, diluted in 400 μl of ChIP buffer, was used per immunoprecipitation; CREB3L2-bound DNA was immunoprecipitated by overnight incubation at 4° C. with anti-CREB3L2 serum (2 μg; HPA015068, Atlas Antibodies). Immunoprecipitates were captured using Protein G magnetic beads, and washed with low- and high-salt buffers, as directed. Elution was performed at 65° C. and 1,200 rpm for 30 minutes using a thermomixer, protein-DNA cross-links reversed by treatment with Proteinase K for 2 hours at 65° C., and DNA purification achieved by using a column-based system.

ChIP-seq library preparation and sequencing reactions were conducted at GENEWIZ, Inc. (South Plainfield, NJ, USA). ChIP samples were quantified using Qubit 2.0 Fluorometer (Life Technologies) and the DNA integrity checked with 4200 TapeStation (Agilent Technologies). NEB NextUltra DNA Library Preparation kit was used following the manufacturer's recommendations (Illumina, San Diego, CA, USA). Briefly, the ChIP DNA was end repaired and adapters were ligated after adenylation of the 3′ ends. Adapter-ligated DNA was size selected, followed by clean up, and limited cycle PCR enrichment. The ChIP library was validated using Agilent TapeStation and quantified using Qubit 2.0 Fluorometer as well as real time PCR. During library preparation, immunoprecipitated samples were normalized to input DNA, i.e., chromatin cross-linked and fragmented side by side with immunoprecipitated DNA using the same conditions. The sequencing libraries were multiplexed and clustered on one lane of a flowcell. After clustering, the flowcell was loaded on the Illumina HiSeq instrument according to manufacturer's instructions. Sequencing was performed using a 2×150 Paired End (PE) configuration. Image analysis and base calling were conducted by the HiSeq Control Software (HCS). Raw sequence data generated from Illumina HiSeq was converted into fastq files and de-multiplexed using Illumina's bcl2fastq v2.17 software. One mismatch was allowed for index sequence identification.

ChIP-seq sequencing data were processed and analyzed within the Galaxy web platform (Afgan et al. 2018), using the public server at usegalaxy.org. First, FastQC was run to evaluate overall sequencing quality (unique reads >90%). Second, library adapters and low-quality reads were removed using Trimmomatic v0.36. Third, reads were mapped to the hg38 reference genome with Bowtie v0.12.7, and non-uniquely mapped reads filtered out. Forth, unmapped and low quality (MAPQ <20) reads were excluded with samtools v1.2. Fifth, peak calling was performed with MACS2 v2.1.1 with minimum false discovery rate (FDR) cutoff for peak detection fixed at 0.05, lower and upper mfold bounds defined as 5 and 50, respectively, and extension size set at 144. Finally, peaks were exported to the UCSC genome browser for visualization after conversion to bigwig format. CREB3L2 gene ontology (GO) term enrichment was analyzed using ClueGO v2.5.4 within the Cytoscape platform (v3.6.1) (Shannon et al. 2003; Bindea et al. 2009). Protein interaction network was constructed using the STRING v10.5 database (Lassot et al. 2001), and results subsequently exported into Cytoscape.

### Chemically Induced Proximity: Reagent Preparation

C-terminal FRB (T82L mutant) or FKBP (wild-type) fusions, N-terminally tagged with HA or V5 epitopes were synthesized by Genewiz and transferred into pGL4.20[/uc2/Puro] (Promega) using SacI and FseI sites. Human CREB3L2 (residues 2-384, corresponding to its transcriptionally active form) and ATF4 (S219A stabilization mutant (McLean et al. 2010)) were utilized in transgene design; control Renilla luciferase sequence was obtained from pGL4.75 (Promega). Successful insertions were screened by Sanger sequencing. A/C heterodimerizer (C-16-(S)-7-methylindolerapamycin [Aβ₂₁₉₆₇]) was purchased from Takara.

### ChIPmera Chromatin Immunoprecipitation and Analysis

HEK293T cells (#CRL-3216), plated in 150 mm dishes (CLS430599, Corning), were maintained in DMEM supplemented with 10% fetal bovine serum plus antibiotics (50 U ml⁻¹ penicillin-streptomycin), and transfected using Lipofectamine 3000 (Thermo Fisher). Amounts of DNA delivery were optimized to achieve comparable expression levels between the different homodimer and heterodimer configurations (ATF4 transgenes: 18.35 μg; CREB3L2 transgenes: 4.5 μg; luciferase transgenes: 2.3 μg), as ATF4 and CREB3L2 have significantly different half-lives. While lipofectamine complexes were incubating, we performed a complete media change, which now included the A/C heterodimerizer at 500 nM.

Twenty-four hours post-transfection, we proceeded by crosslinking protein-DNA contacts with 1% (v/v) formaldehyde (#28908, Thermo Fisher) for 10 minutes at room temperature. After quenching cross-linking reaction with glycine, cells were washed twice with ice-cold PBS, harvested by scrapping in PBS with protease inhibitors, and centrifuged at 2,000 g and 4° C., as per manufacturer's instructions (SimpleChIP Plus Kit [#9005], Cell Signaling Technology). Chromatin fragments (mainly 1-3 nucleosomes in size) were obtained by incubation with micrococcal nuclease (3.5 μl [equivalent to 7,000 gel units] in 200 μl; M0247S, NEB) for 45 minutes in a 37° C.-water bath. Nuclear membranes were subsequently broken up by three rounds of 20-second, 15% amplitude pulses using a Sonic Dismembrator Model 500 (Fisher Scientific), and lysates clarified by centrifugation. Adequate digestion was assessed by agarose gel electrophoresis. For each condition, digested chromatin was split into two tubes and immunoprecipitated overnight at 4° C. with end-over-end rotation using pre-washed magnetic beads conjugated with anti-HA- or anti-V5 antibodies (anti-HA beads: P188836, Thermo Fisher; anti-V5 beads: NC0777490, MBL International); 35 μl of anti-HA beads and 25 μl of anti-V5 beads were utilized per immunoprecipitation. Beads were captured on a magnetic stand and washed with low- and high-salt buffers, as directed. Elution was performed at 65° C. and 1,200 rpm for 30 minutes using a thermomixer, protein-DNA cross-links reversed by treatment with Proteinase K for 2 hours at 65° C., and DNA column-purified. A representative 2% input sample was prepared by combining chromatin from the different backgrounds.

ChIP-seq library preparation and sequencing reactions were conducted at GENEWIZ, Inc., as described above. A total of 24 samples were submitted for analysis, comprising parallel HA and V5 immunoprecipitations, from 2 independent replicates, and sequencing libraries multiplexed and clustered on two lanes of a flowcell. Sequencing was performed using a 2×150 Paired End (PE) configuration.

As above, ChIP-seq sequencing data were processed and analyzed within the Galaxy web platform (Afgan et al. 2018). First, library adapters and low quality reads were removed using Trim Galore (version 0.6.3) with the following settings: phred quality score threshold=20, overlap with adapter sequence required to trim a sequence=2, maximum allowed error rate=0.1. Also during this step, reads shorter than 36 bp were discarded. Second, reads were mapped to the hg38 reference genome with Bowtie 2 v2.3.4.1. Third, unmapped and low quality (MAPQ <20) reads were excluded with samtools v1.8. Forth, files were converted to bigWig format using bamCoverage v3.3.0 and signals visualized in UCSC genome browser. Fifth, after running MACS predicted function, peak calling was performed with MACS2 v2.1.1.2 with minimum false discovery rate (FDR) cutoff for peak detection fixed at 0.01 and luciferase homodimers defined as controls. Sixth, MACS2 output was filtered to exclude peaks with fold enrichments lower than 5. Seventh, differential binding analysis was performed on pooled replicated samples using DiffBind package v2.10.0 with FDR threshold set at 0.01. Eighth, genomic context was analyzed using ChIPseeker v1.18.0 against GENCODE v32/GRCh38 genome assembly (September 2019 release) and gene annotations assigned by GREAT v4.0.4 (McLean et al. 2010); cut off for promoter regions was defined as ±3 kb from a transcription start site. HA and V5-ChIP-seq signal overlap assessment employed the bedtools WindowBed function (v2.29.0). GO analysis was performed using the Gene Ontology Consortium database (geneontology.org) (The Gene Ontology, C. The Gene Ontology Resource: 20 years and still Going strong, 2019; Ashburner et al. 2000). The MEME suite was employed for motif discovery (Bailey et al. 2009; Machanick and Bailey, 2011).

### Luciferase Reporter Assays

VPS26B, VPS29, VPS35, SNX1, SNX3, RAB7A, EHD1, CHOP, SEC24A and PTBP1 promoter/enhancer regions were amplified from human genomic DNA (G3041, Promega) by PCR (Table 5) and inserted into pGL4.20[/uc2/Puro] using XhoI- and HindIII- or Sac- and XhoI-flanked products. This was achieved by first amplifying the genomic region of interest by PCR, followed by a second, ‘nested’ reaction to introduce restriction sites. Primer design was guided by ChIPmera and CREB3L2 ChIP-seq signals. Successful insertions were screened by Sanger sequencing. Human CHOP enhancer/promoter region (−647 to +91), originally derived from Addgene plasmid #36035 (Oh et al. 2012), was transferred into pGL4.20[/uc2/Puro] (E6751, Promega) using the BgIII and HindIII sites. Luciferase reporter constructs (300 ng) were delivered by transient transfection to HEK293T cells (#CRL-3216, ATCC; grown in 6-well plates) together with specific CREB3L2/ATF4 dimer configurations (achieved by chemically induced proximity; ATF4 transgenes: 1,000 ng; CREB3L2 transgenes: 250 ng; luciferase transgenes: 125 ng), and luminescence measured 24 hours later using ONE-Glo Luciferase Assay System (E6110, Promega). Just before the addition of lipofectamine complexes, new media was added with A/C heterodimerizer at 500 nM. Lysis proceeded at room temperature on an orbital shaker for 5 minutes, as instructed by the manufacturer, and duplicate 200 μl fractions transferred to a white polystyrene 96-well plate. Luminescence signals were measured at room temperature on an Infinite M200 instrument (Tecan) and normalized to cell number, measured independently (RealTime-Glo MT Cell Viability Assay, G9711, Promega).

TABLE 5
List of primers used to engineer luciferase reporters.

| Gene target | Primer sequence (5′ to 3′ direction) |
|---|---|
| SEC24A | GACAAAGGCCTCCTTCTTGG (SEQ ID NO: 35) |
| | GAGGGAGGAGGCTAACGACT (SEQ ID NO: 36) |
| | ATACATGAGCTCGACAAAGGCCTCCTTCTTGG |
| | (SEQ ID NO: 37) |
| | CAGGTCCTCGAGGAGGGAGGAGGCTAACGACT |
| | (SEQ ID NO: 38) |
| VPS26B | CGCCGTGCTCAACTTTCA (SEQ ID NO: 39) |
| | CCTAACGCTCCTGGGACTC (SEQ ID NO: 40) |
| | GATCACCTCGAGCGCCGTGCTCAACTTTCA (SEQ ID NO: 41) |
| | CTGATCAAGCTTCCTAACGCTCCTGGGACTC (SEQ ID NO: 42) |
| VPS29 | CAAACTCTGTGACGAAGGTGC (SEQ ID NO: 43) |
| | TCAGCTGTGCTCAAAACAATAC (SEQ ID NO: 44) |
| | ATACATGAGCTCCAAACTCTGTGACGAAGGTGC |
| | (SEQ ID NO: 45) |
| | CAGGTCCTCGAGTCAGCTGTGCTCAAAACAATAC |
| | (SEQ ID NO: 46) |
| VPS35 | CGCATCTGGATTGGTTAGGA (SEQ ID NO: 47) |
| | AGTCAAATACTTGGATTTTATCAATGG (SEQ ID NO: 48) |
| | GATCACCTCGAGCGCATCTGGATTGGTTAGGA |
| | (SEQ ID NO: 49) |
| | CTGATCAAGCTTAGTCAAATACTTGGATTTTATCAATGG |
| | (SEQ ID NO: 50) |
| SNX1 | CAGCAGCCTCGGGATTGAT (SEQ ID NO: 51) |
| | AGATACACTTTTAAGGATGGCGA (SEQ ID NO: 52) |
| | GATCACCTCGAGCAGCAGCCTCGGGATTGAT (SEQ ID NO: 53) |
| | CTGATCAAGCTTAGATACACTTTTAAGGATGGCGA |
| | (SEQ ID NO: 54) |
| SNX3 | AAACGGCTCACTAGCTGGAA (SEQ ID NO: 55) |
| | GAGTGCAATGGCAGATCTCA (SEQ ID NO: 56) |
| | GATCACCTCGAGAAACGGCTCACTAGCTGGAA |
| | (SEQ ID NO: 57) |
| | CTGATCAAGCTTGAGTGCAATGGCAGATCTCA |
| | (SEQ ID NO: 58) |
| RAB7A | TCTATTGGGAGACTGCCTTTC (SEQ ID NO: 59) |
| | GCGGAAGTACCTGTCACGAG (SEQ ID NO: 60) |
| | GATCACCTCGAGTCTATTGGGAGACTGCCTTTC |
| | (SEQ ID NO: 61) |
| | CTGATCAAGCTTGCGGAAGTACCTGTCACGAG |
| | (SEQ ID NO: 62) |
| EHD1 | TGCTGACCCAGCTGAACATA (SEQ ID NO: 63) |
| | GTGGGGAAGCCTTCTCTTCT (SEQ ID NO: 64) |
| | GATCACCTCGAGTGCTGACCCAGCTGAACATA |
| | (SEQ ID NO: 65) |
| | CTGATCAAGCTTGTGGGGAAGCCTTCTCTTCT (SEQ ID NO: 66) |

*Underlined sequences denote gene-specific sequences

### ATF4 ChIP-Seq Analysis

Raw sequencing files were downloaded from the ENCODE portal (Dunham et al. 2012; Davis et al. 2018) (https://www.encodeproject.org/) with the following identifiers: ENCFF081 USS, ENCFF069VNL, ENCFF565KLI, ENCFF6821GK, belonging to two ATF4 ChIP-seq technical replicates. Control ChIP-seq files were: ENCFF002EFF, ENCFF002EFH, ENCFF002EFD, and ENCFF002EFA, similarly from two independent replicates. As above, ChIP-seq sequencing data were processed and analyzed within the Galaxy web platform (Afgan et al. 2018). First, library adapters and low quality reads were removed using Trimmomatic v0.36.6. Also during this step, reads shorter than 36 bp were discarded. Second, reads were mapped to the hg38 reference genome with Bowtie v1.1.2, and non-uniquely mapped reads filtered out. Third, unmapped and low quality (MAPQ <20) reads were excluded with samtools v1.8. Forth, files were converted to bam format using Galaxy's SAM-to-BAM converter v 2.1.1. Fifth, peak calling was performed with MACS2 v2.1.1 with minimum false discovery rate (FDR) cutoff for peak detection fixed at 0.005, lower and upper mfold bounds defined as 10 and 50, respectively. Sixth, MACS2 output was filtered to exclude peaks with fold enrichments lower than 5. Seventh, overlapping peaks (minimum 50 bp) between ATF4 ChIP-seq replicates were identified using Operate on Genomic Intervals' Join tool, v1.0.0. Eighth, we downloaded ENCODE's replicate consistency irreproducible discovery rate (IDR) analysis file, ENCFF6821GK, and crosschecked this to the filtered MACS2 output to produce a high confidence peak file. Ninth, genomic context (GENCODE v21/GRCh38 genome assembly, October 2014 release) was analyzed using ChIPseeker v1.18. Finally, a GO term enrichment study was performed using the Gene Ontology Consortium database (geneontology.org) (The Gene Ontology, C. The Gene Ontology Resource: 20 years and still Going strong, 2019; Ashburner et al. 2000). Analysis by cellular component revealed the endosome to be among the most enriched GO terms; this endosome-associated gene set was analyzed a second time to obtain a more detailed GO categorization.

shRNA Preparation and Delivery

shERWOOD-UltramiR shRNAs targeting R. norvegicus Creb3/2 (TLRSU1400-362339) and Atf4 (TLRSU1400-79255) mRNAs were acquired from transOMIC Technologies. For expression in primary neuronal cells, the original CMV promoter was substituted with that of human ubiquitin C gene (UBC) using the ClaI and AgeI sites. shRNA lentiviral particles were produced in HEK293T cells and titers measured with qPCR Lentivirus Titration Kit (Applied Biological Materials). Briefly, envelope, packaging and shRNA-carrying lentiviral vectors (3:7:10 ratio) were delivered using Lipofectamine 3000; 6 hours post-transfection, medium was changed to Neurobasal containing B27 and L-glutamine. Viral supernatant was collected after 36 hours, passed through a 0.45 μm PES filter, aliquoted, and stored at −80° C. Viral particles were added to neuronal cultures at a multiplicity of infection of 16.

### RNA Extraction and Quantitative Real-Time PCR

At the time of collection, cells were washed with ice-cold Hank's Balanced Salt Solution (HBSS; Thermo Fischer) and lysed with TRIzol reagent (#15596026, Thermo Fisher) by scrapping. The reaction was allowed to proceed on ice for 5 minutes, at which point samples were centrifuged at 12,000 g and 4° C. and supernatants transferred to new tubes before a 1:1 (v/v) dilution with molecular biology-grade 100% ethanol (Millipore Sigma). The RNA extraction protocol was continued using the Direct-zol RNA MicroPrep kit (Zymo Research), in accordance to manufacturer's instructions, and included an on-column DNA digestion step with DNase I. RNA was typically eluted in 50 μl of water. Real-time quantification of target RNA species was performed in triplicate on a StepOnePlus Real-Time qPCR system using the Luna Universal One-Step RT-qPCR kit (New England Biolabs); reverse transcription and PCR amplification were primed with TaqMan hydrolysis probes (Table 6; Thermo Fisher). The ΔΔCt method was employed for obtaining relative gene expression data points; reference gene stability across conditions was tested in preliminary experiments using the geNorm module in qbase+ (Biogazelle).

Axonal or somatic total RNA was similarly isolated by TRizol extraction coupled with Direct-zol RNA MicroPrep kit purification from 25 microfluidic chambers per condition, reverse transcribed (SuperScript III, Thermo Fisher), and cDNA preamplified for 20 cycles using the TaqMan PreAmp Master Mix kit (Thermo Fisher), according to manufacturer's instructions. Real-time PCR quantification was performed on a StepOnePlus system using the TaqMan Gene Expression Master Mix. Serial dilution calibration curves were calculated to assess overall sample quality and amplification efficiency. Ct values were interpolated from these curves and expression levels normalized to input RNA.

TABLE 6
TaqMan assays for qPCR analysis of gene expression.

| Gene | Species | Assay ID |
|---|---|---|
| Creb3/2 | Rat | Rn01455999_m1 |
| Atf4 | Rat | Rn00824644_g1 |
| Vps26a | Rat | Rn01433541_m1 |
| Vps26b | Rat | Rn02111368_s1 |
| Vps29 | Rat | Rn01480547_m1 |
| Vps29 | Rat | Rn01480546_m1 |
| Vps35 | Rat | Rn01538117_m1 |
| Vps35 | Rat | Rn01538113_m1 |
| Snx1 | Rat | Rn01418446_m1 |
| Snx3 | Rat | Rn01441118_m1 |
| Rab7a | Rat | Rn00576640_m1 |
| Ehd1 | Rat | Rn06302200_s1 |
| App | Rat | Rn00570673_m1 |
| Tubb3 | Rat | Rn01431594_m1 |
| Pgk1 | Rat | Rn00821429_g1 |
| Gapdh | Rat | Rn01775763_g1 |

### Cell Surface Protein Biotinylation

Rat cortical neurons were cultured in 6-well plates (650,000 cells/well) as described above and transduced with control or Creb3/2 shRNAs using lentiviral particles at DIV4. On DIV8, cells were quickly washed twice with ice-cold PBS and incubated with biotinylation reagent for 30 minutes at 4° C. on an orbital shaker, according to manufacturer's instructions (Pierce Cell Surface Protein Isolation Kit, Thermo Fisher) with volume adjustments. At this point, biotinylation reaction was quenched and cells gently scrapped into solution. Per condition/replicate, ca. 2×10⁶ neurons were pooled from three wells, and centrifuged at 500 g and 4° C. Labeling and quenching reactions were performed inside an environmental cold room maintained at 4° C. Pelleted cells were resuspended in lysis buffer supplemented with protease inhibitors, subjected to five 1-second pulses (10% amplitude) using a Sonic Dismembrator Model 500 (Thermo Scientific), and incubated on ice for 30 minutes. Cell lysates were then centrifuged at 10,000 g for 2 minutes and applied to NeutrAvidin agarose columns for 60 minutes a room temperature with end-over-end mixing. Before washing, column flow-throughs (i.e., the non-bound, internal fractions) were collected and saved for further analysis. Finally, columns were incubated for 60 minutes with end-over-end mixing in 200 μl of 2×Laemmli buffer, centrifuged at 1000 g for 2 minutes, and eluates denatured at 80° C. for 5 minutes. Surface and cytoplasmic fractions were probed by western blot.

### Neuronal Culture Supernatant Collection and Measurement Amyloid Peptides

Rat cortical neurons were cultured in 6-well plates (650,000 cells/well, in 2 ml of culture medium) as described above and transduced with control or Creb3/2 shRNAs using lentiviral particles at DIV4. On the day of infection, a medium change (half volume) was done, and shRNA-treated cells incubated for an additional 4 days before supernatant collection. At this point, culture supernatants were transferred to 15-ml falcon tubes, spun at 2000 g and 4° C. for 5 minutes, aliquoted, and stored at −80° C. A 96-well sandwich immunoassay (V-PLEX Aβ Peptide Panel 1 kit, #4G8, Meso Scale Discovery) was employed in the measurement of β-amyloid species. Manufacturer's guidelines were followed thoroughly during plate preparation, and samples diluted 1:1 with Diluent 35 (provided as part of the kit) to avoid matrix saturation. All biological replicates (n=7) were measured in parallel. Signal readings were performed on a Sector Imager 2400 instrument (Meso Scale Discovery). For assessing sAPPα levels in culture supernatants, we utilized a sandwich ELISA assay (sAPPα [Mouse/Rat] [highly sensitive], #27419, Immuno-Biological Laboratories).

### General Statistical Analysis

Each experiment was repeated at least three times unless otherwise indicated. Details of biological replication and statistical analysis are indicated in figure legends or main text. Data were analyzed with Prism (GraphPad). For all tests, a significance level (a) of 0.05 was used.

### Example 2

### CREB3L2 is a Locally Produced Dimerization Partner of ATF4 in Aβ₁₋₄₂-Treated Axons

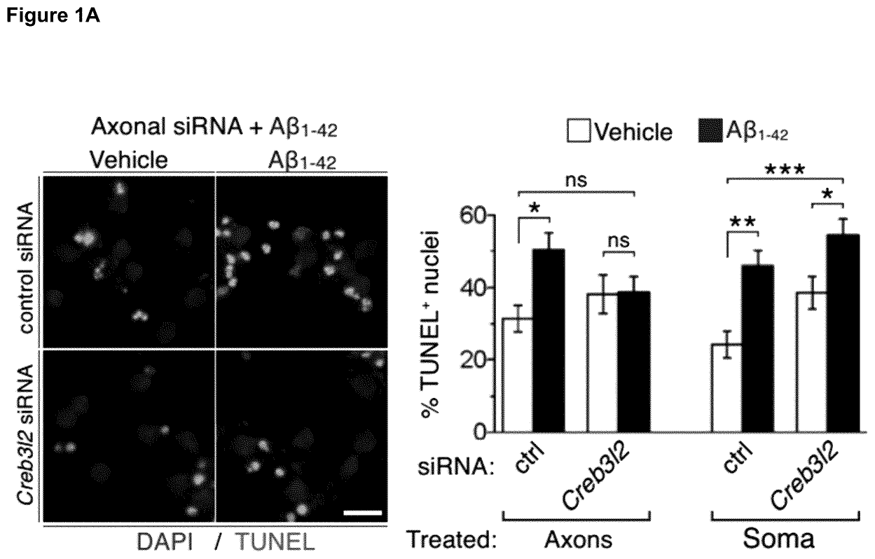

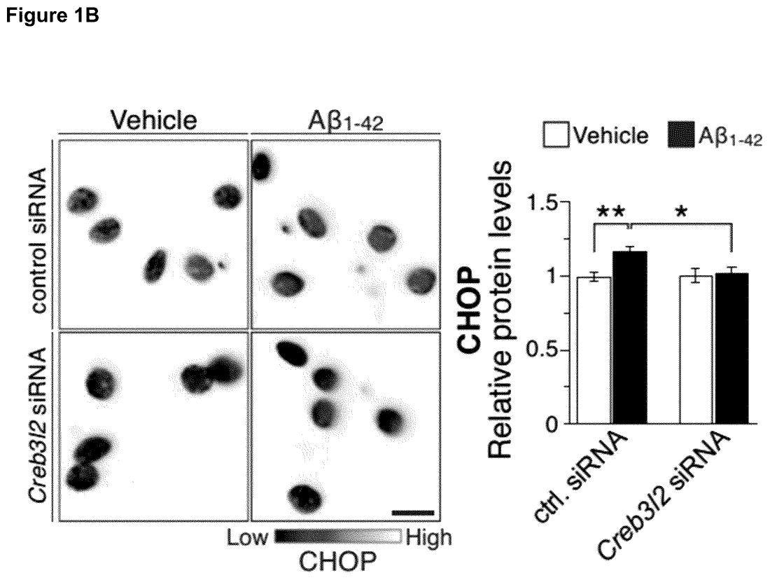

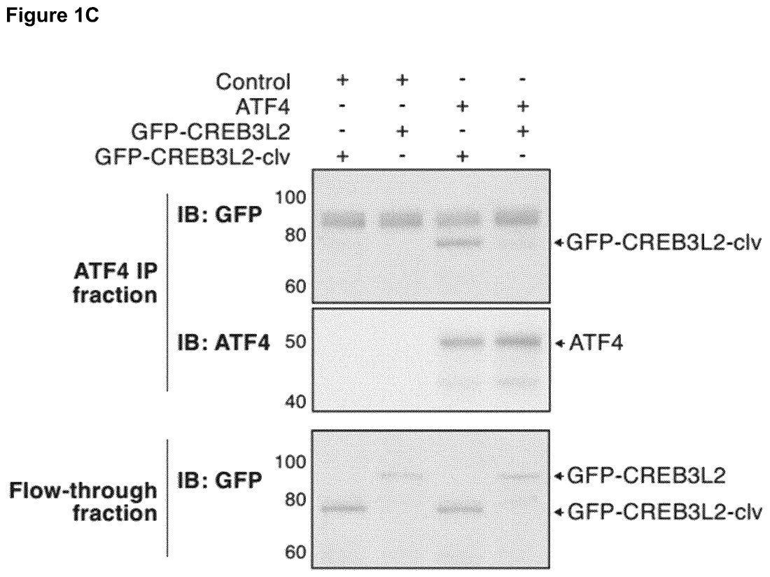

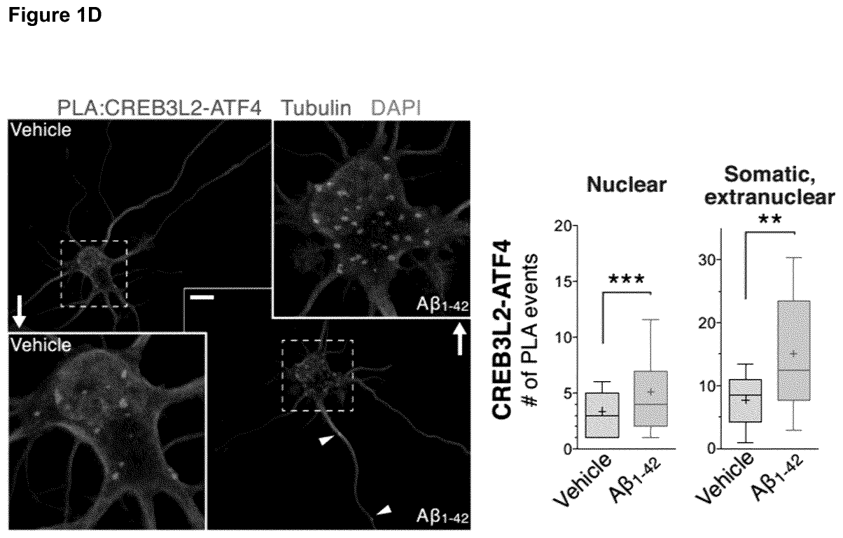

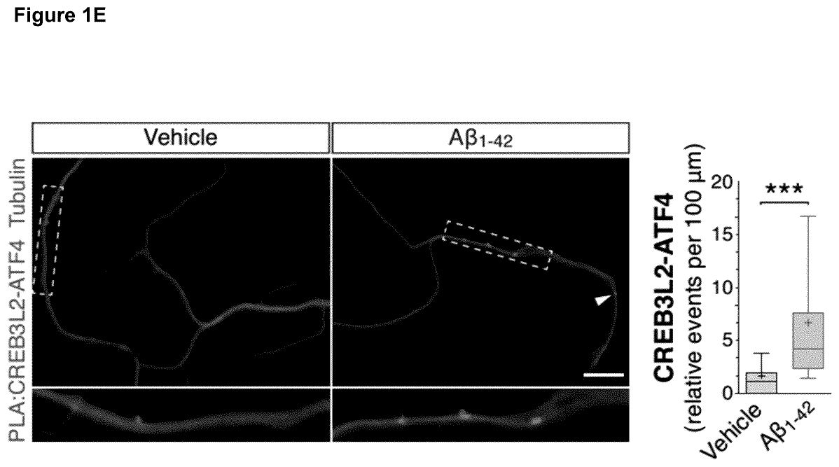

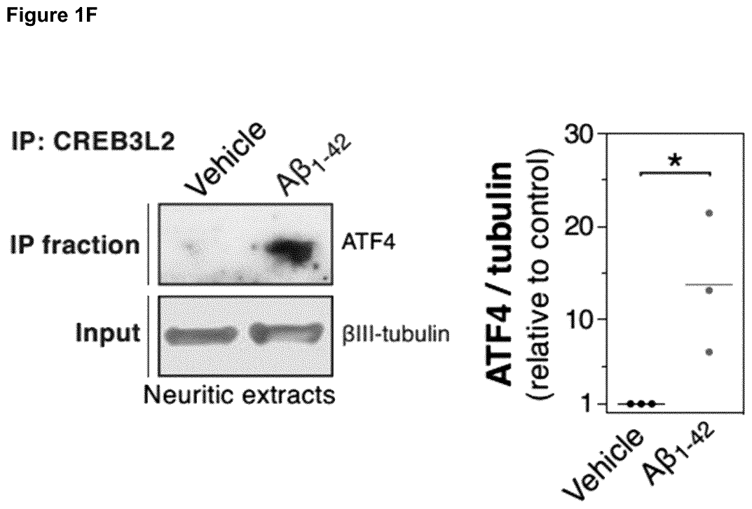

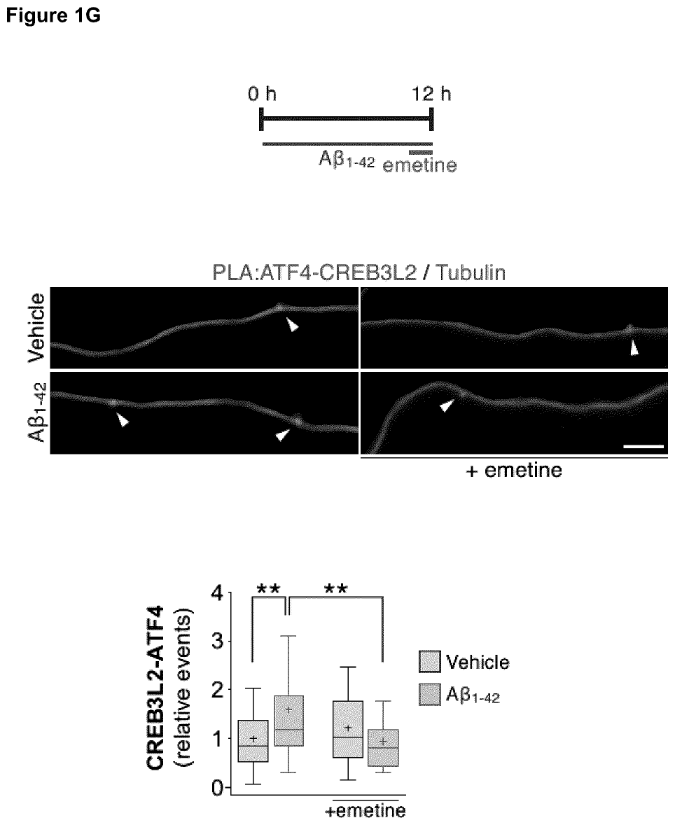

Neurodegenerative insults and peripheral injury can be communicated from axons to the neuronal soma via TFs (Baleriola et al.; Ben-Yaakov et al.; Ying et al.). For example, neurodegeneration in response to axonally sensed oligomeric Aβ₁₋₄₂ requires the local production and retrograde transport of the TF ATF4 followed by the ATF4-dependent expression of the proapoptotic TF CHOP (Baleriola et al., 2014). Curiously, ATF4 is constitutively expressed in neurons, including axons (Shigeoka et al., 2016), and regulates synaptic plasticity and memory formation (Chen et al., 2003; Pasini et al., 2015). Why is ATF4 different in the context of β-amyloid pathology? A potential solution for this conundrum might be that Aβ₁₋₄₂-dependent local synthesis of ATF4 allows the formation of specific TF complexes through the coordinated production of dimerization partners of ATF4. To identify such potential ATF4-binding partners, we delivered siRNAs specifically to axons of hippocampal neurons cultured in microfluidic chambers (FIG. 22A) and screened for candidates involved in the retrograde spread of Aβ₁₋₄₂ pathology. Like ATF4 (Baleriola et al., 2014), knockdown of the TF CREB3L2 specifically in axons prevented induction of apoptosis and CHOP upregulation after axonal treatment with oligomeric Aβ₁₋₄₂ (FIGS. 1A, 1B and 22B), and chromatin immunoprecipitation coupled with quantitative PCR (ChIP-qPCR) revealed significantly increased binding of CREB3L2 to the promoter/enhancer domain of Chop after treatment with Aβ₁₋₄₂ (FIG. 7C).

Co-immunoprecipitation experiments with in vitro translated and overexpressed proteins revealed that CREB3L2 and ATF4 can heterodimerize (FIGS. 1C and 7D). Using a proximity ligation assay (PLA; Baan et al., 2010; Soderberg et al., 2006), we detected CREB3L2-ATF4 dimers inside and around the nuclei but also in neurites of hippocampal neurons (FIG. 1D). Upon addition of Aβ₁₋₄₂, the number of CREB3L2-ATF4 PLA puncta was significantly greater in all neuronal structures analyzed (FIG. 1D), and local application of Aβ₁₋₄₂ increased CREB3L2-ATF4 interaction in axons (FIG. 1E). Similarly, ATF4 readily co-immunoprecipitated with CREB3L2 from lysates of neurites treated with Aβ₁₋₄₂ but not control neurites (FIGS. 1F and 7E).

Creb3/2 and Atf4 mRNAs are recruited into axons following exposure to Aβ₁₋₄₂ (FIGS. 7F and 7G; Baleriola et al., 2014; Walker et al. 2018), suggesting that CREB3L2-ATF4 heterodimerization may be regulated by local translation mechanisms. Consistent with this, levels for ATF4 and CREB3L2 were increased in neurites upon local treatment with Aβ₁₋₄₂ (FIG. 7H), and using a short puromycin pulse coupled with PLA coincidence detection (puro-PLA; tom Dieck et al., 2015), we detected higher levels of de novo CREB3L2 synthesis in Aβ₁₋₄₂-treated axons (FIG. 7I). In addition, inhibition of axonal protein synthesis prevented the Aβ₁₋₄₂-dependent increase in axonal CREB3L2-ATF4 PLA puncta (FIG. 1G), revealing that, like ATF4 (Baleriola et al., 2014), CREB3L2 is locally synthesized in axons in response to oligomeric Aβ₁₋₄₂, and new protein synthesis is required for complex formation in Aβ₁₋₄₂-treated neurons.

### Example 3

### S2P Mediates CREB3L2-ATF4 Complex Activation in Axons

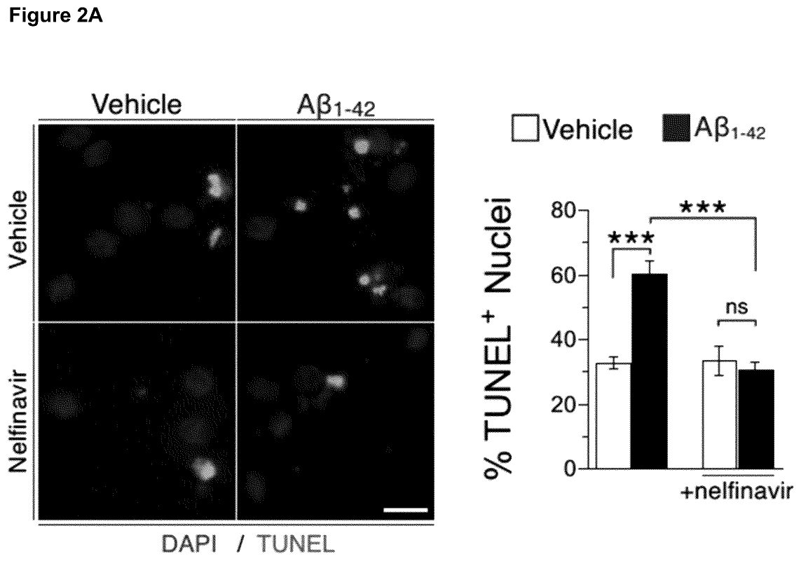

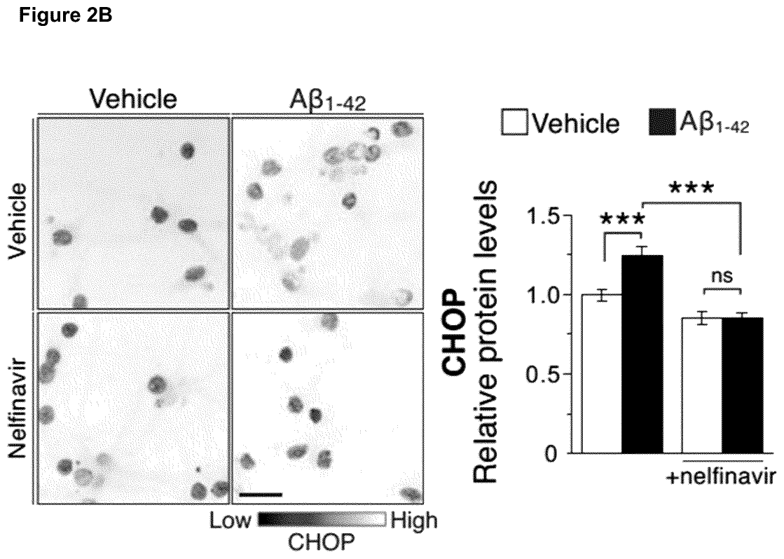

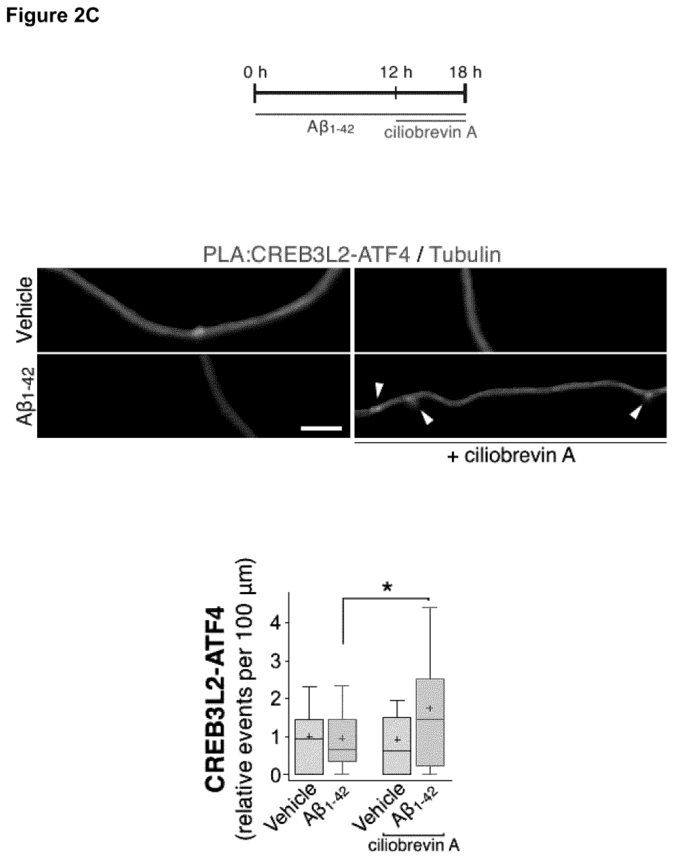

CREB3L2 is produced as a transcriptionally inactive ER-bound TF precursor that can only translocate to the nucleus after being proteolytically processed (FIG. 8A; Kondo et al., 2007). We found that CREB3L2 transcriptional activation depended on site 2 protease (S2P), whose cleavage activity was required for the nuclear accumulation of CREB3L2 induced by Aβ₁-42 exposure (FIGS. 8B, 8C, 8F and 8G). As S2P is present and functional in axons (McCurdy et al., 2019), we asked whether it also regulated Aβ₁₋₄₂-initiated axon-to-soma responses. Axonal treatment with a S2P inhibitor, nelfinavir (Guan et al., 2012; McCurdy et al., 2019), completely rescued the retrograde degeneration response triggered by local Aβ₁₋₄₂ stimuli (FIG. 2A), abolished the downstream induction of CHOP expression in the soma (FIG. 2B), and caused the accumulation of CREB3L2-ATF4 complexes in axons (FIG. 8H). In addition, inhibition of dynein in axons, a motor of retrograde transport, similarly led to a buildup of CREB3L2-ATF4 in this compartment (FIG. 2C). These experiments also revealed that CREB3L2-ATF4 signals receded to control levels within 18 hours of Aβ₁₋₄₂ exposure, suggesting that axon-derived CREB3L2-ATF4 complexes mediate a temporally delimited signaling event. Collectively, these datasets define a pathway whereby Aβ₁₋₄₂ regulates CREB3L2-ATF4 complex activation in axons via local translation- and S2P-mediated mechanisms followed by dynein-dependent retrograde transport of the complex to the nucleus.

### Example 4

### CREB3L2-ATF4 Complexes are Upregulated in LOAD

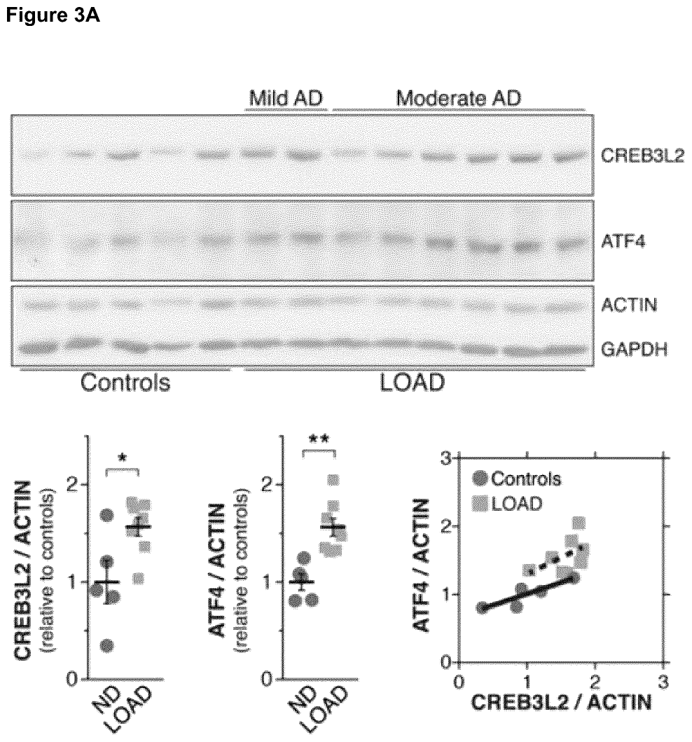

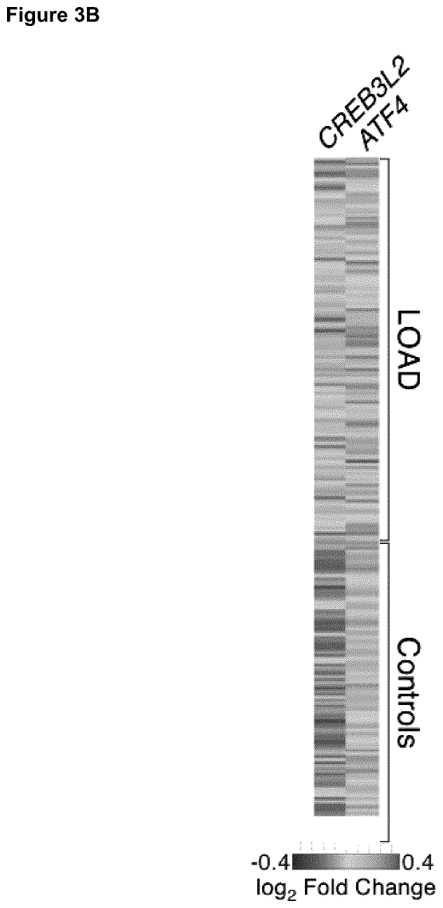

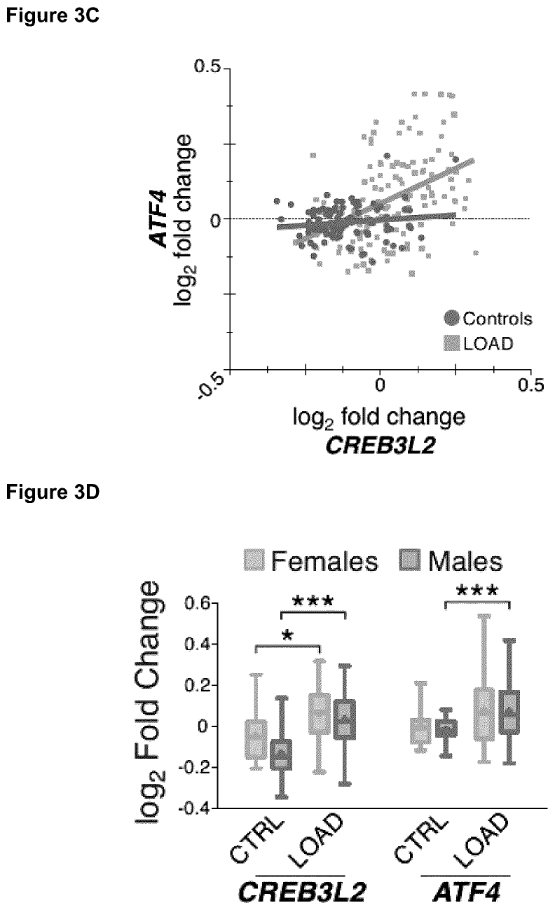

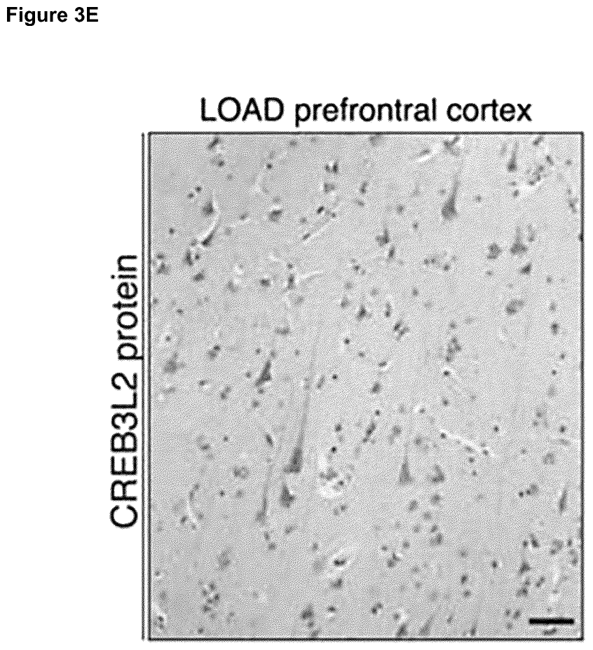

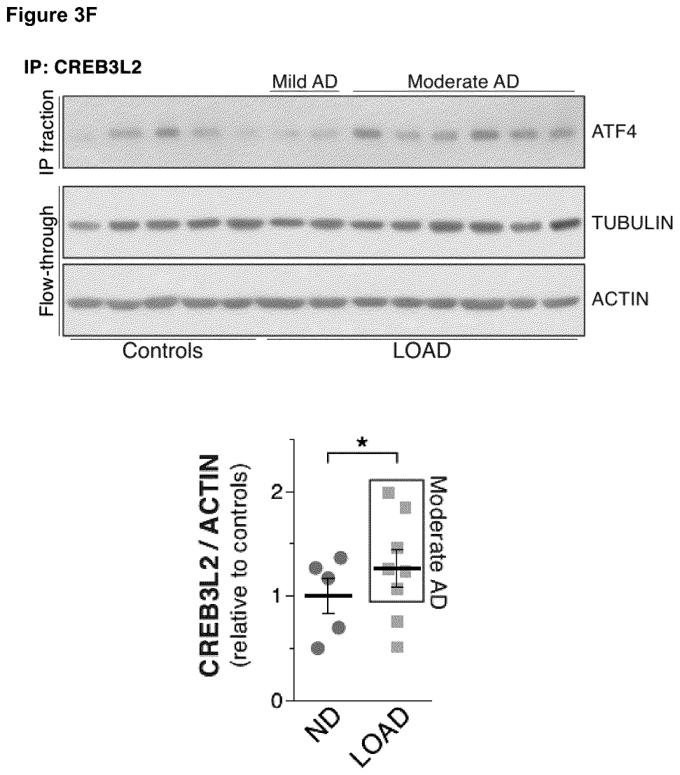

To determine the disease relevance of these findings, we first examined CREB3L2 and ATF4 expression levels in the dorsolateral prefrontal cortex (PFC) of non-demented control and LOAD subjects with mild or moderate pathology. Protein levels for both TFs, as measured by western blot, were significantly increased in LOAD cases, and positively correlated with each other (FIG. 3A). Analysis of a previously published transcriptional profiling dataset of PFC corroborated these results in a larger cohort: CREB3L2 and ATF4 mRNA levels were elevated and their correlation increased in LOAD (FIGS. 3B and 3C; Zhang et al., 2013). No significant differences were seen between LOAD females and males in this group, albeit healthy females showing overall increased levels of CREB3L2 mRNA compared to healthy males (FIG. 3D). Furthermore, age was not a risk factor for increased CREB3L2 and ATF4 expression (FIGS. 9A-9D; Miller et al., 2017). Despite their structural similarities (Chan et al., 2011), other members of the CREB3-like family of TFs (CREB3/LUMAN, CREB3L3/CREB-H, and CREB3L4/AlbZIP) are normally expressed in LOAD PFC, with the exception of CREB3L1/OASIS, which was downregulated (FIG. 9E). Also, in LOAD PFC, CREB3L2 predominantly localized to neurons (FIGS. 3E and 9F-9H). We further tested for the presence of CREB3L2-ATF4 complexes in human post-mortem PFC by co-immunoprecipitation. A significant enrichment of ATF4 in CREB3L2 immunoprecipitates was measured in the moderate disease samples, while mild LOAD cases showed no increased complex formation compared to controls (FIG. 3F). These findings reveal that CREB3L2-ATF4 heterodimers are upregulated in the PFC of LOAD patients with moderate disease, providing direct support for the pathophysiological significance of their interaction.

### Example 5

### The CREB3L2-ATF4 Transcriptional Program is Enriched in CREB3L2 Target Genes

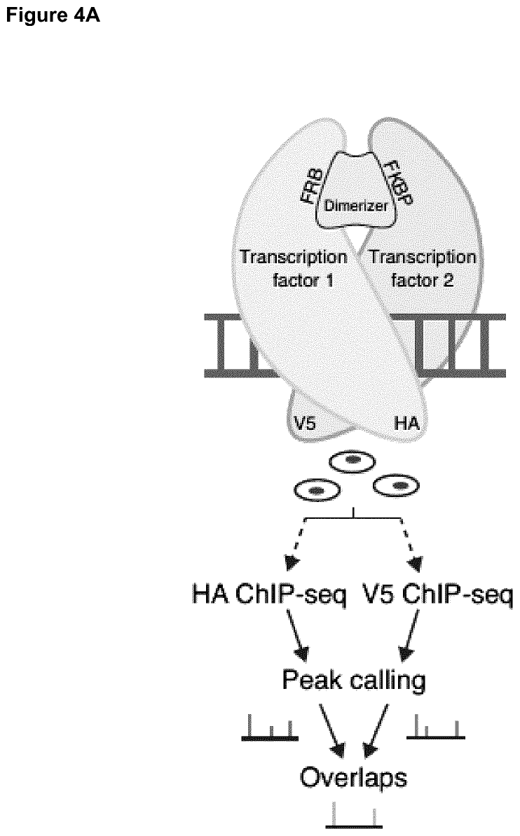

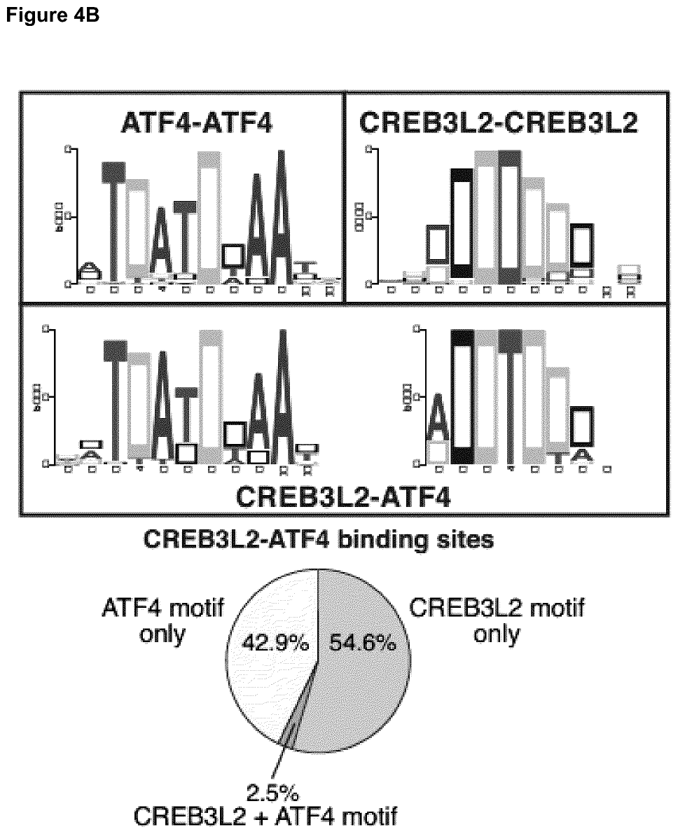

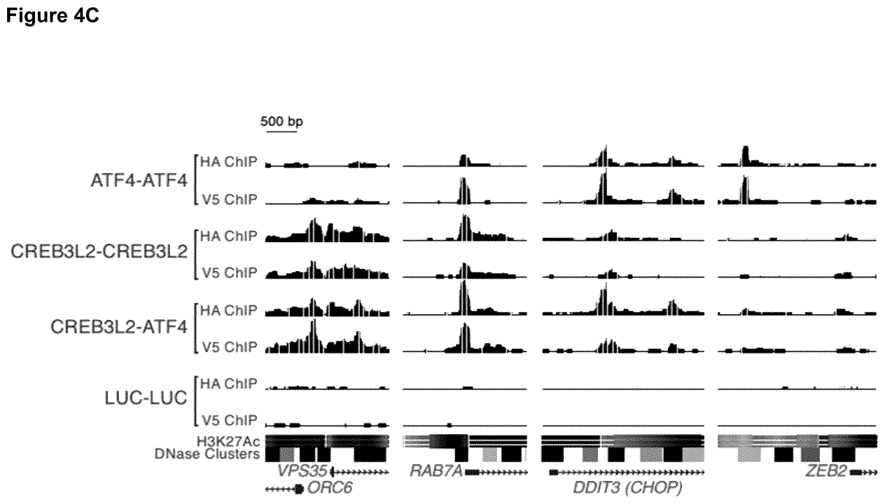

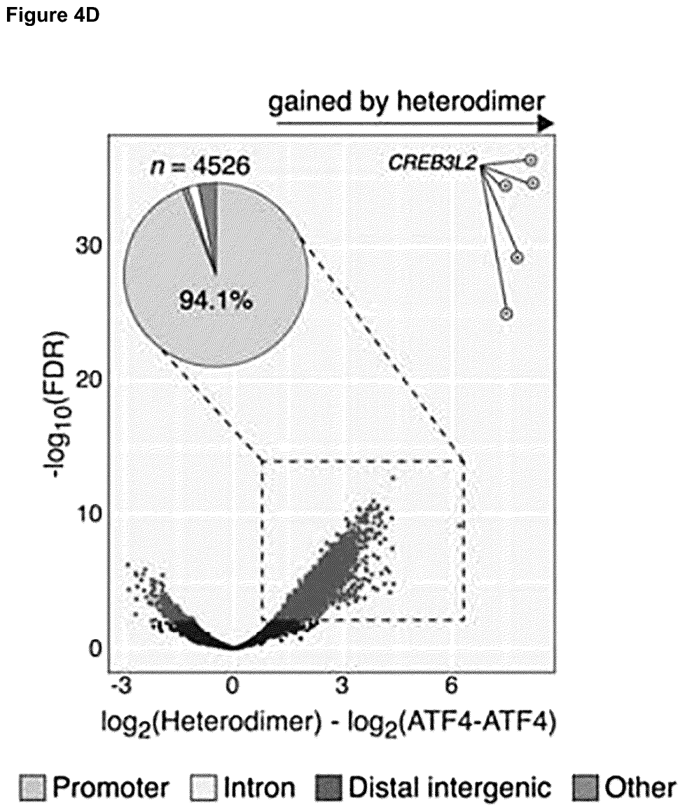

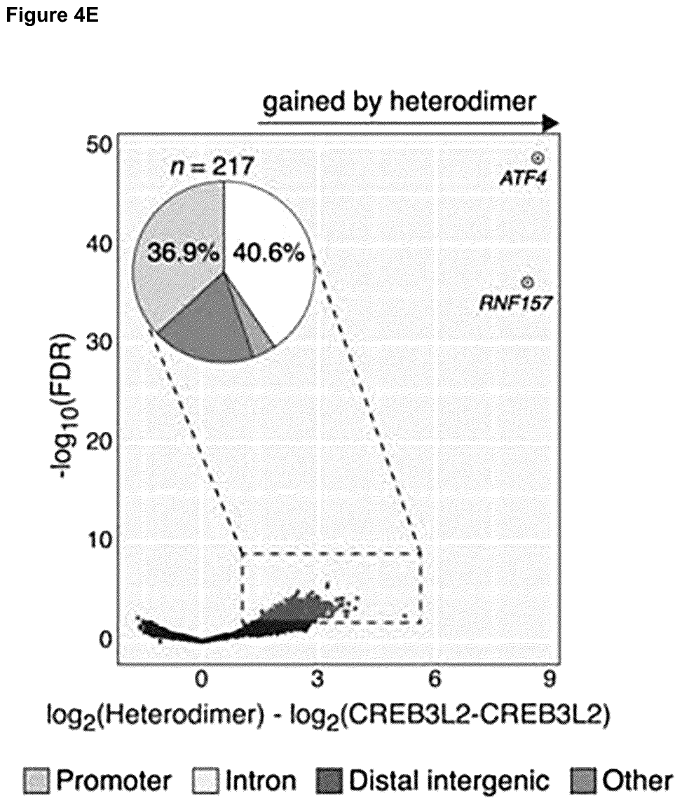

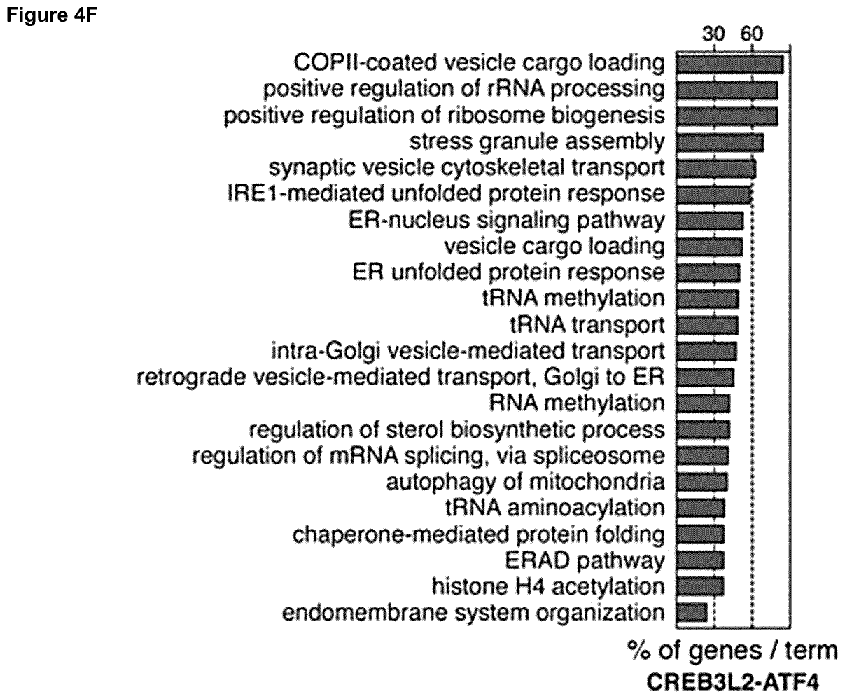

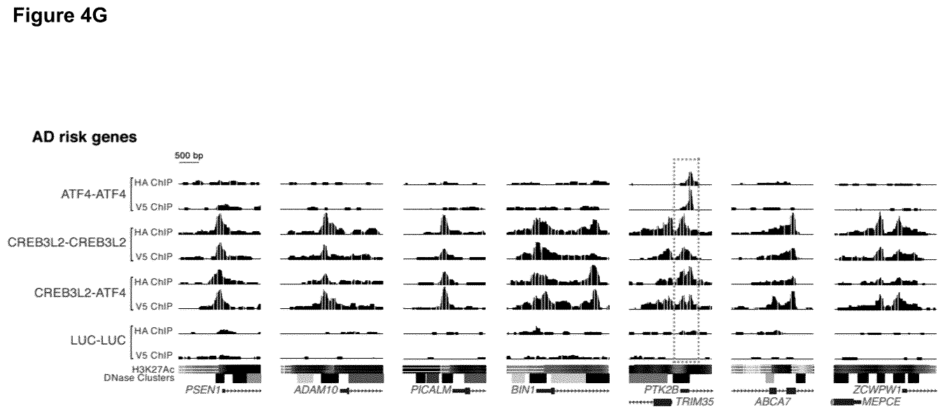

LOAD is characterized by transcriptional alterations in specific groups of co-expressed genes, pointing to deregulated or adaptive network regulators as important drivers of pathology (Mathys et al., 2019; Mostafavi et al., 2018; Zhang et al., 2013). As an upregulated LOAD TF complex, CREB3L2-ATF4 could play a role in promoting these changes during disease progression, which prompted us to investigate its specific transcriptional program. While significant developments have been described recently, current methods are not tailored to identifying binding sites of specific TF dimers on a genome-wide scale in vivo (Hass et al., 2015; Isakova et al., 2017; Jolma et al., 2015). With this in mind, we developed a novel approach to analyze genome-wide patterns of DNA-binding of specific TF dimers in a cellular context. Specifically, we tagged CREB3L2 and ATF4 with FK-binding domains and made use of chemically induced proximity to promote the formation of specific homo- or heterodimers (Stanton et al., 2018). Our design further included HA or V5 epitopes to enable the immunoprecipitation of each component of the dimer and identification of genomic binding sites by ChIP-sequencing (ChIP-seq) (FIGS. 4A and 10A-10B). We call this system ‘ChIPmera’, since it is based on a molecular chimera composed of two TFs.

ChIPmera analysis revealed that CREB3L2-ATF4 heterodimer-bound sequences were enriched in either CREB3L2 or ATF4 recognition sites (FIGS. 4B and 10E), implying that this association does not determine new DNA binding site preferences. Indeed, heterodimer-bound genomic domains were also targeted by CREB3L2 and/or ATF4 homodimers (FIG. 4C). Some genes regulated by ATF4 homodimers were not bound by the heterodimer, while generally all CREB3L2 homodimer hits were found in the heterodimer ChIP (FIGS. 4C-4E). Overall, the CREB3L2-ATF4 heterodimer shared more of its transcriptional signature with CREB3L2 than ATF4, which can be understood from the larger number of genomic sites occupied by CREB3L2 homodimers (6,517 versus 1,727). Also informative was the genomic distribution of CREB3L2 and ATF4 binding sites: whereas CREB3L2 was found to predominantly accumulate in the vicinity of transcription start sites (TSS; 92.9% of all signals), the ATF4 homodimer was more evenly distributed throughout the genome, with a significant proportion of its peaks located in introns (36.2%) and distal intergenic regions (19.4%), and only 41.6% found within promoter regions. In turn, this pattern was reflected on CREB3L2-ATF4 heterodimers, which, like CREB3L2 homodimers, mainly targeted promoters (91.4%).

Gene ontology (GO) terms for CREB3L2-ATF4 included a combination of biological functions individually associated with CREB3L2 or ATF4 (FIGS. 4F and 10F-10H), even if in large part skewed towards the CREB3L2 program. COPII vesicle-associated processes, such as vesicle coating and loading, among other functions related to intracellular trafficking, were exclusively linked with CREB3L2 homodimers (FIG. 10F), and these were also enriched categories in the CREB3L2-ATF4 dataset (FIGS. 4F and 10H; Barbosa et al., 2013; Khetchoumian et al., 2019). ATF4 homodimers, on the other hand, included a number of terms related to cellular stress responses, which the CREB3L2-ATF4 heterodimer likewise incorporated (FIGS. 4F and 10G-10H). Interestingly, we found that a large number of the most established AD risk genes were targeted by the CREB3L2-ATF4 heterodimer via the CREB3L2 program. These included ABCA7, ADAM10, BIN1, FERMT2, MEF2C, PDL3, PICALM, PSEN1, PTK2B, and ZCWPW1, many of which have been directly related to AD susceptibility through their impact on the endocytic pathway and APP homeostasis (FIG. 4G; Scheltens et al., 2016; Verheijen and Sleegers, 2018). Together, our analyses uncover the CREB3L2-enriched transcriptional program of the CREB3L2-ATF4 heterodimer and reveal direct links with key risk factors of AD pathology. On their own, they also stand as proof-of-concept for ChIPmera in its ability to identify genomic sites bound by TF dimers.

### Example 6

### The Retromer Complex is Transcriptional Targeted by CREB3L2-ATF4

To obtain further insight into the pathological function of CREB3L2-ATF4 complexes, we next used ChIP-seq to study these TFs in the LOAD brain. Two disease cases (both females, with moderate pathology, aged +89 years) were chosen for analysis on the basis of high PFC CREB3L2 and ATF4 expression in addition to reduced post-mortem processing intervals. ChIP-seq on human postmortem tissue has not been widely reported, probably due to inherent technical challenges associated with biobanked material. Still, we were able to resolve 228 genomic sites enriched in CREB3L2 from one LOAD brain, assignable to a set of 179 non-random, highly interconnected protein-coding genes (P=1.22×10⁻⁷; FIG. 5A), indicative of a functionally coherent gene cluster. Consistent with our ChIPmera study, further breakdown of CREB3L2-binding site distribution showed that 83.1% were located within ±3 kb of a TSS (FIGS. 5B and 11A), confirming that CREB3L2 binds preferentially to proximal promoter/enhancer regions. In addition, approximately half of these target sites coincided with CREB3L2-ATF4 heterodimer-enriched regions, and up to 78.8% were part of the CREB3L2 homodimer dataset. Likewise, gene ontology terms related to intracellular trafficking, such as COPII-associated processes, were found to be associated with CREB3L2 in LOAD (FIGS. 5C-5D and 11B). Significantly, we also observed an enrichment of genes encoding components of the retromer (FIGS. 5C-5D), a multimodular protein complex responsible for endosomal protein sorting whose deregulation and dysfunction is linked to LOAD pathogenesis (Cullen and Steinberg, 2018; Small and Petsko, 2015). Retromer-associated genes proximal to CREB3L2 peaks included SNX1, RAB7A and SNX3 (FIGS. 5D and 5F), all of which harbor AD-associated gene variants (Vardarajan et al., 2012).

Next, we asked whether CREB3L2 targets showed expression changes in LOAD PFC and found 25 genes with highly significant deregulation profiles (P<1×10⁻²⁰; FIG. 11C; Zhang et al., 2013). These included the splicing regulator PTBP1 (FIG. 11D), linked to aberrant splicing in AD and previously identified as a longitudinal biomarker for Parkinson's disease (Raj et al., 2018a, b; Santiago and Potashkin, 2015), as the most significantly upregulated CREB3L2 gene target in LOAD. Two retromer complex subunits, SNX3 and RAB7A, which mediate its association with the early and late endosome (Cullen and Steinberg, 2018), respectively, were also part of this group, consistent with the known role of retromer dysfunction in LOAD pathogenesis (FIGS. 5D, 5F and 11C).

Differently, our efforts to immunoprecipitate ATF4-bound chromatin from LOAD postmortem tissue were inconclusive, presumably for issues related to antibody-matrix incompatibility. Instead, we examined an ENCODE Consortium ATF4 ChIP-seq from human K562 cells and found that retromer and other endosome-related gene ontology terms were also significantly enriched in this dataset (FIG. 5E). ATF4 populated genomic regions in the vicinity of VPS29, SNX5, SNX3, RAB7A and EHD1, denoting a partial overlap with CREB3L2-regulated genes (FIG. 5F). Moreover, 3 out of the 5 components comprising the Mon1-Ccz1 complex, which functions as the guanine exchange factor (GEF) for RAB7 (Nordmann et al., 2010), were also part of ATF4 regulatory network (FIG. 5E).

Analysis of ChIPmera signals in the vicinity of retromer-associated genes further clarified the role of CREB3L2 and ATF4 in their regulation. CREB3L2 homodimers spanned elements of the cargo-selective, tubulation and membrane-recruiting modules (FIGS. 4C, 5G and 5H). By contrast, ATF4 homodimers were only found enriched in the RAB7A locus (FIGS. 4C, 5F and 5H), indicating that ATF4 binds to retromer genes primarily in a heterodimer configuration. In line with this observation, the CREB3L2-ATF4 heterodimer associated with VPS26B, VPS35, SNX2, SNX5, SNX3, RAB7A and EHD1 (FIGS. 4C, 5G and 5H). It is also noteworthy that ChIPmera signals coincided with various initially unresolved LOAD CREB3L2 ChIP-seq peaks (e.g., VPS26B and EHD1; FIG. 5G). Together, these results identify the retromer complex as a significant pathophysiological target of the CREB3L2-ATF4 complex. They additionally underscore the importance of considering TF interactions when assessing specific DNA-binding programs.

### Example 7

### Widespread Retromer Deregulation in LOAD

While it is increasingly clear that retromer dysfunction contributes to the progression of AD, the molecular mechanisms underlying its impairment are currently not understood. Our finding that the CREB3L2-ATF4 heterodimer targets a number of retromer-associated genes suggests that it might function as a master regulator driving AD pathology via transcriptional misregulation. To explore this, we analyzed the LOAD PFC transcriptome for potential correlations between retromer, CREB3L2 and ATF4 (Zhang et al., 2013). We discovered that retromer deregulation in LOAD is much more extensive than previously recognized (FIGS. 6A-6B), i.e. not restricted to the VPS26-VPS29-VPS35 cargo-selective core as anticipated, but spreading across all of its different subcomplexes. These changes were particularly pervasive amongst subunits of the cargo-selective and membrane-recruiting subcomplexes, which were consistently found to be downregulated in the diseased brain, and had VPS29 ranking as the most significantly altered retromer gene (FIGS. 6A-6B). Also, perturbed expression of brain-enriched VPS26B rather than the more ubiquitous VPS26A paralogue was observed (FIGS. 6A-6B). Differently, sorting nexins SNX1 and SNX6, as well as EHD1, implicated in endosome membrane tubulation (Naslavsky and Caplan, 2011), were upregulated in LOAD (FIGS. 6A-6B). We additionally noted that a number of retromer subunits correlated strongly with CREB3L2 (Pearson r≥±0.65), both positively and negatively (FIG. 6C). These included VPS26B, VPS29 and VPS35, core retromer components involved in the selection of cargoes (Cullen and Steinberg, 2018), as well as EHD1, SNX1 and SNX6. Using independent datasets (Friedman et al., 2018; Webster et al., 2009), we found that these general trends were similarly present in two other AD-related brain areas analyzed, the fusiform gyrus and temporal cortex (FIGS. 12 and 13). Overall, these analyses confirm and extend previous findings, revealing that LOAD is characterized by widespread retromer transcriptional changes that encompass its multiple subcomplexes.

### Example 8

### ATF4 Enhances the Transcriptional Activity of the CREB3L2-ATF4 Heterodimer

The finding that in LOAD brain CREB3L2 upregulation correlates with expression changes of several retromer-associated genes suggested a link between CREB3L2 and pathological retromer deregulation. Knowing the CREB3L2-ATF4 complex to transcriptionally target a number of retromer subunits, we considered whether this association interfered with retromer gene regulation. To test this, we compared the activation responses of firefly luciferase reporters of retromer expression between homodimer or heterodimer backgrounds using chemically induced proximity to drive specific CREB3L2/ATF4 dimer configurations in HEK293 cells. Despite their widespread enrichment over retromer gene loci, these analyses revealed that CREB3L2 homodimers were generally weak activators of retromer reporter responses (averaging 1.7-fold increments over baseline; FIGS. 6D-6G and 14A-14B). This was not due to a general inability of CREB3L2 homodimers to activate transcription, as appreciable gains in activity were achieved with reporters of VPS29 (ca. 4-fold) and the non-retromer COPII vesicle coat subunit SEC24A (ca. 5-fold) expression (FIGS. 14A and 14C). By contrast, CREB3L2-ATF4 heterodimers showed significantly more robust responses (an average of 14.1-fold over baseline), indicating that ATF4 potentiates the transcriptional activity of the CREB3L2-ATF4 complex (FIGS. 6D-6G and 14A-14B). For example, a reporter of EHD1 expression, the most significantly upregulated retromer gene in LOAD (FIGS. 6A-6B), showed a much stronger activation profile in response to CREB3L2-ATF4 heterodimers than CREB3L2 homodimers (ca. 14.9-fold versus 0.84-fold over baseline, respectively; FIG. 6F). This effect was perhaps best demonstrated by RAB7A, a downregulated LOAD gene that is targeted by ATF4 homodimers, in addition to CREB3L2 homodimers and CREB3L2-ATF4 heterodimers (FIGS. 4C and 5F). We measured the strongest increase in RAB7A reporter activity with ATF4 homodimers (ca. 25-fold above baseline), minimal induction with CREB3L2 homodimers (less than 1-fold), and an intermediate response with CREB3L2-ATF4 heterodimers (ca. 10-fold; FIG. 6G). It is noteworthy that similar patterns of transcriptional activation were observed for reporters of genes not linked with the retromer machinery, such as the splicing regulator PTBP1 and CHOP (FIGS. 6H and 14D) indicating that these effects may be a general consequence of ATF4 dimerization with CREB3L2. The CREB3L2-ATF4 complex thus appears to represent a gain-of-function for CREB3L2 and a partial loss of function for ATF4 in terms of its transcriptional output. When extrapolated to LOAD, these observations also present an underlying rationale for understanding how retromer deregulation can unfold during disease pathogenesis.

### Example 9

### CREB3L2 Deficiency Promotes Amyloidogenic APP Processing

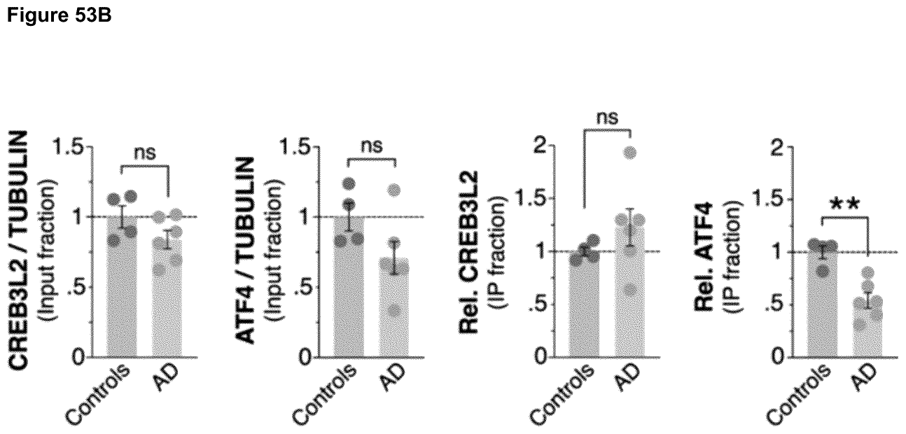

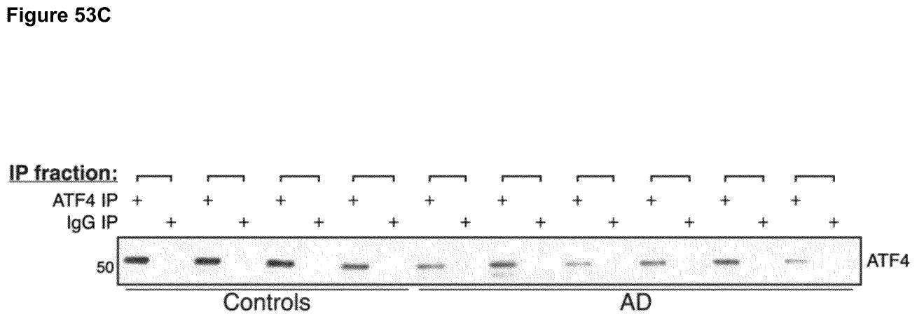

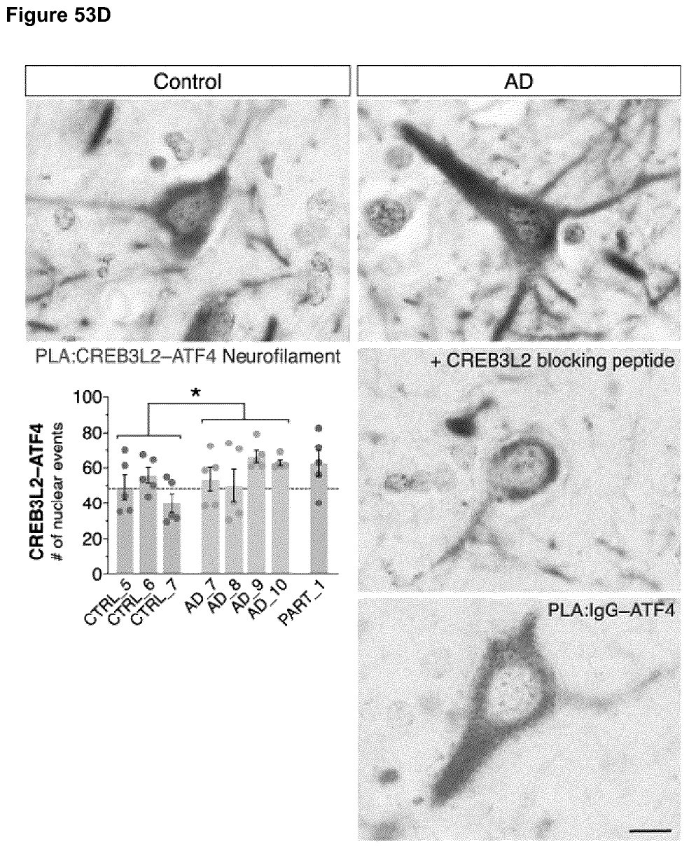

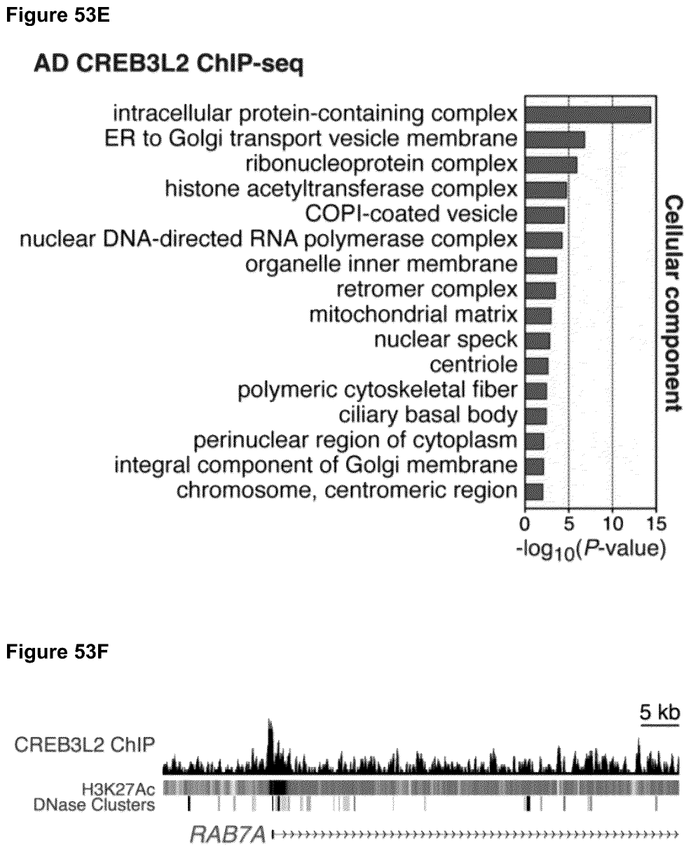

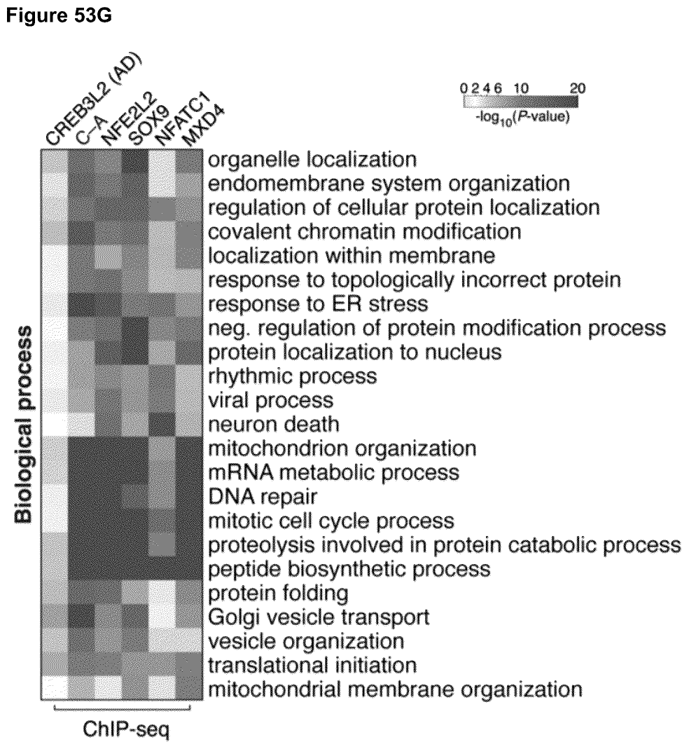

A primary role of retromer is centered on retrieving newly endocytosed integral cell surface proteins from degradation in the lysosome by directing cargoes back to the plasma membrane (Cullen and Steinberg, 2018). Among these, APP recycling is particularly relevant during AD pathogenesis, as its missorting and dwell time in the endosome downstream of retromer dysfunction potentiates the generation of neurotoxic β-amyloid. CREB3L2 and ATF4 might thus be important in maintaining APP homeostasis through their transcriptional regulation of retromer genes. Here, however, we focused exclusively on CREB3L2, as ATF4 was found to interfere with normal APP mRNA expression levels in neuronal cells (FIGS. 14I-14K). We first used a surface biotinylation labeling protocol coupled with avidin-based affinity capture to examine APP cellular localization and relative levels in Creb3/2-depleted neurons, which show diminished retromer mRNA and protein expression (FIGS. 14F-14H). Analysis of surface proteomes and flow-through fractions by western blot revealed an equivalent amount of APP residing at the plasma membrane in both conditions, but reduced levels of intracellular APP in Creb3/2-suppressed neurons relative to control cells (FIG. 61). Next, we measured Aβ₁₋₄₂ and generally benign Aβ₁₋₄₀ peptides in culture supernatants and observed higher Aβ₁₋₄₂/Aβ₁₋₄₀ ratios as a result of CREB3L2 deficiency (FIG. 6J; Nakamura et al., 2018), indicating that CREB3L2 is necessary to keep pro-amyloidogenic cleavage of APP in check. By contrast, non-amyloidogenic APP processing, which takes place primarily at the cell surface (Zhang and Song, 2013), appeared unaffected, as no differences were detected in soluble APPα (sAPPα) levels, an extracellular product of α-secretase cleavage, between control and Creb3/2-deficient neurons (FIG. 6K). We further observed that APP carboxy terminal fragments (CTFs), α-CTF and β-CTF, generated by α- and β-secretase, respectively, were significantly reduced in Creb3/2 knockdowns (FIG. 6L). Globally, these results underlie a requirement for CREB3L2 in balancing non-amyloidogenic versus pro-amyloidogenic APP processing. Although we cannot exclude additional contributing factors, such as a direct transcriptional effect on other modulators of APP metabolism, our findings are consistent with the expected outcome of retromer dysfunction and, ultimately, mechanistically connect CREB3L2 deregulation and APP homeostasis.

### Example 10

### Discussion

Transcriptional changes are widespread in disease and directly linked to the appearance and maintenance of pathological phenotypes (Lee and Young, 2013). Identifying the transcriptional cues responsible for gene network deregulation is thus crucial for a deeper understanding of disease mechanisms and potentially the identification of novel therapeutic targets. Our finding that CREB3L2 and ATF4 form a pathologically relevant complex underscores the importance of considering TF combinatorial relationships in order to understand gene expression and disease etiology. As TF dimerization and other higher-order associations are widespread for many TF families (Amoutzias et al., 2008), but remain largely unmapped (Lambert et al., 2018b), it is expected that significant biological insights can be gained from future investigations of TF interactions in normal and disease states. Here, ChIPmera, a novel methodology we introduced to interrogate specific TF combinations on their association with the genome, could prove particularly useful, especially when coupled with improved strategies for systematic protein-protein interaction scrutiny on a cellular level.

TF dimerization is recognized as a way of cells generating variability in DNA-binding profiles and hence differential gene regulation programs (Reiter et al., 2017). Akin to this, the CREB3L2-ATF4 heterodimer forms a gain-of-function relationship, whereby ATF4 acquires a large number of new genomic targets from CREB3L2, and the latter benefits from the stronger transcriptional output of ATF4. Importantly, CREB3L2-ATF4 signaling can be initiated in the neuronal periphery in response to local amyloid insults via the coordinated synthesis of CREB3L2 and ATF4, acting as a long-range sensor of neurodegenerative stimuli. This is consistent with the increasingly recognized role of neuronal projections as conduits of AD pathology (Baleriola et al., 2014; Liu et al., 2008; Pascoal et al., 2019). CREB3L2-ATF4 complex formation might thus be promoted as a way of encoding the source and nature of the input, similarly to NPAS4 heterodimers and their stimulus-specific induction by different patterns of neuronal activity (Brigidi et al., 2019).

The logic behind the CREB3L2-ATF4 heterodimer can also be understood from the combined CREB3L2 and ATF4 transcriptional programs. For example, both TFs are ER stress transducers (Hetz and Papa, 2018; Kondo et al., 2007), but regulate different branches of the unfolded protein response: CREB3L2 impacts primarily genes of the IRE1 pathway, while ATF4 controls elements of the PERK and ATF6 arms. As a complex, the CREB3L2-ATF4 heterodimer integrates components of all three branches of the UPR, making for a master regulator of cellular proteostasis. It is noteworthy that the retromer protects against accumulation of misfolded proteins and is essential to mitigate UPR activation (Dhungel et al., 2015), a biochemical feature of AD and other neurodegenerative disorders (Hetz and Saxena, 2017). In this sense, retromer deregulation might be a side effect of the heighted response to stress enforced by CREB3L2-ATF4 upregulation. Moreover, by impairing retromer function, Aβ₁₋₄₂-initiated CREB3L2-ATF4 signaling can contribute over time to aggravate neuronal amyloid load, in a feed-forward mechanism of disease.

Finally, the notion that specific TF interactions can perturb homeostatic balances presents in itself a unique therapeutic opportunity—while directly silencing a transcription factor is likely to impact the entirety of its regulatory program, preventing specific associations holds promise of fewer undesired consequences. Precise interference with TF dimerization has already been achieved using dominant-negative peptide inhibitors and small non-peptide molecules (Gerdes et al., 2006; Lambert et al., 2018a), indicating that the targeted modulation of TF interactions could emerge as an amenable therapeutic strategy.

### Example 11

### A Transcriptional Mechanism for Retromer Misregulation in Alzheimer's Disease

Neurodegenerative disorders, including Alzheimer's disease (AD), are characterized by pathological gene expression misregulation, but the upstream mechanisms triggering these changes remain largely unknown. Many transcription factors (TF) function as obligate dimers and the transcriptional effects of a given TF can be modulated by its choice of binding partner. Currently, it remains unexplored whether and how differential TF dimerization contributes to disease pathogenesis. We report that the bZIP TFs CREB3L2 and ATF4 heterodimerize specifically in AD brain and neurons exposed to β-amyloid in a local translation-dependent manner. Using ChIP-seq, human AD brain expression studies, and RNA-seq analyses of neurons with chemically induced CREB3L2-ATF4 signaling, we define the transcriptome-wide changes caused by this disease-linked TF heterodimer. Among other relevant AD pathways, CREB3L2-ATF4 disrupts the expression profile of the retromer, an endosomal sorting complex whose impairment is associated with disease progression. While CREB3L2 is itself a physiological regulator of retromer gene expression in neurons, its β-amyloid-induced dimerization with ATF4 leads to a partial loss-of-function that recapitulates the transcriptional signature of the retromer in AD and overall promotes characteristic disease phenotypes. Together, our findings show that differential TF heterodimerization can encode disease stimuli and elicit pathogenic gene expression changes in neurons.

### Introduction

Late-onset Alzheimer's disease (AD) is a devastating neurodegenerative disorder with a complex biological presentation (Long and Holtzman, 2019). Recent genomic studies have, however, put a spotlight on endosomal trafficking, the innate immune response, and cholesterol metabolism for their pivotal role in pathogenesis. This view is also supported by expression analyses of the AD brain, directly implicating the transcriptional misregulation of these pathways in AD pathophysiology. Clarifying how these changes are orchestrated is therefore key to a mechanistic understanding of the condition. While some ‘hub’ transcriptional regulators have already been identified, a coherent model is unlikely to emerge without accounting for the collaborative nature of transcriptional control processes. These include prominently context-dependent transcription factor (TF) interactions in the form of homo- and heterodimers, each with potentially unique regulatory responses (Amoutzias et al. 2008; Lambert et al. 2018).

Within the endosomal system, evidence points to a malfunctioning retromer, a multimodule protein complex that coordinates cargo-sorting events, as a driver of AD (Small and Petsko, 2015; Nixon, 2017; Cullen and Steinberg, 2018; Simoes et al. 2020). Retromer's role in pathogenesis was first suggested by the finding that two of its core cargo-selection subunits, VPS26 and VPS35, are deficiently expressed in the brain of affected individuals (Small et al. 2005). Since then, genetic and functional studies have confirmed that retromer-mediated endosomal recycling is disrupted in AD and other neurodegenerative disorders (Small and Petsko, 2015). Significantly, the trafficking and processing of amyloid precursor protein (APP) are altered by retromer impairment in neurons, while recent observations have additionally established links to the development of tau pathology. Still, despite much progress in understanding the consequences of retromer dysfunction, it remains unknown why retromer misregulation occurs in AD in the first place.

Here, we report the identification and characterization of a pathological TF heterodimer, CREB3L2-ATF4, associated with late-onset AD. We discovered that heterodimer formation is triggered by β-amyloid and confirmed its enrichment in the brain of AD patients. To dissect its role in disease, we developed a new chemogenetic methodology, ChIPmera, which resolves the DNA-binding profile of dimeric TFs in their cellular context. We found that the retromer machinery is a transcriptional target of CREB3L2-ATF4 and uncovered a mechanistic link between heterodimer appearance in AD and disrupted retromer gene expression.

### Results

### Aβ₄₂ Promotes CREB3L2-ATF4 Heterodimer Formation

We have previously reported that in the AD brain, exposure of axons to oligomeric Aβ₄₂ (a neurotoxic β-amyloid peptide derived from APP) causes pathogenic changes and neurodegeneration that spread retrogradely via the local production of the basic-region leucine zipper (bZIP) TF ATF4 (Baleriola et al. 2014; Walker et al. 2018). Intriguingly, ATF4 is constitutively expressed in neurons, including axons (Shigeoka et al. 2016), and contributes to synaptic plasticity and memory formation (Chen et al. 2003; Pasini et al. 2015). What accounts for ATF4 being different in the context of β-amyloid pathology? Since bZIP TFs function as obligate dimers and, alongside Atf4, other bZIP transcripts are expressed in axons (Baleriola et al. 2014; Ji and Jaffrey, 2014), we hypothesized that local Aβ₄₂ stimuli might trigger the formation of unique heterodimers through the coordinated production of ATF4 and a second TF. To identify such potential ATF4-binding partners, we delivered siRNAs specifically to axons of hippocampal neurons cultured in microfluidic chambers and screened for candidates involved in the retrograde spread of Aβ₄₂ pathology. One of the candidates, CREB3L2, recapitulated ATF4-mediated effects: its axonal knockdown mitigated cell death downstream of local exposure to oligomeric Aβ₄₂ (FIG. 15A and FIGS. 22A-22C) and, like ATF4, it bound to and was necessary for somatic activation of Chop (FIG. 15B and FIG. 22D), a pro-death effector of axonal ATF4-mediated Aβ₄₂ insults9. We also observed that interfering with somatodendritic CREB3L2 expression did not improve cell survival upon Aβ₄₂ treatment, indicating that CREB3L2 plays a distinct role in mediating axonal Aβ₄₂ responses (FIG. 15A).

While current knowledge indicates that CREB3 TFs exclusively dimerize within their family and particularly favor homodimerization, our observations raised the possibility that both CREB3L2 and ATF4 could work as part of the same Aβ₄₂-initiated signaling complex. Consistent with this idea, co-immunoprecipitation studies with both in vitro translated and overexpressed proteins revealed that CREB3L2 and ATF4 heterodimerize and do so in a direct manner without requiring post-translational modifications or additional cofactors (FIG. 15C and FIG. 22E). Moreover, in response to Aβ₄₂ stimulation, endogenous ATF4 readily co-immunoprecipitated with CREB3L2 from neurites treated with Aβ₄₂ but not vehicle-treated controls (FIG. 15D and FIG. 22F). We additionally visualized their Aβ₄₂-induced interaction in axons by proximity ligation assay (PLA; FIG. 15E) and found that CREB3L2-ATF4 heterodimer formation required new protein synthesis, since co-treatment with emetine, a ribosome inhibitor, completely prevented their upregulation (FIG. 15F). In line with these observations, we measured an increase in ATF4 and CREB3L2 protein levels in neurites upon local treatment with Aβ₄₂ (FIG. 22G) and, akin to ATF4 (Baleriola et al. 2014; Walker et al. 2018), de novo CREB3L2 synthesis was detected in Aβ₄₂-treated axons using a short puromycin pulse coupled with PLA coincidence detection (puro-PLA) (tom Dieck et al. 2015) (FIG. 22H). CREB3L2-ATF4 signaling was not, however, exclusive to axons—it was also detected in the somatic compartment, both inside and outside nuclei, of dissociated neurons stimulated with Aβ₄₂ (FIG. 15G). Together, these findings identify CREB3L2 and ATF4 as direct interaction partners and uncover that their heterodimerization is promoted by Aβ₄₂.

### S2P Cleavage Mediates CREB3L2-ATF4 Heterodimer Activation

CREB3L2 is produced as a transcriptionally inactive endoplasmic reticulum-transmembrane protein that only translocates to the nucleus after being proteolytically processed (Kondo et al. 2007) (FIG. 23A). While control neurites expressed CREB3L2 only in this precursor form, its cleaved product (active TF) was detectable following Aβ₄₂ stimulation (FIG. 22G). The cleavage of CREB3L2 and its release from the ER is dependent on S2P, a protease that is present and functional in axons (McCurdy et al. 2019), and indeed S2P activity was required for the nuclear accumulation of CREB3L2 induced by Aβ₄₂ exposure in dissociated neurons (FIGS. 23B-23H). This led us to ask whether axonal S2P was required for the neurodegenerative cascade triggered by axonal Aβ₄₂. Under microfluidic isolation, axonal treatment with an S2P inhibitor, nelfinavir (McCurdy et al. 2019; Guan et al. 2012), phenocopied the effects of CREB3L2 knockdown: it completely rescued the retrograde degeneration response triggered by local Aβ₄₂ stimuli (FIG. 15H) and abolished the downstream induction of CHOP expression in the soma (FIG. 15I). Nelfinavir treatment also caused the accumulation of CREB3L2-ATF4 heterodimers in axons (FIG. 23I), likely reflecting that S2P inhibition does not prevent CREB3L2-ATF4 heterodimerization. In addition, axonal inhibition of dynein, a motor of retrograde transport, similarly led to the buildup of CREB3L2-ATF4 in this compartment (FIG. 15J), further indicating that the heterodimer actively translocates to the nucleus after formation. These experiments also revealed that CREB3L2-ATF4 signals receded to control levels within 18 hours of Aβ₄₂ exposure, suggesting that axon-derived CREB3L2-ATF4 heterodimers are part of a temporally delimited signaling event (FIG. 15J). Collectively, our results define a pathway whereby Aβ₄₂ regulates CREB3L2-ATF4 heterodimer activation in axons via local translation- and S2P-mediated mechanisms followed by dynein-dependent retrograde transport of the heterodimer to the nucleus.

### CREB3L2-ATF4 Heterodimers are Characteristic of AD

Having found that Aβ₄₂ triggers CREB3L2-ATF4 heterodimerization in neurons, we next asked whether it occurred in human AD brain. Specifically, we preformed co-immunoprecipitation from samples of non-demented control and late-onset AD cases with clinical diagnoses of mild or moderate pathology. For each individual, we profiled tissue from the dorsolateral prefrontal cortex (PFC; Brodmann area 9/10), a vulnerable brain region during AD progression with an essential role in cognition. A significant enrichment of ATF4 in CREB3L2 immunoprecipitates was present in moderate disease samples, while clinically mild AD cases did not show increased heterodimer levels compared to controls (FIG. 16A). In situ PLA on AD PFC further revealed that CREB3L2-ATF4 signals were located in neurons, predominantly inside the nucleus, but also in axons, as well as in non-neuronal cells (FIG. 16B and FIGS. 24A-24C).

CREB3L2 and ATF4 protein expression levels were significantly increased in these same disease cases and positively correlated with each other (FIG. 16C), a finding corroborated independently by our analysis of a large-scale transcriptional study of the AD PFC19 (FIG. 16D-16F). There were no significant differences between AD females and males in this dataset, albeit healthy females showing overall increased levels of CREB3L2 mRNA compared to healthy males (FIG. 16F). Despite their similarities, other members of the CREB3 family were normally expressed in AD PFC, except for CREB3L1/OASIS, which was downregulated (FIG. 24D). These findings provide direct support for the pathophysiological significance of the CREB3L2-ATF4 heterodimer in late-onset AD and indicate that CREB3L2-ATF4 appearance in PFC is linked to disease progression.

### ChIPmera Identifies DNA-Binding Patterns of Dimeric TFs Across the Genome

Pathological gene expression changes develop concurrently with AD, implying a failure of transcriptional regulatory mechanisms in maintaining network homeostasis (Zhang et al. 2013; Mathys et al. 2019; Mostafavi et al. 2018; Bossers et al. 2010). As an AD-associated TF heterodimer, CREB3L2-ATF4 could contribute to these changes, which prompted us to investigate its specific transcriptional program. However, while recently described developments have clarified with unprecedented detail the binding properties of TF dimers (Isakova et al. 2017; Jolma et al. 2015; Hass et al. 2015), these methods are not tailored to their study in a cellular context. With this in mind, we developed an approach to investigate the genome-wide DNA-binding patterns of specific TF dimers. We fused CREB3L2 and ATF4 with FK-binding domains and utilized chemically induced proximity to promote the formation of specific homo- or heterodimers in HEK293 cells (Stanton et al. 2018). Each TF monomer was also tagged with a unique epitope (HA or V5) to enable its capture and subsequent identification of genomic binding sites by ChIP-sequencing (ChIP-seq) (FIG. 17A and FIGS. 25A-25B). We call this system ‘ChIPmera’ since it is based on a molecular chimera composed of two TFs.

Our evaluation of control Renilla luciferase homodimers, equally promoted by chemically induced proximity, revealed the ChIPmera protocol was mostly without intrinsic background noise (FIG. 25D). Also, maximal CREB3L2 homodimer signals were present, on average, within 15.1 base pairs (bp) of each other, a figure closely matched by coincident CREB3L2-ATF4 peaks (14.6 bp); ATF4 pairs were even more proximal, binding 11.3 bp apart, indicating that this methodology offers very good overall DNA-binding site resolution (FIG. 24C).

We found that CREB3L2-ATF4-bound DNA sequences were centrally enriched in either CREB3L2 or ATF4 canonical recognition sites (FIG. 17B-17C and FIG. 25E), implying that this heterodimeric association does not determine new DNA binding specificities. Indeed, genomic regions bound by CREB3L2-ATF4 were also targeted by CREB3L2-CREB3L2, ATF4-ATF4, or both (FIG. 17C). Still, not all genes regulated by ATF4 homodimers were bound by the heterodimer, while generally CREB3L2-ATF4 heterodimer hits were also part of the CREB3L2 homodimer dataset (FIGS. 17C, 17F and 17G). Indeed, globally, the CREB3L2-ATF4 heterodimer shared more of its transcriptional signature with CREB3L2 than ATF4 (FIGS. 17F and 17G). Also informative was the genomic distribution of CREB3L2-ATF4: like CREB3L2 homodimers, the heterodimer predominated in the vicinity (s 3 kb) of transcription start sites (TSS, henceforth defined 195 as proximal promoters/enhancers; 91.4% of all signals; FIG. 24E).

### Trafficking and Endocytic Pathway AD Risk Genes are CREB3L2-ATF4 Targets

Enriched gene ontology (GO) terms among CREB3L2-ATF4-bound genes included a combination of biological functions individually associated with CREB3L2 or ATF4 (FIG. 17H and FIGS. 26A-26C), even if in large part skewed towards the CREB3L2 program. Golgi-associated processes, such as vesicle coating and loading, among other functions related to intracellular trafficking, were exclusively linked with CREB3L2 homodimers (FIG. 25F), and these were also enriched categories in the CREB3L2-ATF4 dataset (Khetchoumian et al. 2019; Melville et al. 2011) (FIG. 17H and FIGS. 26A-26C). ATF4 homodimers, on the other hand, incorporated several terms related to cellular stress responses, which the CREB3L2-ATF4 heterodimer equally shared (FIG. 17H and FIGS. 26A-26C). Interestingly, while both TFs are ER stress transducers (Kondo et al. 2007; Hetz and Papa, 2018), our analysis revealed that they regulate different branches of the unfolded protein response (UPR): CREB3L2 primarily binds genes of the IRE1 pathway, whereas ATF4 controls elements of the PERK and ATF6 arms (FIGS. 25A and 25B). As a heterodimer, CREB3L2-ATF4 integrates components of all three UPR branches (FIG. 26C and data not shown). CREB3L2 and ATF4 are themselves targets of CREB3L2-ATF4, suggestive of an auto-regulatory mechanism (FIGS. 17F and 17G and FIG. 26D). We additionally noted that a surprisingly high number of AD risk genes (12 out of 29) were targeted by the CREB3L2-ATF4 heterodimer via the CREB3L2 program. These included ABCA7, ADAM10, ALPK2, BIN1, FERMT2, KAT8, MEF2C, PDL3, PICALM, PSEN1, PTK2B, and ZCWPW1, many of which directly related to AD susceptibility through their impact on the endocytic pathway and APP homeostasis (Scheltens et al. 2016; Verheijen and Sleegers, 2018) (FIG. 17I). Together, our analyses uncover the CREB3L2-enriched transcriptional program of the CREB3L2-ATF4 heterodimer and reveal direct links with key pathways associated with cellular proteostasis and AD pathogenesis. They also serve as proof-of-concept for ChIPmera in its ability to identify genomic sites bound by TF dimers.

### The Retromer Complex is a Target of CREB3L2-ATF4

We next investigated the transcriptional response of CREB3L2-ATF4 in the context of human disease using ChIP-seq. Two AD disease cases (both females, with moderate pathology, aged >89 years) were chosen for analysis based on high PFC CREB3L2 and ATF4 expression in addition to reduced post-mortem processing intervals. To the best of our knowledge, ChIP-seq of point-source TFs on human brains has not been reported, probably due to inherent technical challenges associated with biobanked material. While our efforts to immunoprecipitate ATF4-bound chromatin from AD brain were inconclusive, we were able to resolve 228 genomic sites enriched in CREB3L2 from one AD brain, assignable to a set of 179 functionally coherent, highly interconnected protein-coding genes (P=1.22×10⁻⁷) (FIG. 18A). In agreement with our ChIPmera study, further breakdown of CREB3L2-binding site distribution showed that 83.1% were located within ±3 kilobases (kb) of a TSS (FIG. 18B and FIG. 27A), confirming that CREB3L2 binds preferentially to proximal promoter/enhancer regions. In addition, approximately half of these target sites coincided with CREB3L2-ATF4 heterodimer-enriched regions, and up to 78.8% were part of the CREB3L2 homodimer dataset. Likewise, GO terms related to intracellular trafficking and the secretory pathway, such as COPII-mediated vesicle transport, were associated with CREB3L2 in AD (FIGS. 18C-18D and FIG. 27B). Significantly, the retromer complex came up as an enriched function, with SNX1, RAB7A, and SNX3 among the genes proximal to CREB3L2 (FIGS. 18C-18D and FIG. 27C).

We asked next whether CREB3L2 targets showed expression changes in AD PFC and found 25 genes with markedly altered transcriptional profiles (P<1×10⁻²⁰) (FIG. 18E). These included the splicing regulator PTBP1, linked to aberrant splicing in AD and previously identified as a longitudinal biomarker for Parkinson's disease (Santiago and Potashkin, 2015; Raj et al. 2018), as the most significantly upregulated CREB3L2 gene target in AD. Consistent with links to AD, two retromer subunits, SNX3 and RAB7A, which mediate its association with the early and late endosome6, respectively, were found among the downregulated cohort (FIG. 18E), as was EIPR1, another component of the endosomal retrieval machinery (Topalidou et al. 2016). Notably, endosome-to-Golgi retrograde transport was the only enriched GO term in this list (P=8.39×10⁻⁵), prompting us to consider in greater detail a possible connection between CREB3L2 and retromer/endosomal dysfunction in AD.

Reexamination of ChIPmera signals in the vicinity of retromer genes further supported a potentially important regulatory role held by CREB3L2 (and ATF4). The CREB3L2 program was found to span elements of the cargo-selective, tubulation, and membrane-recruiting modules (FIG. 17C and FIG. 18F), which the CREB3L2-ATF4 heterodimer mostly shared. Besides SNX1, RAB7A, and SNX3, the CREB3L2-ATF4 heterodimer associated with core retromer subunits VPS26B and VPS35, as well as SNX2, SNX5, and SNX27, all of which are likewise CREB3L2-CREB3L2 targets (FIG. 17C and FIG. 18F). Differently, as a homodimer, ATF4 was only significantly enriched in the RAB7A locus (FIG. 17C), strongly suggesting that it can bind retromer genes almost exclusively in a heterodimer configuration. It is also noteworthy that ChIPmera signals precisely coincided with various unresolved CREB3L2 ChIP-seq peaks (e.g., VPS26B and VPS35) (FIG. 18F), indicating that more retromer components than initially determined are collectively regulated by CREB3L2 in AD. Together, these results identify the retromer as a pathologically relevant transcriptional target of the CREB3L2-ATF4 heterodimer.

### CREB3L2 is Necessary for Normal Retromer Expression and APP Processing

Together, our findings so far suggested that CREB3L2 is a transcriptional regulator of retromer gene expression. To begin to test this, we knocked down Creb3/2 in rat cortical neurons using RNAi and determined retromer levels by western blot four days post-infection (FIG. 28A). Except for Ehd1, all retromer subunits evaluated significantly declined in expression after depletion of CREB3L2, including each element of the Vps26-Vps29-Vps35 core, consistent with CREB3L2 functioning as a constitutive transcriptional activator for these genes under basal conditions (FIG. 19A). In addition, retromer mRNA expression, as quantified by RT-qPCR, closely mirrored our measurements at the protein level (FIG. 19B), and we were further able to validate CREB3L2 binding to retromer genomic elements in neurons using ChIP-qPCR (FIG. 19C), overall indicating that CREB3L2 works primarily via transcriptional mechanisms to regulate retromer expression in these cells.

A primary retromer function is the retrieval of newly endocytosed integral cell surface proteins from degradation in the lysosome by directing them instead back to the plasma membrane6. For example, APP recycling is particularly relevant during AD pathogenesis, as its missorting and dwell time in the endosome downstream of retromer dysfunction potentiates the generation of neurotoxic β-amyloid. CREB3L2 could thus be important in maintaining APP homeostasis through its transcriptional regulation of retromer expression. With that in mind, and having first confirmed that App mRNA expression was unaltered (FIG. 28A), we employed a surface biotinylation labeling protocol coupled with avidin-based affinity capture to examine APP cellular localization and relative levels in Creb3/2-depleted neurons. Analysis of surface proteomes and flow-through fractions by western blot revealed an equivalent amount of APP residing at the plasma membrane in both conditions but reduced intracellular levels in Creb3/2-suppressed neurons relative to control cells (FIG. 19D). Also, we observed that intracellular APP carboxy-terminal fragments (CTFs), generated by a- or P-secretase cleavage, were significantly reduced in Creb3/2 knockdowns (FIG. 19E). By contrast, in culture supernatants, the levels of Aβ₄₂ and Aβ₄₀ peptides, as well as soluble APPα (sAPPα), an extracellular product of α-secretase cleavage, were unaffected by Creb3/2 deficiency (FIG. 19F). Collectively, these results reveal a physiological requirement for CREB3L2 in the regulation of APP processing and cellular distribution.

CREB3L2 Correlates with Widespread Retromer Misregulation in AD

While retromer dysfunction is an integral part of AD pathogenesis, the molecular mechanisms underlying its impairment are currently not understood. Our finding that the CREB3L2-ATF4 heterodimer targeted the retromer suggested that its contribution to AD could arise in part via the transcriptional misregulation of this endosomal cargo-sorting complex. This prompted us to analyze publicly available late-onset AD PFC transcriptomes to gain further insight into disease-associated retromer gene expression patterns. We discovered that retromer misregulation is more extensive than previously recognized in affected individuals (FIGS. 19G and 19H) (Guan et al. 2012), i.e., not restricted to the VPS26-VPS29-VPS35 core but evident across all its different subcomplexes. These changes were particularly pervasive amongst subunits of the cargo-selective and membrane-recruiting modules, which were consistently found downregulated in the diseased brain, and had VPS29 ranking as the most significantly altered retromer gene (FIGS. 19G and 19H). Also, perturbed expression of brain-enriched VPS26B rather than the more ubiquitous VPS26A paralogue was observed (FIG. 19G). By contrast, sorting nexins SNX1 and SNX6, as well as EHD1, implicated in endosome membrane tubulation, were upregulated in AD (FIGS. 19G and 19H). We additionally noted that the expression changes of several retromer subunits correlated strongly with CREB3L2 (Pearson r≥±0.65), both positively and negatively (FIG. 19I and FIG. 28B). These included VPS26B, VPS29, and VPS35 (negative association), core retromer components involved in the selection of cargoes6, as well as EHD1, SNX1, and SNX6 (positive association). Using independent datasets (Webster et al. 2009; Friedman et al. 2018), we found that these general trends were similarly present in two other AD-related brain areas analyzed, the fusiform gyrus and temporal cortex (FIG. 28C and FIG. 29). Overall, these analyses revealed that the AD retromer is affected by widespread transcriptional changes. Moreover, our findings are suggestive of a causal connection between retromer misregulation and abnormal CREB3L2 activation in AD.

### CREB3L2-ATF4 Signaling Suppresses Trafficking Pathways

Could CREB3L2-regulated gene expression be disrupted by its association with ATF4? To investigate this, we performed RNA-sequencing (RNA-seq) on primary rat hippocampal neurons with increased dosage of CREB3L2-ATF4 using, as above, chemically induced proximity to promote their heterodimerization (FIG. 20A). Specifically, comparisons were made against a control background composed of Renilla luciferase homodimers and CREB3L2-CREB3L2-expressing cells (FIGS. 20B-20C and FIGS. 30A-30C). A meta-analysis of transcriptomic profiles after CREB3L2-ATF4 activation revealed that the heterodimer globally shapes cellular proteostasis (Zhou et al. 2019) (FIGS. 20D and 20E). It does so by bolstering the expression of genes encoding functions in amino acid biosynthesis and transport, protein translation (e.g., tRNA aminoacylation), as well as response genes downstream of heme-regulated eIF2α kinase HRI, but also by lessening the activation of vesicle trafficking processes, particularly Golgi-related functions, including endosome-to-Golgi retrograde transport, an export pathway mediated by the retromer (FIG. 20D). Overall, we found 372 differently expressed genes (P<0.05) in a head-to-head comparison between CREB3L2-ATF4 and CREB3L2-CREB3L2 backgrounds (FIG. 20F and FIG. 30A), a large proportion of which we previously identified as direct heterodimer targets in the ChIPmera datasets (representation factor=5.1; P<3.29×10⁻⁶⁴, hypergeometric test). Interestingly, the heterodimer is seemingly part of a feedback loop (FIG. 20B): it markedly inhibited Atf4 expression (log fold-change=−0.68, P=3.12×10⁻⁵) and promoted increased Creb3/2 levels (log fold-change=+0.91, P=4.38×10⁻¹³), indicating an intrinsic potential to mitigate CREB3L2-ATF4 signaling over time, akin to other stress response regulators (Hetz and Saxena, 2017). In addition, Chop was among the targets whose expression was significantly activated by CREB3L2-ATF4 (log fold-change=+0.31, P=0.04; FIG. 20B and FIG. 30A), corroborating our original finding that this TF is downstream of the CREB3L2-ATF4-mediated pathway initiated by axonal Aβ₄₂ stimuli (FIG. 15B and FIG. 22D). Together, these studies revealed that increased CREB3L2-ATF4 signaling suppresses the ‘canonical’ CREB3L2 program—i.e., it represents a partial loss-of-function for CREB3L2 in terms of its normal transcriptional output. This is especially evident for cellular trafficking processes, of which CREB3L2 is a master regulator.

### CREB3L2-ATF4 Activation Recapitulates AD Retromer Misregulation

We next examined in detail whether CREB3L2-ATF4 signaling was causally linked to the disruption of retromer gene expression. To this end, we measured retromer transcript and protein profiles in cultured hippocampal neurons using RT-qPCR and western blot four days after induction of CREB3L2-CREB3L2 or CREB3L2-ATF4. Globally, these analyses revealed a general trend towards the downregulation of the retromer machinery as a consequence of CREB3L2-ATF4 activation (mRNA average decrease=−12.9%; protein average decrease=−18.6%), albeit with some nuances (FIGS. 20G and 20H). One clear example of this was Vps35, the cargo-selective core ‘backbone’ subunit, which had significantly reduced levels of mRNA and protein expression in CREB3L2-ATF4 neurons (FIGS. 20G and 20H). CREB3L2-ATF4 likewise downregulated Vps26b mRNA (but curiously not the protein), while the effects on Rab7a and Snx3 expression were particularly evident at the protein level (FIGS. 20G and 20H). Interestingly, we detected a marked increase in Ehd1 transcript levels only in CREB3L2-CREB3L2 neurons, in agreement with the findings that CREB3L2 binds to this gene exclusively in a homodimeric configuration and the strong expression correlation shared by both genes in AD (FIGS. 20G and 20H). Similarly, Vps26a, which is not a transcriptional target of CREB3L2-CREB3L2 or CREB3L2-ATF4, was unaffected in either background (FIGS. 20G and 20H). Together, these observations show that the disruption of retromer regulatory processes mediated by the CREB3L2-ATF4 heterodimer, while pervasive across the various subunits studied, does not occur indiscriminately. Importantly, they also closely mirror AD retromer expression profiles (FIGS. 19G and 19H), revealing that the activation of CREB3L2-ATF4 signaling is sufficient to cause AD-like retromer misregulation.

### β-Amyloid and Tau are Downstream of CREB3L2-ATF4

Finally, we asked whether CREB3L2-ATF4 signaling was disruptive to P-amyloid and tau metabolism, two disease-relevant retromer cargos. We first measured Aβ peptides in the medium of hippocampal neurons expressing CREB3L2-ATF4 using a Meso Scale multiplex immunoassay. Compared to controls, CREB3L2-ATF4 neurons had significantly higher Aβ₄₂/Aβ₄₀ ratios (FIG. 21A), revealing an overall pro-amyloidogenic shift in APP processing. This was accompanied by reduced Aβ₄₀ and Aβ₄₂ levels (FIGS. 21B and 21C), with the decline of Aβ₄₀ being, however, more pronounced than that seen for the aggregation-prone Aβ₄₂ peptide (−71.9% versus −44.5%, respectively). A drop in sAPPα levels, as detected by ELISA, was similarly present in the CREB3L2-ATF4 condition (−19.3%), indicating that both the amyloidogenic and non-amyloidogenic processing pathways are affected by the heterodimer (FIG. 21D). Furthermore, we observed by western blot that intracellular APP-CTFs were significantly lower in CREB3L2-ATF4 neurons, although the scale of the disruption at this point of the APP processing pathway was subtle in comparison with our findings in culture supernatants (FIG. 21E). It is noteworthy that these changes were not a result of a direct transcriptional effect of the heterodimer on App, as its mRNA expression was unaffected by the heterodimer (FIG. 21F). They are also unlikely to be due to overt neuronal cell death caused by CREB3L2-ATF4, since lactate dehydrogenase (LDH) release into the medium, a marker of cytotoxicity, while increased over controls, was similar in magnitude to that measured for CREB3L2-CREB3L2 cells (FIG. 21G), which show normal APP metabolism (FIGS. 21A-21E). We conducted in parallel a Meso Scale analysis of extracellular tau, whose secretion is promoted when retromer-dependent endosomal recycling is disrupted. A 62.9% higher accumulation of tau was found in the CREB3L2-ATF4 background over control levels (FIG. 21H), a result even more striking considering that Mapt mRNA, which encodes tau, was downregulated in these neurons (FIG. 21I; incidentally, reduced MAPT expression is also observed in AD). Taken together, our findings reveal that the formation of CREB3L2-ATF4 heterodimers not only affects the expression of retromer genes, but importantly also negatively impacts AD-relevant cargos trafficked in a retromer-dependent manner. Moreover, the pro-amyloidogenic shift in APP processing and the increased secretion of tau caused by the heterodimer supports a function for CREB3L2-ATF4 signaling in the progression of β-amyloid and tau pathologies in the AD brain.

### Discussion

Transcriptional changes are widespread in disease and directly linked to the appearance and maintenance of pathological phenotypes (Lee and Young, 2013). Identifying the transcriptional inputs responsible for gene network misregulation is thus crucial for a deeper understanding of disease mechanisms and potentially the identification of novel therapeutic targets. Collectively, our finding that CREB3L2 and ATF4 form a pathologically relevant heterodimer in AD emphasizes the importance of considering TF combinatorial relationships in order to understand gene expression and disease etiology. As TF dimerization and other higher-order associations are widespread for many TF families (Amoutzias et al. 2008), it is expected that significant biological insights can be gained from future investigations of TF interactions in normal and disease states. Here, ChIPmera, a novel methodology we introduced to interrogate specific TF combinations on their association with the genome, could prove particularly useful, especially when coupled with improved strategies for systematic scrutiny of protein-protein interactions.

TF dimerization is recognized as a way of cells generating variability in DNA-binding profiles and hence differential gene expression programs (Reiter et al. 2017). In line with this idea, we found that the CREB3L2-ATF4 heterodimer jointly regulates transcriptional targets of both CREB3L2 and ATF4, which results in a broader set of biological functions being influenced by its upregulation. However, in the case of CREB3L2, the CREB3L2-ATF4 heterodimer can perhaps be best understood as a partial loss-of-function interaction, given that its association with ATF4 dampens the activation of the core CREB3L2 transcriptional program. Key examples of this include the downregulation of the secretory pathway and other vesicle trafficking-related processes, such as the retromer machinery. Notably, in respect to these signature targets of CREB3L2 regulation, both CREB3L2-CREB3L2 and CREB3L2-ATF4 dimers bind to the same genomic regions—i.e., the two dimers compete with one another for access to these sites, the upregulation of CREB3L2-ATF4 displacing CREB3L2-CREB3L2 inputs, and vice-versa. Rather than producing an additive effect, our findings indicate that increased CREB3L2-ATF4 signaling actually elicits an inhibitory outcome similar to a dominant negative mechanism, leading to an overall suppression of the CREB3L2 program.

Still, our analyses also make clear that the CREB3L2-ATF4 heterodimer goes beyond a mere CREB3L2 loss-of-function, since its upregulation does not fully recapitulate the consequences of knocking down CREB3L2 in neurons. Indeed, despite both conditions disrupting retromer expression patterns in a way that closely resembles those seen in AD, the APP processing pathway is impacted to a greater extent by CREB3L2-ATF4 activation. This indicates that it is the combination of CREB3L2 and ATF4 programs (the former downregulated, the latter upregulated) that may be in fact pathological.

It is noteworthy that proper retromer function protects against the accumulation of misfolded proteins in the ER and is essential to mitigate UPR activation (Dhungel et al. 2015), a biochemical feature of AD and other neurodegenerative disorders (Hetz and Saxena, 2017).

The finding that specific TF interactions can perturb homeostatic networks presents in itself a unique therapeutic opportunity—while directly silencing a TF is likely to impact the entirety of its regulatory program, preventing specific pathological associations holds promise of fewer undesired consequences. Precise interference with TF dimerization has already been achieved using dominant-negative peptide inhibitors and small non-peptide molecules (Gerdes et al. 2006; Lambert et al. 2018), indicating that the targeted modulation of TF interactions could emerge as an amenable therapeutic strategy. In this regard, it is encouraging to note that recent interventions employing retromer-enhancing agents have shown positive outcomes in rodent models of AD (Mecozzi et al. 2014; Li et al. 2019). Modulation of CREB3L2-ATF4 signaling could thus be explored to normalize retromer function and, more broadly, to restore the transcriptional network disrupted by its upregulation.

### Data and Materials Availability:

ChIPmera datasets are available at the Gene Expression Omnibus repository https://www.ncbi.nlm.nih.gov/geo/(accession no. GSE147205).

### Materials and Methods

### Neuronal Culture

Same as that described above in Example 1.

### Aβ₄₂ Peptide Oligomerization and Treatment

Same as that described above in Example 1.

Axonal siRNA Transfection

Same as that described above in Example 1.

### Cell Death Assay (TUNEL)

Same as that described above in Example 1.

### CHOP Immunocytochemistry and Image Analysis

Same as that described above in Example 1.

### Chromatin Immunoprecipitation and Quantitative PCR (ChIP-qPCR)

Same as that described above in Example 1.

### Generation of GFP-Tagged CREB3L2

Same as that described above in Example 1.

### Rabbit Reticulocyte Lysate Translation System and Immunoprecipitation

Same as that described above in Example 1.

### Western Blot Analyses

Same as that described above in Example 1.

### Proximity Ligation Assay on Primary Neuronal Cultures

Same as that described above in Example 1.

### In Situ Visualization of Newly Synthesized Proteins (Puro-PLA)

Same as that described above in Example 1.

### Transwell Neuronal Culture and Co-Immunoprecipitation

Same as that described above in Example 1.

### Human Brain Sample Procurement and Processing

Same as that described above in Example 1.

### Analysis of LOAD-Associated and Related Transcriptional Profiles

Same as that described above in Example 1.

### Immunohistochemical Analysis of CREB3L2 in LOAD Prefrontal Cortex

Same as that described above in Example 1.

### CREB3L2-ATF4 Co-Immunoprecipitation in Human Brain Tissue

Same as that described above in Example 1.

### Proximity Ligation Assay on Late-Onset AD Cortical Tissue

A standard prerequisite of the PLA protocol in its kit format is the availability of specific primary antibodies raised in different hosts. As we were unable to locate a compatible IHC-validated pair of CREB3L2 and ATF4 antibodies, we resorted to using the Duolink Probemaker kits (DU092009 and DU092010, Sigma) to directly conjugate CREB3L2 (HPA015068, Atlas Antibodies) and ATF4 (ab184909, Abcam) antibodies, both of which raised in rabbit, with PLA PLUS and MINUS oligonucleotides. However, this approach requires that the antibodies are in solubilized a carrier- and preservative-free buffer; to achieve this, both antibodies were dialyzed by employing a Slide-A-Lyzer device with a 10K molecular weight cutoff (#69570, Thermo Scientific) made to float on a glass beaker containing 200 ml of PBS for 2 hours. The whole protocol was performed inside a cold room to minimize degradation and, in the case of the anti-CREB3L2 antibody, was followed by a concentration step (#88513, Thermo Scientific). The conjugation reaction was performed overnight at room temperature, and CREB3L2-ATF4 heterodimers stained using Duolink In situ Brightfield Detection reagents (DU092012; Millipore Sigma). As per manufacturer's instructions, the PLA Probe Diluent included in the Probemaker kit was used in substitution of the PLA Antibody Diluent in the PLA protocol. Up until the blocking step, tissue pre-treatment steps were equivalent to those described for the immunohistochemistry detection of CREB3L2 expression in human brain. Co-staining with anti-MAP2 (1:2000; ab5392, Abcam) or anti-Neurofilament (1:400; heavy chain subunit; #N0142, Millipore Sigma) antibodies was performed afterward, and signals developed using the Vector Blue Alkaline Phosphatase substrate kit (SK-5300, Vector Laboratories). To increase detection sensitivity, we additionally employed the Vectastain ABC-AP system (AK-5002, Vector Laboratories) in the staining procedure before signal development. Finally, sections were dehydrated with ethanol (50%>70%>95%>100%), cleared with Histo-Clear (64110-01, Electron Microscopy Sciences), and mounted in Vectamount (H-5000, Vector Laboratories). Slides were imaged on an Olympus BX53 microscope equipped with an Olympus DP72 camera and a UPlan FL n 20×/0.50 objective (Olympus). cellSens Standard v1.13 (Olympus) was used as the acquisition software.

### CREB3L2 ChIP-Sequencing in AD Prefrontal Cortex

Same as that described above in Example 1.

### Chemically Induced Proximity: Reagent Preparation

C-terminal FRB (T82L mutant) or FKBP (wild-type) fusions, N-terminally tagged with HA or V5 epitopes, respectively, were synthesized by Genewiz and transferred into pGL4.75[hR/uc/CMV] (Promega) using Sac and FseI sites. Human CREB3L2 (residues 2-384, corresponding to its transcriptionally active form) and full-length ATF4 (S219A stabilization mutant were utilized in transgene design⁵³); control Renilla luciferase sequence was obtained from pGL4.75 (Promega). Successful insertions were screened by Sanger sequencing. For neuronal expression, these same transgenes were cloned into a modified FUGW plasmid (Addgene #14883, gift by David Baltimore) in which the GFP open reading frame was substituted by a custom multiple cloning site (CTCTAGAGGATCCCCGGGTACCGGTGGCGCGCCGCTTAGCGTTAACGCTAGCCG GACCGCCTGCAGGAGGCCTGCCCGGGCATTTAAATGAATTCAAC; SEQ ID NO: 67, fragment synthesized by Genewiz) using BamHI and EcoRI sites. Lentiviral particles were harvested from HEK293T cells after Lipofectamine 3000-mediated transfection with lentiviral and packaging plasmids (pCMVD R8.9 and pHCMV VSVg); 6 hours post-transfection, the medium was changed to Neurobasal containing B27 and L-glutamine. Viral supernatant was collected 18 hours later, centrifuged 500 g for 5 minutes, passed through a 0.45 μm PES filter, aliquoted, and stored at −80° C. A/C heterodimerizer (C-16-(S)-7-methylindolerapamycin [Aβ₂₁₉₆₇]) was purchased from Takara.

### ChIPmera—Chromatin Immunoprecipitation and Data Analysis

Same as that described above in Example 1.

shRNA Preparation and Delivery

Same as that described above in Example 1.

### RNA Extraction and Quantitative Real-Time PCR

Same as that described above in Example 1.

### Cell Surface Protein Biotinylation

Same as that described above in Example 1.

### RNA-Sequencing

Prior to RNA collection, neuronal cultures were washed with ice-cold HBSS (Thermo Fischer) and lysed with TRIzol reagent (#15596026, Thermo Fisher) by scrapping. The reaction was allowed to proceed on ice for 5 minutes, at which point samples were centrifuged at 12,000 g and 4° C. and supernatants transferred to new tubes before a 1:1 (v/v) dilution with molecular biology-grade 100% ethanol (Millipore Sigma). The RNA extraction protocol was continued using the Direct-zol RNA MicroPrep kit (Zymo Research), in accordance to manufacturer's instructions, and included an on-column DNA digestion step with DNase I. RNA was eluted in 30 μl of water. RNA-seq library preparation and sequencing reactions were conducted at GENEWIZ, Inc. (South Plainfield, NJ, USA). RNA sequencing libraries were prepared using the NEBNext Ultra RNA Library Prep Kit for Illumina following manufacturer's instructions (NEB). Briefly, mRNAs were first enriched with oligo(dT) beads. Enriched mRNAs were fragmented for 15 minutes at 94° C. First-strand and second-strand cDNAs were subsequently synthesized. cDNA fragments were end-repaired and adenylated at 3′-ends, and universal adapters ligated to cDNA fragments, followed by index addition and library enrichment by limited-cycle PCR. The sequencing libraries were validated on an Agilent TapeStation (Agilent Technologies) and quantified using a Qubit 2.0 Fluorometer (Life Technologies) as well as quantitative PCR (KAPA Biosystems). The sequencing libraries (15 in total) were clustered on 1 lane of a flowcell. After clustering, the flowcell was loaded onto an Illumina HiSeq instrument (4000 or equivalent) according to manufacturer's instructions. The samples were sequenced using a 2×150 bp Paired-End (PE) configuration. Image analysis and base calling were conducted by HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification. RNA-seq sequencing data were processed and analyzed within the Galaxy web platform, using the public server at usegalaxy.org. First, library adapters and low-quality reads were removed using Trimmomatic (version 0.38) with the following settings: initial ILLUMINACLIP step to cut adapters and other Illumina-specific sequences from the reads, seedMismatches=2, palindromeClipThreshold=30, simpleClipThreshold=10, minAdapterLength=8, keepBothReads=True, AVGQUAL >20, SLIDINGWINDOW: windowSize=4 and requiredQuality=20, and MINLEN=50. Second, reads were mapped to the Rattus norvegicus (Rn) 6.0 reference genome and gene model (downloaded from Ensembl) with RNA STAR (version 2.7.5b) using default settings except: sjdbOverhang=149, outFilterType=True, alignintronMax=1000000, alignMatesGapMax=1000000, and alignSJoverhangMin=8; additionally, unmapped reads, alignments that had junctions with inconsistent strands, alignments across unannotated non-canonical junctions and all alignments across non-canonical junctions were excluded from output. Third, featureCounts (version 1.6.4+galaxy2) was run to quantify reads mapping to exons with the following parameters: ‘create gene length file’=True, ‘count fragments instead of reads’=True, ‘only allow fragments with both reads aligned’=True, ‘exclude chimeric fragments’=True, ‘GFF feature type filter’=exon, ‘GFF gene identifier’=gene_id, ‘on feature level’=False, ‘allow reads to map to multiple features’=False, ‘minimum mapping quality per read’=10; otherwise default tool settings. Forth, differentially expressed features were determined using default DESeq2 (version 2.11.40.6+galaxy1) settings, except: ‘output normalized counts table’=True. Fifth, DESeq2 output was filtered to extract only the most differentially expressed genes (adjusted P-value <0.05) between conditions. Sixth, the Galaxy tool ‘Annotate DESeq2/DEXSeq output tables' (version 1.1.0) was employed to retrieve gene annotations. Subsequently, to visualize gene expression profiles over samples, Z-scores were computed from normalized gene counts and plotted using the heatmap2 tool. Comparative gene ontology enrichment meta-analyses were performed with differential expression lists through the Metascape web portal (Zhou et al. 2019).

### Neuronal Culture Supernatant Collection and Measurements

Culture supernatants were transferred to 15-ml falcon tubes, spun at 2000 g and 4° C. for 5 minutes, aliquoted, and stored at −80° C. A sandwich immunoassay (V-PLEX Aβ Peptide Panel 1 kit, #4G8, Meso Scale Discovery) was employed in the measurement of β-amyloid species. Manufacturer's guidelines were followed thoroughly during plate preparation, and samples diluted 1:1 with Diluent 35 (provided as part of the kit) to avoid matrix saturation. All biological replicates were measured in parallel. Signal readings were performed on a Sector Imager 2400 instrument (Meso Scale Discovery). For assessing sAPPα levels in culture supernatants, we utilized a sandwich ELISA assay (sAPPα [Mouse/Rat] [highly sensitive], #27419, Immuno-Biological Laboratories) and samples were diluted 10-fold. Extracellular tau levels were assessed using the Phospho(Thr231)/Total Tau Kit from Meso Scale Discovery following the protocol provided by the manufacturer. It is noteworthy, however, that phospho-tau signals were deemed too low to provide any meaningful assessment of phospho-tau levels, likely a consequence of no phosphatase inhibitor being added to the supernatants at the time of collection, and were discarded. LDH release was evaluated using an LDH-Glo Cytotoxicity Assay from Promega.

### General Statistical Analysis

Same as that described above in Example 1.

### Example 12

### Transcriptional Deregulation in Parkinson's Disease by ATF4 Transcription Factor Heterodimers

This study is to determine how ATF4 transcription factor heterodimers regulate gene expression changes in Parkinson's disease (PD).

### Background

Gene expression analysis of substantia nigra from PD patients has identified numerous differentially expressed genes (DEGs) involved in cellular pathways such as mitochondrial function, synapse organization, and macroautophagy. However, how changes in gene expression are regulated remains unknown. ATF4 is a stress-responsive, obligate heterodimeric transcription factor (TF) that has been implicated in PD. Most studies of the role of TFs in disease have focused on identifying changes in TF expression level and correlating that with function. However, we hypothesized that changes in heterodimerization patterns could also result in disease-specific transcriptional deregulation. This project has focused on understanding how ATF4 transcription factor heterodimer complexes affect PD pathogenesis by altering gene expression.

### Design/Methods:

We focused primarily on a novel TF heterodimer consisting of ATF4 and another stress-responsive TF, CREB3L2 (FIGS. 31A-31B). We developed a novel molecular technique called ChIPmera to identify a large dataset consisting of all possible transcriptional targets of this complex and then compared this dataset with a PD patient-derived gene expression dataset to identify a network of PD-associated DEGs that are also potential targets of the ATF4-CREB3L2 complex (FIGS. 32A-32B). Network analysis was performed to identify cellular pathways whose dysregulation in PD may be mediated by this ATF4 heterodimeric complex.

### Results:

We identified 255 genes that are both dysregulated in PD and potential transcriptional targets of the ATF4-CREB3L2 TF complex. Network and functional enrichment analysis of these genes identified several clusters associated with different cellular pathways, including those well-known to be dysregulated in PD, such as mitochondrion organization, and less well-studied pathways such as RNA splicing (FIGS. 33A-33D).

### Conclusion:

These results demonstrate a novel approach for dissecting the link between specific TF complexes, differential gene expression, and cellular dysfunction in PD.

### Validation of ChIPmera/PD DEG Intersectional Analysis

We have obtained ATF4 and CREB3L2 conditional knock-out mice. We plan to validate the effect of ATF4 and CREB3L2 TFs on differential gene expression and cellular dysfunction in PD, using primary cultured midbrain neurons from these animals. This will include: 1. qPCR for changes in gene expression. 2. Cell biologic assays of mitochondrial function, mRNA processing, intracellular transport, and other pathways identified through this analysis in in vitro models of PD.

### Example 13

### CREB3L2-ATF4 Heterodimerization Defines a Transcriptional Hub of Alzheimer's Disease Gene Expression Linked to Neuropathology

Gene expression is changed by disease, but how these molecular responses arise and contribute to pathophysiology remains less understood. We discover that β-amyloid, a trigger of Alzheimer's disease (AD), promotes the formation of pathological CREB3L2-ATF4 transcription factor heterodimers in neurons. Through a multi-level approach based on AD datasets and a novel chemogenetic method that resolves the genomic binding profile of dimeric transcription factors (ChIPmera), we find that CREB3L2-ATF4 activates a transcription network that interacts with roughly half of the genes differentially expressed in AD, including subsets associated with β-amyloid and tau neuropathologies. CREB3L2-ATF4 activation drives tau hyperphosphorylation and secretion in neurons, in addition to misregulating the retromer, an endosomal complex linked to AD pathogenesis. We additionally provide evidence for increased heterodimer signaling in AD brain and identify dovitinib as a candidate molecule for normalizing β-amyloid-mediated transcriptional responses. The findings overall reveal differential TF dimerization as a mechanism linking disease stimuli to the development of pathogenic cellular states.

### Introduction

Alzheimer's disease (AD) is a progressive neurodegenerative disorder with an increasing worldwide prevalence. The preclinical phase of AD, which can last 10-20 years, is characterized by the gradual accumulation of β-amyloid and tau aggregates in the brain, together with neuroinflammation and synaptic alterations (Long and Holtzman, 2019). Several lines of evidence indicate that β-amyloid deposition precedes and accelerates tau pathology, the latter correlating with the onset of cognitive decline (Long and Holtzman, 2019; Hanseeuw et al., 2019; Sato et al., 2018). Concurrently, gene expression changes across specific pathways tied to pathophysiology are observed (Zhang et al., 2013; Mostafavi et al., 2018; Bossers et al., 2010; Mathys et al., 2019), highlighting an important role for altered transcriptional regulators in AD. What causes these changes, how they interact with β-amyloid and tau pathologies, and whether they are drivers of disease or a response to it remain, however, unclear.

Far from a binary on/off switch, gene expression has emerged as a nuanced, dynamic and collaborative process involving various transcriptional layers (Buccitelli and Selbach, 2020). It follows that AD-associated expression changes can only be fully explained in light of this regulatory interdependency. Transcription factor (TF) dimerization, a common feature among many TF families, is a salient but mostly overlooked case in point, in that it can generate enormous variability in DNA-binding specificities and transcriptional activities (Amoutzias et al., 2008; Reiter et al., 2017; Rodriguez-Martinez et al., 2017; Brigidi et al., 2019). Nonetheless, network analyses of gene co-expression profiles typically employed in AD research are not designed to capture these synergistic TF combinations, resulting in a fragmented mechanistic understanding of the gene programs underlying AD progression and, most likely, missed therapeutic opportunities.

Here, we report the discovery and characterization of a pathological TF heterodimer, CREB3L2-ATF4, linked to AD pathogenesis. CREB3L2-ATF4 heterodimerization is potentiated by β-amyloid in neurons, and we confirmed that their association is enriched in AD brain. To probe its role in pathogenesis, we engineered a new chemogenetic methodology, ChIPmera, which resolves the DNA-binding specificities of dimeric TFs in their cellular context. We found that the CREB3L2-ATF4 heterodimer regulates a transcription network linked to AD gene expression and triggers characteristic cellular features of the condition, including tau hyperphosphorylation, a primary driver of neurodegeneration in AD (Long and Holtzman, 2019). Overall, the findings reveal that TF dimerization can contribute to the disruption of gene networks and the exacerbation of disease processes.

### Results

### Aβ₄₂ Promotes CREB3L2-ATF4 Heterodimerization

The basic-region leucine zipper (bZIP) TF ATF4, an integral part of the unfolded protein response (Hetz et al., 2020), is constitutively expressed in neurons and contributes to synaptic plasticity and memory formation (Shigeoka et al., 2016; Chen et al., 2003; Pasini et al., 2015). ATF4 is also associated with various neurodegenerative disorders, including AD, and is known to activate both pro-survival and pro-death signaling pathways in a context-dependent manner (Sun et al., 2013; Pitale et al., 2017). We have previously reported that axonally synthesized ATF4 mediates pathogenic transcriptional changes and neurodegeneration triggered by soluble oligomeric Aβ₄₂ (Baleriola et al., 2014; Walker et al., 2018), a neurotoxic β-amyloid peptide linked to the onset of AD (Long and Holtzman, 2019). What accounts for the different functions displayed by ATF4 in these various settings? In particular, how does ATF4 function downstream of Aβ₄₂ as a driver of AD pathogenesis? Since bZIP TFs operate as obligate dimers (Amoutzias et al., 2008), we hypothesized that differential heterodimerization could modulate ATF4's unique mode of action in response to Aβ₄₂ by allowing for a distinct transcriptional output. To identify such potential ATF4-binding partners, we delivered siRNAs to axons of hippocampal neurons cultured in microfluidic chambers and screened for genes involved in the retrograde spread of β-amyloid pathology. Specifically, we focused on candidate mRNAs that, like Atf4, were previously found to be recruited into axons upon exposure to Aβ₄₂ (FIGS. 43A and 43B) (Baleriola et al., 2014) and evaluated the effect of their local knockdown by two criteria: prevention of CHOP induction, a pro-death effector TF downstream of ATF4-mediated neurodegeneration (Baleriola et al., 2014), and mitigation of cell death. The bZIP TF CREB3L2 passed on both counts, in that its knockdown prevented the somatic activation of Chop (FIG. 35A) and reduced Aβ₄₂-promoted cell death (FIG. 35B), akin to the effects of ATF4 suppression (Baleriola et al., 2014). By contrast, silencing Hif1a, another axon-localizing bZIP TF mRNA (Baleriola et al., 2014), did not protect against Aβ₄₂ (FIG. 43C). As another approach to interfere with CREB3L2 generation and validate our initial screen, we locally inhibited S2P, a protease required for the transcriptional activation of this TF (McCurdy et al., 2019). Like CREB3L2 knockdown, S2P pharmacological inhibition abolished the induction of CHOP expression and ameliorated the retrograde degeneration response triggered by axonal Aβ₄₂ stimuli (FIGS. 43D and 43E). Subsequent chromatin immunoprecipitation (ChIP-qPCR) analyses in dissociated neurons further showed that CREB3L2 directly bound a proximal promoter/enhancer region of Chop and that this association was potentiated by Aβ₄₂ (FIG. 43F).

These observations raised the possibility that CREB3L2 and ATF4 act in the same Aβ₄₂-initiated signaling pathway. Indeed, co-immunoprecipitation studies with either in vitro translated or overexpressed tagged proteins revealed that CREB3L2 and ATF4 form stable heterodimers via their leucine zipper domains (FIGS. 35C and 35D), the canonical dimerization motif of bZIP TFs. Moreover, in axon-dendritic preparations, ATF4 readily co-immunoprecipitated with CREB3L2 in response to Aβ₄₂ stimulation, differently from vehicle-treated control neurites (FIG. 35E and FIG. 43G). We additionally visualized their interaction by proximity ligation assay (PLA) and found that Aβ₄₂ promoted increased signals for CREB3L2-ATF4 in axons and somas within 12 hours of treatment (FIGS. 35F and 35G). CREB3L2-ATF4 dimers were predominantly detected in the somatic compartment (−78% of all events), both inside and outside nuclei, of control and Aβ₄₂-treated neurons (FIGS. 35G, 35H and 43H). We note, however, that axon-derived CREB3L2-ATF4 likely contributes to this somatic pool in a non-negligible way, given that inhibition of dynein, the protein motor of axonal retrograde transport, led to a buildup of CREB3L2-ATF4 signals in axons treated with Aβ₄₂ (FIG. 35I). These analyses also revealed that axonal CREB3L2-ATF4 interactions receded to control levels within 18 hours of Aβ₄₂ exposure, suggesting that the surge in axonal CREB3L2-ATF4 signaling is temporally limited, primarily occurring in the first 12 hours of stimulation (FIG. 35I). In addition, we found that Aβ₄₂-promoted axonal CREB3L2-ATF4 heterodimerization is dependent on local protein synthesis, as treatment with emetine, an inhibitor of ribosome activity, in the last 60 minutes of a 12-hour Aβ₄₂ protocol limited their association in axons to control levels (FIG. 43I). Quantification of CREB3L2-ATF4 signals in a mouse model of Aβ₄₂ deposition (5×FAD; B6SJLF1/J background; 10-week-old animals) revealed a significant accumulation of the heterodimer in the hippocampal dentate gyrus, specifically in the molecular layer (ML) and inner polymorphic layer (IPL) in relation to age-matched controls (FIG. 36), providing in vivo evidence that CREB3L2-ATF4 heterodimerization is also increased by Aβ₄₂ in mouse brain. By contrast, a-synuclein fibrils, protein aggregates tied to the progression of Parkinson's disease (Volpicelli-Daley et al., 2011), produced no increments in CREB3L2-ATF4 heterodimerization when incubated with cultured hippocampal neurons for 10 days (FIGS. 43J and 43K), suggesting a certain level of specificity for Aβ₄₂ as a modulator of this transcriptional pathway.

Together, our findings identify CREB3L2 as a dimerization partner of ATF4 and show that Aβ₄₂, an early trigger of AD pathogenesis (Long and Holtzman, 2019), potentiates their heterodimerization.

### ChIPmera Resolves Genomic Binding Patterns of Dimeric TFs

While AD-associated gene expression changes have been characterized in detail (Zhang et al., 2013; Mostafavi et al., 2018; Bossers et al., 2010; Mathys et al., 2019), they occur via mostly unknown mechanisms. As an Aβ₄₂-regulated TF heterodimer, CREB3L2-ATF4 could contribute to these transcriptional responses, prompting us to investigate its DNA-binding program. However, despite recent methodological developments allowing for an unprecedented understanding of the binding specificities of TF dimers (Isakova et al., 2017; Jolma et al., 2015), these protocols are not tailored to their study in a cellular context and have a steep technical barrier to entry. Instead, we developed an approach that builds upon the well-established ChIP-sequencing (ChIP-seq) protocol. In our workaround, CREB3L2 and ATF4 were fused with FKBP/FRP domains, and specific homo- or heterodimers were promoted in HEK293 cells using chemically induced proximity (Stanton et al., 2018). In addition, each TF monomer was also tagged with a unique epitope (HA or V5) to facilitate its capture and purification of bound chromatin (FIG. 37A and FIGS. 44A-44D). We call this system ‘ChIPmera’ since it is based on a molecular chimera composed of two TFs.

Preliminary reporter assays with chemically induced CREB3L2-ATF4 heterodimers showed that their pairing makes up a functional unit capable of significantly driving Chop activation, as interference with CREB3L2 or ATF4 using dominant negative bZIP-like inhibitor peptides completely prevented CREB3L2-ATF4-induced reporter expression gains above baseline levels (FIG. 44E) (Sun et al., 2019; Gerdes et al., 2006). Our evaluation of control Renilla luciferase homodimers, equally promoted by chemically induced proximity, further revealed that the ChIPmera protocol produces nearly no background noise (FIG. 44F), averaging just 12 peaks across all replicates. In addition, maximal CREB3L2 homodimer signals were present within 15.1 base pairs (bp) of each other, a figure closely matched by coincident CREB3L2-ATF4 peaks (14.6 bp); ATF4 pairs were even more proximal, binding, on average, 11.3 bp apart (FIG. 44G). These results indicate that our methodology offers very good overall DNA-binding site resolution.

Unbiased motif searches using CentriMo revealed that CREB3L2-ATF4-bound DNA fragments were centrally enriched in either CREB3L2 or ATF4 canonical recognition sites (FIG. 37B and FIG. 44H) (Machanick and Bailey, 2011). These analyses also failed to detect novel motifs associated with CREB3L2-ATF4, suggesting that the heterodimer does not determine new DNA-binding specificities. In this regard, it is noteworthy that the CREB3L2 motif is analogous to the central tetramer of the cyclic AMP-response element (5′-TGACGTCA-3′) bound with high affinity by various members of the CREB/ATF family, including ATF4 (Hai et al., 1989), which likely explains the permissive binding complementarity demonstrated by both TFs (FIG. 37B and FIG. 44H). We further found that genomic regions enriched in CREB3L2-ATF4 were also targeted by CREB3L2-CREB3L2 and/or ATF4-ATF4 (FIGS. 37C-37E). Still, only a fraction of genes regulated by ATF4 homodimers were bound by the heterodimer (FIGS. 37C and 37F), while most CREB3L2-ATF4 hits were also part of the CREB3L2 homodimer dataset (FIGS. 37C and 37G). Indeed, globally, the CREB3L2-ATF4 heterodimer shared far more of its transcriptional signature with CREB3L2 than ATF4 (FIGS. 37F and 37G), a particularly interesting observation considering that homodimerization may be the default physiological (i.e., non-pathological) configuration of CREB3L2 (Vinson et al., 2002). Also informative was the genomic distribution of CREB3L2-ATF4: like CREB3L2 homodimers, the heterodimer predominated in the vicinity (±3 kb) of transcription start sites, accounting for 91.4% of all signals (FIG. 44I).

### Proteostasis and Trafficking are Targeted by CREB3L2-ATF4

Next, we performed a gene ontology (GO) enrichment analysis to explore the functional profile of CREB3L2-ATF4. Top statistically overrepresented GO terms among CREB3L2-ATF4-bound genes included RNA metabolism, protein translation and turnover, ER stress, mitochondrial organization, DNA repair, and intracellular vesicular trafficking (FIG. 37H). Overall, while the heterodimer combined biological functions individually associated with CREB3L2 and ATF4 (or both), the CREB3L2 program was the predominant factor determining its specificity. We additionally noted that a surprisingly high number of CREB3L2-ATF4 signals mapped within AD risk loci (Andrews et al., 2020). These included in their vicinity ABCA7, ADAM10, ADAMTS1, BCKDK, BIN1, CELF1, CSTF1, CD2AP, EED, FERMT2, HESX1, IQCK, KAT8, MEF2C, PICALM, PSMC3, OARD1, and ZCWPW1, many of which directly relate to AD susceptibility through their impact on the endocytic pathway and, particularly, APP homeostasis (FIG. 37C and FIG. 45A) (Scheltens et al., 2016).

Genes uniquely regulated by the CREB3L2-ATF4 heterodimer (i.e., not shared with the CREB3L2 homodimer; FIG. 37G) were associated with the unfolded protein and oxidative stress responses, the proteosome, and cell adhesion, among others (FIG. 45B). Up to 91.7% of these signals coincided with ChIP-seq ATF4 peaks previously characterized by the ENCODE Consortium, indicating that they represent specific components of the ATF4 program that CREB3L2-ATF4 integrates. Their genomic distribution was also more diversified than most other CREB3L2-ATF4 signals, with only 36.9% found within ±3 kb of a transcription start site (FIG. 45C). Interestingly, CREB3L2 and ATF4 were themselves strongly bound by the CREB3L2-ATF4 heterodimer (FIG. 45D), suggestive of an auto-regulatory mechanism.

Collectively, our analyses uncover the CREB3L2-enriched DNA-binding program of the CREB3L2-ATF4 heterodimer and reveal direct links with key pathways associated with cellular proteostasis and trafficking. They also serve as proof-of-concept for ChIPmera in its ability to identify genomic sites bound by specific TF dimers within their cellular environment.

### CREB3L2-ATF4 Orchestrates AD-Linked Transcription Network

To gain further insight into the pathological role of CREB3L2-ATF4, we performed RNA-sequencing (RNA-seq) on primary rat hippocampal neurons with increased dosage of CREB3L2-ATF4 using, as above, chemically induced proximity to promote their dimerization (FIG. 38A and FIGS. 46A-46E). Significant hits (P<0.05) were then compared against the AD transcriptome (FIG. 38B). Of 879 differentially expressed genes (DEGs) downstream of CREB3L2-ATF4 activation, 221 (approximately 25%; representation factor=2.0, P<2.2×10⁻²⁶, hypergeometric test) were identified as DNA-binding targets of the heterodimer in our ChIPmera study (FIG. 38B), indicating that CREB3L2-ATF4 has direct and indirect (i.e., downstream) transcriptional effects. These 221 genes were subsequently evaluated against a previously published late-onset AD transcriptome in which the dorsolateral prefrontal cortex was profiled at bulk-tissue level (Zhang et al., 2013), revealing a subgroup of 53 genes directly regulated by CREB3L2-ATF4 with disease-associated differential expression (significance cutoff defined as P<1×10⁻¹⁵; FIG. 38B and FIG. 47A). It included the tumor necrosis factor receptor TNFRSF1A, which contributes to AD pathogenesis by mediating neuronal cell death (Steeland et al., 2018), as the most significantly increased CREB3L2-ATF4 target gene (P=1.26×10⁻³⁵). In addition, 4 upregulated TFs—NFE2L2 (commonly known as NRF2; P=9.09×10⁻³⁰), SOX9 (P=2.24×10⁻²⁷), NFATC1 (P=4.74×10⁻²¹) and MXD4 (P=6.16×10⁻¹⁵)—were part of the AD-associated transcriptional program specifically mediated by CREB3L2-ATF4 (FIG. 38B and FIGS. 47A-47D) (Lambert et al., 2018). Except for MXD4, these TFs were not differently expressed in CREB3L2-CREB3L2 neurons (FIG. 46A), constituting a unique aspect of the CREB3L2-ATF4 program. Indeed, while the DNA-binding programs of CREB3L2-CREB3L2 and CREB3L2-ATF4 dimers overlap to a large degree (FIG. 37G), our RNA-seq analyses showed that their transcriptional responses diverged significantly both in terms of genes and biological processes affected by their activation (FIGS. 46B-46D), underscoring the distinct functional identity of CREB3L2-ATF4. It is also noteworthy that CHOP was one of the direct targets whose expression was significantly increased by CREB3L2-ATF4 in neurons (log fold-change=+0.31, P=0.04), corroborating our observations in Aβ₄₂-neurons (FIGS. 35A, 43E and 43F); however, its upregulation profile in AD did not meet our stringent significance cutoff and was not considered further in our analysis.

### CREB3L2-ATF4 Transcription Network is Functionally Tied to AD Pathophysiology

The finding that CREB3L2-ATF4 controls a transcription network activated in AD speaks to a broader role for the heterodimer in modulating disease-linked gene expression, prompting us to explore its regulatory and functional relationships. To this end, we combined ChIP-seq analyses from the ENCODE Consortium (the exception being SOX9, whose transcriptional program we mined from published literature) (Ohba et al., 2015) with the ChIPmera CREB3L2-ATF4 readout and contrasted these datasets against the AD transcriptome (top 3,000 DEGs; bulk tissue-level) as well as our own RNA-seq results using the Metascape platform (FIGS. 38C, 38D and 47E) (Zhou et al., 2019). Doing so allowed us to identify which co-regulated gene modules within this transcriptional circuit were altered in AD. Notably, processes related to mitochondria, apoptotic signaling, the endosome, autophagy, and synaptic transmission, each with well-described links to AD pathophysiology (De Strooper and Karran, 2016; Nixon, 2017), were enriched across the wider CREB3L2-ATF4 regulatory network (FIG. 38D). This analysis also highlighted important nuances within the network for a system-level interpretation of AD-linked gene misregulation. For example, while both mitochondrial and synaptic GO terms are downregulated AD-related functions, the former is co-regulated by all TFs in this network, whereas the latter is exclusively related to the NFATC1 program (FIG. 38D). Proteolysis, on the other hand, is targeted by the 4 ‘downstream’ TFs but not directly by CREB3L2-ATF4 (FIG. 38D), illustrating how the heterodimer can have meaningful effects beyond its core DNA-binding program. Strikingly, when considering all the TF-target gene relationships within this network, we found that 52.2% of DEGs in AD brain (top 3,000) were connected to at least one regulator (representation factor=1.4, P<2.63×10⁻⁶⁵ hypergeometric test; FIG. 38C).

To compensate for potential biases in cell composition associated with bulk AD brain transcriptomes, we extended these analyses using neuron-specific gene expression profiles obtained from dorsolateral prefrontal cortex single-nucleus RNA-seq (snRNA-seq) datasets (FIG. 48A) (Mathys et al., 2019). We observed strong links to AD transcriptional responses in excitatory neurons, with 62.8% of downregulated DEGs targeted by one or more regulators (representation factor=1.7, P<1.91×10⁻³, hypergeometric test; FIG. 48B). Upregulated DEGs also showed appreciable levels of overlap with CREB3L2-ATF4 and its wider network (43.5%; representation factor=1.2, P<0.031, hypergeometric test), but it is worth stressing that gene repression constitutes 74.7% of the expression signature of these cells in AD (n=565/756; FIG. 48B) (Mathys et al., 2019). CREB3L2-ATF4 alone directly interacts with 25.1% of all significantly altered genes in AD excitatory neurons (representation factor=2.1, P<1.57×10⁻²⁴, hypergeometric test). Among others, functional enrichment analyses highlighted processes related to intracellular transport in connection to the heterodimer's regulatory activity in these cells (FIGS. 48C and 48D), in line with our results in cultured neurons with induced CREB3L2-ATF4 dimerization (FIG. 38A and FIGS. 46A-46D). Differently, no enriched functional terms were uncovered in AD inhibitory neurons due to the low number of DEGs (n=51) identified in this subpopulation. In any case, we found an equally high, statistically significant degree of interaction between the CREB3L2-ATF4 network and downregulated DEGs in inhibitory cells (59.8%, representation factor=1.6, P<0.001, hypergeometric test), suggesting a role for CREB3L2-ATF4 in promoting gene repression in AD neurons.

Together, these analyses provide a data-driven, unbiased view of AD-relevant cellular dysfunctions to which the CREB3L2-ATF4 heterodimer potentially contributes via its transcriptional program.

### CREB3L2-ATF4 Activation Recapitulates AD Retromer Misregulation

Next, we sought to characterize how the heterodimer might influence specific AD gene expression responses. Among other functions, endosome-related processes were consistently enriched across our various datasets (FIGS. 37H, 46C and 46E), including when analyzed in the context of AD-associated transcriptional changes (FIGS. 38D, 48C and 48D). Interestingly, we encountered multiple direct links to the retromer, a master endosomal cargo-sorting complex whose dysfunction is implicated in the pathogenesis of AD and other neurodegenerative disorders (Cullen and Steinberg, 2018; Small and Petsko, 2015; Small et al., 2005). For example, the CREB3L2-ATF4 program includes various subunits of the retromer cargo-selective, tubulation, and membrane-recruiting modules, which CREB3L2-CREB3L2 mostly shares (FIGS. 39A and 39B). The retromer machinery additionally receives extensive inputs from the wider NRF2-SOX9-NFATC1-MXD4 network (FIGS. 39A and 39B), further hinting at a potentially important role for this pathway in mediating retromer gene expression mechanisms. Retromer dysfunction in AD results, at least partly, from the deficient expression of two core subunits, VPS26 and VPS35, in the brain of affected individuals (Small et al., 2005). Still, while the pro-amyloidogenic effects of a malfunctioning retromer are well-characterized (Small and Petsko, 2015; Small et al., 2005; Li et al., 2020; Wen et al., 2011), it remains unresolved why retromer transcriptional misregulation occurs in AD in the first place. This gap prompted us to examine more closely a potential connection between CREB3L2-ATF4 and retromer-mediated endosomal dysfunction.

First, by inspecting AD-associated retromer gene expression patterns in the dorsolateral prefrontal cortex (Zhang et al., 2013), we discovered that retromer misregulation was more extensive than previously recognized (FIGS. 39C and 39D), i.e., not restricted to VPS26 and VPS35 but evident across its different modules. These changes were particularly pervasive amongst subunits of the cargo selective and membrane-recruiting modules, which were downregulated in the diseased brain, and had VPS29 ranking as the most significantly altered retromer gene (log fold-change=−0.14, P=3.09×10⁻²⁴). Also, perturbed expression of brain-enriched VPS26B rather than the more ubiquitous VPS26A paralogue was observed (FIG. 39C) (Simoes et al., 2021). By contrast, sorting nexins SNX1 and SNX6, as well as EHD1, implicated in endosome membrane tubulation, were upregulated in AD (FIG. 39C). Strikingly, the transcriptional changes of several retromer subunits correlated strongly with CREB3L2 expression (Pearson r≥0.65), both positively and negatively (FIGS. 39C and 39D). These included VPS26B, VPS29, and VPS35 (negative co-expression association), core retromer components involved in the selection of cargoes (Cullen and Steinberg, 2018), as well as EHD1, SNX1, and SNX6 (positive association). Other brain regions affected by AD develop comparable retromer misregulation profiles (FIG. 49 and FIG. 50A) (Webster et al., 2009; Friedman et al., 2018), indicating that these effects are not unique to the prefrontal cortex. snRNA-seq datasets similarly ranked VPS29 as the most robust retromer DEG in AD excitatory neurons, followed, to a lesser extent, by SNX3, RAB7A, and VPS35 (FIG. 50B) (Mathys et al., 2019). Together, our analyses reveal that the retromer machinery in AD is impacted by widespread transcriptional alterations and suggest links between CREB3L2 and the breakdown of retromer regulatory mechanisms.

In line with this idea, CREB3L2 knockdown reduced the expression of various retromer subunits at both mRNA and protein levels in neurons (FIG. 39E and FIG. 51A). We further confirmed by ChIP-qPCR that CREB3L2 binds to DNA regulatory elements in the vicinity of several retromer genes in these cells (FIG. 39F), consistent with CREB3L2 functioning as a constitutive transcriptional activator for the neuronal retromer. Earlier, we had noted in our RNA-seq datasets that CREB3L2-ATF4 heterodimerization dysregulated various trafficking processes, including endosome-to-Golgi retrograde transport, an export pathway mediated by the retromer machinery (FIG. 46C) (Cullen and Steinberg, 2018). This observation suggested that CREB3L2's normal function might be impaired by its association with ATF4, potentially leading to a breakdown of retromer regulation. To test this, we measured retromer transcript and protein profiles in neurons using RT-qPCR and western blot following CREB3L2-ATF4 induction. These studies revealed a general trend towards the downregulation of the retromer machinery due to CREB3L2-ATF4 activation (mRNA average change=−12.9%; protein average change=−18.6%; FIGS. 39G and 39H). One prominent example was Vps35, retromer's ‘backbone’ subunit, which developed significantly reduced mRNA and protein levels (FIGS. 39G and 39H). CREB3L2-ATF4 heterodimers likewise led to the downregulation of Vps26b mRNA (FIG. 39G), while the effects on Vps29, Rab7a, and Snx3 expression were particularly evident at the protein level (FIG. 39H), suggesting that post-transcriptional mechanisms may also be at play. We detected a marked increase in Ehd1 expression levels only in CREB3L2-CREB3L2 neurons (FIGS. 39G and 39H), in agreement with the finding that CREB3L2 binds to this gene exclusively as a homodimer (FIG. 39A). Similarly, Vps26a, which is not a transcriptional target of either CREB3L2-CREB3L2 or CREB3L2-ATF4 (FIG. 39A), was unaffected in either background (FIGS. 39G and 39H), showing that the disruption of retromer regulatory processes by CREB3L2-ATF4 does not occur indiscriminately. CREB3L2-ATF4 activation was also seemingly detrimental to normal retromer function in neurons, as indicated by the increased degradation of cation-independent mannose 6-phosphate receptor (CI-M6PR; FIG. 51B), the canonical target of retromer-mediated endosome-to-Golgi retrieval (Arighi et al., 2004; Bennett et al., 2018).

Collectively, our findings reveal that the neuronal retromer is impacted by CREB3L2-ATF4 via a transcriptional response in many respects comparable to that seen in AD, suggesting a mechanism for its functional impairment. They additionally provide a concrete example of how the heterodimer can produce gene expression disruptions linked to relevant AD cellular dysfunctions.

CREB3L2-ATF4 Interacts with β-Amyloid and Tau Neuropathologies

Because CREB3L2-ATF4 is regulated by β-amyloid, an upstream component of the Alzheimer's pathological cascade (Long and Holtzman, 2019), we next asked how the heterodimer might globally relate to this neuropathology and other characteristic disease phenotypes. To this end, we leveraged gene-trait molecular networks previously elucidated by Mostafavi and colleagues linking AD-associated transcriptomic patterns to disease-relevant endpoints (e.g., β-amyloid burden or cognitive decline) and assessed their interaction with CREB3L2-ATF4 (Mostafavi et al., 2018). These data were originally derived from participants enrolled in the Religious Orders Study (ROS) or the Rush Memory and Aging Project (MAP), two large-scale longitudinal cohort studies of aging and dementia (Bennett et al., 2018). We first examined gene expression changes in the dorsolateral prefrontal cortex conditioned by β-amyloid and tau neuropathologies and looked for direct overlaps with the CREB3L2-ATF4 transcription network.

We subsequently used GO annotations to determine which biological processes within the CREB3L2-ATF4 network were most significantly enriched in connection to these traits (FIG. 40A and FIGS. 52A-52C). Doing so revealed that mitochondria and energy-related functions were negatively associated with β-amyloid burden (FIG. 40A and FIG. 52A), and that gene expression modulators, such as histone modification and chromatin organization, showed the strongest positive associations with tau pathology (FIG. 40A and FIG. 52B), overall indicating that CREB3L2-ATF4 heterodimerization may to some degree influence these well-known pathophysiological relationships (Klein et al., 2019; Nativio et al., 2020; Reddy and Beal, 2008). CREB3L2-ATF4-regulated gene modules associated with cognitive decline showed excellent agreement with the β-amyloid cohort (FIGS. 52A and 52C), similarly highlighting mitochondria-related functions amongst the strongest associations. Cellular trafficking terms were also significantly enriched in both datasets (FIGS. 52A and 52C), consistent with these pathways encompassing a key aspect of how CREB3L2-ATF4 contributes to AD pathophysiology.

Following up on these observations, we asked whether CREB3L2-ATF4 interacted with APP and tau regulation. We first measured soluble Aβ peptides in the medium of rat hippocampal neurons expressing CREB3L2-ATF4 heterodimers using a Meso Scale multiplex immunoassay. Compared to controls, CREB3L2-ATF4 neurons had significantly higher Aβ₄₂/Aβ₄₀ ratios (FIG. 40B), indicative of a shift in APP processing. This was accompanied by reduced Aβ₄₂ and Aβ₄₀ levels (FIG. 40B), with the decline of Aβ₄₀ being, however, more pronounced than that seen for the aggregation-prone Aβ₄₂ peptide (−71.9% versus −44.5%, respectively). These changes were not a result of a direct transcriptional effect of CREB3L2-ATF4 activation on App, as neither its mRNA nor protein expression were affected by the heterodimer (FIG. 40C and FIG. 52D). We also observed a drop in sAPPα levels (−19.3%; FIG. 52E), indicating that both the amyloidogenic and non-amyloidogenic APP processing pathways are impacted by the heterodimer, albeit at different degrees.

To assess tau metabolism, we initially focused on neuronal tau phosphorylation patterns, as hyperphosphorylated forms of this protein are associated with increased tau aggregation in AD (Wang and Mandelkow, 2016). Using antibodies that recognize specific tau phospho-epitopes, we found that the CREB3L2-ATF4 heterodimer led to significantly higher phosphorylation of various disease-relevant sites (FIGS. 40D, 52F and 52G). At Ser202/Thr205, analyzed with the same AT8 antibody employed in Braak staging (Alafuzoff et al., 2008; Braak et al., 2006), a ˜53.4% increase over control neurons was observed by western blot (FIG. 40D). Similarly, phosphorylation levels at Ser396/Ser404, which comprise one of the earliest AD-related abnormal tau processing events (Mondragón-Rodriguez et al., 2014), were 31.1% higher in CREB3L2-ATF4 neurons as detected with the PHF-1 antibody (FIG. 40D) (Otvos, Jr. et al., 1994; Greenberg et al., 1992). Comparable measurements (+23.7%) were obtained with a second antibody against phosphorylated Ser404 (T7444), confirming that this residue is affected by the heterodimer (FIG. 40D). We additionally encountered an upward (but statistically not significant) trend in tau phosphorylation levels with CREB3L2 homodimers (FIG. 40D and FIG. 52F), suggesting that CREB3L2 upregulation alone, as seen in the aftermath of CREB3L2-ATF4 activation (FIGS. 38A, 40C and 45C), may also to some degree modulate tau dyshomeostasis.

While tau is predominantly an intracellular protein, it is also known to be released by neurons and contribute to the spread of pathology (Wang and Mandelkow, 2016), prompting us to evaluate whether CREB3L2-ATF4 might similarly influence tau secretion. Using a Meso Scale platform, we found that extracellular tau accumulation in CREB3L2-ATF4 cultures was 62.9% above control levels (FIG. 40E). By contrast, this measure was unchanged in CREB3L2-CREB3L2 neurons (FIG. 40E). Further analyses using enzyme-linked immunosorbent assays (ELISAs) failed to detect Neurofilament-light (a biomarker of neurodegeneration) and Map2 (another member of the microtubule-associated protein family) in these samples across all conditions (FIG. 52H), indicating that tau secretion is actively promoted in CREB3L2-ATF4 neurons as opposed to its extracellular accumulation being a consequence of neuronal death.

How does CREB3L2-ATF4 activation influence tau metabolism? Analysis of neuronal RNA-seq datasets revealed that various subunits of the holoenzyme protein phosphatase 2A (PP2A) were disrupted at the transcriptional level by CREB3L2-ATF4 (FIG. 52I) (Wang and Mandelkow, 2016). PP2A accounts for ˜70% of tau-directed phosphatase activity in the human brain, and expression changes are proposed as a reason for its impairment in AD (Wang and Mandelkow, 2016). For example, we found that Ppp2ca mRNA, which encodes a catalytic subunit of PP2A downregulated in AD (log fold-change=−0.16, P=2.49×10⁻²⁵) (Zhang et al., 2013), was likewise reduced by CREB3L2-ATF4 activation in neurons (log fold-change=−0.15, P=0.016; FIG. 52I), raising the possibility that PP2A function is negatively affected by CREB3L2-ATF4. Indeed, PP2A phosphatase activity in purified extracts of CREB3L2-ATF4 neurons was significantly reduced in relation to controls (−10.1%; FIG. 40F), mechanistically in line with the altered tau phosphorylation patterns observed in these cells (FIG. 40D). However, globally, a STRING enrichment analysis using gene expression changes mediated by the heterodimer as input identified 17 additional DEGs with links to tau (P=1.14×10⁻⁷), including Clusterin, an important risk gene for late-onset AD implicated in tau aggregate seeding (FIG. 52J) (Andrews et al., 2020; Yuste-Checa et al., 2021). We cannot exclude, therefore, that other pathways may contribute to tau dyshomeostasis downstream of CREB3L2-ATF4.

Together, our findings reveal that CREB3L2-ATF4 drives abnormal tau phosphorylation and secretion in neurons, two key aspects linked to the development and spread of tau pathology, overall supporting a model whereby the heterodimer is regulated by and functionally interacts with AD neuropathologies (FIG. 40G).

### CREB3L2-ATF4 Heterodimers are Present in AD Brain

The analyses so far offer evidence of a potentially important role for CREB3L2-ATF4 in AD pathophysiology, which motivated us to pursue additional corroboration that their heterodimerization mediates a disease-relevant transcriptional mechanism. We first asked whether CREB3L2-ATF4 was found in the human brain and to what extent its heterodimerization levels were different in AD by performing co-immunoprecipitation in samples of non-demented control and disease cases (Table 7). Akin to our earlier bioinformatic analyses, we chose to evaluate tissue originating from the dorsolateral prefrontal cortex (Brodmann area 9), a cerebral region linked to higher cognitive functions affected by AD (Mathys et al., 2019). These studies revealed that proportionally higher levels of CREB3L2 were present in ATF4 co-immunoprecipitates from AD brains than those found in controls (131% average enrichment, P=0.013; FIG. 41A), particularly in individuals with advanced tau pathology (Braak stage V; FIG. 53A). This was accompanied by only marginal increases in CREB3L2/ATF4 ratios in AD input fractions (23%, P=0.34; FIG. 41A and FIG. 53B), denoting a specific and robust enrichment of CREB3L2 in ATF4 co-immunoprecipitates. The ATF4 immunoprecipitation reaction was highly efficient, as judged by the near absence of this protein in the flow-through fraction and the complete lack of any ATF4 accumulation in control IgG reactions (FIG. 41A and FIG. 53C). These findings suggest that an increased share of ATF4 is ‘occupied’ by CREB3L2 in AD brains, indicating that an overall shift in ATF4 and CREB3L2 dimerization patterns occurs in association with this neurodegenerative condition.

Employing PLA analyses in AD prefrontal cortex, we further found that CREB3L2-ATF4 heterodimers were present in neurons, predominantly inside the nucleus, but also in axons, as well as in other cells (FIG. 41B). In addition, comparisons against control cases showed that nuclear CREB3L2-ATF4 signals were enriched in AD neurons by approximately 20% (FIG. 53D; Table 8), an effect size that, while statistically significant, is likely attenuated by local differences in brain P-amyloid accumulation, which we did not account for in this assessment. These data reveal that neurons are a source of CREB3L2-ATF4 heterodimers in AD brain.

TABLE 7
Neuropathological evaluation (co-immunoprecipitation).

| | Sample | | | Cold | Frozen | | | |
|---|---|---|---|---|---|---|---|---|
| Classification | ID | Age | Sex | PMI^{a} | PMI^{b} | A* | B* | C* |
| Control | CTRL_1 | 54 | Female | 6:41 | 16:36 | A0 | B2 | Not |
| | | | | | | | | eligible |
| Control | CTRL_2 | 62 | Male | | 5:24 | A0 | B1 | Not |
| | | | | | | | | eligible |
| Control | CTRL_3 | 67 | Male | 11:10 | 15:10 | A0 | B1 | Not |
| | | | | | | | | eligible |
| Control | CTRL_4 | 89+ | Male | 4:47 | 11:17 | A2 | B2 | C1 |
| AD | ALZ_1 | 89+ | Male | | 11:26 | A2 | B3 | C2 |
| AD | ALZ_2 | 68 | Male | 1:00 | 13:30 | A2 | B3 | C3 |
| AD | ALZ_3 | 63 | Male | 3:10 | 12:10 | A2 | B3 | C3 |
| AD | ALZ_4 | 63 | Male | | 22:24 | A3 | B3 | C3 |
| AD | ALZ_5 | 89+ | Female | 4:30 | 6:20 | A1 | B2 | C1 |
| AD | ALZ_6 | 89+ | Male | 4:00 | 27:30 | A2 | B2 | C2 |

^{a}Cold PMI: Postmortem interval calculated from the reported time of death to the time the patient was brought into the cold room.
^{b}Frozen PMI: Postmortem interval calculated from the reported time of death to the mean time the brain was processed.
*Neuropathological classification follows ABC criteria.

Cases are listed in the same order used for loading co-immunoprecipitation gels in FIG. 41A.

TABLE 8
Neuropathological evaluation (proximity ligation assay).

| | Sample | | | Frozen | Clinical | Neuropathological | | | |
|---|---|---|---|---|---|---|---|---|---|
| Classification | ID | Age | Sex | PMI^{a} | diagnosis | diagnosis | A* | B* | C* |
| Control | CTRL_5 | 67 | Female | 20.7 | Mesenteric | No diagnostic | | | |
| | | | | | ischemia | abnormality | | | |
| Control | CTRL_6 | 53 | Female | 15.4 | Vasculopathy | Hemorrhagic | | | |
| | | | | | | infarct, | | | |
| | | | | | | remote/chronic, | | | |
| | | | | | | cerebellum | | | |
| Control | CTRL_7 | 38 | Female | 23.7 | Cirrhosis | Cerebellar | | | |
| | | | | | of the liver | vermian | | | |
| | | | | | (ethanol) | atrophy, severe | | | |
| PART | PART_1 | 68 | Male | 22 | Peritoneal | Primary age- | A | B | C |
| | | | | | bleeding | related | 0 | 1 | 0 |
| | | | | | | tauopathy | | | |
| AD | ALZ_7 | 83 | Female | 18 | Advanced | AD | A | B | C |
| | | | | | dementia | neuropathologic | 3 | 2 | 2 |
| | | | | | | changes, | | | |
| | | | | | | intermediate | | | |
| AD | ALZ_8 | 82 | Male | 36.7 | Alzheimer's | AD | A | B | C |
| | | | | | & COVID-19 | neuropathologic | 2 | 2 | 2 |
| | | | | | | changes, | | | |
| | | | | | | intermediate | | | |
| AD | ALZ_9 | 90 | Female | 8.9 | Alzheimer's | AD | A | B | C |
| | | | | | & COVID-19 | neuropathologic | 2 | 3 | 2 |
| | | | | | | changes, | | | |
| | | | | | | moderate | | | |
| AD | ALZ_10 | 74 | Male | 11.8 | Dementia | AD | A | B | C |
| | | | | | (Alzheimer's | neuropathologic | 3 | 3 | 3 |
| | | | | | type), | changes, | | | |
| | | | | | seizures, | severe | | | |
| | | | | | anxiety | | | | |

^{a}Frozen PMI (hours): Postmortem interval calculated from the reported time of death to the mean time the brain was processed.
*Neuropathological classification follows ABC criteria.

CRE1B33L2 Transcriptionally Overlaps with CREB3L2-ATF4 in AD

Next, we sought to substantiate our characterization of the CREB3L2-ATF4 transcriptional program with direct human evidence. Two individuals (both females with moderate AD pathology, aged >89 years; Table 9) were chosen for ChIP-seq analysis based on high prefrontal cortex CREB3L2 and ATF4 expression in addition to reduced post-mortem processing intervals. To our knowledge, ChIP-seq of point-source TFs in human brains has not been reported, likely due to the inherent technical challenges associated with biobanked material. While our efforts to immunoprecipitate ATF4-bound chromatin were unfruitful, we resolved 228 genomic sites enriched in CREB3L2, assignable to a set of 179 functionally coherent protein-coding genes (STRING network enrichment: P=1.22×10⁻⁷).

In agreement with our ChIPmera study, further breakdown of CREB3L2 binding showed that 83.1% of these signals were located within ±3 kb of a transcription start site (FIGS. 41C and 41D), validating our earlier observation that CREB3L2 preferentially engages with proximal promoter/enhancer regions (FIG. 37F). Likewise, GO terms related to intracellular trafficking, including endosomal transport, as well as ER stress, proteostasis, RNA metabolism, mitochondrial organization, and DNA repair were associated with CREB3L2 in AD (FIG. 41E and FIG. 53E). Notably, the retromer complex also came up as an enriched function (FIGS. 41F, 41G, 53E and 53F), confirming that this endocytic sorting pathway is a relevant regulatory target of CREB3L2 in AD brain. In addition, approximately half of the sites bound by CREB3L2 coincided with CREB3L2-ATF4-enriched regions (representation factor=4.1, P<1.1 ×10⁻⁴, hypergeometric test) and up to 78.8% were part of the CREB3L2 homodimer repertoire (representation factor=3.5, P<1.1×10⁻⁵⁸, hypergeometric test). Functional annotation of this overlapping gene subset revealed that trafficking categories, such as endosomal transport, as well as ER stress, mitochondrial organization, and proteostasis, were common to both CREB3L2-ATF4 and AD CREB3L2 datasets (FIG. 41H and FIG. 53G).

ChIPmera and the bioinformatic analyses that followed have thus captured a core set of disease-relevant pathways strikingly congruous with those regulated by CREB3L2 in AD brain. Although to some extent correlative, the findings support the pathophysiological significance of our proposed model regarding the nature of the CREB3L2-ATF4 program, especially since it is CREB3L2 that largely defines the regulatory landscape of the heterodimer (FIGS. 37F and 37G).

TABLE 9
Neuropathological evaluation (ChIP-seg).

| | | | | | | NIA- | | |
|---|---|---|---|---|---|---|---|---|
| | | | | | | Reagan | Braak | CERAD |
| | Sample | | | Cold | Frozen | consensus | NFT | Plaque |
| Classification | ID | Age | Sex | PMI^{a} | PMI^{b} | criteria | Stage | Score |
| AD | ALZ_11 | 89+ | Female | 0:45 | 16:30 | High | V | C |
| AD | ALZ_12 | 89+ | Female | 1:25 | 20:10 | Intermediaet | V | A |

^{a}Cold PMI: Postmortem interval calculated from the reported time of death to the time nthe patient was brought into the cold room.
^{b}Frozen PMI: Postmortem interval calculated from the reported time of death to the mean time the brain was processed.

### Gene Expression as a Driver and Intervention Target in Aβ₄₂ Neurodegeneration

Because CREB3L2-ATF4 interacts with a substantial subset of the AD transcriptome and recapitulates aspects of disease progression, disrupting its activity may mitigate the detrimental effects mediated by Aβ₄₂ and hence potentially improve disease outcomes. Targeted interference with bZIP TFs is well-established and has found applicability in cancer models (Sun et al., 2019; Gerdes et al., 2006). Briefly, bZIPs homo- and/or heterodimerize by forming a parallel coiled-coil (the ‘leucine zipper’) and bind DNA via a proximal region rich in basic (i.e., positively charged) amino acids (Amoutzias et al., 2008; Reiter et al., 2017; Vinson et al., 2002). Replacing the latter with an acidic (i.e., negatively charged) sequence creates very efficient dominant-negative bZIP sponges (aZIPs), which simultaneously prevent dimerization and DNA binding of target TFs. Based on this principle, we designed CREB3L2 and ATF4 aZIPs and tested their ability to rescue Aβ₄₂-induced neuronal cell death after viral delivery. Remarkably, both peptides significantly improved cell viability in neurons exposed to Aβ₄₂ across a 48-hour stimulation protocol in relation wild-type neurons (FIG. 42A). These findings suggest that CREB3L2 and ATF4 are central effectors of Aβ₄₂ neurodegeneration.

While TFs have traditionally been considered poor therapeutic targets (Su and Henley, 2021), gene expression is emerging as a powerful platform for drug discovery and repurposing efforts (Dönerta§ et al., 2018; Williams et al., 2019). This is made possible by resources like The Connectivity Map (CMap; Lamb et al., 2006), which has characterized more than one million gene expression signatures for a range of drugs and other perturbations. With CMap, changes in gene expression linked to a disease process can be compared for similarity to drug-induced perturbations and those with the most negative correlations followed up as therapeutic leads. A query of the CMap database using the CREB3L2-ATF4 transcriptome that included both upregulated and downregulated genes (top 150 DEGs, 75 from each arm) identified dovitinib, a pan-receptor tyrosine kinase inhibitor (Trudel et al., 2005), as the most significant hit (connectivity score=−0.65; −log₁₀[false discovery rate]=15.65). Interestingly, this molecule had been previously classified as a top repurposing candidate for AD based on two independent analyses (Rodriguez et al., 2021; Issa et al., 2016), prompting us to test it in neurons challenged with A342. Using RNA-seq, we found 203 DEGs (P<0.05) after a 24-hour Aβ₄₂ stimulation protocol (average absolute log fold-change=0.29), 131 of which showed corrective shifts 0.10 with dovitinib co-treatment, and a subset of 30 genes surpassing 0.25 differences in head-to-head comparisons (FIG. 42B). Vgf was the most significantly altered gene by Aβ₄₂ (log fold-change [A342]=0.57), also undergoing the largest overall correction towards control baseline levels with dovitinib (log fold-change [A342+dovitinib]=0.07) (FIG. 42B). The results indicate that dovitinib can mitigate, at least to some extent, the early-phase transcriptional response downstream of A342. Encouragingly, this drug can cross the blood-brain barrier and has a well-characterized safety profile (Schafer et al., 2016), in addition to being non-toxic to neurons (Rodriguez et al., 2021), which bodes well for future preclinical work.

Altogether, in this study, we have discovered a TF heterodimer regulated by β-amyloid that interacts with a molecular network linked to disease phenotypes and have confirmed that key components of this transcriptional pathway are present in the AD brain. We additionally provide evidence that gene expression may be a promising Intervention Target for AD Therapies.

### DISCUSSION

Our findings collectively support the conclusion that CREB3L2 and ATF4 form a pathologically important association in AD and highlight TF combinatorial relationships as a relevant disease mechanism. As TF interactions are widespread phenomena (Amoutzias et al., 2008; Reiter et al., 2017), significant pathophysiological insights are likely within reach of future investigations. ChIPmera, a methodology we developed to interrogate dimeric TFs, could prove particularly useful in this regard, especially when coupled with much-needed improved strategies for detecting context-dependent TF associations. Indeed, CREB3L2 and ATF4 have no known genetic variants associated with AD risk and were likely to have been disregarded as candidates of study had we focused uniquely on their expression profiles, given that they would not be considered obvious top picks by this measure alone (Zhang et al., 2013; Andrews et al., 2020). Instead, it is their heterodimerization that makes them remarkable in the context of AD.

Despite not encompassing the full complexity of AD, the amyloid cascade hypothesis remains the predominant model of pathogenesis (Musiek et al., 2015) (Long and Holtzman, 2019; Musiek and Holtzman, 2015). It postulates that β-amyloid deposition is a key instigator of the ensuing degenerative process involving tau aggregation, neuron loss, and cognitive impairment (Musiek and Holtzman, 2015). Elucidating the mechanisms by which β-amyloid precipitates this chain of events is of critical significance (Musiek and Bennett, 2021), not least because current evidence strongly suggests that tau pathology is the primary mediator of neurodegeneration in AD (Long and Holtzman, 2019). Remarkably, CREB3L2-ATF4 bridges both hallmark AD neuropathologies, being regulated by β-amyloid and promoting aspects of tau dyshomeostasis typical of AD. Our findings thus indicate that gene expression changes are not merely responsive to but function as actual drivers of AD pathology. Given the scope of the heterodimer's transcriptional program, it is doubtful that its effects can be ascribed to a single dysfunctional pathway (e.g., retromer or PP2A) but more likely emerge from globally interdependent disturbances spanning different cellular processes. This is ultimately why AD and other conditions with multifactorial etiologies may be ideally suited for therapeutic interventions focusing on gene expression (Rodriguez et al., 2021), as these open the possibility of correcting cellular imbalances even when the underlying pathogenic mechanisms are not entirely understood.

We recognize that some inferences we make in this study regarding CREB3L2-ATF4 are based on correlation analyses. We have accommodated this limitation by using human datasets from different sources and favoring unbiased readouts whenever possible. Ultimately, the identification of dovitinib attests to the strength of the approach. Indeed, we and others have closed in on the same molecule using different strategies and datasets (Rodriguez et al., 2021; Issa et al., 2016), which provides a strong indication that CREB3L2-ATF4 captures a core gene expression signature of AD. Focused development of a small molecule that specifically inhibits their heterodimerization will be needed to allow more direct validations and fundamentally prove the role of CREB3L2-ATF4.

In conclusion, we report that TF heterodimerization can encode pathogenic stimuli and reconfigure transcription networks associated with disease processes. Our study provides a novel mechanistic perspective for understanding gene expression programs in the context of AD and suggests a transcriptional link between P-amyloid and tau pathologies, the two hallmark brain lesions that characterize this neurodegenerative condition.

### Materials and Methods

### Neuronal Culture

Same as that described above in Example 1.

### Aβ₄₂ Peptide Oligomerization and Treatment

Same as that described above in Example 1.

Axonal siRNA Transfection

Same as that described above in Example 1.

### Cell Death Assay (TUNEL)

Same as that described above in Example 1.

### CHOP Immunocytochemistry and Image Analysis

Same as that described above in Example 1.

### Chromatin Immunoprecipitation and Quantitative PCR (ChIP-qPCR)

Same as that described above in Example 1.

### In Vitro Translation and CREB3L2-ATF4 Co-Immunoprecipitation

Same as that described above in Example 1 under “Rabbit reticulocyte lysate translation system and immunoprecipitation.”

### Western Blot Analyses

Same as that described above in Example 1.

### Proximity Ligation Assay on Primary Neuronal Cultures

Same as that described above in Example 1.

### Transwell Neuronal Culture and Co-Immunoprecipitation

Same as that described above in Example 1.

### Analysis of AD-Associated and Related Transcriptional Profiles

Same as that described above in Example 1 under “Analysis of LOAD-associated and related transcriptional profiles”.

### CREB3L2-ATF4 Co-Immunoprecipitation in Human Brain Tissue

Protein A magnetic beads (#S1425S, New England Biolabs) were washed in PBS containing 0.1% BSA and incubated at 4° C. for 1 hour with rotation. Following two rinses with PBS, beads were resuspended in lysis buffer, mixed for 4 hours with anti-ATF4 antibody (1 μg per immunoprecipitation; ab184909, Abcam), and washed three times with lysis buffer. At this point, we proceeded by covalently cross-linking the immobilized antibodies to Protein A beads using bis(sulfosuccinimidyl)suberate (BS3; #21586, Thermo Fisher) following manufacturer's guidelines. Frozen dorsolateral prefrontal cortex tissue (approximately 80 mg per IP; Table 7) was processed in ice-cold lysis buffer (20 mM Tris-Cl pH 8, 137 mM NaCl, 1% Nonidet P-40 (NP-40), 2 mM EDTA, supplemented with protease and phosphatase inhibitors (cOmplete cocktail tablets, Roche). Sample volumes were weight-adjusted in a sample-by-sample manner and tissue extracts were incubated for 2 hours at 4° C. with end-over-end rotation. During this incubation, a 10-minute bath sonication step was performed to improve extraction efficiency. After centrifugation at 12,000 rpm and 4° C., pellets were discarded and supernatants transferred to new tubes. Equal amounts of antibody-bead conjugates were mixed with lysates overnight at 4° C. with constant rotation and washed a total of four times with ice-cold lysis buffer. Immunoprecipitation input and the supernatant resulting from the first wash step (‘flow-through’ fraction) were saved for further analyses. Immunoprecipitates were eluted in 50 μl of 0.2 M glycine buffer (pH 2.5) and allowed to react for 5 minutes at 4° C. with rotation after a short vortexing step. Eluates were transferred to a new tube, and the elution protocol repeated. Pooled eluates were neutralized by the addition of 20 μl of 1 M Tris-Cl (pH 9.0), and Laemmli buffer-treated samples heated at 80° C. for 5 minutes. CREB3L2 signals were visualized using anti-CREB3L2 serum (HPA015068, Atlas Antibodies) and a light chain-specific monoclonal secondary antibody (211-032-171, Jackson ImmunoResearch); successful ATF4 immunoprecipitation was confirmed using anti-ATF4 sera (ab184909, Abcam, and #11815, Cell Signaling Technology).

### 5×FAD Transgenic Animals—Care and Proximity Ligation Assay

All experiments were reviewed and monitored by the IACUC at Columbia University in accordance with NIH guidelines for the humane treatment of animals. Hemizygous 5-weeks-old B6SJL-Tg(APPSwFILon,PSEN1*M146L*L286V)6799Vas/Mmjax (5×FAD) mice (MMRRC Stock No: 34840-JAX) were purchased from Jackson Laboratories. This strain overexpresses both mutant human APP with the Swedish (K670N, M671L), Florida (1716V), and London (V7171) Familial Alzheimer's Disease (FAD) mutations and human presenilin 1 (PS1) harboring two FAD mutations, M146L and L286V. Age- and sex-matched wild-type B6SJLF1/J mice (Stock No: 100012) were acquired from Jackson Laboratories at the same time. Both groups were maintained in our breeding colony until 10 weeks of age. Mice were euthanized following ketamine (80-100 mg/kg) and xylazine (5-10 mg/kg) administration, perfused with normal saline (Mckesson, #37-6280), and fixed with 4% paraformaldehyde (vol/vol). Brains were post-fixed overnight in 4% paraformaldehyde, washed in PBS, transferred to 30% sucrose, and finally embedded for cryostat sectioning (12-μm thick coronal cuts). Epitope unmasking was done for 20 minutes in steaming 0.01 M sodium citrate buffer (0.05% Tween 20, pH 6.0), which was followed by three 10-minute PBS-T washes. A standard prerequisite of the PLA protocol in its kit format is the availability of specific primary antibodies raised in different hosts. As we were unable to locate a compatible pair of CREB3L2 and ATF4 antibodies, we resorted to using the Duolink Probemaker kits (DU092009 and DU092010, Sigma) to directly conjugate two rabbit-raised antibodies, anti-CREB3L2 (HPA015068, Atlas Antibodies) and anti-ATF4 (ab184909, Abcam), with PLA PLUS and MINUS oligonucleotides. This approach requires that both antibodies are solubilized in a carrier- and preservative-free buffer; to achieve this, we dialyzed the antibodies by employing a Slide-A-Lyzer device with a 10K molecular weight cutoff (#69570, Thermo Scientific) made to float on a glass beaker containing 200 ml of PBS for 2 hours. The whole protocol was performed inside a cold room to minimize degradation and, in the case of the anti-CREB3L2 antibody, was followed by a concentration step (#88513, Thermo Scientific). The conjugation reaction was performed overnight at room temperature, and CREB3L2-ATF4 heterodimers stained using Duolink In situ Far-red Detection reagents (DU092013; Millipore Sigma). As per manufacturer's instructions, the PLA Probe Diluent included in the Probemaker kit was used in substitution of the PLA Antibody Diluent in the PLA protocol. Tissue sections were preserved in Duolink In situ Mounting Medium with DAPI (DU082040, Millipore Sigma). Images were acquired on a LSM800 confocal microscope (Zeiss) with a Plan-Apochromat 40×/1.3 Oil DIC M27 oil objective (Zeiss). Imaging settings were kept constant between conditions. PLA interactions were unbiasedly analyzed in Fiji using the ‘Analyze Particles’ function after auto-thresholding (‘Yen’ method). We excluded one 5×FAD animal due to technical difficulties in the tissue preparation phase; sample size was determined by power analysis.

### Chemically Induced Proximity: Reagent Preparation

Same as that described above in Example 11.

### ChIPmera—Chromatin Immunoprecipitation and Data Analysis

Same as that described above in Example 1.

shRNA Preparation and Delivery

Same as that described above in Example 1.

### RNA Extraction and Quantitative Real-Time PCR

Same as that described above in Example 1.

### RNA-Sequencing

Same as that described above in Example 11.

Integration of Wider CREB3L2-ATF4 Transcription Network with AD Profiles

Overlapping hits among CREB3L2-ATF4 datasets were found using the Venn diagram module accessible at http://genevenn.sourceforge.net/. Processed ChIP-seq files (identifiers: ENCFF794DLT [NFE2L2], ENCFF516MEQ [NFATC1], ENCFF353RDB [MXD4]), prepared and analyzed by the ENCODE Consortium, were retrieved from https://www.encodeproject.org/. The DNA-binding program of SOX9 was mined from published literature and only ‘Class I’ sites were considered, catalogued as such by Ohba et al. based on their clustering around transcription start sites (Ohba et al., 2015). Gene annotations of ranked peaks were assigned by GREAT v4.0.4 using a ±3 kb transcription start site cutoff. For each dataset, only the top (i.e., strongest) 3000 hits within this cutoff were carried forward to accommodate the requirements of the Metascape analysis pipeline (Zhou et al., 2019), which was used to produce the comparative gene ontology meta-analysis. AD-associated gene expression changes were obtained from publicly available datasets (Zhang et al., 2013; Mathys et al., 2019). Top 3,000 differentially expressed genes in AD prefrontal cortex (bulk RNA-seq dataset), excluding conflicting entries, were grouped according to expression profile, totaling 1267 upregulated and 1692 downregulated genes (lowest adjusted P-value=9.93×10⁻²⁰).

### Neuronal Culture Supernatant Collection and Measurements

Culture supernatants were transferred to 15-ml falcon tubes, spun at 2000 g and 4° C. for 5 minutes, aliquoted, and stored at −80° C. A sandwich immunoassay (V-PLEX Aβ Peptide Panel 1 kit, #4G8, Meso Scale Discovery) was employed in the measurement of β-amyloid species. Manufacturer's guidelines were followed thoroughly during plate preparation, and samples diluted 1:1 with Diluent 35 (provided as part of the kit) to avoid matrix saturation. All biological replicates were measured in parallel. Signal readings were performed on a Sector Imager 2400 instrument (Meso Scale Discovery). For assessing sAPPα levels in culture supernatants, we utilized a sandwich ELISA assay (sAPPα [Mouse/Rat] [highly sensitive], #27419, Immuno-Biological Laboratories) and samples were diluted 10-fold. Extracellular tau levels were assessed using the Phospho(Thr231)/Total Tau Kit from Meso Scale Discovery following the protocol provided by the manufacturer. Neurofilament-light and Map2 were measured by ELISA using PathScan Total Neurofilament-L Sandwich ELISA kit (#99175, Cell Signaling Technology) and Abcam's SimpleStep MAP2 ELISA kit (ab253229), respectively.

### Analysis of Tau Phosphorylation

DIV1 rat hippocampal neurons were infected with lentiviruses carrying FRB/FKBP-tagged ATF4 or CREB3L2 transgenes. One day later, the heterodimerizer was added to cultures diluted in Neurobasal growth medium at 100 nM, and cells allowed to mature until DIV10. Additional rounds of heterodimerizer supplementation (100 nM, diluted in growth medium) were made every other day. On DIV10, cells were washed in ice-cold HBSS and lysed in 2×Laemmli buffer (130 mM Tris-Cl pH 6.8, 0.1 mM dithiothreitol, 20% (v/v) glycerol and 4% sodium dodecyl sulfate diluted in water) by scrapping. Fresh lysates were boiled at 85° C. for 5 minutes and immediately analyzed by western blot. Antibodies: anti-Tau (1:1,000; Clone HT7, MN1000, Invitrogen), anti-phospho-Tau (pSer²⁰²/pThr²⁰⁵; 1:4,000; AT8, Invitrogen), anti-phospho-Tau (pSer³⁹⁶/pSer⁴⁰⁴; PHF-1, formerly available through Dr. Peter Davies), anti-phospho-Tau (pSer⁴⁰⁴; 1:1,1000; T7444, Millipore Sigma).

### PP2A Activity Assay

Hippocampal neurons were initially lysed in accordance with the instructions provided in Serine/Threonine Phosphatase Assay Kit (V2460; Promega). Lysates were then centrifuged at 1×10⁵ g at 4° C. for 1 hour in phosphatase storage buffer (2 mM EGTA, 5 mM EDTA, 0.5 mM PMSF, 150 mM NaCl, 1% Triton X-100, 50 mM Tris-HCl pH 7.4, and 0.5% protease inhibitor cocktail). Sephadex G-25 spin columns were used to remove free phosphate found endogenously, followed by incubation for 1 h at 37° C. in PP2A reaction buffer (250 mM imidazole pH 7.2, 1 mM EGTA, 0.1% P-mercaptoethanol, 0.5 mg/ml BSA) supplemented with Ser/Thr phosphopeptide. The reaction was stopped by adding 50 μl of molybdate dye/additive mixture. After 30 minutes, and absorbance was measured at 600 nm in a 96-well microplate reader (Tecan). PP2A activity measurements were normalized to total DNA content in biological replicates using a CyQUANT assay (Thermo Fisher). Protein phosphatases 2B and 2C (PP2B and PP2C) show very low to no detectable activity in the presence of EGTA (PP2B) and EDTA (PP2C); it is also noteworthy that the phosphopeptide utilized in this assay is a poor substrate for protein phosphatase 1.

### Human Brain Sample Procurement

Post-mortem human material was obtained through the New York Brain Bank at Columbia University and the Neuropathology Brain Bank at Mount Sinai according to institutional guidelines governed by approved protocols. Neuropathological evaluations (Table 7, Table 8, and Table 9) included assignment of CERAD, Braak, NIA-Reagan, or ABC scores. Dorsolateral prefrontal cortex tissue specimens were derived from Brodmann area 9.

### CREB3L2-ATF4 Co-Immunoprecipitation in Human Brain Tissue

Protein A magnetic beads (#S1425S, New England Biolabs) were washed in PBS containing 0.1% BSA and incubated at 4° C. for 1 hour with rotation. Following two rinses with PBS, beads were resuspended in lysis buffer, mixed for 4 hours with anti-ATF4 antibody (1 μg per immunoprecipitation; ab184909, Abcam), and washed three times with lysis buffer. At this point, we proceeded by covalently cross-linking the immobilized antibodies to Protein A beads using bis(sulfosuccinimidyl)suberate (BS3; #21586, Thermo Fisher) following manufacturer's guidelines. Frozen dorsolateral prefrontal cortex tissue (approximately 80 mg per IP; Table 7) was processed in ice-cold lysis buffer (20 mM Tris-Cl pH 8, 137 mM NaCl, 1% Nonidet P-40 (NP-40), 2 mM EDTA, supplemented with protease and phosphatase inhibitors (cOmplete cocktail tablets, Roche). Sample volumes were weight-adjusted in a sample-by-sample manner and tissue extracts were incubated for 2 hours at 4° C. with end-over-end rotation. During this incubation, a 10-minute bath sonication step was performed to improve extraction efficiency. After centrifugation at 12,000 rpm and 4° C., pellets were discarded and supernatants transferred to new tubes. Equal amounts of antibody-bead conjugates were mixed with lysates overnight at 4° C. with constant rotation and washed a total of four times with ice-cold lysis buffer. Immunoprecipitation input and the supernatant resulting from the first wash step (‘flow-through’ fraction) were saved for further analyses. Immunoprecipitates were eluted in 50 μl of 0.2 M glycine buffer (pH 2.5) and allowed to react for 5 minutes at 4° C. with rotation after a short vortexing step. Eluates were transferred to a new tube, and the elution protocol repeated. Pooled eluates were neutralized by the addition of 20 μl of 1 M Tris-Cl (pH 9.0), and Laemmli buffer-treated samples heated at 80° C. for 5 minutes. CREB3L2 signals were visualized using anti-CREB3L2 serum (HPA015068, Atlas Antibodies) and a light chain-specific monoclonal secondary antibody (211-032-171, Jackson ImmunoResearch); successful ATF4 immunoprecipitation was confirmed using anti-ATF4 sera (ab184909, Abcam, and #11815, Cell Signaling Technology).

### Proximity Ligation Assay in AD Prefrontal Cortex

CREB3L2-ATF4 heterodimers were visualized using Duolink In situ Brightfield Detection reagents (DU092012; Millipore Sigma). CREB3L2 and ATF4 PLA probes were prepared as described for the detection of CREB3L2-ATF4 heterodimers in 5×FAD mice. Per manufacturer's instructions, the PLA Probe Diluent included in the Probemaker kit was used in substitution of the PLA Antibody Diluent in the PLA protocol. Before deparaffinization with xylene, slides were placed in a 60° C. oven for 1 hour; we proceeded by rehydrating slides using a graded ethanol series (100%>95%>70%>50%>water), plus two 10-minute PBS-T (0.1% Tween 20) washes. Epitope unmasking was done for 20 minutes in steaming Tris-EDTA buffer (10 mM Tris base, 1 mM EDTA, 0.05% Tween 20, pH 9.0), followed by three 5-minute PBS-T rinses. We quenched endogenous peroxidases slides with 1% hydrogen peroxide for 30 minutes before blocking. Co-staining of neurofilament (1:400; heavy chain subunit; #N0142, Millipore Sigma) was performed afterwards using the Vector Blue Alkaline Phosphatase substrate kit (SK-5300, Vector Laboratories). To increase detection sensitivity, we additionally employed the Vectastain ABC-AP system (AK-5002, Vector Laboratories) before signal development. Finally, sections were dehydrated in a graded ethanol series (50%>70%>95%>100%), cleared with Histo-Clear (64110-01, Electron Microscopy Sciences), mounted in Vectamount (H-5000, Vector Laboratories), and airdried for 24 hours before proceeding with imaging. Human dorsolateral prefrontal cortex specimens (Brodmann area 8/9; Table 8) were manually counted by an experimenter ‘blind’ to the underlying diagnosis. Technical controls: PLA Probe Rabbit IgG Isotype Control MINUS (DU087004; Millipore Sigma) and CREB3L2 blocking peptide (APrEST73339; Atlas Antibodies). For each case, CREB3L2-ATF4 measurements were interspersed between 5 randomly selected tissue sub-regions; specifically, 10 neurons within layers III-V were analyzed in each sub-region, for a total of 50 independent measurements per brain.

### Preparation of α-Synuclein Pre-Formed Fibrils

Recombinant human α-synuclein monomers (#RP-003, Porteus) were used to generate pre-formed fibrils as previously described (Volpicelli-Daley et al., 2011). Briefly, monomers were diluted to a concentration of 5 mg/mL in PBS and shaken at 1000 rpm in an Eppendorf Thermomixer C for 7 days at 37° C. to generate fibrils. Fibrils were aliquoted and stored at −80° C. Prior to treatment of hippocampal neurons, aliquots were thawed at room temperature, diluted to a concentration of 0.1 μg/μL in PBS, and sonicated in a QSonica 700 sonicator with cup horn at 30% amplitude for a total of 22.5 minutes (3 seconds on, 2 seconds off). Sonicated fibrils were added to hippocampal neurons on DIV 4 at a concentration of 5 mg/mL for 10 days. Intraneuronal pathology was confirmed by staining against phospho-α-synuclein (pSer¹²⁹; 1:250; #23706, Cell Signaling Technology).

### CI-M6PR Cycloheximide Chase

Control and CREB3L2-ATF4-expressing rat hippocampal neurons, supplemented every 2 days with 100 nM heterodimerizer, were grown until DIV10 before treatment with cycloheximide (40 μg/ml, #C4859, Millipore Sigma) for 2-6 hours, as previously described (Arighi et al., 2004). Cells were lysed in 2×Laemmli buffer (130 mM Tris-Cl pH 6.8, 0.1 mM dithiothreitol, 20% (v/v) glycerol and 4% sodium dodecyl sulfate diluted in water) by scraping, boiled at 85° C. for 5 minutes, and analyzed by western blot using an antibody against CI-M6PR (1:30,000; ab124767, Abcam).

### CREB3L2 ChIP-Sequencing in AD Prefrontal Corte

Autopsy cases #ALZ_11 and #ALZ_12 (Table 9), both females with moderate AD pathology, were chosen for ChIP-seq analysis based on 1) high CREB3L2 and ATF4 expression level, 2) CREB3L2-ATF4 complex accumulation and 3) reduced postmortem processing intervals. Frozen minced brain tissue (approximately 150 mg per immunoprecipitation and a total of 300 mg per case) was transferred to a conical tube containing 6 ml of PBS supplemented with protease inhibitors (PBS+PI), and protein-DNA cross-linking allowed to develop for 20 minutes at room temperature using formaldehyde at a final concentration of 1.5% (v/v). Cross-linking reaction was then quenched with glycine (5 minutes at room temperature), as per manufacturer's instructions (SimpleChIP Plus Kit [#9005], Cell Signaling Technology). After rinsing in ice-cold PBS+PI, we proceeded by disaggregating tissue using an ice-cold Dounce homogenizer (7 ml total capacity) until a single-cell suspension was obtained, which was followed by a 2,000 g and 4° C. centrifugation step. The resulting supernatants were discarded. Chromatin fragments (mainly 1-3 nucleosomes in size) were obtained by partial digestion with micrococcal nuclease (MNase; 2 μl in 500 μl) incubated for 13 minutes at 37° C. in a Thermomixer R (Eppendorf) programmed for frequent mix cycles. Nuclear membranes were broken up by three rounds of 20-second, 15% amplitude pulses using a Sonic Dismembrator Model 500 (Fisher Scientific), and lysates subsequently clarified by centrifugation. Adequate digestion was confirmed by agarose gel electrophoresis. Approximately 6 μg of chromatin, diluted in 400 μl of ChIP buffer, was used per immunoprecipitation; CREB3L2-bound DNA was immunoprecipitated by overnight incubation at 4° C. with anti-CREB3L2 serum (2 μg; HPA015068, Atlas Antibodies). Immunoprecipitates were captured using Protein G magnetic beads and washed with low- and high-salt buffers, as directed. Elution was performed at 65° C. and 1,200 rpm for 30 minutes using a thermomixer, protein-DNA cross-links reversed by treatment with Proteinase K for 2 hours at 65° C., and DNA purification achieved by using a column-based system. ChIP-seq library preparation and sequencing reactions were conducted at GENEWIZ, Inc. (South Plainfield, NJ, USA). During library preparation, immunoprecipitated samples were normalized to input DNA, i.e., chromatin cross-linked and fragmented side by side with immunoprecipitated DNA using the same conditions. The sequencing libraries were multiplexed and clustered on one lane of a flowcell. Sequencing was performed using a 2×150 Paired-End (PE) configuration. Image analysis and base calling were conducted by the HiSeq Control Software (HCS). Raw sequence data generated from Illumina HiSeq were converted into fastq files and de-multiplexed using Illumina's bcl2fastq v2.17 software. One mismatch was allowed for index sequence identification. ChIP-seq sequencing data were processed and analyzed within the Galaxy web platform, using the public server at usegalaxy.org. First, we run FastQC to evaluate overall sequencing quality (unique reads >90%). Second, library adapters and low-quality reads were removed using Trimmomatic v0.36. Third, reads were mapped to the hg38 reference genome with Bowtie v0.12.7, and non-uniquely mapped reads filtered out. Forth, unmapped and low quality (MAPQ <20) reads were excluded with samtools v1.2. Fifth, peak calling was performed with MACS2 v2.1.1 with minimum false discovery rate (FDR) cutoff for peak detection fixed at 0.05, lower and upper mfold bounds defined as 5 and 50, respectively, and extension size set at 144. Finally, peaks were exported to the UCSC genome browser for visualization after conversion to bigwig format. CREB3L2 gene ontology (GO) term enrichment was analyzed using ClueGO v2.5.4 within the Cytoscape platform (v3.6.1).

CREB3L2 and ATF4 aZIPs: Reagent Preparation and Neuronal Viability Analysis

Specific aZIPs sequences were synthesized by Genewiz. CREB3L2 aZIP: 5′-GGATCCGCCACCATGGACTACAAAGATGATGACGACAAGCACATGGCCAGCATGA CCGGGGGCCAGCAGATGGGAAGAGACCCTGATTTGGAACAAAGGGCAGAGGAGC TGGCCCGGGAGAACGAAGAACTGGAGAAGGAAGCTGAGGAACTTGAGCAGGAGC TCGCTGAACTTCGGAAGAAGGTGGAGGTGCTGGAGAACACCAACAGGACTCTCCT TCAGCAACTTCAGAAGCTTCAGACTTTGGTGATGGGGAAGGTCTCTCGAACCTGCA AGTTAGCTGGTACACAGACTGGCACCTGCCTCATGGTCGTTGTGCTTTAAGAATTC-3′. (SEQ ID NO: 68) ATF4 aZIP: 5′-GGATCCGCCACCATGGACTACAAAGATGATGACGACAAGCACATGGCCAGCATGA CCGGGGGCCAGCAGATGGGAAGAGACCCTGATTTGGAACAAAGGGCAGAGGAGC TGGCCCGGGAGAACGAAGAACTGGAGAAGGAAGCTGAGGAACTTGAGCAGGAGC TCGCTGAACTCACTGGCGAGTGTAAAGAGCTAGAAAAGAAGAACGAGGCTCTGAA AGAGAAGGCAGATTCTCTCGCCAAAGAGATTCAGTATCTAAAAGACCTGATAGAAG AGGTCCGTAAGGCAAGGGGGAAGAAGAGAGTTCCTTAAGAATTC-3′. (SEQ ID NO: 69) aZIP transgenes were cloned into the modified FUGW plasmid described in ‘Chemically induced proximity: reagent preparation’ using BamHI+EcoRI sites. Lentiviral particles were delivered at DIV1, 24 hours post-dissection. Neuronal cultures were allowed to mature until DIV15 and treated with Aβ₄₂ at 750 nM for 48 hours. Cell viability was assayed using RealTime-Glo MT Cell Viability Assay (Promega) following manufacturer's guidelines; reaction was allowed to proceed for 1 hour before taking luminescence readings (SpectraMax iD5, Molecular Devices). Also see FIGS. 34A-34D.

### Statistical Analysis

Each experiment was independently repeated at least three times unless otherwise indicated. Individual measurements were taken from distinct samples. Details of biological replication and statistical analysis are indicated in figure legends or main text. For all tests, a significance level (a) of 0.05 was used. Datasets were analyzed with Prism (GraphPad). Representation factors and associated probabilities were calculated using the formulas described in http://nemates.org/MA/; 25,000 was assumed as the total number genes in the human genome.

### DOCUMENTS CITED

1. Afgan, E. et al. The Galaxy platform for accessible, reproducible and collaborative biomedical analyses: 2018 update. Nucleic Acids Res 46, W537-W544, doi:10.1093/nar/gky379 (2018).
2. Alafuzoff, I. et al., Staging of neurofibrillary pathology in Alzheimer's disease: a study of the BrainNet Europe Consortium. Brain Pathol 18, 484-496 (2008).
3. Amoutzias, G. D., Robertson, D. L., Van de Peer, Y., and Oliver, S. G. (2008). Choose your partners: dimerization in eukaryotic transcription factors. Trends Biochem Sci 33, 220-229.
4. Andrews, S. J., B. Fulton-Howard, A. Goate, Interpretation of risk loci from genome-wide association studies of Alzheimer's disease. Lancet Neurol 19, 326-335 (2020).
5. Arighi, C. N., L. M. Hartnell, R. C. Aguilar, C. R. Haft, J. S. Bonifacino Role of the mammalian retromer in sorting of the cation-independent mannose 6-phosphate receptor. Journal of Cell Biology 165, 123-133 (2004).
6. Ashburner, M. et al. Gene ontology: tool for the unification of biology. The Gene Ontology Consortium. Nat Genet 25, 25-29, doi:10.1038/75556 (2000).
7. Baan, B., Pardali, E., ten Dijke, P., and van Dam, H. (2010). In situ proximity ligation detection of c-Jun/Aβ-1 dimers reveals increased levels of c-Jun/Fra1 complexes in aggressive breast cancer cell lines in vitro and in vivo. Mol Cell Proteomics 9, 1982-1990.
8. Bailey, T. L. et al. MEME SUITE: tools for motif discovery and searching. Nucleic Acids Res 37, W202-208, doi:10.1093/nar/gkp335 (2009).
9. Baleriola, J. et al. Axonally synthesized ATF4 transmits a neurodegenerative signal across brain regions. Cell 158, 1159-1172, doi:10.1016/j.cell.2014.07.001 (2014).
10. Barbosa, S., Fasanella, G., Carreira, S., Llarena, M., Fox, R., Barreca, C., Andrew, D., and O'Hare, P. (2013). An orchestrated program regulating secretory pathway genes and cargos by the transmembrane transcription factor CREB-H. Traffic 14, 382-398.
11. Bennett, D. A. et al., Religious Orders Study and Rush Memory and Aging Project. Journal of Alzheimer's disease.: JAD 64, S161-S189 (2018).
12. Ben-Yaakov, K., Dagan, S. Y., Segal-Ruder, Y., Shalem, O., Vuppalanchi, D., Willis, D. E., Yudin, D., Rishal, I., Rother, F., Bader, M., et al. (2012). Axonal transcription factors signal retrogradely in lesioned peripheral nerve. EMBO J 31, 1350-1363.
13. Bindea, G. et al. ClueGO: a Cytoscape plug-in to decipher functionally grouped gene ontology and pathway annotation networks. Bioinformatics 25, 1091-1093, doi:10.1093/bioinformatics/btp101 (2009).
14. Bossers, K. et al., Concerted changes in transcripts in the prefrontal cortex precede neuropathology in Alzheimer's disease. Brain 133, 3699-3723 (2010).
15. Braak, H. & Braak, E. Neuropathological staging of Alzheimer-related changes. Acta Neuropathol 82, 239-259 (1991).
16. Braak, H., Braak, E. & Bohl, J. Staging of Alzheimer-related cortical destruction. Eur Neurol 33, 403-408, doi:10.1159/000116984 (1993).
17. Braak, H., I. Alafuzoff, T. Arzberger, H. Kretzschmar, K. Del Tredici, Staging of Alzheimer disease-associated neurofibrillary pathology using paraffin sections and immunocytochemistry. Acta neuropathologica 112, 389-404 (2006).
18. Brigidi, G. S., Hayes, M. G. B., Delos Santos, N. P., Hartzell, A. L., Texari, L., Lin, P. A., Bartlett, A., Ecker, J. R., Benner, C., Heinz, S., et al. (2019). Genomic Decoding of Neuronal Depolarization by Stimulus-Specific NPAS4 Heterodimers. Cell 179, 373-391 e327.
19. Brohee, S. & Helden, J. van. Evaluation of clustering algorithms for protein-protein interaction networks. Bmc Bioinformatics 7, 488 (2006).
20. Buccitelli, C., M. Selbach, mRNAs, proteins and the emerging principles of gene expression control. Nature Reviews Genetics 21, 630-644 (2020).
21. Chan, C. P., Kok, K. H., and Jin, D. Y. (2011). CREB3 subfamily transcription factors are not created equal: Recent insights from global analyses and animal models. Cell Biosci 1, 6.
22. Chen, A., Muzzio, I. A., Malleret, G., Bartsch, D., Verbitsky, M., Pavlidis, P., Yonan, A. L., Vronskaya, S., Grody, M. B., Cepeda, I., et al. (2003). Inducible enhancement of memory storage and synaptic plasticity in transgenic mice expressing an inhibitor of ATF4 (CREB-2) and C/EBP proteins. Neuron 39, 655-669.
23. Consensus recommendations for the postmortem diagnosis of Alzheimer's disease. The National Institute on Aging, and Reagan Institute Working Group on Diagnostic Criteria for the Neuropathological Assessment of Alzheimer's Disease. Neurobiol Aging 18, S1-2 (1997).
24. Cullen, P. J., and Steinberg, F. (2018). To degrade or not to degrade: mechanisms and significance of endocytic recycling. Nat Rev Mol Cell Biol 19, 679-696.
25. Davis, C. A. et al. The Encyclopedia of DNA elements (ENCODE): data portal update. Nucleic Acids Research 46, D794-D801, doi:10.1093/nar/gkx1081 (2018).
26. De Strooper, B., E. Karran, The cellular phase of Alzheimer's disease. Cell 164, 603-615 (2016).
27. Desai, S. P., Freeman, D. M. & Voldman, J. Plastic masters-rigid templates for soft lithography. Lab Chip 9, 1631-1637, doi:10.1039/b822081f (2009).
28. Dhungel, N., Eleuteri, S., Li, L. B., Kramer, N. J., Chartron, J. W., Spencer, B., Kosberg, K., Fields, J. A., Stafa, K., Adame, A., et al. (2015). Parkinson's disease genes VPS35 and EIF4G1 interact genetically and converge on alpha-synuclein. Neuron 85, 76-87.
29. Dönerta§, H. M., M. Fuentealba Valenzuela, L. Partridge, J. M. Thornton, Gene expression-based drug repurposing to target aging. Aging Cell 17, e12819 (2018).
30. Dunham, I. et al. An integrated encyclopedia of DNA elements in the human genome. Nature 489, 57-74, doi:10.1038/naturel 1247 (2012).
31. Frank, C. L., X. Ge, Z. Xie, Y. Zhou, L. H. Tsai, Control of activating transcription factor 4 (ATF4) persistence by multisite phosphorylation impacts cell cycle progression and neurogenesis. J Biol Chem 285, 33324-33337 (2010).
32. Friedman, B. A. et al., Diverse Brain Myeloid Expression Profiles Reveal Distinct Microglial Activation States and Aspects of Alzheimer's Disease Not Evident in Mouse Models. Cell Rep 22, 832-847 (2018).
33. Gerdes, M. J. et al., Activator protein-1 activity regulates epithelial tumor cell identity. Cancer research 66, 7578-7588 (2006).
34. Greenberg, S. G., P. Davies, J. D. Schein, L. I. Binder, Hydrofluoric acid-treated tau PHF proteins display the same biochemical properties as normal tau. Journal of Biological Chemistry 267, 564-569 (1992).
35. Greenberg, S. M. & Vonsattel, J. P. Diagnosis of cerebral amyloid angiopathy. Sensitivity and specificity of cortical biopsy. Stroke 28, 1418-1422 (1997).
36. Guan, M., Fousek, K., and Chow, W. A. (2012). Nelfinavir inhibits regulated intramembrane proteolysis of sterol regulatory element binding protein-1 and activating transcription factor 6 in castration-resistant prostate cancer. FEBS J 279, 2399-2411.
37. Hai, T. & Curran, T. Cross-family dimerization of transcription factors Fos/Jun and ATF/CREB alters DNA binding specificity. Proc Natl Acad Sci USA 88, 3720-3724, doi:10.1073/pnas.88.9.3720 (1991).
38. Hai, T. W., F. Liu, W. J. Coukos, M. R. Green, Transcription factor ATF cDNA clones: an extensive family of leucine zipper proteins able to selectively form DNA-binding heterodimers. Genes Dev 3, 2083-2090 (1989).
39. Hanseeuw, B. J. et al., Association of Amyloid and Tau With Cognition in Preclinical Alzheimer Disease: A Longitudinal Study. JAMA Neurol, (2019).
40. Hass, M. R., Liow, H. H., Chen, X., Sharma, A., Inoue, Y. U., Inoue, T., Reeb, A., Martens, A., Fulbright, M., Raju, S., et al. (2015). SpDamID: Marking DNA Bound by Protein Complexes Identifies Notch-Dimer Responsive Enhancers. Mol Cell 59, 685-697.
41. Hengst, U., Deglincerti, A., Kim, H. J., Jeon, N. L. & Jaffrey, S. R. Axonal elongation triggered by stimulus-induced local translation of a polarity complex protein. Nat Cell Biol 11, 1024-1030, doi:10.1038/ncbl916 (2009).
42. Hetz, C., and Papa, F. R. (2018). The Unfolded Protein Response and Cell Fate Control. Mol Cell 69, 169-181.
43. Hetz, C., and Saxena, S. (2017). ER stress and the unfolded protein response in neurodegeneration. Nat Rev Neurol 13, 477-491.
44. Hetz, C., K. Zhang, R. J. Kaufman, Mechanisms, regulation and functions of the unfolded protein response. Nature Reviews Molecular Cell Biology 21, 421-438 (2020).
45. Isakova, A. et al., SMiLE-seq identifies binding motifs of single and dimeric transcription factors. Nat Methods 14, 316-322 (2017).
46. Issa, N. T. et al., DrugGenEx-Net: a novel computational platform for systems pharmacology and gene expression-based drug repurposing. BMC Bioinformatics 17, 202 (2016).
47. Jolma, A. et al., DNA-dependent formation of transcription factor pairs alters their binding specificity. Nature 527, 384-388 (2015).
48. Kelly, J., Moyeed, R., Carroll, C., Albani, D. & Li, X. Gene expression meta-analysis of Parkinson's disease and its relationship with Alzheimer's disease. Mol Brain 12, 16 (2019).
49. Khetchoumian, K., Balsalobre, A., Mayran, A., Christian, H., Chenard, V., St-Pierre, J., and Drouin, J. (2019). Pituitary cell translation and secretory capacities are enhanced cell autonomously by the transcription factor Creb312. Nat Commun 10, 3960.
50. Klein, H.-U. et al., Epigenome-wide study uncovers large-scale changes in histone acetylation driven by tau pathology in aging and Alzheimer's human brains. Nature Neuroscience 22, 37-46 (2019).
51. Kondo, S., Saito, A., Hino, S., Murakami, T., Ogata, M., Kanemoto, S., Nara, S., Yamashita, A., Yoshinaga, K., Hara, H., et al. (2007). BBF2H7, a novel transmembrane bZIP transcription factor, is a new type of endoplasmic reticulum stress transducer. Mol Cell Biol 27, 1716-1729.
52. Lamb, J. et al., The Connectivity Map: Using Gene-Expression Signatures to Connect Small Molecules, Genes, and Disease. Science 313, 1929-1935 (2006).
53. Lambert, M., Jambon, S., Depauw, S., and David-Cordonnier, M. H. (2018a). Targeting Transcription Factors for Cancer Treatment. Molecules 23.
54. Lambert, S. A., Jolma, A., Campitelli, L. F., Das, P. K., Yin, Y., Albu, M., Chen, X., Taipale, J., Hughes, T. R., and Weirauch, M. T. (2018b). The Human Transcription Factors. Cell 175, 598-599.
55. Lassot, I. et al., ATF4 degradation relies on a phosphorylation-dependent interaction with the SCF(betaTrCP) ubiquitin ligase. Mol Cell Biol 21, 2192-2202 (2001).
56. Lee, T. I., and Young, R. A. (2013). Transcriptional regulation and its misregulation in disease. Cell 152, 1237-1251.
57. Lesnick, T. G. et al. A Genomic Pathway Approach to a Complex Disease: Axon Guidance and Parkinson Disease. Plos Genet 3, e98 (2007).
58. Li, J.-G., J. Chiu, D. Praticò, Full recovery of the Alzheimer's disease phenotype by gain of function of vacuolar protein sorting 35. Molecular Psychiatry 25, 2630-2640 (2020).
59. Liu, Y., Yoo, M. J., Savonenko, A., Stirling, W., Price, D. L., Borchelt, D. R., Mamounas, L., Lyons, W. E., Blue, M. E., and Lee, M. K. (2008). Amyloid pathology is associated with progressive monoaminergic neurodegeneration in a transgenic mouse model of Alzheimer's disease. J Neurosci 28, 13805-13814.
60. Long, J. M., and Holtzman, D. M. (2019). Alzheimer Disease: An Update on Pathobiology and Treatment Strategies. Cell 179, 312-339.
61. Machanick, P. & Bailey, T. L. MEME-ChIP: motif analysis of large DNA datasets. Bioinformatics 27, 1696-1697, doi:10.1093/bioinformatics/btr189 (2011).
62. Mathys, H., Davila-Velderrain, J., Peng, Z., Gao, F., Mohammadi, S., Young, J. Z., Menon, M., He, L., Abdurrob, F., Jiang, X., et al. (2019). Single-cell transcriptomic analysis of Alzheimer's disease. Nature 570, 332-337.
63. McCurdy, E. P., Chung, K. M., Benitez-Agosto, C. R., and Hengst, U. (2019). Promotion of Axon Growth by the Secreted End of a Transcription Factor. Cell Rep 29, 363-377 e365.
64. McLean, C. Y. et al. GREAT improves functional interpretation of cis-regulatory regions. Nat Biotechnol 28, 495-501, doi:10.1038/nbt.1630 (2010).
65. Miller, J. A. et al. Transcriptional landscape of the prenatal human brain. Nature 508, 199-206, doi:10.1038/nature13185 (2014).
66. Miller, J. A., Guillozet-Bongaarts, A., Gibbons, L. E., Postupna, N., Renz, A., Beller, A. E., Sunkin, S. M., Ng, L., Rose, S. E., Smith, K. A., et al. (2017). Neuropathological and transcriptomic characteristics of the aged brain. Elife 6.
67. Mirra, S. S. et al. The Consortium to Establish a Registry for Alzheimer's Disease (CERAD). Part II. Standardization of the neuropathologic assessment of Alzheimer's disease. Neurology 41, 479-486, doi:10.1212/wnl.41.4.479 (1991).
68. Mondragon-Rodriguez, S., G. Perry, J. Luna-Muñoz, M. C. Acevedo-Aquino, S. Williams, Phosphorylation of tau protein at sites Ser(396-404) is one of the earliest events in Alzheimer's disease and Down syndrome. Neuropathol Appl Neurobiol 40, 121-135 (2014).
69. Moran, L. B. et al. Whole genome expression profiling of the medial and lateral substantia nigra in Parkinson's disease. Neurogenetics 7, 1-11 (2006).
70. Mostafavi, S., Gaiteri, C., Sullivan, S. E., White, C. C., Tasaki, S., Xu, J., Taga, M., Klein, H. U., Patrick, E., Komashko, V., et al. (2018). A molecular network of the aging human brain provides insights into the pathology and cognitive decline of Alzheimer's disease. Nat Neurosci 21, 811-819.
71. Musiek, E. S., D. A. Bennett, Aducanumab and the “post-amyloid” era of Alzheimer research? Neuron 109, 3045-3047 (2021).
72. Musiek, E. S., D. M. Holtzman, Three dimensions of the amyloid hypothesis: time, space and ‘wingmen’. Nat Neurosci 18, 800-806 (2015).
73. Nakamura, A., Kaneko, N., Villemagne, V. L., Kato, T., Doecke, J., Dore, V., Fowler, C., Li, Q. X., Martins, R., Rowe, C., et al. (2018). High performance plasma amyloid-beta biomarkers for Alzheimer's disease. Nature 554, 249-254.
74. Naslavsky, N., and Caplan, S. (2011). EHD proteins: key conductors of endocytic transport. Trends Cell Biol 21, 122-131.
75. Nativio, R. et al., An integrated multi-omics approach identifies epigenetic alterations associated with Alzheimer's disease. Nature Genetics 52, 1024-1035 (2020).
76. Newman, J. R., and Keating, A. E. (2003). Comprehensive identification of human bZIP interactions with coiled-coil arrays. Science 300, 2097-2101.
77. Nixon, R. A., Amyloid precursor protein and endosomal-lysosomal dysfunction in Alzheimer's disease: inseparable partners in a multifactorial disease. FASEB J 31, 2729-2743 (2017).
78. Nordmann, M., Cabrera, M., Perz, A., Brocker, C., Ostrowicz, C., Engelbrecht-Vandre, S., and Ungermann, C. (2010). The Mon1-Ccz1 complex is the GEF of the late endosomal Rab7 homolog Ypt7. Curr Biol 20, 1654-1659.
79. Oh, R. S. et al. Functional RNA interference (RNAi) screen identifies system A neutral amino acid transporter 2 (SNAT2) as a mediator of arsenic-induced endoplasmic reticulum stress. J Biol Chem 287, 6025-6034, doi:10.1074/jbc.M111.311217 (2012).
80. Ohba, S., X. He, H. Hojo, Andrew P. McMahon, Distinct Transcriptional Programs Underlie Sox9 Regulation of the Mammalian Chondrocyte. Cell Reports 12, 229-243 (2015).
81. Otvos, Jr., L. et al., Monoclonal antibody PHF-1 recognizes tau protein phosphorylated at serine residues 396 and 404. J Neurosci Res 39, 669-673 (1994).
82. Park, J. W., Vahidi, B., Taylor, A. M., Rhee, S. W. & Jeon, N. L. Microfluidic culture platform for neuroscience research. Nat Protoc 1, 2128-2136, doi:10.1038/nprot.2006.316 (2006).
83. Pascoal, T. A., Mathotaarachchi, S., Kang, M. S., Mohaddes, S., Shin, M., Park, A. Y., Parent, M. J., Benedet, A. L., Chamoun, M., Therriault, J., et al. (2019). Abeta-induced vulnerability propagates via the brain's default mode network. Nat Commun 10, 2353.
84. Pasini, S., Corona, C., Liu, J., Greene, L. A., and Shelanski, M. L. (2015). Specific downregulation of hippocampal ATF4 reveals a necessary role in synaptic plasticity and memory. Cell Rep 11, 183-191.
85. Pitale, P. M., O. Gorbatyuk, M. Gorbatyuk, Neurodegeneration: Keeping ATF4 on a Tight Leash. Front Cell Neurosci 11, 410 (2017).
86. Raj, T., Li, Y. I., Wong, G., Humphrey, J., Wang, M., Ramdhani, S., Wang, Y. C., Ng, B., Gupta, I., Haroutunian, V., et al. (2018a). Integrative transcriptome analyses of the aging brain implicate altered splicing in Alzheimer's disease susceptibility. Nat Genet 50, 1584-1592.
87. Raj, T., Li, Y. I., Wong, G., Humphrey, J., Wang, M., Ramdhani, S., Wang, Y. C., Ng, B., Gupta, I., Haroutunian, V., et al. (2018b). Integrative transcriptome analyses of the aging brain implicate altered splicing in Alzheimer's disease susceptibility. Nat Genet.
88. Reddy, P. H., M. F. Beal, Amyloid beta, mitochondrial dysfunction and synaptic damage: implications for cognitive decline in aging and Alzheimer's disease. Trends in Molecular Medicine 14, 45-53 (2008).
89. Reiter, F., Wienerroither, S., and Stark, A. (2017). Combinatorial function of transcription factors and cofactors. Curr Opin Genet Dev 43, 73-81.
90. Rodriguez, S. et al., Machine learning identifies candidates for drug repurposing in Alzheimer's disease. Nature Communications 12, 1033 (2021).
91. Rodriguez-Martinez, J. A., A. W. Reinke, D. Bhimsaria, A. E. Keating, A. Z. Ansari, Combinatorial bZIP dimers display complex DNA-binding specificity landscapes. Elife 6, (2017).
92. Santiago, J. A., and Potashkin, J. A. (2015). Network-based metaanalysis identifies HNF4A and PTBP1 as longitudinally dynamic biomarkers for Parkinson's disease. Proc Natl Acad Sci USA 112, 2257-2262.
93. Sato, C. et al., Tau Kinetics in Neurons and the Human Central Nervous System. Neuron 97, 1284-1298.e1287 (2018).
94. Schafer, N. et al., Phase I trial of dovitinib (TK1258) in recurrent glioblastoma. J Cancer Res Clin Oncol 142, 1581-1589 (2016).
95. Scheltens, P. et al., Alzheimer's disease. Lancet 388, 505-517 (2016).
96. Scheltens, P., Blennow, K., Breteler, M. M., de Strooper, B., Frisoni, G. B., Salloway, S., and Van der Flier, W. M. (2016). Alzheimer's disease. Lancet 388, 505-517.
97. Seaman, M. N. J., Cargo-selective endosomal sorting for retrieval to the Golgi requires retromer. The Journal of cell biology 165, 111-122 (2004).
98. Shannon, P. et al. Cytoscape: a software environment for integrated models of biomolecular interaction networks. Genome Res 13, 2498-2504, doi:10.1101/gr.1239303 (2003).
99. Shigeoka, T., Jung, H., Jung, J., Turner-Bridger, B., Ohk, J., Lin, J. Q., Amieux, P. S., and Holt, C. E. (2016). Dynamic Axonal Translation in Developing and Mature Visual Circuits. Cell 166, 181-192.
100. Simoes, S. et al., Alzheimer's vulnerable brain region relies on a distinct retromer core dedicated to endosomal recycling. Cell Reports 37, (2021).
101. Small, S. A. et al., Model-guided microarray implicates the retromer complex in Alzheimer's disease. Ann Neurol 58, 909-919 (2005).
102. Small, S. A., and Petsko, G. A. (2015). Retromer in Alzheimer disease, Parkinson disease and other neurological disorders. Nat Rev Neurosci 16, 126-132.
103. Small, S. A., Simoes-Spassov, S., Mayeux, R., and Petsko, G. A. (2017). Endosomal Traffic Jams Represent a Pathogenic Hub and Therapeutic Target in Alzheimer's Disease. Trends Neurosci 40, 592-602.
104. Soderberg, O., Gullberg, M., Jarvius, M., Ridderstrale, K., Leuchowius, K. J., Jarvius, J., Wester, K., Hydbring, P., Bahram, F., Larsson, L. G., et al. (2006). Direct observation of individual endogenous protein complexes in situ by proximity ligation. Nat Methods 3, 995-1000.
105. Stanton, B. Z., Chory, E. J., and Crabtree, G. R. (2018). Chemically induced proximity in biology and medicine. Science 359.
106. Steeland, S. et al., Counteracting the effects of TNF receptor-1 has therapeutic potential in Alzheimer's disease. EMBO Mol Med 10, (2018).
107. Su, B. G., M. J. Henley, Drugging Fuzzy Complexes in Transcription. Frontiers in Molecular Biosciences 8, (2021).
108. Sun, X. et al., ATF4 protects against neuronal death in cellular Parkinson's disease models by maintaining levels of parkin. J Neurosci 33, 2398-2407 (2013).
109. Sun, X. et al., Dominant-negative ATF5 rapidly depletes survivin in tumor cells. Cell Death Dis 10, 709 (2019).
110. Szklarczyk, D. et al. STRING v10: protein-protein interaction networks, integrated over the tree of life. Nucleic Acids Res 43, D447-452, doi:10.1093/nar/gku1003 (2015).
111. Szklarczyk, D. et al. STRING v11: protein-protein association networks with increased coverage, supporting functional discovery in genome-wide experimental datasets. Nucleic Acids Res 47, gky1131 (2018).
112. Taylor, A. M. et al. A microfluidic culture platform for CNS axonal injury, regeneration and transport. Nat Methods 2, 599-605, doi:10.1038/nmeth777 (2005).
113. The Gene Ontology, C. The Gene Ontology Resource: 20 years and still Going strong. Nucleic Acids Res 47, D330-D338, doi:10.1093/nar/gky1055 (2019).
114. Toepke, M. W., D. J. Beebe, PDMS absorption of small molecules and consequences in microfluidic applications. Lab on a Chip 6, 1484-1486 (2006).
115. tom Dieck, S., Kochen, L., Hanus, C., Heumuller, M., Bartnik, I., Nassim-Assir, B., Merk, K., Mosler, T., Garg, S., Bunse, S., et al. (2015). Direct visualization of newly synthesized target proteins in situ. Nat Methods 12, 411-414.
116. Trudel, S. et al., CHIR-258, a novel, multitargeted tyrosine kinase inhibitor for the potential treatment of t(4;14) multiple myeloma. Blood 105, 2941-2948 (2005).
117. Vardarajan, B. N., Bruesegem, S. Y., Harbour, M. E., Inzelberg, R., Friedland, R., St George-Hyslop, P., Seaman, M. N., and Farrer, L. A. (2012). Identification of Alzheimer disease-associated variants in genes that regulate retromer function. Neurobiol Aging 33, 2231 e2215-2231 e2230.
118. Verheijen, J., and Sleegers, K. (2018). Understanding Alzheimer Disease at the Interface between Genetics and Transcriptomics. Trends Genet 34, 434-447.
119. Vilarino-Guell, C., Wider, C., Ross, O. A., Dachsel, J. C., Kachergus, J. M., Lincoln, S. J., Soto-Ortolaza, A. I., Cobb, S. A., Wilhoite, G. J., Bacon, J. A., et al. (2011). VPS35 mutations in Parkinson disease. Am J Hum Genet 89, 162-167.
120. Vinson, C. et al., Classification of human B-ZIP proteins based on dimerization properties. Mol Cell Biol 22, 6321-6335 (2002).
121. Volpicelli-Daley, L. A. et al., Exogenous α-synuclein fibrils induce Lewy body pathology leading to synaptic dysfunction and neuron death. Neuron 72, 57-71 (2011).
122. Walker, C. A., Randolph, L. K., Matute, C., Alberdi, E., Baleriola, J., and Hengst, U. (2018). Abeta1-42 triggers the generation of a retrograde signaling complex from sentinel mRNAs in axons. EMBO Rep 19, e45435.
123. Wang, X., Zhao, Y., Zhang, X., Badie, H., Zhou, Y., Mu, Y., Loo, L. S., Cai, L., Thompson, R. C., Yang, B., et al. (2013). Loss of sorting nexin 27 contributes to excitatory synaptic dysfunction by modulating glutamate receptor recycling in Down's syndrome. Nat Med 19, 473-480.
124. Wang, Y., E. Mandelkow, Tau in physiology and pathology. Nature Reviews Neuroscience 17, 22-35 (2016).
125. Webster, J. A. et al., Genetic control of human brain transcript expression in Alzheimer disease. Am J Hum Genet 84, 445-458 (2009).
126. Wen, L. et al., VPS35 haploinsufficiency increases Alzheimer's disease neuropathology. J Cell Biol 195, 765-779 (2011).
127. Williams, G. et al., Drug repurposing for Alzheimer's disease based on transcriptional profiling of human iPSC-derived cortical neurons. Translational Psychiatry 9, 220 (2019).
128. Wolff, J. (2009). Plasma membrane tubulin. Biochim Biophys Acta 1788, 1415-1433.
129. Ying, Z., Misra, V., and Verge, V. M. (2014). Sensing nerve injury at the axonal ER: activated Luman/CREB3 serves as a novel axonally synthesized retrograde regeneration signal. Proc Natl Acad Sci USA 111, 16142-16147.
130. Yuste-Checa, P. et al., The extracellular chaperone Clusterin enhances Tau aggregate seeding in a cellular model. Nature Communications 12, 4863 (2021).
131. Zhang, B., Gaiteri, C., Bodea, L. G., Wang, Z., McElwee, J., Podtelezhnikov, A. A., Zhang, C., Xie, T., Tran, L., Dobrin, R., et al. (2013). Integrated systems approach identifies genetic nodes and networks in late-onset Alzheimer's disease. Cell 153, 707-720.
132. Zhang, X., and Song, W. (2013). The role of APP and BACE1 trafficking in APP processing and amyloid-beta generation. Alzheimers Res Ther 5, 46.
133. Zheng, B. et al. PGC-1 a, A Potential Therapeutic Target for Early Intervention in Parkinson's Disease. Sci Transl Med 2, 52ra73-52ra73 (2010).
134. Zhou, Y. et al., Metascape provides a biologist-oriented resource for the analysis of systems-level datasets. Nat Commun 10, 1523 (2019).
135. Zimprich, A., Benet-Pages, A., Struhal, W., Graf, E., Eck, S. H., Offman, M. N., Haubenberger, D., Spielberger, S., Schulte, E. C., Lichtner, P., et al. (2011). A mutation in VPS35, encoding a subunit of the retromer complex, causes late-onset Parkinson disease. Am J Hum Genet 89, 168-175.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A method for treating or ameliorating the effects of a neurodegenerative disease in a subject, comprising:
(a) determining the level of CREB3L2-ATF4 transcription factor (TF) complex in a sample obtained from the subject; and
(b) administering to the subject an effective amount of an agent that modulates the association between CREB3L2 and ATF4, if the level of CREB3L2-ATF4 complex determined in step (a) is significantly different from that of a control subject.

2. The method of claim 1, wherein the neurodegenerative disease is associated with retromer complex dysfunction, altered β-amyloid metabolism, tau hyperphosphorylation, or combinations thereof.

3. The method of claim 1, wherein the neurodegenerative disease is selected from the group consisting of Alzheimer's Disease, Parkinson's Disease, Frontotemporal Lobar Degeneration, Down's Syndrome, Hereditary Spastic Paraplegia, Neuronal Ceroid Lipofuscinoses, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, and Progressive supranuclear palsy.

4. The method of claim 1, wherein the neurodegenerative disease is Alzheimer's disease or Parkinson's disease.

5. The method of claim 1, wherein the neurodegenerative disease is late-onset Alzheimer's disease (LOAD).

6. The method of claim 1, wherein the subject is a mammal.

7. The method of claim 5, wherein the mammal is a human.

8. The method of claim 1, wherein the agent that modulates the association between CREB3L2 and ATF4 does not ablate CREB3L2 signaling.

9. The method of claim 1, wherein the agent is dovitinib.

10. A method for restoring retromer complex function in a subject, comprising administering to the subject an effective amount of an agent that modulates CREB3L2 expression.

11. The method of claim 10, wherein the modulation of CREB3L2 expression comprises modulating the association between CREB3L2 and ATF4.

12. The method of claim 10, wherein the modulation of CREB3L2 expression results in at least one retromer-associated gene in the subject being deregulated by CREB3L2.

13. The method of claim 12, wherein the at least one retromer-associated gene is selected from the group consisting of SNX3, SNX27, RAB7A, SNX1, VPS29, SNX5, VPS26B, SNX2, EHD1, SNX6, VPS26A, VPS35, and combinations thereof.

14. The method of claim 12, wherein the at least one retromer-associated gene is selected from the group consisting of VPS26B, VPS35, SNX2, SNX5, SNX3, RAB7A, EHD1, and combinations thereof.

15. A method for determining the progression of a neurodegenerative disease in a subject, comprising:
(a) determining the level of CREB3L2-ATF4 transcription factor (TF) complex in a sample obtained from the subject; and
(b) concluding that the neurodegenerative disease in the subject is progressing, if the level of CREB3L2-ATF4 complex determined in step (a) is significantly increased from that of a control subject.

16. The method of claim 15, wherein the neurodegenerative disease is associated with retromer complex dysfunction, altered β-amyloid metabolism, tau hyperphosphorylation, or combinations thereof.

17. The method of claim 15, wherein the neurodegenerative disease is selected from the group consisting of Alzheimer's Disease, Parkinson's Disease, Frontotemporal Lobar Degeneration, Down's Syndrome, Hereditary Spastic Paraplegia, Neuronal Ceroid Lipofuscinoses, Amyotrophic lateral sclerosis, Friedreich's ataxia, Multiple sclerosis, Huntington's Disease, Transmissible spongiform encephalopathy, Charcot-Marie-Tooth disease, Dementia with Lewy bodies, Corticobasal degeneration, and Progressive supranuclear palsy.

18. The method of claim 15, wherein the neurodegenerative disease is Alzheimer's disease or Parkinson's disease.

19. The method of claim 15, wherein the neurodegenerative disease is late-onset Alzheimer's disease (LOAD).

20. The method of claim 15, wherein the subject is a mammal.

21. The method of claim 20, wherein the mammal is a human.

22. The method of claim 15, wherein the sample is obtained from the dorsolateral prefrontal cortex (PFC) of the subject.

23. A method for identifying the DNA-binding profile of a dimeric transcription factor complex in vivo, comprising:
(a) generating a DNA construct of a first transcription factor comprising: (i) fusing a specific first dimerization domain to the C-terminal of the first transcription factor; and (ii) adding a first N-terminal epitope tag to the first transcription factor;
(b) generating a DNA construct of a second transcription factor comprising: (i) fusing a specific second dimerization domain to the C-terminal of the second transcription factor, wherein the second dimerization domain is different from the first dimerization domain; and (ii) adding a second N-terminal epitope tag to the second transcription factor, wherein the second N-terminal epitope tag is different from the first N-terminal epitope tag;
(c) identifying a bivalent ligand that recognizes both dimerization domains from steps (a) and (b);
(d) co-transfecting a host cell with the DNA constructs generated in steps (a)-(b) and co-expressing polypeptides encoded by the DNA constructs in the presence of the bivalent ligand identified in step (c) to form the dimeric transcription factor complex; and
(e) identifying the DNA-binding profile of the dimeric transcription factor complex by determining the complex's binding sites to the genome using ChIP-sequencing (ChIP-seq).

24. The method of claim 23, wherein the dimerization domain is selected from the group consisting of FKBP, Calcineurin A (CNA), CyP-Fas, FRB, GyrB, GAI, GID1, SNAP-tag, HaloTag, eDHFR, Bcl-xL, and Fab(AZ1).

25. The method of claim 23, wherein the bivalent ligand is selected from the group consisting of FK1012, FK506, FKCsA, Rapamycin, Coumermycin, Gibberellin, HaXS, TMP-HTag, and ABT-737.

26. The method of claim 23, wherein the co-transfection in step (d) is a transient co-transfection.

27. The method of claim 26, wherein the transient co-transfection can be carried out by a liposome-mediated method, a non-liposomal method, a viral delivery method, or electroporation.

28. The method of claim 23, wherein the host cell is selected from the group consisting of HEK293, COS, CHO, and BHK cells.

29. The method of claim 23, wherein the host cell is HEK293 cell.

30. A method for restoring amyloid precursor protein (APP) homeostasis in a subject in need thereof, comprising:
(a) determining the Aβ₁₋₄₂/Aβ₁₋₄₀ ratio in a sample obtained from the subject; and
(b) administering to the subject an effective amount of an agent that increases the expression level of CREB3L2 or prevents the dimerization of CREB3L2 with ATF4, if the Aβ₁₋₄₂/Aβ₁₋₄₀ ratio determined in step (a) is significantly higher that a predetermined reference.

31. A method for restoring tau metabolism in a subject in need thereof, comprising administering to the subject an effective amount of an agent that modulates the association between CREB3L2 and ATF4.

32. The method of claim 31, wherein the modulation is to reduce level of CREB3L2-ATF4 heterodimer in the subject.

33. The method of claim 31, wherein the modulation of CREB3L2-ATF4 association restores expression level of holoenzyme protein phosphatase 2A (PP2A).

34. The method of claim 31, wherein the modulation of CREB3L2-ATF4 association restores expression levels of genes selected from the group consisting of SCG3, CLU, HSPA2, P4HB, HSPB1, PHGDH, MBNL2, PPP2CA, CELF3, AKT1, PKN1, CFL1, DBN1, MAOB, IGF1, NTRK2, AIF1, SLC1A3, and cominations thereof.

35. The method of claim 31, wherein the restoration of tau metabolism comprises reduction of phosphorylation at Ser404.

36. A composition comprising a nucleotide of SEQ ID NO: 68 or SEQ ID NO: 69.

37. A method for preventing CREB3L2-ATF4 heterodimerization in a subject, comprising administering to the subject an effective amount of the composition according to claim 36.

38. A method for rescuing Aβ₄₂-induced neuronal cell death in a subject, comprising administering to the subject an effective amount of the composition according to claim 36.


SEQUENCE LISTING (69 sequences)

SEQ ID NO: 1 - RNA, 25 nt, synthetic construct
  misc_feature 1..25: note = siRNA
  source 1..25: mol_type = other RNA
cgagggctatcccattccaaccaaa

SEQ ID NO: 2 - RNA, 25 nt, synthetic construct
  misc_feature 1..25: note = siRNA
  source 1..25: mol_type = other RNA
gctaacagatgatggtgacatgatt

SEQ ID NO: 3 - DNA, 20 bp, synthetic construct
  misc_feature 1..20: note = primer
  source 1..20: mol_type = other DNA
acttccgggtccgagataac

SEQ ID NO: 4 - DNA, 21 bp, synthetic construct
  misc_feature 1..21: note = primer
  source 1..21: mol_type = other DNA
gtgtccaggagcctaccaatc

SEQ ID NO: 5 - DNA, 20 bp, synthetic construct
  misc_feature 1..20: note = primer
  source 1..20: mol_type = other DNA
gaggaagcaaggatttgtgc

SEQ ID NO: 6 - DNA, 20 bp, synthetic construct
  misc_feature 1..20: note = primer
  source 1..20: mol_type = other DNA
gtgagatcaggtgcgaaggt

SEQ ID NO: 7 - DNA, 20 bp, synthetic construct
  misc_feature 1..20: note = primer
  source 1..20: mol_type = other DNA
actcggctttcccttaggag

SEQ ID NO: 8 - DNA, 20 bp, synthetic construct
  misc_feature 1..20: note = primer
  source 1..20: mol_type = other DNA
gaggaaaaggagtggtcacg

SEQ ID NO: 9 - DNA, 20 bp, synthetic construct
  misc_feature 1..20: note = primer
  source 1..20: mol_type = other DNA
cgcactcactgaactgccta

SEQ ID NO: 10 - DNA, 19 bp, synthetic construct
  misc_feature 1..19: note = primer
  source 1..19: mol_type = other DNA
gtggagagggagaagacgt

SEQ ID NO: 11 - DNA, 20 bp, synthetic construct
  misc_feature 1..20: note = primer
  source 1..20: mol_type = other DNA
ggcacagtacctccccagat

SEQ ID NO: 12 - DNA, 20 bp, synthetic construct
  misc_feature 1..20: note = primer
  source 1..20: mol_type = other DNA
cgggagtctaggcagttcag

SEQ ID NO: 13 - DNA, 20 bp, synthetic construct
  misc_feature 1..20: note = primer
  source 1..20: mol_type = other DNA
caatgagacgacgagtttgc

SEQ ID NO: 14 - DNA, 19 bp, synthetic construct
  misc_feature 1..19: note = primer
  source 1..19: mol_type = other DNA
gaggaatttctcgcagcac

SEQ ID NO: 15 - DNA, 20 bp, synthetic construct
  misc_feature 1..20: note = primer
  source 1..20: mol_type = other DNA
gactttatgtgggccaatcg

SEQ ID NO: 16 - DNA, 18 bp, synthetic construct
  misc_feature 1..18: note = primer
  source 1..18: mol_type = other DNA
acaagcagcagcgcctac

SEQ ID NO: 17 - DNA, 20 bp, synthetic construct
  misc_feature 1..20: note = primer
  source 1..20: mol_type = other DNA
ggcgccagtgaaaatatcct

SEQ ID NO: 18 - DNA, 18 bp, synthetic construct
  misc_feature 1..18: note = primer
  source 1..18: mol_type = other DNA
gggaggtggtggctgtag

SEQ ID NO: 19 - DNA, 19 bp, synthetic construct
  misc_feature 1..19: note = primer
  source 1..19: mol_type = other DNA
gactccggatccattccag

SEQ ID NO: 20 - DNA, 20 bp, synthetic construct
  misc_feature 1..20: note = primer
  source 1..20: mol_type = other DNA
gctcgcggcactgtctataa

SEQ ID NO: 21 - DNA, 20 bp, synthetic construct
  misc_feature 1..20: note = primer
  source 1..20: mol_type = other DNA
tcacagtgaggcactggact

SEQ ID NO: 22 - DNA, 20 bp, synthetic construct
  misc_feature 1..20: note = primer
  source 1..20: mol_type = other DNA
accccggaaatgattttagc

SEQ ID NO: 23 - DNA, 19 bp, synthetic construct
  misc_feature 1..19: note = primer
  source 1..19: mol_type = other DNA
gccttgggctttatggtca

SEQ ID NO: 24 - DNA, 20 bp, synthetic construct
  misc_feature 1..20: note = primer
  source 1..20: mol_type = other DNA
gtctcgtgacaggcacttcc

SEQ ID NO: 25 - DNA, 20 bp, synthetic construct
  misc_feature 1..20: note = primer
  source 1..20: mol_type = other DNA
caccatattgggccaagaac

SEQ ID NO: 26 - DNA, 20 bp, synthetic construct
  misc_feature 1..20: note = primer
  source 1..20: mol_type = other DNA
gttccaaagggggacactct

SEQ ID NO: 27 - DNA, 19 bp, synthetic construct
  misc_feature 1..19: note = primer
  source 1..19: mol_type = other DNA
gtctgtacgccggtccttg

SEQ ID NO: 28 - DNA, 20 bp, synthetic construct
  misc_feature 1..20: note = primer
  source 1..20: mol_type = other DNA
ggagacagagctggctgcta

SEQ ID NO: 29 - DNA, 35 bp, synthetic construct
  misc_feature 1..35: note = primer
  source 1..35: mol_type = other DNA
attagtgaattctgaggtgctggagagcggggagc

SEQ ID NO: 30 - DNA, 34 bp, synthetic construct
  misc_feature 1..34: note = primer
  source 1..34: mol_type = other DNA
cggaccggatccttagaaagtggtgttcactctt

SEQ ID NO: 31 - DNA, 35 bp, synthetic construct; identical to SEQ ID NO: 29
  misc_feature 1..35: note = primer
  source 1..35: mol_type = other DNA

SEQ ID NO: 32 - DNA, 37 bp, synthetic construct
  misc_feature 1..37: note = primer
  source 1..37: mol_type = other DNA
caatcgggatccttacagcaccacaaccatgaggcag

SEQ ID NO: 33 - DNA, 39 bp, synthetic construct
  misc_feature 1..39: note = primer
  source 1..39: mol_type = other DNA
gcaaacctggcgatctacgaggaacattctcccccagag

SEQ ID NO: 34 - DNA, 39 bp, synthetic construct
  misc_feature 1..39: note = primer
  source 1..39: mol_type = other DNA
gatcgccaggtttgcggatctcaccacggaggctgtgta

SEQ ID NO: 35 - DNA, 20 bp, synthetic construct
  misc_feature 1..20: note = primer
  source 1..20: mol_type = other DNA
gacaaaggcctccttcttgg

SEQ ID NO: 36 - DNA, 20 bp, synthetic construct
  misc_feature 1..20: note = primer
  source 1..20: mol_type = other DNA
gagggaggaggctaacgact

SEQ ID NO: 37 - DNA, 32 bp, synthetic construct
  misc_feature 1..32: note = primer
  source 1..32: mol_type = other DNA
atacatgagctcgacaaaggcctccttcttgg

SEQ ID NO: 38 - DNA, 32 bp, synthetic construct
  misc_feature 1..32: note = primer
  source 1..32: mol_type = other DNA
caggtcctcgaggagggaggaggctaacgact

SEQ ID NO: 39 - DNA, 18 bp, synthetic construct
  misc_feature 1..18: note = primer
  source 1..18: mol_type = other DNA
cgccgtgctcaactttca

SEQ ID NO: 40 - DNA, 19 bp, synthetic construct
  misc_feature 1..19: note = primer
  source 1..19: mol_type = other DNA
cctaacgctcctgggactc

SEQ ID NO: 41 - DNA, 30 bp, synthetic construct
  misc_feature 1..30: note = primer
  source 1..30: mol_type = other DNA
gatcacctcgagcgccgtgctcaactttca

SEQ ID NO: 42 - DNA, 31 bp, synthetic construct
  misc_feature 1..31: note = primer
  source 1..31: mol_type = other DNA
ctgatcaagcttcctaacgctcctgggactc

SEQ ID NO: 43 - DNA, 21 bp, synthetic construct
  misc_feature 1..21: note = primer
  source 1..21: mol_type = other DNA
caaactctgtgacgaaggtgc

SEQ ID NO: 44 - DNA, 22 bp, synthetic construct
  misc_feature 1..22: note = primer
  source 1..22: mol_type = other DNA
tcagctgtgctcaaaacaatac

SEQ ID NO: 45 - DNA, 33 bp, synthetic construct
  misc_feature 1..33: note = primer
  source 1..33: mol_type = other DNA
atacatgagctccaaactctgtgacgaaggtgc

SEQ ID NO: 46 - DNA, 34 bp, synthetic construct
  misc_feature 1..34: note = primer
  source 1..34: mol_type = other DNA
caggtcctcgagtcagctgtgctcaaaacaatac

SEQ ID NO: 47 - DNA, 20 bp, synthetic construct
  misc_feature 1..20: note = primer
  source 1..20: mol_type = other DNA
cgcatctggattggttagga

SEQ ID NO: 48 - DNA, 27 bp, synthetic construct
  misc_feature 1..27: note = primer
  source 1..27: mol_type = other DNA
agtcaaatacttggattttatcaatgg

SEQ ID NO: 49 - DNA, 32 bp, synthetic construct
  misc_feature 1..32: note = primer
  source 1..32: mol_type = other DNA
gatcacctcgagcgcatctggattggttagga

SEQ ID NO: 50 - DNA, 39 bp, synthetic construct
  misc_feature 1..39: note = primer
  source 1..39: mol_type = other DNA
ctgatcaagcttagtcaaatacttggattttatcaatgg

SEQ ID NO: 51 - DNA, 19 bp, synthetic construct
  misc_feature 1..19: note = primer
  source 1..19: mol_type = other DNA
cagcagcctcgggattgat

SEQ ID NO: 52 - DNA, 23 bp, synthetic construct
  misc_feature 1..23: note = primer
  source 1..23: mol_type = other DNA
agatacacttttaaggatggcga

SEQ ID NO: 53 - DNA, 31 bp, synthetic construct
  misc_feature 1..31: note = primer
  source 1..31: mol_type = other DNA
gatcacctcgagcagcagcctcgggattgat

SEQ ID NO: 54 - DNA, 35 bp, synthetic construct
  misc_feature 1..35: note = primer
  source 1..35: mol_type = other DNA
ctgatcaagcttagatacacttttaaggatggcga

SEQ ID NO: 55 - DNA, 20 bp, synthetic construct
  misc_feature 1..20: note = primer
  source 1..20: mol_type = other DNA
aaacggctcactagctggaa

SEQ ID NO: 56 - DNA, 20 bp, synthetic construct
  misc_feature 1..20: note = primer
  source 1..20: mol_type = other DNA
gagtgcaatggcagatctca

SEQ ID NO: 57 - DNA, 32 bp, synthetic construct
  misc_feature 1..32: note = primer
  source 1..32: mol_type = other DNA
gatcacctcgagaaacggctcactagctggaa

SEQ ID NO: 58 - DNA, 32 bp, synthetic construct
  misc_feature 1..32: note = primer
  source 1..32: mol_type = other DNA
ctgatcaagcttgagtgcaatggcagatctca

SEQ ID NO: 59 - DNA, 21 bp, synthetic construct
  misc_feature 1..21: note = primer
  source 1..21: mol_type = other DNA
tctattgggagactgcctttc

SEQ ID NO: 60 - DNA, 20 bp, synthetic construct
  misc_feature 1..20: note = primer
  source 1..20: mol_type = other DNA
gcggaagtacctgtcacgag

SEQ ID NO: 61 - DNA, 33 bp, synthetic construct
  misc_feature 1..33: note = primer
  source 1..33: mol_type = other DNA
gatcacctcgagtctattgggagactgcctttc

SEQ ID NO: 62 - DNA, 32 bp, synthetic construct
  misc_feature 1..32: note = primer
  source 1..32: mol_type = other DNA
ctgatcaagcttgcggaagtacctgtcacgag

SEQ ID NO: 63 - DNA, 20 bp, synthetic construct
  misc_feature 1..20: note = primer
  source 1..20: mol_type = other DNA
tgctgacccagctgaacata

SEQ ID NO: 64 - DNA, 20 bp, synthetic construct
  misc_feature 1..20: note = primer
  source 1..20: mol_type = other DNA
gtggggaagccttctcttct

SEQ ID NO: 65 - DNA, 32 bp, synthetic construct
  misc_feature 1..32: note = primer
  source 1..32: mol_type = other DNA
gatcacctcgagtgctgacccagctgaacata

SEQ ID NO: 66 - DNA, 32 bp, synthetic construct
  misc_feature 1..32: note = primer
  source 1..32: mol_type = other DNA
ctgatcaagcttgtggggaagccttctcttct

SEQ ID NO: 67 - DNA, 98 bp, synthetic construct
  misc_feature 1..98: note = GFP ORF
  source 1..98: mol_type = other DNA
ctctagaggatccccgggtaccggtggcgcgccgcttagcgttaacgctagccggaccgcctgcaggaggcctgcccgggcatttaaatgaattcaac

SEQ ID NO: 68 - DNA, 330 bp, synthetic construct
  source 1..330: mol_type = other DNA
ggatccgccaccatggactacaaagatgatgacgacaagcacatggccagcatgaccgggggccagcagatgggaagagaccctgatttggaacaaagggcagaggagctggcccgggagaacgaagaactggagaaggaagctgaggaacttgagcaggagctcgctgaacttcggaagaaggtggaggtgctggagaacaccaacaggactctccttcagcaacttcagaagcttcagactttggtgatggggaaggtctctcgaacctgcaagttagctggtacacagactggcacctgcctcatggtcgttgtgctttaagaattc

SEQ ID NO: 69 - DNA, 318 bp, synthetic construct
  source 1..318: mol_type = other DNA
ggatccgccaccatggactacaaagatgatgacgacaagcacatggccagcatgaccgggggccagcagatgggaagagaccctgatttggaacaaagggcagaggagctggcccgggagaacgaagaactggagaaggaagctgaggaacttgagcaggagctcgctgaactcactggcgagtgtaaagagctagaaaagaagaacgaggctctgaaagagaaggcagattctctcgccaaagagattcagtatctaaaagacctgatagaagaggtccgtaaggcaagggggaagaagagagttccttaagaattc